(19) United States
(12) Patent Application Publication
(10) Pub. No.: US 2010/0227859 A1
(43) Pub. Date: Sep. 9, 2010

(54) TRICYCLIC COMPOUNDS AS MATRIX METALLOPROTEINASE INHIBITORS

(76) Inventors: Wei LI, Acton, MA (US); Jianchang Li, Acton, MA (US); Yuchuan Wu, Acton, MA (US); Junjun Wu, Billerica, MA (US); Rajeev Hotchandani, Watertown, MA (US); Steve Yikkai Tam, Wellesley, MA (US); Tarek Suhayl, Mansour, NY (US); Joseph P. Sypek, Waban, MA (US); Iain McFadyen, Medford, MA (US)

Correspondence Address: WYETH LLC;PATENT LAW GROUP, 5 GIRALDA FARMS, MADISON, NJ, 07940, US

(21) Appl. No.: 12/598,868

(22) PCT Filed: May 5, 2008
(86) PCT No.: PCT/US08/62593

§ 371 Date: Apr. 16, 2010

Related U.S. Application Data
(60) Provisional application No. 60/927,563, filed on May 4, 2007.

(51) Int. Cl. A61K 31/5377; C07D 407/04; A61K 31/343; C07D 413/14; C07D 417/14; A61K 31/497; A61P 19/02; A61P 9/10; A61P 11/06; A61P 35/00; A61P 3/04; A61P 3/10

(52) U.S. Cl. 514/232.8; 549/461; 514/468; 544/140; 544/369; 514/254.02

(57) ABSTRACT

The present teachings relate to compounds of formula I: and pharmaceutically acceptable salts and esters thereof, wherein R1, R2, R3, R4, X, and Y are as defined herein. The present teachings also provide methods of making the compounds of formula I and methods of inhibiting matrix metalloproteinases, in particular, MMP-12, that may be involved in pathological disorders found in mammals, including a human.

### FIELD

The present teachings relate to tricyclic compounds that are capable of inhibiting matrix metalloproteinases. The present teachings also relate to methods for the preparation of the tricyclic compounds, and the methods of their use.

### INTRODUCTION

Matrix metalloproteinases (MMPs) are a family of more than 20 zinc-dependent proteases that possess the ability to degrade extracellular matrix (ECM) components that are associated with normal tissue remodeling as well as tissue destruction. The expression and activity of MMPs is tightly controlled because of the degradative nature of these enzymes. Loss in the regulation of MMPs can result in the pathological destruction of connective tissue, leading to various diseases or disorders. For example, disruption of the balance between MMPs and tissue inhibitors of metalloproteinases (TIMPs), which regulate the activity of MMPs, is manifest pathologically as rheumatoid and osteoarthritis, atherosclerosis, heart failure, fibrosis, pulmonary emphysema, and tumor growth, invasion and metastasis. As such, MMPs have been actively targeted in the development of therapeutic agents, particularly those directed towards arthritis and oncology (e.g., Woessner, J. F. (1991), FASEB J., 5: 2145-2154; and Coussens, L. M. (2002), Science, 295(5564): 2387-2392).

MMPs can be broadly classified into collagenases (MMP-1, MMP-8, and MMP-13), gelatinases (MMP-2 and MMP-9), stromelysins (MMP-3, MMP-10, and MMP-11), elastases (MMP-7 and MMP-12) and membrane-associated MMPs (MMP-14 through MMP-25). The gelatinases have been shown to be most intimately involved with the growth and spread of tumors, while the collagenases have been associated with the pathogenesis of arthritis. (e.g., Ellenrieder, V. et. al. (2000), Int. J. Cancer, 85(1):14-20; Singer, C. F. et. al., (2002), Breast Cancer Res. Treat., 72(1):69-77; Nikkola, J. et. al., (2005), Clin. Cancer Res., 11: 5158-5166; Lubbe, W. J. et. al., (2006), Clin. Cancer Res., 12: 1876-1882; Dean, D. D. (1991), Sem. Arthritis Rheum., 20(6 Suppl 2): 2-11; and Jackson, C. et. al., (2001), Inflamm. Res., 50: 183-186). There is further evidence suggesting that gelatinases are involved in the rupture of plaques associated with atherosclerosis (e.g., Dollery, C. M. et. al., (1995), Cir. Res., 77: 863-868; and Kuzuya, M. et. al., (2006), Arterioscler. Thromb. Vasc. Biol., 26(5): 1120-1125). MMPs also have been implicated in various other diseases including restenosis, MMP-mediated osteopenias, inflammatory diseases of the central nervous system, skin aging, septic arthritis, corneal ulceration, abnormal wound healing, bone disease, proteinuria, aneurysmal aortic disease, degenerative cartilage loss following traumatic joint injury, demyelinating diseases of the nervous system, cirrhosis of the liver, colitis, glomerular disease of the kidney, premature rupture of fetal membranes, inflammatory bowel disease, periodontal disease, age-related macular degeneration, diabetic retinopathy, proliferative vitreoretinopathy, retinopathy of prematurity, ocular inflammation, keratoconus, Sjogren's syndrome, myopia, ocular tumors, ocular angiogenesis/neovascularization, and corneal graft rejection.

Macrophage metalloelastase (MMP-12) like many MMPs, is able to degrade many ECM components. Different animal model studies have provided evidence that MMP-12 is an important mediator of various diseases. For example, studies investigating macrophage involvement in rheumatoid arthritis found an elevated level of MMP-12 expressed in synovial tissues and fluids from patients with rheumatoid arthritis. This observation suggests that inhibition of MMP-12 has potential in the treatment of rheumatoid arthritis (e.g. Liu, M. et. al., (2004), Arthritis & Rheumatism, 50(10): 3112-3117). Other studies have linked MMP-12 to promotion of atherosclerotic plaque instability, lesion development in multiple sclerosis, secondary injury in spinal cord injuries, and heat-induced skin damages (e.g., Johnson, J. L. (2005), PNAS, 102(43): 15575-15580; Vos, C. M. P. et. al., (2003), J. Neuroimmunology, 138: 106-114; Wells, J. E. A. et. al., (2003), J. Neuroscience, 23(31): 10107-10115; and Chen, Z. et. al., (2003), J. Invest. Dermat., 124: 70-78). Evidence also suggests that MMP-12 expression could be a prognostic indicator for early tumor relapse, with MMP-12 serving as a viable target for various types of cancer (e.g., Hofmann, H. S. et. al., (2005), Clin. Cancer Res., 11(3): 1086-1092; Kerkela, E. et. al., (2000), J. Invest. Dermatol., 114(6): 1113-1119; and Vihinen, P. et. al., (2005), Curr. Cancer Drug Target, 5: 203-220). Additionally, MMP-12 was found to contribute to corneal wound healing (e.g. Lyu, J. et. al., (2005), J. Biol. Chem., 280(22): 21653-21660). The use of MMP-12 modulators as a diagnostic tool, with potential also for the treatment of various metabolic disorders including obesity and diabetes, has also been investigated. (e.g., U.S. Patent Application Publication No. 2003/0157110).

MMPs have also been implicated as the major class of proteolytic enzymes that induce airway remodeling (e.g., Suzuki, R. Y. et. al., (2004), Treat. Respir. Med., 3: 17-27), a condition found, for example, in asthma and chronic obstructive pulmonary disease (COPD). MMP-12, in particular, has been demonstrated to play a significant role in airway inflammation and remodeling. Immunohistochemical studies of bronchoalveolar lavage (BAL) cells and bronchial lung biopsies from patients with moderate to severe COPD have been shown to have a greater level of expression of MMP-12 than in controls (e.g. Molet, S. et. al., (2005), Inflamm. Res., 54(1): 31-36). Other studies have demonstrated an increased MMP-12 expression and enzyme activity in sputum induced from patients with mild-moderate COPD compared to non-smokers, former smokers, or current smokers (e.g. Demedts, I. K. et. al., (2006), Thorax, 61: 196-201).

Other studies have suggested that inhibition of MMPs may be applicable in the treatment of diseases where MMPs are implicated. A wide range of diseases or disorders may result from diminished or loss of control of regulation of matrix metalloproteinases, such as multiple sclerosis, atherosclerotic plaque rupture, restenosis, aortic aneurism, heart failure, periodontal disease, corneal ulceration, burns, decubital ulcers, chromic ulcers or wounds, cancer metastasis, tumor angiogenesis, arthritis and automimmune and inflammatory diseases arising from tissue invasion by leukocytes (e.g. Picard, J. A., et. al., WO98/09957; O'Brien, P. M. et. al., WO09/09934)

We present herein, compounds useful as MMP inhibitors, in particular, inhibitors of MMP-12, which can be useful in treating a variety of pathological conditions and/or disorders associated with imbalances in the regulation of matrix metalloproteinases.

### SUMMARY

The present teachings relate to compounds of formula I:

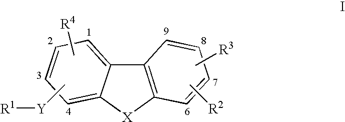

wherein R¹, R², R³, R⁴, X, and Y are as defined herein. Salts and esters of the compounds of formula I, particularly those that are acceptable for use as pharmaceuticals are also included herein.

The present teachings also relate to compositions that comprise one or more compounds of formula I, including the salts and esters thereof. The compositions may be formulated with carriers and/or excipients suitable for use as pharmaceuticals. The present teachings also provide methods of making and using the compounds of formula I including the salts and esters thereof. The present teachings also provide methods of inhibiting MMPs and treating pathological conditions, diseases or disorders mediated wholly or in part by matrix metalloproteinases. Examples of such conditions, diseases or disorders include, various inflammatory diseases (e.g., rheumatoid arthritis, osteoarthritis, atherosclerosis, multiple sclerosis, fibrosis, asthma, and chronic obstructive pulmonary diseases), metabolic disorders (e.g., obesity and diabetes), tumor growth (e.g., lung cancer and skin cancer), and spinal cord injuries. Methods of treatment may include inhibiting one or more matrix metalloproteinases by administering an effective amount of one or more compounds of formula I or the salts, and/or esters thereof, in an amount sufficient to mediate a therapeutic effect to a mammal, including humans, afflicted with the condition, disease or disorder.

### DETAILED DESCRIPTION

The present teachings provide compounds of formula I:

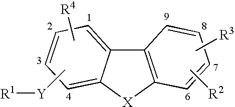

X may be O, S, S(O) or S(O)₂. In some embodiments, X may be O. In other embodiments, X may be S. In further embodiments, X may be S(O) or S(O)₂.

R¹—Y is a substituent on the tricyclic core and may be at position C2 or C3, as indicated by the numbering in formula I.

R¹ may, in various embodiments, be an N-linked, free carboxyl or carboxyl-protected, natural or non-natural amino acid containing at least one alpha-amino hydrogen. R¹ may be a D- or L-amino acid. In some embodiments, R¹ may be a D- or L-alpha-amino acid. In further embodiments, R¹ may be an N-linked valine. In yet further embodiments, R¹ may be an N-linked D-valine or L-valine. In other embodiments, R¹ may be a D- or L-beta-amino acid.

In some embodiments, R¹ may be an N-linked, natural or non-natural amino acid containing at least one alpha-amino hydrogen, wherein the carboxyl group may be in the form of a free carboxyl, as a carboxylic acid or as a carboxylic acid salt. In further embodiments, the carboxylic acid salt may be, for example, a sodium or potassium carboxylic acid salt. In other embodiments, R¹ may be an N-linked, natural or non-natural amino acid wherein the carboxyl group may be protected by carboxyl-protecting groups.

In some embodiments, R¹ may be an N-linked, natural or non-natural amino acid containing at least one alpha-amino hydrogen, wherein the amino-NH proton of the amino acid may be further substituted, for example with NH-protecting groups, or derivatised as an amino acid salt, for example, an ammonium salt.

Y is S(O) or S(O)₂.

Independently of Y, R¹ may be W—V—NH—, wherein:
W is a) —C(O)R¹³, b) —S(O)ₘR¹³, c) —S(O)ₘOR¹³, d) —S(O)ₘNR¹³R¹⁴, e) —C(O)OR¹³, f) —C(O)NR¹³R¹⁴, g) —C(S)R¹³, h) —C(S)OR¹⁴, i) —NR¹³R¹⁴, j) —C(NR¹³)NR¹³R¹⁴, k) —P(O)(OR¹³)₂, or l) —B(OR¹³)₂;
V is —CR¹³R¹⁵—, —CH₂CR¹³R¹⁵—, —(CH═CR¹⁵)—, or —BHR¹⁵—;
R¹³ and R¹⁴, at each occurrence, independently are a) H, b) —OH, c) —SH, d) —S(O)₂OH, e) —C(O)OH, f) —C(O)NH₂, g) —C(S)NH₂, h) —O—C₁₋₁₀ alkyl, i) —S(O)ₘ—C₁₋₁₀ alkyl, j) —S(O)ₘ—OC₁₋₁₀ alkyl, k) —C(O)—C₁₋₁₀ alkyl, l) —C(O)—OC₁₋₁₀ alkyl, m) —C(O)NH—C₁₋₁₀ alkyl, n) —C(O)N(C₁₋₁₀ alkyl)₂, o) —C(S)NH—C₁₋₁₀ alkyl, p) —C(S)N(C₁₋₁₀ alkyl)₂, q) a C₁₋₁₀ alkyl group, r) a C₂₋₁₀ alkenyl group, s) a C₂₋₁₀ alkynyl group, t) a C₁₋₁₀ haloalkyl group, u) a C₃₋₁₄ cycloalkyl group, v) a C₆₋₁₄ aryl group, w) a 3-14 membered cycloheteroalkyl group, or x) a 5-14 membered heteroaryl group, wherein each of the C₁₋₁₀ alkyl group, the C₂₋₁₀ alkenyl group, the C₂₋₁₀ alkynyl group, the C₁₋₁₀ haloalkyl group, the C₃₋₁₄ cycloalkyl group, the C₆₋₁₄ aryl group, the 3-14 membered cycloheteroalkyl group, and the 5-14 membered heteroaryl group optionally is substituted with 1-4 —Z—R¹⁶ groups;
R¹⁵ is H or a side chain of a natural or non-natural amino acid; and
R¹⁶, at each occurrence, independently is a) halogen, b) —CN, c) —NO₂, d) oxo, where two R¹⁶ on a single carbon can be replaced e) —OH, f) —O—C₁₋₁₀ alkyl, g) —NH₂, h) —NH(C₁₋₁₀ alkyl), i) —N(C₁₋₁₀ alkyl)₂, j) —S(O)ₘH, k) —S(O)ₘ—C₁₋₁₀ alkyl, l) —S(O)₂OH, m)—S(O)ₘ—OC₁₋₁₀ alkyl, n) —CHO, o) —C(O)—C₁₋₁₀ alkyl, p) —C(O)OH, q) —C(O)—OC₁₋₁₀ alkyl, r) —C(O)NH₂, s) —C(O)NH—C₁₋₁₀ alkyl, t) —C(O)N(C₁₋₁₀ alkyl)₂, u) —C(S)NH₂, v) —C(S)NH—C₁₋₁₀ alkyl, w) —C(S)N(C₁₋₁₀ alkyl)₂, x) —S(O)ₘNH₂, y) —S(O)ₘNH(C₁₋₁₀ alkyl), z) —S(O)ₘN(C₁₋₁₀ alkyl)₂, aa) —Si(C₁₋₁₀ alkyl)₃, ab) a C₁₋₁₀ alkyl group, ac) a C₂₋₁₀ alkenyl group, ad) a C₂₋₁₀ alkynyl group, ae) a C₁₋₁₀ haloalkyl group, af) a C₃₋₁₄ cycloalkyl group, ag) a C₆₋₁₄ aryl group, ah) a 3-14 membered cycloheteroalkyl group, or ai) a 5-14 membered heteroaryl group; and
Z and m are as defined herein.

In some embodiments, W may be —C(O)R¹³, —C(O)OR¹³, or —C(O)NR¹³R¹⁴, wherein R¹³ and R¹⁴ are as defined herein. In certain embodiments, W may be —C(O)OR¹³ and V may be —CR¹³R¹⁵—; wherein R¹³ and R¹⁵ are as defined herein. In particular embodiments, R¹⁵ may be an isopropyl group.

R² is a substituent at position C7 or C8 of formula I, selected from a) —C(O)OR⁶, b) —C(S)OR⁶, c) —C(S)R⁷, d) —C(S)NR⁷R⁸, e) —C(NR⁷)R⁷, f) —C(NR⁷)OR⁶, g) —C(NR⁷)NR⁷R⁸, h) a C₂₋₁₀ alkenyl group, i) a C₂₋₁₀ alkynyl group, j) a C₁₋₁₀ haloalkyl group, k) a C₃₋₁₄ cycloalkyl group, l) a 3-14 membered cycloheteroalkyl group and m) a 5-14 membered heteroaryl group, wherein the 3-14 membered cycloheteroalkyl group, or the 5-14 membered heteroaryl group is linked to the tricyclic core via a carbon ring atom, and each of h)-m) optionally is substituted with 1-4 —Z—R⁹ groups. In further embodiments, R² is optionally substituted with 1-3 —Z—R⁹ groups. In yet further embodiments, R² is optionally substituted with 1-2 —Z—R⁹ groups.

In some embodiments, R² is a substituent at position C7 or C8 of formula I, selected from a) —C(O)OR⁶, b) —C(S)OR⁶, c) —C(S)R⁷, d) —C(S)NR⁷R⁹, e) —C(NR⁷)R⁷, f) —C(NR⁷)OR⁶, g) —C(NR⁷)NR⁷R⁹, h) a C₂₋₁₀ alkenyl group, i) a C₂₋₁₀ alkynyl group, j) a C₁₋₁₀ haloalkyl group, k) a C₃₋₁₄ cycloalkyl group, l) a 3-14 membered cycloheteroalkyl group and m) a 5-14 membered heteroaryl group, wherein the 3-14 membered cycloheteroalkyl group, or the 5-14 membered heteroaryl group is linked to the tricyclic core via a carbon ring atom, and each of h)-m) optionally is substituted with 1-4 —Z—R⁹ groups, 1-3 —Z—R⁹ groups or 1-2 —Z—R⁹; and wherein
R⁷ and R⁸, at each occurrence, independently are a) H, b) —OH, c) —NH₂, d) —S(O)ₘH, e) —S(O)ₘOH, f) —C(O)OH, g) —C(O)NH₂, h) —C(S)NH₂, i) —C(NH)NH₂, j) —OC₁₋₁₀ alkyl, k) —NH—C₁₋₁₀ alkyl, l) —N(C₁₋₁₀ alkyl)₂, m) —S(O)ₘ—C₁₋₁₀ alkyl, n) —S(O)ₘ—OC₁₋₁₀ alkyl, o) —C(O)—C₁₋₁₀ alkyl, p) —C(O)—OC₁₋₁₀ alkyl, q) —C(O)NH—C₁₋₁₀ alkyl, r) —C(O)N(C₁₋₁₀ alkyl)₂, s) —C(S)NH—C₁₋₁₀ alkyl, t) —C(S)N(C₁₋₁₀ alkyl)₂, u) —C(NH)—C₁₋₁₀ alkyl, v) —C(NH)—OC₁₋₁₀ alkyl, w) —C(NH)NH—C₁₋₁₀ alkyl, x) —C(NH)N(C₁₋₁₀ alkyl)₂, y) —C(NC₁₋₁₀ alkyl)-C₁₋₁₀ alkyl, z) —C(NC₁₋₁₀ alkyl)-OC₁₋₁₀ alkyl, aa) —C(NC₁₋₁₀ alkyl)NH—C₁₋₁₀ alkyl, ab) —C(NC₁₋₁₀ alkyl)N(C₁₋₁₀ alkyl)₂, ac) a C₁₋₁₀ alkyl group, ad) a C₂₋₁₀ alkenyl group, ae) a C₂₋₁₀ alkynyl group, af) a C₁₋₁₀ haloalkyl group, ag) a C₃₋₁₄ cycloalkyl group, ah) a C₆₋₁₄ aryl group, ai) a 3-14 membered cycloheteroalkyl group, or aj) a 5-14 membered heteroaryl group, wherein each of the C₁₋₁₀ alkyl group, the C₂₋₁₀ alkenyl group, the C₂₋₁₀ alkynyl group, the C₁₋₁₀ haloalkyl group, the C₃₋₁₄ cycloalkyl group, the C₆₋₁₄ aryl group, the 3-14 membered cycloheteroalkyl group, and the 5-14 membered heteroaryl group optionally is substituted with 1-4 —Z—R⁹ groups; and wherein R⁷ and R⁸, when attached to a nitrogen, and together with the nitrogen to which they are attached, can form a 3- to 7-membered heterocycle containing 1-3 heteroatoms wherein up to two of the carbon atoms of the heterocycle can be replaced with —N(H)—, —N(C₁-C₆alkyl)-, —N(C₆-C₁₄aryl)-, —S—, —SO—, —S(O)₂—, or —O—;
R⁹, at each occurrence, independently is a) halogen, b) —ON, c) —NO₂, d) oxo, where two R¹⁶on a single carbon can be replaced e) —O—Z—R¹⁰, f) —NR¹⁰—Z—R¹¹, g) —N(O)R¹⁰—Z—R¹¹, h) —S(O)ₘR¹⁰, i) —S(O)ₘO—Z—R¹⁰, j) —S(O)ₘNR¹⁰—Z—R¹¹, k) —C(O)R¹⁰ l) —C(O)O—Z—R¹⁰, m) —C(O)NR¹⁰—Z—R¹¹, n) —C(S)NR¹⁰—Z—R¹¹, o) —C(NR¹⁰)R¹⁰, p) —C(NR¹⁰)O—Z—R¹⁰, q) —C(NR¹⁰)NR¹⁰—Z—R¹¹, r) —Si(C₁₋₁₀ alkyl)₃, s) a C₁₋₁₀ alkyl group, t) a C₂₋₁₀ alkenyl group, u) a C₂₋₁₀ alkynyl group, v) a C₁₋₁₀ haloalkyl group, w) a C₃₋₁₄ cycloalkyl group, x) a C₆₋₁₄ aryl group, y) a 3-14 membered cycloheteroalkyl group, or z) a 5-14 membered heteroaryl group, wherein each of the C₁₋₁₀ alkyl group, the C₂₋₁₀ alkenyl group, the C₂₋₁₀ alkynyl group, the C₁₋₁₀ haloalkyl group, the C₃₋₁₄ cycloalkyl group, the C₆₋₁₄ aryl group, the 3-14 membered cycloheteroalkyl group, and the 5-14 membered heteroaryl group optionally is substituted with 1-4 —Z—R¹² groups; and
R¹⁰ and R¹¹, at each occurrence, independently are a) H, b) —OH, c) —NH₂, d) —S(O)ₘH, e) —S(O)ₘOH, f) —C(O)OH, g) —C(O)NH₂, h) —C(S)NH₂, i) —C(NH)NH₂, j) —OC₁₋₁₀ alkyl, k) —NH—C₁₋₁₀ alkyl, l) —N(C₁₋₁₀ alkyl)₂, m) —S(O)ₘ—C₁₋₁₀ alkyl, n) —S(O)ₘ—OC₁₋₁₀ alkyl, o) —C(O)—C₁₋₁₀ alkyl, p) —C(O)—OC₁₋₁₀ alkyl, q) —C(O)NH—C₁₋₁₀ alkyl, r) —C(O)N(C₁₋₁₀ alkyl)₂, s) —C(S)NH—C₁₋₁₀ alkyl, t) —C(S)N(C₁₋₁₀ alkyl)₂, u) —C(NH)—C₁₋₁₀ alkyl, v) —C(NH)—OC₁₋₁₀ alkyl, w) —C(NH)NH—C₁₋₁₀ alkyl, x) —C(NH)N(C₁₋₁₀ alkyl)₂, y) —C(NC₁₋₁₀ alkyl)-C₁₋₁₀ alkyl, z) —C(NC₁₋₁₀ alkyl)-OC₁₋₁₀ alkyl, aa) —C(NC₁₋₁₀ alkyl)NH—C₁₋₁₀ alkyl, ab) —C(NC₁₋₁₀ alkyl)N(C₁₋₁₀ alkyl)₂, ac) a C₁₋₁₀ alkyl group, ad) a C₂₋₁₀ alkenyl group, ae) a C₂₋₁₀ alkynyl group, af) a C₁₋₁₀ haloalkyl group, ag) a C₃₋₁₄ cycloalkyl group, ah) a C₆₋₁₄ aryl group, ai) a 3-14 membered cycloheteroalkyl group, or aj) a 5-14 membered heteroaryl group, wherein each of the C₁₋₁₀ alkyl group, the C₂₋₁₀ alkenyl group, the C₂₋₁₀ alkynyl group, the C₁₋₁₀ haloalkyl group, the C₃₋₁₄ cycloalkyl group, the C₆₋₁₄ aryl group, the 3-14 membered cycloheteroalkyl group, and the 5-14 membered heteroaryl group optionally is substituted with 1-4 —Z—R¹² groups;
R¹², at each occurrence, independently is a) halogen, b) —CN, c) —NO₂, d) oxo, where two R¹⁶on a single carbon can be replaced e) —OH, f) —NH₂, g) —NH(C₁₋₁₀ alkyl), h) —N(C₁₋₁₀ alkyl)₂, i) —S(O)ₘH, j) —S(O)ₘ—C₁₋₁₀ alkyl, k) —S(O)ₘOH, l) —S(O)ₘOC₁₋₁₀ alkyl, m) —CHO, r) —C(O)NH—C₁₋₁₀ alkyl, s) —C(O)N(C₁₋₁₀ alkyl)₂, t) —C(NH)H, u) —C(NH)—C₁₋₁₀ alkyl, v) —C(NH)OH, w) —C(NH)—OC₁₋₁₀ alkyl, x) —C(NH)NH₂, y) —C(NH)NH—C₁₋₁₀ alkyl, z) —C(NH)N(C₁₋₁₀ alkyl)₂, aa) —C(NC₁₋₁₀ alkyl)H, ab) —C(NC₁₋₁₀ alkyl)-C₁₋₁₀ alkyl, ac) —C(NC₁₋₁₀ alkyl)OH, ad) —C(NC₁₋₁₀ alkyl)-OC₁₋₁₀ alkyl, ae) —C(NC₁₋₁₀ alkyl)NH₂, af) —C(NC₁₋₁₀ alkyl)NH—C₁₋₁₀ alkyl, ag) —C(NC₁₋₁₀ alkyl)N(C₁₋₁₀ alkyl)₂, ah) —C(S)NH₂, ai) —C(S)NH—C₁₋₁₀ alkyl, aj) —C(S)N(C₁₋₁₀ alkyl)₂, ak) —S(O)ₘNH₂, al) —S(O)ₘNH(C₁₋₁₀ alkyl), am) —S(O)ₘN(C₁₋₁₀ alkyl)₂, an) —Si(C₁₋₁₀ alkyl)₃, ap) a C₁₋₁₀ alkyl group, aq) a C₂₋₁₀ alkenyl group, ar) a C₂₋₁₀ alkynyl group, as) a C₁₋₁₀ haloalkyl group, at) a C₃₋₁₄ cycloalkyl group, au) a C₆₋₁₄ aryl group, av) a 3-14 membered cycloheteroalkyl group, or aw) a 5-14 membered heteroaryl group; wherein each of ap) to av) is optionally substituted with 1-4 groups selected from halogen, —CN, —NO₂, —OH, —O(C₁₋₁₀ alkyl), —NH₂, —NH(C₁₋₁₀ alkyl), and —N(C₁₋₁₀ alkyl)₂;
Z, at each occurrence, independently is a) a divalent C₁₋₁₀ alkyl group, b) a divalent C₂₋₁₀ alkenyl group, c) a divalent C₂₋₁₀ alkynyl group, d) a divalent C₁₋₁₀ haloalkyl group, or e) Z— is a bond; and
m, at each occurrence, independently is 0, 1, or 2.

In some embodiments, R² may be —C(NR⁷)R⁷ or —C(NR⁷)NR⁷R⁸.

In some embodiments, R² may be —C(NH)R⁷, —C(NCH₃)R⁷, —C(NCH₂CH₃)R⁷, —C(NCH(CH₃)₂)R⁷, —C(NH)NR⁷R⁸, —C(NCH₃)NR⁷R⁸, —C(NCH₂CH₃)NR⁷R⁸, or —C(NCH(CH₃)₂)NR⁷R⁸.

In some embodiments, R² may be a group selected from N-isopropylcarbamimidoyl, N-hydroxycarbamimidoyl, N-methoxycarbamimidoyl, N-methylcarbamimidoyl, N-ethyl carbamimidoyl, N-phenylcarbamimidoyl, N-benzylcarbamimidoyl, N,N-diethyl carbamimidoyl, N-methyl-N-isopropylcarbamimidoyl, N-ethyl-N′-ethylcarbamimidoyl, N-methylamido, N-ethyl amido and imino(pyrrolidin-1-yl)methyl, each optionally substituted with 1-4 —Z—R¹² groups. In further embodiments, R² is optionally substituted with 1-3 —Z—R⁹ groups. In yet further embodiments, R² is optionally substituted with 1-2 —Z—R⁹ groups.

In some embodiments, R² may be a group selected from C₂₋₁₀ alkenyl and C₂₋₁₀ alkynyl, wherein each group is optionally substituted with —O—Z—R¹⁰, —NR¹⁰—Z—R¹¹, —C(O)R¹⁰, —C(O)O—Z—R¹⁰, —C(O)NR¹⁰—Z—R¹¹, C₃₋₁₄ cycloalkyl, C₆₋₁₄ aryl, 3-14 membered cycloheteroalkyl, or 5-14 membered heteroaryl, wherein each of the C₃₋₁₄ cycloalkyl, the C₆₋₁₄ aryl, the 3-14 membered cycloheteroalkyl, and the 5-14 membered heteroaryl is optionally substituted with 1-4 —Z—R¹² groups. In further embodiments, R² is optionally substituted with 1-3 —Z—R⁹ groups. In yet further embodiments, R² is optionally substituted with 1-2 —Z—R⁹ groups.

In some embodiments, R² may be a group is selected from 2-cyclopropylethenyl, 2-cyclobutylethenyl, 2-cyclopentylethenyl, 2-cyclohexyl ethenyl, 2-cycloheptylethenyl, methoxy carbonylethynyl, diethylaminoethynyl, 3-methoxypropynyl, 3-dimethylaminopropynyl, 3-N,N-diethylaminopropynyl and (1-methylimidazol-2-yl)ethynyl, each of which optionally is substituted with 1-4 —Z—R¹² groups. In further embodiments, R² is optionally substituted with 1-3 —Z—R⁹ groups. In yet further embodiments, R² is optionally substituted with 1-2 —Z—R⁹ groups.

In some embodiments, R² may be a group selected from C₃₋₁₄ cycloalkyl and 3-14 membered cycloheteroalkyl, each of which optionally is substituted with 1-4 —Z—R⁹ groups. In further embodiments, R² is optionally substituted with 1-3 —Z—R⁹ groups. In yet further embodiments, R² is optionally substituted with 1-2 —Z—R⁹ groups.

In some embodiments, R² may be a group selected from cis-1-propenyl, trans-1-propenyl, cis-2-propenyl, trans-2-propenyl, cyclopropyl, cyclobutyl, cyclopentyl, cyclohexyl, cycloheptyl, cyclopentenyl, cyclohexenyl, 4,5-dihydro-1H-imidazol-2-yl, 4,5-dihydrooxazol-2-yl, 4,5-dihydrothiazol-2-yl, and 1,2,3,6-tetrahydropyridin-4-yl, each of which optionally is substituted with 1-4 —Z—R⁹ groups. In further embodiments, R² is optionally substituted with 1-3 —Z—R⁹ groups. In yet further embodiments, R² is optionally substituted with 1-2 —Z—R⁹ groups.

In some embodiments, R² may be a 5-14 membered heteroaryl group optionally substituted with 1-4 —Z—R⁹ groups. In further embodiments, R² is optionally substituted with 1-3 —Z—R⁹ groups. In yet further embodiments, R² is optionally substituted with 1-2 —Z—R⁹ groups.

In some embodiments, R² may be a 5-6 membered heteroaryl group having 1-4 ring members independently selected from O, S, and N, and wherein the 5-6 membered heteroaryl group optionally is substituted with 1-4 —Z—R⁹ groups. In further embodiments, R² is optionally substituted with 1-3 —Z—R⁹ groups. In yet further embodiments, R² is optionally substituted with 1-2 —Z—R⁹ groups.

In some embodiments, R² may be selected from furanyl, thienyl, pyrrolyl, oxazolyl, thiazolyl, imidazolyl, oxadiazolyl, thiadiazolyl, triazolyl, pyridinyl, pyrimidinyl, pyrazinyl, isoxazolyl, isoxadiazolyl, pyrazolyl, and tetrazolyl, each of which optionally is substituted with 1-4 —Z—R⁹ groups. In further embodiments, R² is optionally substituted with 1-3 —Z—R⁹ groups. In yet further embodiments, R² is optionally substituted with 1-2 —Z—R⁹ groups.

In some embodiments, R² may be a furanyl or isoxazolyl or oxadiazolyl, group, each of which optionally is substituted with 1-4 —Z—R⁹ groups. In further embodiments, R² is optionally substituted with 1-3 —Z—R⁹ groups. In yet further embodiments, R² is optionally substituted with 1-2 —Z—R⁹ groups.

In some embodiments, R² may be a thienyl or thiazolyl, group, each of which optionally is substituted with 1-4 —Z—R⁹ groups. In further embodiments, R² is optionally substituted with 1-3 —Z—R⁹ groups. In yet further embodiments, R² is optionally substituted with 1-2 —Z—R⁹ groups.

In some embodiments, R² may be a pyrrolyl, imidazolyl, triazolyl or tetrazolyl group, each of which optionally is substituted with 1-4 —Z—R⁹ groups. In further embodiments, R² is optionally substituted with 1-3 —Z—R⁹ groups. In yet further embodiments, R² is optionally substituted with 1-2 —Z—R⁹ groups.

In some embodiments, R² may be substituted with 1-4, 1-3 or 1-2 substituents selected from halogen, C₁₋₁₀ alkyl, C₁₋₁₀ haloalkyl, C₃₋₁₄ cycloalkyl, C₆₋₁₄ aryl, 3-14 membered cycloheteroalkyl, and 5-14 membered heteroaryl. In further embodiments, R² is optionally substituted with 1-3 substituents selected from halogen, C₁₋₁₀ alkyl, C₁₋₁₀ haloalkyl, C₃₋₁₄ cycloalkyl, C₆₋₁₄ aryl, 3-14 membered cycloheteroalkyl, and 5-14 membered heteroaryl. In yet further embodiments, R² is optionally substituted with 1-2 substituents selected from halogen, C₁₋₁₀ alkyl, C₁₋₁₀ haloalkyl, C₃₋₁₄ cycloalkyl, C₆₋₁₄ aryl, 3-14 membered cycloheteroalkyl, and 5-14 membered heteroaryl.

In some embodiments, R² may be substituted with 1-4, 1-3 or 1-2 substituents selected from halogen, formyl, C₁₋₁₀ alkyl, C₁₋₁₀ haloalkyl, C₁₋₁₀ alkoxy, cyclopropyl, cyclobutyl, cyclopentyl, cyclohexyl, cyclopentenyl, cyclohexenyl, phenyl, halophenyl, trifluorophenyl, benzyl, pyrrolidinyl, tetrahydrofuranyl, furanyl, thienyl, pyrrolyl, imidazolyl, pyridinyl, pyrimidinylisoxazolyl, isoxadiazolyl, pyrazolyl, tetrazolyl and benzofuranyl; and each of the substituents may be optionally substituted with 1-4 —Z—R⁹ groups. In further embodiments, each of the substituents is optionally substituted with 1-3 —Z—R⁹ groups. In yet further embodiments, each of the substituents is optionally substituted with 1-2 —Z—R⁹ groups.

In further embodiments, each of the C₃₋₈ cycloalkyl, the C₆₋₈ aryl, the 3-8 membered cycloheteroalkyl, and the 5-8 membered heteroaryl group is independently selected from cyclopentyl, cyclopentenyl, cyclohexyl, cyclohexenyl, phenyl, and pyridinyl.

In certain embodiments, R² can be selected from:

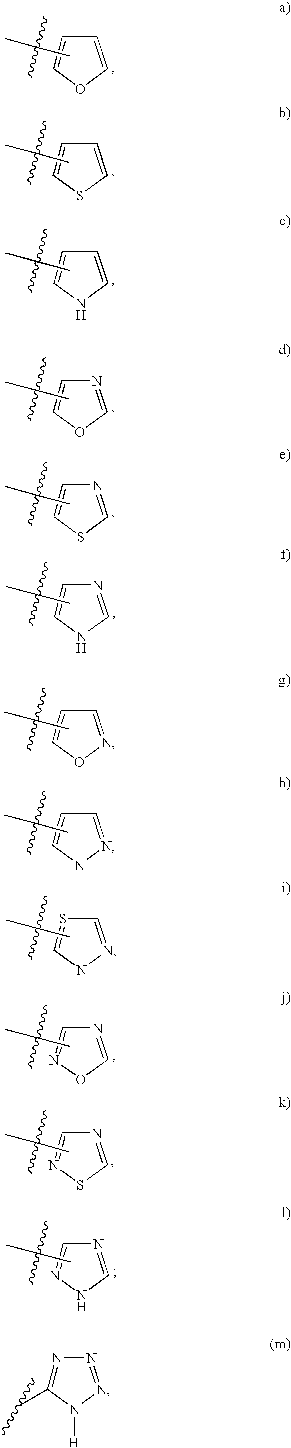

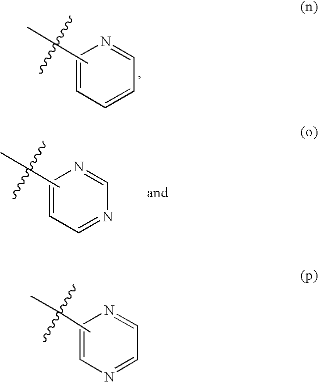

wherein each of a)-l) can be optionally substituted with 1-4 —Z—R⁹ groups, wherein R⁹ and Z are as defined herein. In further embodiments, R² is optionally substituted with 1-3 —Z—R⁹ groups. In yet further embodiments, R² is optionally substituted with 1-2 —Z—R⁹ groups.

In some embodiments, R² may be an 8-14 membered heteroaryl group comprising a 5-6 membered heteroaryl ring fused with 1-2 rings independently selected from C₃₋₈ cycloalkyl, phenyl, 3-8 membered cycloheteroalkyl, and 5-8 membered heteroaryl, wherein the 5-6 membered heteroaryl group is selected from furanyl, thienyl, pyrrolyl, oxazolyl, thiazolyl, imidazolyl, oxadiazolyl, thiadiazolyl, triazolyl, pyridinyl, pyrimidinyl, pyrazinyl, isoxazolyl, pyrazolyl, and tetrazolyl; and wherein the 8-14 membered heteroaryl group is optionally substituted with 1-4 —Z—R⁹ groups. In further embodiments, R² is optionally substituted with 1-3 —Z—R⁹ groups. In yet further embodiments, R² is optionally substituted with 1-2 —Z—R⁹ groups.

In further embodiments, R² may be selected from benzoxazolyl, benzothiazolyl, benzimidazolyl, benzofuranyl, benzothienyl, indolyl, benzoindolyl, dibenzofuranyl, and dibenzothienyl.

In some embodiments, R² may be a 2-oxo-1H-benzo[d][1,3]oxazinyl group optionally substituted with 1-3 —Z—R⁹ groups. In further embodiments, R² is optionally substituted with 1-2 —Z—R⁹ groups.

In some embodiments, R² may be an 8-14 membered polycyclic heteroaryl group having 1-4 ring members independently selected from O, S, and N, wherein the 8-14 membered bicyclic heteroaryl group may be optionally substituted with 1-4 —Z—R⁹ groups, wherein R⁹ and Z are as defined herein. In further embodiments, R² is optionally substituted with 1-3 —Z—R⁹ groups. In yet further embodiments, R² is optionally substituted with 1-2 —Z—R⁹ groups.

In further embodiments, R² may be an 8-14 membered polycyclic heteroaryl group that includes a 5-6 membered heteroaryl group fused with 1-2 groups independently selected from a C₃₋₈ cycloalkyl group, a C₆₋₈ aryl group, a 3-8 membered cycloheteroalkyl group, and a 5-8 membered heteroaryl group, wherein the 5-6 membered heteroaryl group may be selected from:

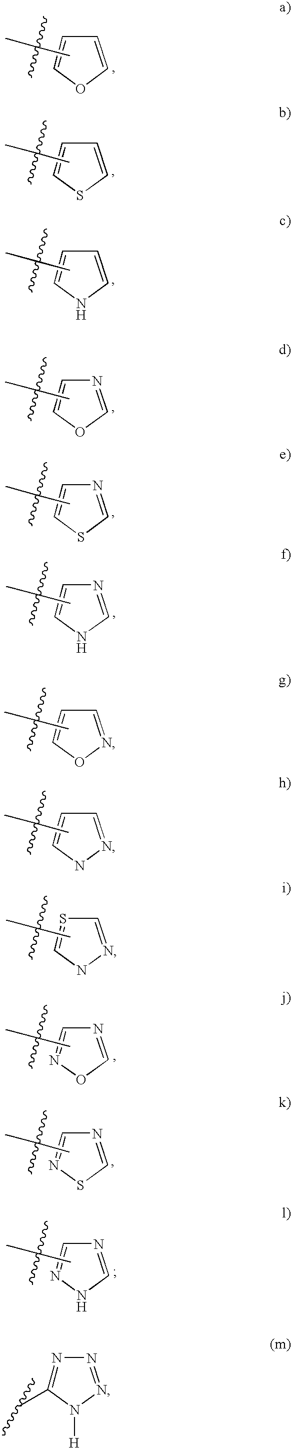

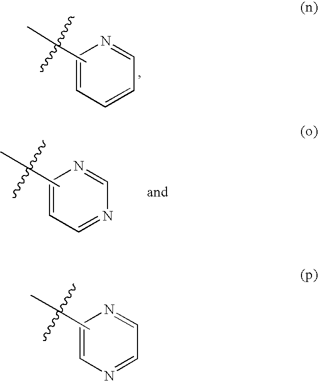

wherein the 8-14 membered polycyclic heteroaryl group may be optionally substituted with 1-4 —Z—R⁹ groups, wherein R⁹ and Z are as defined herein. In further embodiments, R² is optionally substituted with 1-3 —Z—R⁹ groups. In yet further embodiments, R² is optionally substituted with 1-2 —Z—R⁹ groups.

In some examples, the 5-6 membered heteroaryl group can be a thiazolyl group or a furanyl group, each of which can be optionally substituted with 1-4 —Z—R⁹ groups, wherein R⁹ and Z are as defined herein. In further embodiments, R² is optionally substituted with 1-3 —Z—R⁹ groups. In yet further embodiments, R² is optionally substituted with 1-2 —Z—R⁹ groups.

Examples of the C₃₋₈ cycloalkyl group, the C₆₋₈ aryl group, the 3-8 membered cycloheteroalkyl group, and the 5-8 membered heteroaryl group that fuses with the 5-6 membered heteroaryl group to form the 8-14 membered heteroaryl group can include a cyclopentyl group, a cyclopentenyl group, a cyclohexyl group, a cyclohexenyl group, a phenyl group, and a pyridinyl group. In these embodiments, R² can be a benzoxazolyl group, a benzothiazolyl group, a benzimidazolyl group, a benzofuranyl group, a benzothienyl group, an indolyl group, a benzoindolyl group, a dibenzofuranyl group, or a dibenzothienyl group, wherein each of these groups can be optionally substituted with 1-4 —Z—R⁹ groups, wherein R⁹ and Z are as defined herein. R³ and R⁴ independently may be a) H, b) —CN, c) —NO₂, d) halogen, e) —OR⁶, f) —NR⁷R⁸, g) —S(O)ₘR⁷, h) —S(O)ₘOR⁶, i) —C(O)R⁷, j) —C(O)OR⁶, k) —C(O)NR⁷R⁸, l) —C(S)R⁷, m) —C(S)OR⁶, n) —C(S)NR⁷R⁸, o) —C(NR⁷)R⁷, p) —C(NR⁷)OR⁶, q) —C(NR⁷)NR⁷R⁸, r) a C₁₋₁₀ alkyl group, s) a C₂₋₁₀ alkenyl group, t) a C₂₋₁₀ alkynyl group, u) a C₁₋₁₀ haloalkyl group, v) a C₃₋₁₄ cycloalkyl group, w) a C₆₋₁₄ aryl group, x) a 3-14 membered cycloheteroalkyl group, or y) a 5-14 membered heteroaryl group, wherein each of r)-y) optionally is substituted with 1-4 —Z—R⁹ groups; and R² may be connected to the tricyclic core via a ring carbon, wherein the ring carbon may be a carbon atom forming the heterocyclic ring, or a carbon atom on the ring fused to the heterocyclic ring. In further embodiments, R² is optionally substituted with 1-3 —Z—R⁹ groups. In yet further embodiments, R² is optionally substituted with 1-2 —Z—R⁹ groups.

In some embodiments, R³ may be hydrogen. In some embodiments, R⁴ may be hydrogen. In some embodiments, R³ and R⁴ are hydrogen.

In some embodiments, the compound of formula I may be selected from:

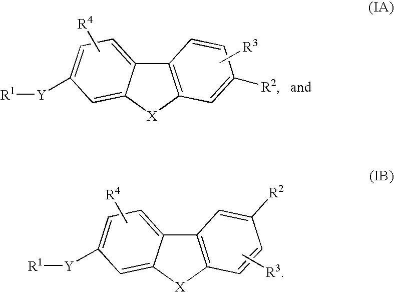

In some embodiments, the compound of formula I may be selected from:

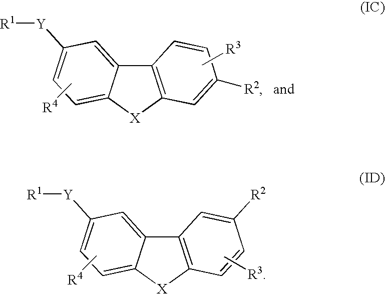

The invention includes compounds of formula IE, wherein R³ and R⁴ in formula I are both hydrogen, as depicted below:

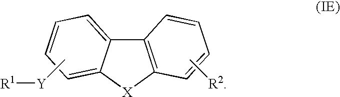

In some embodiments, the invention relates to compounds of formula IE, or a pharmaceutically acceptable salt or ester thereof, wherein: X is O, S, S(O), or S(O)₂; R¹—Y is a substituent at position C2 or C3 of formula IE; Y is S(O), or S(O)₂; R¹ is an N-linked valine with a free or protected carboxyl C-terminus, and R² is phenyl or benzo[d][1,3]dioxole, each optionally substituted with 1-5 groups selected from halogen, CF₃, C₁-C₆ alkyl or O(C₁-C₆ alkoxy).

In further embodiments, the compound may be selected from the group consisting of: (S)-2-(8-(benzo[d][1,3]dioxol-5-yl)dibenzo[b,d]furan-3-sulfonamido)-3-methylbutanoic acid; (S)-3-methyl-2-(8-phenyldibenzo[b,d]furan-3-sulfonamido) butanoic acid; (S)-2-(8-(4-methoxyphenyl)dibenzo[b,d]furan-3-sulfonamido)-3-methyl butanoic acid; (S)-3-methyl-2-(8-(4-(trifluoromethyl)phenyl)dibenzo[b,d]furan-3-sulfonamido)butanoic acid; (R)-3-methyl-2-(7-(4-(trifluoromethyl)phenyl) dibenzo[b,d]furan-2-sulfonamido)butanoic acid; (S)-3-methyl-2-(7-phenyldibenzo[b,d]thiophene-3-sulfonamido)butanoic acid; and (R)-3-methyl-2-(7-phenyldibenzo[b,d]thiophene-3-sulfonamido)butanoic acid.

Compounds of the present teachings include the compounds presented in Table 1 below:

TABLE 1

| Compd | |
|---|---|
| No | Name |
| 1 | (R)-2-(8-(4,4-dimethyl-2-oxo-2,4-dihydro-1H-benzo[d][1,3]oxazin-6- |
| | yl)dibenzo[b,d]furan-3-sulfonamido)-3-methylbutanoic acid |
| 2 | (S)-2-(8-(3-(dimethylamino)prop-1-ynyl)dibenzo[b,d]furan-3-sulfonamido)-3- |
| | methylbutanoic acid |
| 3 | (S)-3-methyl-2-(8-(pyridin-3-yl)dibenzo[b,d]furan-3-sulfonamido)butanoic acid |
| 4 | (S)-2-(8-(4,4-dimethyl-2-oxo-2,4-dihydro-1H-benzo[d][1,3]oxazin-6- |
| | yl)dibenzo[b,d]furan-3-sulfonamido)-3-methylbutanoic acid |
| 5 | (S)-3-methyl-2-(8-(4-methylthiophen-3-yl)dibenzo[b,d]furan-3-sulfonamido)butanoic |
| | acid |
| 6 | (S)-2-(8-(3-methoxy-3-oxoprop-1-ynyl)dibenzo[b,d]furan-3-sulfonamido)-3- |
| | methylbutanoic acid |
| 7 | (S)-2-(8-(furan-3-yl)dibenzo[b,d]furan-3-sulfonamido)-3-methylbutanoic acid |
| 8 | (S)-2-(8-(1H-pyrrol-2-yl)dibenzo[b,d]furan-3-sulfonamido)-3-methylbutanoic acid |
| 9 | (S)-2-(8-(3,5-dimethylisoxazol-4-yl)dibenzo[b,d]furan-3-sulfonamido)-3-methylbutanoic |
| | acid |
| 10 | (S)-2-(8-(6-methoxypyridin-3-yl)dibenzo[b,d]furan-3-sulfonamido)-3-methylbutanoic |
| | acid |
| 11 | (S)-3-methyl-2-(8-(pyridin-3-yl)dibenzo[b,d]thiophene-3-sulfonamido)butanoic acid |
| 12 | (S)-2-(8-(benzo[b]thiophen-3-yl)dibenzo[b,d]furan-3-sulfonamido)-3-methylbutanoic |
| | acid |
| 13 | (S)-2-(8-(benzo[b]thiophen-2-yl)dibenzo[b,d]furan-3-sulfonamido)-3-methylbutanoic |
| | acid |
| 14 | (S)-3-methyl-2-(8-(quinolin-6-yl)dibenzo[b,d]furan-3-sulfonamido)butanoic acid |
| 15 | (S)-3-methyl-2-(8-((1-methyl-1H-imidazol-5-yl)ethynyl)dibenzo[b,d]furan-3- |
| | sulfonamido)butanoic acid |
| 16 | (S)-3-methyl-2-(8-(pyridin-4-yl)dibenzo[b,d]furan-3-sulfonamido)butanoic acid |
| 17 | (S)-3-methyl-2-(8-(5-methylthiophen-2-yl)dibenzo[b,d]furan-3-sulfonamido)butanoic |
| | acid |
| 18 | (S)-3-methyl-2-(8-(1-methyl-1H-pyrazol-4-yl)dibenzo[b,d]furan-3-sulfonamido)butanoic |
| | acid |
| 19 | (S)-2-(8-(3,5-dimethyl-1H-pyrazol-4-yl)dibenzo[b,d]furan-3-sulfonamido)-3- |
| | methylbutanoic acid |
| 20 | (S)-2-(8-(1-isopentyl-1H-pyrazol-4-yl)dibenzo[b,d]furan-3-sulfonamido)-3- |
| | methylbutanoic acid |
| 21 | (S)-3-methyl-2-(8-(1-propyl-1H-pyrazol-4-yl)dibenzo[b,d]furan-3-sulfonamido)butanoic |
| | acid |
| 22 | (S)-2-(8-(1-benzyl-1H-pyrazol-4-yl)dibenzo[b,d]furan-3-sulfonamido)-3-methylbutanoic |
| | acid |
| 23 | (S)-2-(8-(1H-pyrazol-4-yl)dibenzo[b,d]furan-3-sulfonamido)-3-methylbutanoic acid |
| 24 | (S)-3-methyl-2-(8-(4-methylthiophen-3-yl)dibenzo[b,d]thiophene-3- |
| | sulfonamido)butanoic acid |
| 25 | (S)-2-(8-(furan-3-yl)dibenzo[b,d]thiophene-3-sulfonamido)-3-methylbutanoic acid |
| 26 | (S)-3-methyl-2-(8-(thiophen-3-yl)dibenzo[b,d]thiophene-3-sulfonamido)butanoic acid |
| 27 | (S)-2-(8-(3,5-dimethylisoxazol-4-yl)dibenzo[b,d]thiophene-3-sulfonamido)-3- |
| | methylbutanoic acid |
| 28 | (S)-3-methyl-2-(8-(thiophen-3-yl)dibenzo[b,d]furan-3-sulfonamido)butanoic acid |
| 29 | (S)-3-methyl-2-(8-(thiophen-2-yl)dibenzo[b,d]furan-3-sulfonamido)butanoic acid |
| 30 | (S)-2-(8-(3-formylfuran-2-yl)dibenzo[b,d]furan-3-sulfonamido)-3-methylbutanoic acid |
| 31 | (S)-2-(8-(3-formylthiophen-2-yl)dibenzo[b,d]furan-3-sulfonamido)-3-methylbutanoic |
| | acid |
| 32 | (S)-2-(8-(5-acetylthiophen-2-yl)dibenzo[b,d]furan-3-sulfonamido)-3-methylbutanoic acid |
| 33 | (S)-3-methyl-2-(8-(4-methylthiophen-2-yl)dibenzo[b,d]furan-3-sulfonamido)butanoic |
| | acid |
| 34 | (S)-2-(8-(2-chlorothiophen-3-yl)dibenzo[b,d]furan-3-sulfonamido)-3-methylbutanoic |
| | acid |
| 35 | (S,E)-2-(8-(2-cyclohexylvinyl)dibenzo[b,d]furan-3-sulfonamido)-3-methylbutanoic acid |
| 36 | (S)-3-methyl-2-(8-(thiazol-2-yl)dibenzo[b,d]furan-3-sulfonamido)butanoic acid |
| 37 | (S)-2-(8-(furan-2-yl)dibenzo[b,d]furan-3-sulfonamido)-3-methylbutanoic acid |
| 38 | (S)-2-(8-(methoxyethynyl)dibenzo[b,d]furan-3-sulfonamido)-3-methylbutanoic acid |
| 39 | (S)-2-(8-((diethylamino)ethynyl)dibenzo[b,d]furan-3-sulfonamido)-3-methylbutanoic |
| | acid |
| 40 | (S)-3-methyl-2-(8-(1-methyl-1H-pyrazol-4-yl)dibenzo[b,d]thiophene-3- |
| | sulfonamido)butanoic acid |
| 41 | (S)-2-(8-(3,5-dimethyl-1H-pyrazol-4-yl)dibenzo[b,d]thiophene-3-sulfonamido)-3- |
| | methylbutanoic acid |
| 42 | (S)-3-methyl-2-(8-(1-propyl-1H-pyrazol-4-yl)dibenzo[b,d]thiophene-3- |
| | sulfonamido)butanoic acid |
| 43 | (S)-2-(8-(1-isopentyl-1H-pyrazol-4-yl)dibenzo[b,d]thiophene-3-sulfonamido)-3- |
| | methylbutanoic acid |
| 44 | (S)-2-(8-(1-benzyl-1H-pyrazol-4-yl)dibenzo[b,d]thiophene-3-sulfonamido)-3- |
| | methylbutanoic acid |
| 45 | (S)-2-(8-(1H-pyrazol-4-yl)dibenzo[b,d]thiophene-3-sulfonamido)-3-methylbutanoic acid |
| 46 | (S)-2-(8-(benzo[b]thiophen-2-yl)dibenzo[b,d]thiophene-3-sulfonamido)-3- |
| | methylbutanoic acid |
| 47 | (S)-2-(8-(5-acetylthiophen-2-yl)dibenzo[b,d]thiophene-3-sulfonamido)-3-methylbutanoic |
| | acid |
| 48 | (S)-2-(8-(3-((dimethylamino)methyl)furan-2-yl)dibenzo[b,d]furan-3-sulfonamido)-3- |
| | methylbutanoic acid |
| 49 | (S)-2-(8-(3-((dimethylamino)methyl)thiophen-2-yl)dibenzo[b,d]furan-3-sulfonamido)-3- |
| | methylbutanoic acid |
| 50 | (S)-2-(8-(5-(1-(dimethylamino)ethyl)thiophen-2-yl)dibenzo[b,d]furan-3-sulfonamido)-3- |
| | methylbutanoic acid |
| 51 | (S)-2-(6-(2-chlorothiophen-3-yl)dibenzo[b,d]thiophene-3-sulfonamido)-3- |
| | methylbutanoic acid |
| 52 | (S)-2-(8-(2-chlorothiophen-3-yl)dibenzo[b,d]thiophene-3-sulfonamido)-3- |
| | methylbutanoic acid |
| 53 | (S)-2-(8-(furan-2-yl)dibenzo[b,d]thiophene-3-sulfonamido)-3-methylbutanoic acid |
| 54 | (S)-2-[8-(6″-Chloro-[2,3′;6′,3″]terpyridin-5-yl)-dibenzothiophene-3-sulfonylamino]-3- |
| | methyl-butanoic acid |
| 55 | (S)-2-(8-(6-methoxypyridin-3-yl)dibenzo[b,d]thiophene-3-sulfonamido)-3- |
| | methylbutanoic acid |
| 56 | (S)-3-methyl-2-(8-(pyridin-4-yl)dibenzo[b,d]thiophene-3-sulfonamido)butanoic acid |
| 57 | (S)-2-(8-(1H-pyrrol-2-yl)dibenzo[b,d]thiophene-3-sulfonamido)-3-methylbutanoic acid |
| 58 | (S,E)-2-(8-(2-cyclohexylvinyl)dibenzo[b,d]thiophene-3-sulfonamido)-3-methylbutanoic |
| | acid |
| 59 | (S)-2-(8-(6′-chloro-2,3′-bipyridin-5-yl)dibenzo[b,d]furan-3-sulfonamido)-3- |
| | methylbutanoic acid |
| 60 | (S)-2-(7-(furan-3-yl)dibenzo[b,d]thiophene-3-sulfonamido)-3-methylbutanoic acid |
| 61 | (S)-2-(8-(6′-chloro-2,3′-bipyridin-5-yl)dibenzo[b,d]thiophene-3-sulfonamido)-3- |
| | methylbutanoic acid |
| 62 | (S)-3-methyl-2-(8-(4-methylthiophen-2-yl)dibenzo[b,d]thiophene-3- |
| | sulfonamido)butanoic acid |
| 63 | (S)-2-(8-(6-chloropyridin-3-yl)dibenzo[b,d]thiophene-3-sulfonamido)-3-methylbutanoic |
| | acid |
| 64 | (S)-2-(8-(6-chloropyridin-3-yl)dibenzo[b,d]furan-3-sulfonamido)-3-methylbutanoic acid |
| 65 | (S)-2-(7-(3-methoxyprop-1-ynyl)dibenzo[b,d]thiophene-3-sulfonamido)-3- |
| | methylbutanoic acid |
| 66 | (S,E)-3-methyl-2-(8-(prop-1-enyl)dibenzo[b,d]furan-3-sulfonamido)butanoic acid |
| 67 | (S,Z)-3-methyl-2-(8-(prop-1-enyl)dibenzo[b,d]furan-3-sulfonamido)butanoic acid |
| 68 | (S)-3-methyl-2-(8-(5-((methylamino)methyl)furan-2-yl)dibenzo[b,d]furan-3- |
| | sulfonamido)butanoic acid |
| 69 | (S)-2-(8-cyclopentenyldibenzo[b,d]furan-3-sulfonamido)-3-methylbutanoic acid |
| 70 | (S)-3-methyl-2-(8-(1,2,3,6-tetrahydropyridin-4-yl)dibenzo[b,d]furan-3- |
| | sulfonamido)butanoic acid |
| 71 | (S)-2-(8-cyclopentyldibenzo[b,d]furan-3-sulfonamido)-3-methylbutanoic acid |
| 72 | (S)-3-methyl-2-(8-(5-methylfuran-2-yl)dibenzo[b,d]furan-3-sulfonamido)butanoic acid |
| 73 | (S)-2-(8-(5-chlorofuran-2-yl)dibenzo[b,d]furan-3-sulfonamido)-3-methylbutanoic acid |
| 74 | (S)-2-(8-(5-chlorothiophen-2-yl)dibenzo[b,d]furan-3-sulfonamido)-3-methylbutanoic |
| | acid |
| 75 | (S)-2-(8-(3,5-dichlorothiophen-2-yl)dibenzo[b,d]furan-3-sulfonamido)-3-methylbutanoic |
| | acid |
| 76 | (S)-2-(8-(N-isopropylcarbamimidoyl)dibenzo[b,d]furan-3-sulfonamido)-3- |
| | methylbutanoic acid |
| 77 | (S)-2-(8-(4,5-dihydro-1H-imidazol-2-yl)dibenzo[b,d]furan-3-sulfonamido)-3- |
| | methylbutanoic acid |
| 78 | (S)-2-(7-(furan-2-yl)dibenzo[b,d]furan-3-sulfonamido)-3-methylbutanoic acid |
| 79 | (S)-2-(7-(furan-3-yl)dibenzo[b,d]furan-3-sulfonamido)-3-methylbutanoic acid |
| 80 | (S)-2-(7-(5-chlorofuran-2-yl)dibenzo[b,d]furan-3-sulfonamido)-3-methylbutanoic acid |
| 81 | (S)-3-methyl-2-(7-(thiophen-2-yl)dibenzo[b,d]furan-3-sulfonamido)butanoic acid |
| 82 | (S)-2-(8-(N-hydroxycarbamimidoyl)dibenzo[b,d]furan-3-sulfonamido)-3-methylbutanoic |
| | acid |
| 83 | (S)-2-(8-(4,5-dihydrooxazol-2-yl)dibenzo[b,d]furan-3-sulfonamido)-3-methylbutanoic |
| | acid |
| 84 | (S)-2-(7-(5-chlorothiophen-2-yl)dibenzo[b,d]furan-3-sulfonamido)-3-methylbutanoic |
| | acid |
| 85 | (S)-2-(7-(3,5-dichlorothiophen-2-yl)dibenzo[b,d]furan-3-sulfonamido)-3-methylbutanoic |
| | acid |
| 86 | (S)-3-methyl-2-(7-(3,4,5-trichlorothiophen-2-yl)dibenzo[b,d]furan-3- |
| | sulfonamido)butanoic acid |
| 87 | (S)-3-methyl-2-(8-(N-phenylcarbamimidoyl)dibenzo[b,d]furan-3-sulfonamido)butanoic |
| | acid |
| 88 | (S)-2-(8-(N-benzylcarbamimidoyl)dibenzo[b,d]furan-3-sulfonamido)-3-methylbutanoic |
| | acid |
| 89 | (S)-2-(8-(2,5-dimethylthiophen-3-yl)dibenzo[b,d]furan-3-sulfonamido)-3-methylbutanoic |
| | acid |
| 90 | (R)-2-(7-(3-methoxyprop-1-ynyl)dibenzo[b,d]thiophene-3-sulfonamido)-3- |
| | methylbutanoic acid |
| 91 | (S)-3-methyl-2-(8-(5-methyl-1,2,4-oxadiazol-3-yl)dibenzo[b,d]furan-3- |
| | sulfonamido)butanoic acid |
| 92 | (S)-3-methyl-2-(8-(5-(trifluoromethyl)-1,2,4-oxadiazol-3-yl)dibenzo[b,d]furan-3- |
| | sulfonamido)butanoic acid |
| 93 | (S)-2-(8-(1,2,4-oxadiazol-3-yl)dibenzo[b,d]furan-3-sulfonamido)-3-methylbutanoic acid |
| 94 | (S)-2-(8-(2-chlorofuran-3-yl)dibenzo[b,d]furan-3-sulfonamido)-3-methylbutanoic acid |
| 95 | (S)-2-(8-(2,5-dichlorofuran-3-yl)dibenzo[b,d]furan-3-sulfonamido)-3-methylbutanoic |
| | acid |
| 96 | (R)-2-(7-(furan-3-yl)dibenzo[b,d]furan-2-sulfonamido)-3-methylbutanoic acid |
| 97 | (R)-3-methyl-2-(7-(thiophen-3-yl)dibenzo[b,d]furan-2-sulfonamido)butanoic acid |
| 98 | (R)-2-(7-(furan-2-yl)dibenzo[b,d]furan-2-sulfonamido)-3-methylbutanoic acid |
| 99 | (R)-3-methyl-2-(7-(4-methylthiophen-3-yl)dibenzo[b,d]furan-2-sulfonamido)butanoic |
| | acid |
| 100 | (R)-2-(7-(benzo[b]thiophen-2-yl)dibenzo[b,d]furan-2-sulfonamido)-3-methylbutanoic |
| | acid |
| 101 | (R)-2-(7-(6-chloropyridin-3-yl)dibenzo[b,d]furan-2-sulfonamido)-3-methylbutanoic acid |
| 102 | (R)-2-(7-(6-methoxypyridin-3-yl)dibenzo[b,d]furan-2-sulfonamido)-3-methylbutanoic |
| | acid |
| 103 | (R)-2-(7-(1H-pyrazol-4-yl)dibenzo[b,d]furan-2-sulfonamido)-3-methylbutanoic acid |
| 104 | (R,E)-2-(7-(2-cyclohexylvinyl)dibenzo[b,d]furan-2-sulfonamido)-3-methylbutanoic acid |
| 105 | (R)-2-(7-(5-acetylthiophen-2-yl)dibenzo[b,d]furan-2-sulfonamido)-3-methylbutanoic acid |
| 106 | (S)-2-(8-(N,N-diethylcarbamimidoyl)dibenzo[b,d]furan-3-sulfonamido)-3-methylbutanoic |
| | acid |
| 107 | (S)-2-(8-(4,5-dihydrothiazol-2-yl)dibenzo[b,d]furan-3-sulfonamido)-3-methylbutanoic |
| | acid |
| 108 | (S)-2-(8-(N-methoxycarbamimidoyl)dibenzo[b,d]furan-3-sulfonamido)-3-methylbutanoic |
| | acid |
| 109 | (S)-2-(8-(N,N′-diethylcarbamimidoyl)dibenzo[b,d]furan-3-sulfonamido)-3- |
| | methylbutanoic acid |
| 110 | (S)-2-(8-(N-isopropyl-N-methylcarbamimidoyl)dibenzo[b,d]furan-3-sulfonamido)-3- |
| | methylbutanoic acid |
| 111 | (S)-2-(8-(5-carbamoylthiophen-2-yl)dibenzo[b,d]furan-3-sulfonamido)-3-methylbutanoic |
| | acid |
| 112 | (S)-5-(7-(N-(1-carboxy-2-methylpropyl)sulfamoyl)dibenzo[b,d]furan-2-yl)thiophene-2- |
| | carboxylic acid |
| 113 | (2S)-2-[8-(5-tert-Butyl-[1,2,4]oxadiazol-3-yl)-dibenzofuran-3-sulfonylamino]-3-methyl- |
| | butanoic acid |
| 114 | (2S)-2-[8-(5-Isopropyl-[1,2,4]oxadiazol-3-yl)-dibenzofuran-3-sulfonylamino]-3-methyl- |
| | butanoic acid |
| 115 | (R)-2-(7-(2,4-dimethoxypyrimidin-5-yl)dibenzo[b,d]furan-2-sulfonamido)-3- |
| | methylbutanoic acid |
| 116 | (R)-2-(7-(1H-pyrrol-2-yl)dibenzo[b,d]furan-2-sulfonamido)-3-methylbutanoic acid |
| 117 | (R)-3-methyl-2-(7-(1-methyl-1H-pyrazol-4-yl)dibenzo[b,d]furan-2-sulfonamido)butanoic |
| | acid |
| 118 | (R)-3-methyl-2-(7-(thiophen-2-yl)dibenzo[b,d]furan-2-sulfonamido)butanoic acid |
| 119 | (R)-2-(7-(benzofuran-2-yl)dibenzo[b,d]furan-2-sulfonamido)-3-methylbutanoic acid |
| 120 | (R)-3-methyl-2-(7-(4-(trifluoromethyl)phenyl)dibenzo[b,d]furan-2-sulfonamido)butanoic |
| | acid |
| 121 | (R)-3-methyl-2-(7-(1-methyl-1H-indol-2-yl)dibenzo[b,d]furan-2-sulfonamido)butanoic |
| | acid |
| 122 | (R)-2-(7-(5-fluoro-1H-indol-2-yl)dibenzo[b,d]furan-2-sulfonamido)-3-methylbutanoic |
| | acid |
| 123 | (2S)-2-[8-(5-Ethyl-[1,2,4]oxadiazol-3-yl)-dibenzofuran-3-sulfonylamino]-3-methyl- |
| | butanoic acid |
| 124 | (S)-2-(8-(5-fluorothiophen-2-yl)dibenzo[b,d]furan-3-sulfonamido)-3-methylbutanoic acid |
| 125 | (2S,2′S)-2,2′-[2,2′-bidibenzo[b,d]furan-7,7′-diylbis(sulfonylimino)]bis(3-methylbutanoic |
| | acid |
| 126 | (S)-3-methyl-2-(8-(4-(trifluoromethyl)thiazol-2-yl)dibenzo[b,d]furan-3- |
| | sulfonamido)butanoic acid |
| 127 | (S)-2-(8-(imino(pyrrolidin-1-yl)methyl)dibenzo[b,d]furan-3-sulfonamido)-3- |
| | methylbutanoic acid |
| 128 | (S)-2-(8-(N-ethylcarbamimidoyl)dibenzo[b,d]furan-3-sulfonamido)-3-methylbutanoic |
| | acid |
| 129 | (R)-2-(7-(furan-2-yl)dibenzo[b,d]thiophene-3-sulfonamido)-3-methylbutanoic acid |
| 130 | (S)-2-(8-(2H-tetrazol-5-yl)dibenzo[b,d]furan-3-sulfonamido)-3-methylbutanoic acid |
| 131 | (S)-3-methyl-2-(8-(5-(trifluoromethyl)thiophen-2-yl)dibenzo[b,d]furan-3- |
| | sulfonamido)butanoic acid |
| 132 | (S)-3-methyl-2-(8-(2-methyl-2H-tetrazol-5-yl)dibenzo[b,d]furan-3-sulfonamido)butanoic |
| | acid |
| 133 | (R)-2-(7-(5-tert-butyl-1,2,4-oxadiazol-3-yl)dibenzo[b,d]furan-2-sulfonamido)-3- |
| | methylbutanoic acid |
| 134 | (S)-2-(8-(3,5-dichlorofuran-2-yl)dibenzo[b,d]furan-3-sulfonamido)-3-methylbutanoic |
| | acid |
| 135 | (S)-3-methyl-2-(7-(5-methylfuran-2-yl)dibenzo[b,d]furan-3-sulfonamido)butanoic acid |
| 136 | (S)-2-(7-(benzo[b]thiophen-2-yl)dibenzo[b,d]furan-3-sulfonamido)-3-methylbutanoic |
| | acid |
| 137 | (S)-3-methyl-2-(7-(thiazol-2-yl)dibenzo[b,d]furan-3-sulfonamido)butanoic acid |
| 138 | (R)-2-(7-(5-isopropyl-1,2,4-oxadiazol-3-yl)dibenzo[b,d]furan-2-sulfonamido)-3- |
| | methylbutanoic acid |
| 139 | (R)-3-methyl-2-(7-(5-methyl-1,2,4-oxadiazol-3-yl)dibenzo[b,d]furan-2- |
| | sulfonamido)butanoic acid |
| 140 | (R)-2-(7-(5-ethyl-1,2,4-oxadiazol-3-yl)dibenzo[b,d]furan-2-sulfonamido)-3- |
| | methylbutanoic acid |
| 141 | (R)-3-methyl-2-(7-(5-(trifluoromethyl)-1,2,4-oxadiazol-3-yl)dibenzo[b,d]furan-2- |
| | sulfonamido)butanoic acid |
| 142 | (S)-3-methyl-2-(7-(5-methylthiophen-2-yl)dibenzo[b,d]furan-3-sulfonamido)butanoic |
| | acid |
| 143 | (S)-2-(7-(benzofuran-2-yl)dibenzo[b,d]furan-3-sulfonamido)-3-methylbutanoic acid |
| 144 | (R)-2-(7-(5-bromothiophen-2-yl)dibenzo[b,d]furan-2-sulfonamido)-3-methylbutanoic |
| | acid |
| 145 | (R)-2-(7-(3,5-dimethylisoxazol-4-yl)dibenzo[b,d]furan-2-sulfonamido)-3-methylbutanoic |
| | acid |
| 146 | (S)-3-methyl-2-(8-(5-methyl-1,3,4-thiadiazol-2-yl)dibenzo[b,d]furan-3- |
| | sulfonamido)butanoic acid |
| 147 | (R)-2-(7-(5-cyclopropyl-1,2,4-oxadiazol-3-yl)dibenzo[b,d]furan-2-sulfonamido)-3- |
| | methylbutanoic acid |
| 148 | (R)-2-(7-(5-cyclobutyl-1,2,4-oxadiazol-3-yl)dibenzo[b,d]furan-2-sulfonamido)-3- |
| | methylbutanoic acid |
| 149 | (R)-2-(7-(5-isobutyl-1,2,4-oxadiazol-3-yl)dibenzo[b,d]furan-2-sulfonamido)-3- |
| | methylbutanoic acid |
| 150 | (R)-3-methyl-2-(7-(5-phenyl-1,2,4-oxadiazol-3-yl)dibenzo[b,d]furan-2- |
| | sulfonamido)butanoic acid |
| 151 | (S)-2-(8-(benzo[d]thiazol-2-yl)dibenzo[b,d]furan-3-sulfonamido)-3-methylbutanoic acid |
| 152 | (S)-3-methyl-2-(8-(pyrimidin-5-yl)dibenzo[b,d]furan-3-sulfonamido)butanoic acid |
| 153 | (S)-2-(8-(2-methoxypyrimidin-5-yl)dibenzo[b,d]furan-3-sulfonamido)-3-methylbutanoic |
| | acid |
| 154 | (S)-2-(7-(5-isopropyl-1,2,4-oxadiazol-3-yl)dibenzo[b,d]furan-3-sulfonamido)-3- |
| | methylbutanoic acid |
| 155 | (S)-2-(7-(5-tert-butyl-1,2,4-oxadiazol-3-yl)dibenzo[b,d]furan-3-sulfonamido)-3- |
| | methylbutanoic acid |
| 156 | (S)-3-methyl-2-(7-(5-methyl-1,2,4-oxadiazol-3-yl)dibenzo[b,d]furan-3- |
| | sulfonamido)butanoic acid |
| 157 | (2S)-3-methyl-2-(8-(1-(2-methylbutyl)-1H-pyrazol-4-yl)dibenzo[b,d]furan-3- |
| | sulfonamido)butanoic acid |
| 158 | (S)-3-methyl-2-(8-(1-(2-morpholinoethyl)-1H-pyrazol-4-yl)dibenzo[b,d]furan-3- |
| | sulfonamido)butanoic acid |
| 159 | (S)-2-(8-(1-isobutyl-1H-pyrazol-4-yl)dibenzo[b,d]furan-3-sulfonamido)-3-methylbutanoic |
| | acid |
| 160 | (S)-3-methyl-2-(8-(1,3,5-trimethyl-1H-pyrazol-4-yl)dibenzo[b,d]furan-3- |
| | sulfonamido)butanoic acid |
| 161 | (S)-3-methyl-2-(8-(5-methyl-3-phenylisoxazol-4-yl)dibenzo[b,d]furan-3- |
| | sulfonamido)butanoic acid |
| 162 | (S)-3-methyl-2-(8-(5-methyl-1-phenyl-1H-pyrazol-4-yl)dibenzo[b,d]furan-3- |
| | sulfonamido)butanoic acid |
| 163 | (S)-3-methyl-2-(8-(4-methyl-2-phenylthiazol-5-yl)dibenzo[b,d]furan-3- |
| | sulfonamido)butanoic acid |
| 164 | (S)-3-methyl-2-(8-(4-methyl-2-(4-(trifluoromethyl)phenyl)thiazol-5-yl)dibenzo[b,d]furan- |
| | 3-sulfonamido)butanoic acid |
| 165 | (S)-2-(7-(4-bromo-5-ethylthiophen-2-yl)dibenzo[b,d]furan-3-sulfonamido)-3- |
| | methylbutanoic acid |
| 166 | (S)-2-(7-(2′,5-diethyl-2,3′-bithiophen-5′-yl)dibenzo[b,d]furan-3-sulfonamido)-3- |
| | methylbutanoic acid |
| 167 | (R)-3-methyl-2-(7-(pyrimidin-5-yl)dibenzo[b,d]furan-2-sulfonamido)butanoic acid |
| 168 | (R)-2-(7-(2-methoxypyrimidin-5-yl)dibenzo[b,d]furan-2-sulfonamido)-3-methylbutanoic |
| | acid |
| 169 | (R)-2-(7-(2,4-dimethylthiazol-5-yl)dibenzo[b,d]furan-2-sulfonamido)-3-methylbutanoic |
| | acid |
| 170 | (2R)-3-methyl-2-(7-(1-(2-methylbutyl)-1H-pyrazol-4-yl)dibenzo[b,d]furan-2- |
| | sulfonamido)butanoic acid |
| 171 | (R)-3-methyl-2-(7-(1-propyl-1H-pyrazol-4-yl)dibenzo[b,d]furan-2-sulfonamido)butanoic |
| | acid |
| 172 | (R)-3-methyl-2-(7-(1-(2-morpholinoethyl)-1H-pyrazol-4-yl)dibenzo[b,d]furan-2- |
| | sulfonamido)butanoic acid |
| 173 | (R)-2-(7-(1-isobutyl-1H-pyrazol-4-yl)dibenzo[b,d]furan-2-sulfonamido)-3-methylbutanoic |
| | acid |
| 174 | (R)-3-methyl-2-(7-(1,3,5-trimethyl-1H-pyrazol-4-yl)dibenzo[b,d]furan-2- |
| | sulfonamido)butanoic acid |
| 175 | (R)-2-(7-(1-benzyl-1H-pyrazol-4-yl)dibenzo[b,d]furan-2-sulfonamido)-3-methylbutanoic |
| | acid |
| 176 | (R)-3-methyl-2-(7-(4-methyl-2-phenylthiazol-5-yl)dibenzo[b,d]furan-2- |
| | sulfonamido)butanoic acid |
| 177 | (R)-3-methyl-2-(7-(4-methyl-2-(4-(trifluoromethyl)phenyl)thiazol-5-yl)dibenzo[b,d]furan- |
| | 2-sulfonamido)butanoic acid |
| 178 | (R)-2-(8-(5-chlorofuran-2-yl)dibenzo[b,d]furan-3-sulfonamido)-3-methylbutanoic acid |
| 179 | (S)-2-(8-(2-chlorothiazol-5-yl)dibenzo[b,d]furan-3-sulfonamido)-3-methylbutanoic acid |
| 180 | (S)-2-(8-(2-chlorothiazol-4-yl)dibenzo[b,d]furan-3-sulfonamido)-3-methylbutanoic acid |
| 181 | (S)-2-(7-(2-chlorothiazol-5-yl)dibenzo[b,d]furan-3-sulfonamido)-3-methylbutanoic acid |
| 182 | (S)-2-(7-(5-chlorofuran-2-yl)dibenzo[b,d]thiophene-3-sulfonamido)-3-methylbutanoic |
| | acid |
| 183 | Absent |
| 184 | Absent |
| 185 | (R)-3-methyl-2-(8-(5-(trifluoromethyl)-1,2,4-oxadiazol-3-yl)dibenzo[b,d]furan-3- |
| | sulfonamido)butanoic acid |
| 186 | (S)-2-(7-(N-hydroxycarbamimidoyl)dibenzo[b,d]furan-3-sulfonamido)-3-methylbutanoic |
| | acid |
| 187 | (S)-2-(7-(5-cyclopropyl-1,2,4-oxadiazol-3-yl)dibenzo[b,d]furan-3-sulfonamido)-3- |
| | methylbutanoic acid |
| 188 | (S)-2-(7-(5-(4-fluorophenyl)-1,2,4-oxadiazol-3-yl)dibenzo[b,d]furan-3-sulfonamido)-3- |
| | methylbutanoic acid |
| 189 | (R)-3-methyl-2-(7-(5-neopentyl-1,2,4-oxadiazol-3-yl)dibenzo[b,d]furan-2- |
| | sulfonamido)butanoic acid |
| 190 | (R)-2-(7-(5-cyclopentyl-1,2,4-oxadiazol-3-yl)dibenzo[b,d]furan-2-sulfonamido)-3- |
| | methylbutanoic acid |
| 191 | (R)-2-(7-(5-(cyclopentylmethyl)-1,2,4-oxadiazol-3-yl)dibenzo[b,d]furan-2-sulfonamido)- |
| | 3-methylbutanoic acid |
| 192 | (R)-2-(7-(5-cyclohexyl-1,2,4-oxadiazol-3-yl)dibenzo[b,d]furan-2-sulfonamido)-3- |
| | methylbutanoic acid |
| 193 | (S)-2-(7-(furan-2-yl)dibenzo[b,d]thiophene-3-sulfonamido)-3-methylbutanoic acid |
| 194 | (S)-2-(8-(benzo[d]oxazol-2-yl)dibenzo[b,d]furan-3-sulfonamido)-3-methylbutanoic acid |
| 195 | (S)-2-(2,2′-bidibenzo[b,d]furan-7-sulfonamido)-3-methylbutanoic acid |
| 196 | (S)-2-(8-(5-ethylthiophen-2-yl)dibenzo[b,d]furan-3-sulfonamido)-3-methylbutanoic acid |
| 197 | (S)-3-methyl-2-(8-(5-propylthiophen-2-yl)dibenzo[b,d]furan-3-sulfonamido)butanoic |
| | acid |
| 198 | (S)-2-(8-(5-tert-butylfuran-2-yl)dibenzo[b,d]furan-3-sulfonamido)-3-methylbutanoic acid |
| 199 | (S)-3-methyl-2-(8-(5-(5-methyl-1,2,4-oxadiazol-3-yl)thiophen-2-yl)dibenzo[b,d]furan-3- |
| | sulfonamido)butanoic acid |
| 200 | (S)-2-(8-(5-chloro-4-(trifluoromethyl)thiazol-2-yl)dibenzo[b,d]furan-3-sulfonamido)-3- |
| | methylbutanoic acid |
| 201 | (S)-2-(8-(2,4-dimethylthiazol-5-yl)dibenzo[b,d]furan-3-sulfonamido)-3-methylbutanoic |
| | acid |
| 202 | (S)-3-methyl-2-(8-(2-methylthiazol-5-yl)dibenzo[b,d]furan-3-sulfonamido)butanoic acid |
| 203 | (S)-2-(8-(6-chlorobenzo[d]thiazol-2-yl)dibenzo[b,d]furan-3-sulfonamido)-3- |
| | methylbutanoic acid |
| 204 | (S)-2-(8-(2-isobutyl-4-methylthiazol-5-yl)dibenzo[b,d]furan-3-sulfonamido)-3- |
| | methylbutanoic acid |
| 205 | Absent |
| 206 | (S)-3-methyl-2-(8-(5-phenyl-3-(trifluoromethyl)-1H-pyrazol-4-yl)dibenzo[b,d]furan-3- |
| | sulfonamido)butanoic acid |
| 207 | (S)-2-(8-(5-(1H-tetrazol-5-yl)thiophen-2-yl)dibenzo[b,d]furan-3-sulfonamido)-3- |
| | methylbutanoic acid |
| 208 | (S)-2-(8-(6-methoxybenzo[d]thiazol-2-yl)dibenzo[b,d]furan-3-sulfonamido)-3- |
| | methylbutanoic acid |
| 209 | (S)-2-(8-(6-fluorobenzo[d]thiazol-2-yl)dibenzo[b,d]furan-3-sulfonamido)-3- |
| | methylbutanoic acid |
| 210 | (S)-3-methyl-2-(8-(6-methylbenzo[d]thiazol-2-yl)dibenzo[b,d]furan-3- |
| | sulfonamido)butanoic acid |
| 211 | (S)-2-(8-(5-(isoxazol-5-yl)thiophen-2-yl)dibenzo[b,d]furan-3-sulfonamido)-3- |
| | methylbutanoic acid |
| 212 | (S)-3-methyl-2-(8-(5-((4-methylpiperazin-1-yl)methyl)thiazol-2-yl)dibenzo[b,d]furan-3- |
| | sulfonamido)butanoic acid |
| 213 | (S)-2-(8-(5-(((cyclopropylmethyl)(propyl)amino)methyl)thiazol-2-yl)dibenzo[b,d]furan-3- |
| | sulfonamido)-3-methylbutanoic acid |
| 214 | (S)-2-(8-(5-((1H-pyrazol-1-yl)methyl)thiazol-2-yl)dibenzo[b,d]furan-3-sulfonamido)-3- |
| | methylbutanoic acid |
| 215 | (S)-2-(8-(5-(hydroxymethyl)thiazol-2-yl)dibenzo[b,d]furan-3-sulfonamido)-3- |
| | methylbutanoic acid |
| 216 | (S)-2-(8-(5-(isoxazol-3-yl)thiophen-2-yl)dibenzo[b,d]furan-3-sulfonamido)-3- |
| | methylbutanoic acid |
| 217 | (S)-2-(8-(4-bromothiazol-2-yl)dibenzo[b,d]furan-3-sulfonamido)-3-methylbutanoic acid |
| 218 | (S)-2-(8-(4-fluorobenzo[d]thiazol-2-yl)dibenzo[b,d]furan-3-sulfonamido)-3- |
| | methylbutanoic acid |
| 219 | (S)-2-(8-(5-fluorobenzo[d]thiazol-2-yl)dibenzo[b,d]furan-3-sulfonamido)-3- |
| | methylbutanoic acid |
| 220 | (S)-2-(8-(5,6-difluorobenzo[d]thiazol-2-yl)dibenzo[b,d]furan-3-sulfonamido)-3- |
| | methylbutanoic acid |
| 221 | (S)-3-methyl-2-(8-(6-(trifluoromethoxy)benzo[d]thiazol-2-yl)dibenzo[b,d]furan-3- |
| | sulfonamido)butanoic acid |
| 222 | (S)-3-methyl-2-(8-(4,5,6-trifluorobenzo[d]thiazol-2-yl)dibenzo[b,d]furan-3- |
| | sulfonamido)butanoic acid |
| 223 | (S)-2-(8-(4-methoxybenzo[d]thiazol-2-yl)dibenzo[b,d]furan-3-sulfonamido)-3- |
| | methylbutanoic acid |
| 224 | (S)-2-(8-(5-chlorothiazol-2-yl)dibenzo[b,d]furan-3-sulfonamido)-3-methylbutanoic acid |
| 225 | (S)-2-(8-(5-methoxybenzo[d]thiazol-2-yl)dibenzo[b,d]furan-3-sulfonamido)-3- |
| | methylbutanoic acid |
| 226 | (S)-2-(7-(benzo[d]thiazol-2-yl)dibenzo[b,d]furan-3-sulfonamido)-3-methylbutanoic acid |
| 227 | (S)-2-(7-(benzo[d]oxazol-2-yl)dibenzo[b,d]furan-3-sulfonamido)-3-methylbutanoic acid |
| 228 | (S)-3-methyl-2-(7-(5-(5-methyl-1,2,4-oxadiazol-3-yl)thiazol-2-yl)dibenzo[b,d]furan-3- |
| | sulfonamido)butanoic acid |
| 229 | (S)-2-(7-(5-ethylthiophen-2-yl)dibenzo[b,d]furan-3-sulfonamido)-3-methylbutanoic acid |
| 230 | (S)-2-(7-(2,4-dimethylthiazol-5-yl)dibenzo[b,d]furan-3-sulfonamido)-3-methylbutanoic |
| | acid |
| 231 | (S)-2-(7-(5-tert-butylfuran-2-yl)dibenzo[b,d]furan-3-sulfonamido)-3-methylbutanoic acid |
| 232 | (S)-3-methyl-2-(7-(5-propylthiophen-2-yl)dibenzo[b,d]furan-3-sulfonamido)butanoic |
| | acid |
| 233 | (S)-2-(7-(5-chloro-4-(trifluoromethyl)thiazol-2-yl)dibenzo[b,d]furan-3-sulfonamido)-3- |
| | methylbutanoic acid |
| 234 | (S)-3-methyl-2-(7-(5-methylthiazol-2-yl)dibenzo[b,d]furan-3-sulfonamido)butanoic acid |
| 235 | (S)-2-(7-(2-isobutyl-4-methylthiazol-5-yl)dibenzo[b,d]furan-3-sulfonamido)-3- |
| | methylbutanoic acid |
| 236 | (S)-3-methyl-2-(7-(6-(trifluoromethyl)benzo[d]thiazol-2-yl)dibenzo[b,d]furan-3- |
| | sulfonamido)butanoic acid |
| 237 | (S)-2-(7-(6-fluorobenzo[d]thiazol-2-yl)dibenzo[b,d]furan-3-sulfonamido)-3- |
| | methylbutanoic acid |
| 238 | Absent |
| 239 | (R)-2-(7-(5-ethylthiophen-2-yl)dibenzo[b,d]furan-2-sulfonamido)-3-methylbutanoic acid |
| 240 | (R)-2-(7-(5-tert-butylfuran-2-yl)dibenzo[b,d]furan-2-sulfonamido)-3-methylbutanoic acid |
| 241 | (S)-2-(7-(5-tert-butylfuran-2-yl)dibenzo[b,d]furan-2-sulfonamido)-3-methylbutanoic acid |
| 242 | (S)-2-(7-(5-ethylthiophen-2-yl)dibenzo[b,d]furan-2-sulfonamido)-3-methylbutanoic acid |
| 243 | (R)-3-methyl-2-(7-(5-propylthiophen-2-yl)dibenzo[b,d]furan-2-sulfonamido)butanoic |
| | acid |
| 244 | (R)-2-(7-(2-isobutylthiazol-5-yl)dibenzo[b,d]furan-2-sulfonamido)-3-methylbutanoic acid |
| 245 | (R)-2-(7-(2-isobutyl-4-methylthiazol-5-yl)dibenzo[b,d]furan-2-sulfonamido)-3- |
| | methylbutanoic acid |
| 246 | (S)-3-methyl-2-(7-(5-propylthiophen-2-yl)dibenzo[b,d]furan-2-sulfonamido)butanoic |
| | acid |
| 247 | (S)-2-(7-(2-isobutyl-4-methylthiazol-5-yl)dibenzo[b,d]furan-2-sulfonamido)-3- |
| | methylbutanoic acid |
| 248 | (S)-2-(8-(2-isobutylthiazol-5-yl)dibenzo[b,d]furan-3-sulfonamido)-3-methylbutanoic acid |
| 249 | (S)-2-(7-(2-isobutylthiazol-5-yl)dibenzo[b,d]furan-3-sulfonamido)-3- |
| | methylbutanoic acid |
| 250 | (S)-2-(7-(1H-tetrazol-5-yl)dibenzo[b,d]furan-3-sulfonamido)-3-methylbutanoic acid |
| 251 | 2-(8-(thiazol-2-yl)dibenzo[b,d]furan-3-sulfonamido)acetic acid |
| 252 | (S)-2-(8-(5-tert-butyl-1,2,4-oxadiazol-3-yl)dibenzo[b,d]furan-3-sulfonamido)-4- |
| | methylpentanoic acid |
| 253 | (R)-2-(8-(5-tert-butyl-1,2,4-oxadiazol-3-yl)dibenzo[b,d]furan-3-sulfonamido)-4- |
| | methylpentanoic acid |
| 254 | (S)-2-(8-(5-tert-butyl-1,2,4-oxadiazol-3-yl)dibenzo[b,d]furan-3-sulfonamido)-2- |
| | phenylacetic acid |
| 255 | (R)-2-(8-(5-tert-butyl-1,2,4-oxadiazol-3-yl)dibenzo[b,d]furan-3-sulfonamido)-2- |
| | phenylacetic acid |
| 256 | (R)-2-(8-(5-tert-butyl-1,2,4-oxadiazol-3-yl)dibenzo[b,d]furan-3-sulfonamido)-3-(1H- |
| | indol-3-yl)propanoic acid |
| 257 | (S)-2-(8-(5-tert-butyl-1,2,4-oxadiazol-3-yl)dibenzo[b,d]furan-3-sulfonamido)-3,3- |
| | dimethylbutanoic acid |
| 258 | (R)-2-(8-(5-tert-butyl-1,2,4-oxadiazol-3-yl)dibenzo[b,d]furan-3-sulfonamido)-3- |
| | methylbutanoic acid |
| 259 | (S)-2-(8-(5-cyclopropyl-1,2,4-oxadiazol-3-yl)dibenzo[b,d]furan-3-sulfonamido)-3- |
| | methylbutanoic acid |
| 260 | (S)-3-methyl-2-(8-(5-(tetrahyd ro-2H-pyran-4-yl)-1,2,4-oxadiazol-3-yl)dibenzo[b,d]furan- |
| | 3-sulfonamido)butanoic acid |
| 261 | (S)-3-methyl-2-(8-(5-neopentyl-1,2,4-oxadiazol-3-yl)dibenzo[b,d]furan-3- |
| | sulfonamido)butanoic acid |
| 262 | (S)-2-(8-(5-cyclobutyl-1,2,4-oxadiazol-3-yl)dibenzo[b,d]furan-3-sulfonamido)-3- |
| | methylbutanoic acid |
| 263 | (S)-2-(8-(5-cyclopentyl-1,2,4-oxadiazol-3-yl)dibenzo[b,d]furan-3-sulfonamido)-3- |
| | methylbutanoic acid |
| 264 | (S)-3-methyl-2-(8-(5-(thiophen-2-yl)-1,2,4-oxadiazol-3-yl)dibenzo[b,d]furan-3- |
| | sulfonamido)butanoic acid |
| 265 | (S)-3-methyl-2-(8-(5-phenyl-1,2,4-oxadiazol-3-yl)dibenzo[b,d]furan-3- |
| | sulfonamido)butanoic acid |
| 266 | (S)-2-(8-(5-benzyl-1,2,4-oxadiazol-3-yl)dibenzo[b,d]furan-3-sulfonamido)-3- |
| | methylbutanoic acid |
| 267 | (S)-2-(8-(5-(methoxymethyl)-1,2,4-oxadiazol-3-yl)dibenzo[b,d]furan-3-sulfonamido)-3- |
| | methylbutanoic acid |
| 268 | (2S)-3-methyl-2-(8-(5-(tetrahydrofuran-3-yl)-1,2,4-oxadiazol-3-yl)dibenzo[b,d]furan-3- |
| | sulfonamido)butanoic acid |
| 269 | (S)-2-(8-(5-(2,4-difluorophenyl)-1,2,4-oxadiazol-3-yl)dibenzo[b,d]furan-3-sulfonamido)- |
| | 3-methylbutanoic acid |
| 270 | (S)-2-(8-(5-(2,4-dichlorophenyl)-1,2,4-oxadiazol-3-yl)dibenzo[b,d]furan-3-sulfonamido)- |
| | 3-methylbutanoic acid |
| 271 | (S)-3-methyl-2-(8-(5-(4-(trifluoromethyl)phenyl)-1,2,4-oxadiazol-3-yl)dibenzo[b,d]furan- |
| | 3-sulfonamido)butanoic acid |
| 272 | (S)-2-(8-(5-(4-fluorophenyl)-1,2,4-oxadiazol-3-yl)dibenzo[b,d]furan-3-sulfonamido)-3- |
| | methylbutanoic acid |
| 273 | (S)-7-(N-(1-carboxy-2-methylpropyl)sulfamoyl)dibenzo[b,d]furan-2-carboxylic acid |
| 274 | 2-(7-(5-tert-butyl-1,2,4-oxadiazol-3-yl)dibenzo[b,d]furan-2-sulfonamido)acetic acid |
| 275 | (R)-2-(7-(5-tert-butyl-1,2,4-oxadiazol-3-yl)dibenzo[b,d]furan-2-sulfonamido)-3- |
| | phenylpropanoic acid |
| 276 | (S)-2-(7-(5-tert-butyl-1,2,4-oxadiazol-3-yl)dibenzo[b,d]furan-2-sulfonamido)-3- |
| | methylbutanoic acid |
| 277 | 2-(7-(5-tert-butyl-1,2,4-oxadiazol-3-yl)dibenzo[b,d]furan-2-sulfonamido)-2- |
| | methylpropanoic acid |
| 278 | (R)-2-(7-(5-tert-butyl-1,2,4-oxadiazol-3-yl)dibenzo[b,d]furan-2-sulfonamido)-4- |
| | methylpentanoic acid |
| 279 | (S)-2-(7-(5-tert-butyl-1,2,4-oxadiazol-3-yl)dibenzo[b,d]furan-2-sulfonamido)-4- |
| | methylpentanoic acid |
| 280 | (S)-2-(7-(5-tert-butyl-1,2,4-oxadiazol-3-yl)dibenzo[b,d]furan-2-sulfonamido)-2-(1H- |
| | indol-3-yl)acetic acid |
| 281 | (S)-2-(7-(5-tert-butyl-1,2,4-oxadiazol-3-yl)dibenzo[b,d]furan-2-sulfonamido)-2- |
| | phenylacetic acid |
| 282 | (S)-2-(7-(5-tert-butyl-1,2,4-oxadiazol-3-yl)dibenzo[b,d]furan-2-sulfonamido)-3,3- |
| | dimethylbutanoic acid |
| 283 | (S)-3-methyl-2-(8-(4-(4-(trifluoromethyl)phenyl)thiazol-2-yl)dibenzo[b,d]furan-3- |
| | sulfonamido)butanoic acid |
| 284 | (S)-2-(8-(4-(4-fluorophenyl)thiazol-2-yl)dibenzo[b,d]furan-3-sulfonamido)-3- |
| | methylbutanoic acid |
| 285 | (R)-3-methyl-2-(7-(thiazol-2-yl)dibenzo[b,d]thiophene-2-sulfonamido)butanoic acid |
| 286 | (R)-2-(7-(benzo[d]thiazol-2-yl)dibenzo[b,d]thiophene-2-sulfonamido)-3-methylbutanoic |
| | acid |
| 287 | (R)-2-(7-(furan-2-yl)dibenzo[b,d]thiophene-2-sulfonamido)-3-methylbutanoic acid |
| 288 | (R)-2-(7-(5-chlorofuran-2-yl)dibenzo[b,d]thiophene-2-sulfonamido)-3-methylbutanoic |
| | acid |
| 289 | (R)-3-methyl-2-(7-(5-phenylthiophen-2-yl)dibenzo[b,d]furan-2-sulfonamido)butanoic |
| | acid |
| 290 | (R)-2-(7-(5-chlorofuran-2-yl)dibenzo[b,d]furan-2-sulfonamido)-3-methylbutanoic acid |
| 291 | (R)-3-methyl-2-(7-(thiazol-2-yl)dibenzo[b,d]furan-2-sulfonamido)butanoic acid |
| 292 | (R)-3-methyl-2-(7-(5-methyl-1,3,4-thiadiazol-2-yl)dibenzo[b,d]furan-2- |
| | sulfonamido)butanoic acid |
| 293 | (R)-2-(7-(benzo[d]thiazol-2-yl)dibenzo[b,d]furan-2-sulfonamido)-3-methylbutanoic acid |
| 294 | (R)-2-(7-(benzo[d]oxazol-2-yl)dibenzo[b,d]furan-2-sulfonamido)-3-methylbutanoic acid |
| 295 | (R)-2-(7-(5-chloro-4-(trifluoromethyl)thiazol-2-yl)dibenzo[b,d]furan-2-sulfonamido)-3- |
| | methylbutanoic acid |
| 296 | (R)-2-(7-(6-methoxybenzo[d]thiazol-2-yl)dibenzo[b,d]furan-2-sulfonamido)-3- |
| | methylbutanoic acid |
| 297 | (R)-2-(7-(6-fluorobenzo[d]thiazol-2-yl)dibenzo[b,d]furan-2-sulfonamido)-3- |
| | methylbutanoic acid |
| 298 | (R)-3-methyl-2-(7-(6-methylbenzo[d]thiazol-2-yl)dibenzo[b,d]furan-2- |
| | sulfonamido)butanoic acid |
| 299 | (R)-2-(7-(4-fluorobenzo[d]thiazol-2-yl)dibenzo[b,d]furan-2-sulfonamido)-3- |
| | methylbutanoic acid |
| 300 | (R)-3-methyl-2-(7-(4,5,6-trifluorobenzo[d]thiazol-2-yl)dibenzo[b,d]furan-2- |
| | sulfonamido)butanoic acid |
| 301 | (R)-3-methyl-2-(7-(6-(trifluoromethoxy)benzo[d]thiazol-2-yl)dibenzo[b,d]furan-2- |
| | sulfonamido)butanoic acid |
| 302 | (R)-3-methyl-2-(7-(6-(trifluoromethyl)benzo[d]thiazol-2-yl)dibenzo[b,d]furan-2- |
| | sulfonamido)butanoic acid |
| 303 | (S)-2-(8-ethynyldibenzo[b,d]furan-3-sulfonamido)-3-methylbutanoic acid |
| 304 | (S)-2-(7-(5-chlorothiophen-2-yl)dibenzo[b,d]thiophene-3-sulfonamido)-3- |
| | methylbutanoic acid |
| 305 | (S)-2-(8-(4,5-dimethylthiazol-2-yl)dibenzo[b,d]furan-3-sulfonamido)-3-methylbutanoic |
| | acid |
| 306 | (S)-2-[7-(5,6-Dihydro-4H-cyclopentathiazol-2-yl)-dibenzofuran-3-sulfonylamino]-3- |
| | methyl-butyric acid |
| 307 | (S)-3-methyl-2-(8-(4,5,6,7-tetrahydrobenzo[d]thiazol-2-yl)dibenzo[b,d]furan-3- |
| | sulfonamido)butanoic acid |
| 308 | (S)-2-(8-(benzo[d][1,3]dioxol-5-yl)dibenzo[b,d]furan-3-sulfonamido)-3-methylbutanoic |
| | acid |
| 309 | (S)-3-methyl-2-(8-phenyldibenzo[b,d]furan-3-sulfonamido)butanoic acid |
| 310 | (S)-2-(8-(4-methoxyphenyl)dibenzo[b,d]furan-3-sulfonamido)-3-methylbutanoic acid |
| 311 | (S)-3-methyl-2-(8-(4-(trifluoromethyl)phenyl)dibenzo[b,d]furan-3-sulfonamido)butanoic |
| | acid |
| 312 | Absent |
| 313 | (S)-3-methyl-2-(7-phenyldibenzo[b,d]thiophene-3-sulfonamido)butanoic acid |
| 314 | (R)-3-methyl-2-(7-phenyldibenzo[b,d]thiophene-3-sulfonamido)butanoic acid |

Another aspect of the invention relates to the compound of formula I, or a pharmaceutically acceptable salt or ester thereof, wherein W is —C(O)OR¹³ and V is —CR¹³R¹⁵— or —CH₂CR¹³R¹⁵—; wherein R¹³ and R¹⁵ are different and the carbon atom to which R¹³ and R¹⁵ is each attached is a chiral center, and wherein at least 75% of the compound is in the form of the S— or an R-enantiomer. In one embodiment, the product may be the compound of formula I, or a pharmaceutically acceptable salt or ester thereof, wherein W is —C(O)OR¹³ and V is —CR¹³R¹⁵— or —CH₂CR¹³R¹⁵—; wherein R¹³ and R¹⁵ are different and the carbon atom to which R¹³ and R¹⁵ is each attached is a chiral center, and wherein at least 75% of the compound is in the form of the R-enantiomer. In another embodiment, the product may be the compound of formula I, or a pharmaceutically acceptable salt or ester thereof, wherein W is —C(O)OR¹³ and V is —CR¹³R¹⁵— or —CH₂CR¹³R¹⁵—; wherein R¹³ and R¹⁵ are different and the carbon atom to which R¹³ and R¹⁵ is each attached is a chiral center, and wherein at least 75% of the compound is in the form of the S-enantiomer. The invention also includes products wherein at least 80%, 85%, 90% or 95% of the compound is in the form of the S- or R-enantiomer.

Salts of the compounds of formula I, which can have an acidic moiety, can be formed using organic and inorganic bases. Both mono and polyanionic salts, depending on the number of acidic hydrogens available for deprotonation are included. Suitable salts formed with bases include metal salts, such as alkali metal or alkaline earth metal salts, for example sodium, potassium, or magnesium salts; ammonia salts and organic amine salts, such as those formed with morpholine, thiomorpholine, piperidine, pyrrolidine, a mono-, di- or tri-lower alkylamine (e.g., ethyl-tert-butyl-, diethyl-, diisopropyl-, triethyl-, tributyl- or dimethylpropylamine), or a mono-, di-, or trihydroxy lower alkylamine (e.g., mono-, di- or triethanolamine). Specific non-limiting examples of inorganic bases include NaHCO₃, Na₂CO₃, KHCO₃, K₂CO₃, Cs₂CO₃, LiOH, NaOH, KOH, NaH₂PO₄, Na₂HPO₄, and Na₃PO₄. Internal salts also can be formed. Similarly, when a compound disclosed herein contains a basic moiety, salts can be formed using organic and inorganic acids. For example, salts can be formed from the following acids: acetic, benzenesulfonic, benzoic, camphorsulfonic, citric, dichloroacetic, ethenesulfonic, formic, fumaric, gluconic, glutamic, hippuric, hydrobromic, hydrochloric, isethionic, lactic, maleic, malic, malonic, mandelic, methanesulfonic, mucic, naphthalenesulfonic, nitric, oxalic, pamoic, pantothenic, phosphoric, phthalic, propionic, succinic, sulfuric, tartaric, and toluenesulfonic, as well as other known pharmaceutically acceptable acids.

Esters of the compounds of formula I can include various pharmaceutically acceptable esters known in the art that can be metabolized into the free acid form (e.g., a free carboxylic acid form) in a mammal. Examples of such esters include alkyl esters (e.g., of 1 to 10 carbon atoms), cycloalkyl esters (e.g., of 3-10 carbon atoms), aryl esters (e.g., of 6-14 carbon atoms, including of 6-10 carbon atoms), and heterocyclic analogues thereof (e.g., of 3-14 ring atoms, 1-3 of which can be selected from oxygen, nitrogen, and sulfur heteroatoms), wherein the alcohol residue can include further substituents. In some embodiments, esters of the compounds disclosed herein can be C₁₋₁₀ alkyl esters, such as methyl esters, ethyl esters, propyl esters, isopropyl esters, butyl esters, isobutyl esters, t-butyl esters, pentyl esters, isopentyl esters, neopentyl esters, and hexyl esters; C₃₋₁₀ cycloalkyl esters, such as cyclopropyl esters, cyclopropylmethyl esters, cyclobutyl esters, cyclopentyl esters, and cyclohexyl esters; or aryl esters, such as phenyl esters, benzyl esters, and tolyl esters.

Also provided in accordance with the present teachings are prodrugs of the compounds disclosed herein. As used herein, “prodrug” refers to a moiety that produces, generates or releases a compound of the present teachings when administered to a mammalian subject. Prodrugs can be prepared by modifying functional groups present in the compounds in such a way that the modifications are cleaved, either by routine manipulation or in vivo, from the parent compounds. Examples of prodrugs include compounds as described herein that contain one or more molecular moieties appended to a hydroxyl, amino, sulfhydryl, or carboxyl group of the compound, and that when administered to a mammalian subject, is cleaved in vivo to form the free hydroxyl, amino, sulfhydryl, or carboxyl group, respectively. Examples of prodrugs can include acetate, formate, and benzoate derivatives of alcohol and amine functional groups in the compounds of the present teachings. Preparation and use of prodrugs is discussed in T. Higuchi and V. Stella, “Pro-drugs as Novel Delivery Systems,” Vol. 14 of the A.C.S. Symposium Series, and in Bioreversible Carriers in Drug Design, ed. Edward B. Roche, American Pharmaceutical Association and Pergamon Press, 1987, the entire disclosures of which are incorporated by reference herein for all purposes.

Another aspect of the invention provides for compositions comprising the compound of formula I, or a pharmaceutically acceptable salt or ester thereof, and a pharmaceutically acceptable carrier or excipient. Examples of such carriers and excipients are well known to those skilled in the art and can be prepared in accordance with acceptable pharmaceutical procedures, such as, for example, those described in Remington: The Science and Practice of Pharmacy, 20th edition, ed. Alfonso R. Gennaro, Lippincott Williams & Wilkins, Baltimore, Md. (2000), the entire disclosure of which is incorporated by reference herein for all purposes. As used herein, “pharmaceutically acceptable” refers to a substance that is acceptable for use in pharmaceutical applications from a toxicological perspective and does not adversely interact with the active ingredient. Accordingly, pharmaceutically acceptable carriers are those that are compatible with the other ingredients in the formulation and are biologically acceptable. Supplementary active ingredients may also be incorporated into the pharmaceutical compositions.

Another aspect of the invention relates to methods for inhibiting one or more matrix metalloproteinases in a mammal comprising administering to the mammal an effective amount of the compound of formula I or mixtures thereof, or a pharmaceutically acceptable salt or ester thereof. In some embodiments, the matrix metalloproteinases comprise MMP-12.

Another aspect of the invention relates to treatment of pathological conditions or disorders arising from an imbalance of cellular regulation, mediated wholly or in part by one or more matrix metallic proteinases. Treatment may be provided by administering to a mammal with the pathological condition or disorder, an effective amount of the compound of formula I or mixture thereof, or a pharmaceutically acceptable salt or ester thereof. Examples of the pathological conditions or disorders may include rheumatoid arthritis, osteoarthritis, atherosclerosis, multiple sclerosis, spinal cord injury, fibrosis, lung cancer, skin cancer, asthma, chronic obstructive pulmonary disorder, obesity, and diabetes. Compounds of the present teachings may be useful for the inhibition, palliation or prevention of a pathological condition or disorder in a mammal, for example, a human. Included in the present teachings are methods of providing to a mammal a medicament that comprises a compound or mixture thereof of the compounds of formula I, in combination or association with a pharmaceutically acceptable carrier. Compounds of the present teachings may be administered alone or in combination with other therapeutically effective compounds or therapies for the treatment or inhibition of the pathological condition or disorder. As used herein, a “therapeutic effect” refers to the an effect whereby the disease, disorder or condition is reduced in severity, palliated or ameliorated, according to clinical (biochemical, physiological, biological or psychological) parameters that may be measurable over a given period of time.

The present teachings also include use of the compounds disclosed herein as active therapeutic substances for the treatment or inhibition of a pathological condition or disorder, for example, a condition mediated wholly or in part by one or more MMPs or characterized by an MMP/TIMP imbalance such as rheumatoid arthritis, osteoarthritis, artherosclerosis, multiple sclerosis, heart failure, spinal cord injuries, skin aging, fibrosis, lung cancer, skin cancer, chronic obstructive pulmonary diseases, asthma, obesity, and diabetes. Accordingly, the present teachings further provide methods of treating these pathological conditions and disorders using the compounds described herein. As used herein, “treating” refers to partially or completely alleviating, inhibiting, and/or ameliorating the condition. In some embodiments, the methods include identifying a mammal having a pathological condition or disorder characterized by an MMP/TIMP imbalance, and administering to the mammal a therapeutically effective amount of a compound as described herein. In some embodiments, the method includes administering to a mammal a pharmaceutical composition that includes a compound disclosed herein in combination or association with a pharmaceutically acceptable carrier.

The present teachings further include use of the compounds disclosed herein as active therapeutic substances for the prevention of a pathological condition or disorder, for example, a condition mediated wholly or in part by one or more MMPs or characterized by an MMP/TIMP imbalance such as rheumatoid arthritis, osteoarthritis, artherosclerosis, multiple sclerosis, heart failure, spinal cord injuries, skin aging, fibrosis, lung cancer, skin cancer, chronic obstructive pulmonary diseases, asthma, obesity, and diabetes. Accordingly, the present teachings further provide methods of preventing these pathological conditions and disorders using the compounds described herein. In some embodiments, the methods include identifying a mammal that could potentially have a pathological condition or disorder characterized by an MMP/TIMP imbalance, and providing to the mammal a therapeutically effective amount of a compound as described herein. In some embodiments, the method includes administering to a mammal a pharmaceutical composition that includes a compound disclosed herein in combination or association with a pharmaceutically acceptable carrier.

Compounds of the present teachings can be administered orally or parenterally, neat or in combination with conventional pharmaceutical carriers. Applicable solid carriers can include one or more substances which can also act as flavoring agents, lubricants, solubilizers, suspending agents, fillers, glidants, compression aids, binders or tablet-disintegrating agents, or encapsulating materials. The compounds can be formulated in conventional manner, for example, in a manner similar to that used for known antiinflammatory agents. Oral formulations containing an active compound disclosed herein can include any conventionally used oral form, including tablets, capsules, buccal forms, troches, lozenges and oral liquids, suspensions or solutions. In powders, the carrier can be a finely divided solid, which is an admixture with a finely divided active compound. In tablets, an active compound can be mixed with a carrier having the necessary compression properties in suitable proportions and compacted in the shape and size desired. The powders and tablets may contain up to 99% of the active compound.

Capsules can contain mixtures of active compound(s) with inert filler(s) and/or diluent(s) such as the pharmaceutically acceptable starches (e.g., corn, potato or tapioca starch), sugars, artificial sweetening agents, powdered celluloses (e.g., crystalline and microcrystalline celluloses), flours, gelatins, gums, and the like.

Useful tablet formulations can be made by conventional compression, wet granulation or dry granulation methods and utilize pharmaceutically acceptable diluents, binding agents, lubricants, disintegrants, surface modifying agents (including surfactants), suspending or stabilizing agents, including magnesium stearate, stearic acid, sodium lauryl sulfate, talc, sugars, lactose, dextrin, starch, gelatin, cellulose, methyl cellulose, microcrystalline cellulose, sodium carboxymethyl cellulose, carboxymethylcellulose calcium, polyvinylpyrrolidine, alginic acid, acacia gum, xanthan gum, sodium citrate, complex silicates, calcium carbonate, glycine, sucrose, sorbitol, dicalcium phosphate, calcium sulfate, lactose, kaolin, mannitol, sodium chloride, low melting waxes, and ion exchange resins. Preferred surface modifying agents include nonionic and anionic surface modifying agents. Representative examples of surface modifying agents include poloxamer 188, benzalkonium chloride, calcium stearate, cetostearl alcohol, cetomacrogol emulsifying wax, sorbitan esters, colloidal silicon dioxide, phosphates, sodium dodecylsulfate, magnesium aluminum silicate, and triethanolamine. Oral formulations herein can utilize standard delay or time-release formulations to alter the absorption of the active compound(s). The oral formulation can also comprise a compound as described herein in water or fruit juice, containing appropriate solubilizers or emulsifiers as needed.

Liquid carriers can be used in preparing solutions, suspensions, emulsions, syrups, elixirs, and for inhaled delivery. A compound described herein can be dissolved or suspended in a pharmaceutically acceptable liquid carrier such as water, an organic solvent, or a mixture of both, or pharmaceutically acceptable oils or fats. The liquid carrier can contain other suitable pharmaceutical additives such as solubilizers, emulsifiers, buffers, preservatives, sweeteners, flavoring agents, suspending agents, thickening agents, colors, viscosity regulators, stabilizers, and osmo-regulators. Examples of liquid carriers for oral and parenteral administration include water (particularly containing additives as described above, e.g., cellulose derivatives such as a sodium carboxymethyl cellulose solution), alcohols (including monohydric alcohols and polyhydric alcohols, e.g., glycols) and their derivatives, and oils (e.g., fractionated coconut oil and arachis oil). For parenteral administration, the carrier can be an oily ester such as ethyl oleate and isopropyl myristate. Sterile liquid carriers are used in sterile liquid form compositions for parenteral administration. The liquid carrier for pressurized compositions can be halogenated hydrocarbon or other pharmaceutically acceptable propellants.

Liquid pharmaceutical compositions, which are sterile solutions or suspensions, can be utilized by, for example, intramuscular, intraperitoneal or subcutaneous injection. Sterile solutions can also be administered intravenously. Compositions for oral administration can be in either liquid or solid form.

Preferably the pharmaceutical composition is in unit dosage form, for example, as tablets, capsules, powders, solutions, suspensions, emulsions, granules, or suppositories. In such form, the pharmaceutical composition can be sub-divided in unit dose(s) containing appropriate quantities of the active compound. The unit dosage forms can be packaged compositions, for example, packeted powders, vials, ampoules, prefilled syringes or sachets containing liquids. Alternatively, the unit dosage form can be a capsule or tablet itself, or it can be the appropriate number of any such compositions in package form. Such unit dosage form may contain from about 1 mg/kg of active compound to about 500 mg/kg of active compound, and can be given in a single dose or in two or more doses. Such doses can be administered in any manner useful in directing the active compound(s) to the recipient's bloodstream, including orally, via implants, parenterally (including intravenous, intraperitoneal and subcutaneous injections), rectally, vaginally, and transdermally. Such administrations can be carried out using the compounds of the present teachings including pharmaceutically acceptable salts thereof, in lotions, creams, foams, patches, suspensions, solutions, and suppositories (rectal and vaginal).

When administered for the treatment or inhibition of a particular disease state or disorder, it is understood that an effective dosage can vary depending upon many factors such as the particular compound utilized, the mode of administration, and severity of the condition being treated, as well as the various physical factors related to the individual being treated. In therapeutic applications, a compound of the present teachings can be provided to a patient already suffering from a disease in an amount sufficient to cure or at least partially ameliorate the symptoms of the disease and its complications. The dosage to be used in the treatment of a specific individual typically must be subjectively determined by the attending physician. The variables involved include the specific condition and its state as well as the size, age and response pattern of the patient.

In some cases, for example those in which the lung is the targeted organ, it may be desirable to administer a compound directly to the airways of the patient, using devices such as metered dose inhalers, breath-operated inhalers, multidose dry-powder inhalers, pumps, squeeze-actuated nebulized spray dispensers, aerosol dispensers, and aerosol nebulizers. For administration by intranasal or intrabronchial inhalation, the compounds of the present teachings can be formulated into a liquid composition, a solid composition, or an aerosol composition. The liquid composition can include, by way of illustration, one or more compounds of the present teachings dissolved, partially dissolved, or suspended in one or more pharmaceutically acceptable solvents and can be administered by, for example, a pump or a squeeze-actuated nebulized spray dispenser. The solvents can be, for example, isotonic saline or bacteriostatic water. The solid composition can be, by way of illustration, a powder preparation including one or more compounds of the present teachings intermixed with lactose or other inert powders that are acceptable for intrabronchial use, and can be administered by, for example, an aerosol dispenser or a device that breaks or punctures a capsule encasing the solid composition and delivers the solid composition for inhalation. The aerosol composition can include, by way of illustration, one or more compounds of the present teachings, propellants, surfactants, and co-solvents, and can be administered by, for example, a metered device. The propellants can be a chlorofluorocarbon (CFC), a hydrofluoroalkane (HFA), or other propellants that are physiologically and environmentally acceptable.

Compounds described herein can be administered parenterally or intraperitoneally. Solutions or suspensions of these compounds and pharmaceutically acceptable salts, hydrates and esters thereof can be prepared in water suitably mixed with a surfactant such as hydroxyl-propylcellulose. Dispersions can also be prepared in glycerol, liquid polyethylene glycols, and mixtures thereof in oils. Under ordinary conditions of storage and use, these preparations typically contain a preservative to inhibit the growth of microorganisms.

The pharmaceutical forms suitable for injection can include sterile aqueous solutions or dispersions and sterile powders for the extemporaneous preparation of sterile injectable solutions or dispersions. In preferred embodiments, the form is sterile and its viscosity permits it to flow through a syringe. The form preferably is stable under the conditions of manufacture and storage and can be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), suitable mixtures thereof, and vegetable oils.

Compounds described herein can be administered transdermally, i.e., administered across the surface of the body and the inner linings of bodily passages including epithelial and mucosal tissues. Such administration can be carried out using the compounds of the present teachings including pharmaceutically acceptable salts, hydrates and esters thereof, in lotions, creams, foams, patches, suspensions, solutions, and suppositories (rectal and vaginal). Topical formulations that deliver active compound(s) through the epidermis can be useful for localized treatment of inflammation and arthritis.

Transdermal administration can be accomplished through the use of a transdermal patch containing an active compound and a carrier that can be inert to the active compound, can be non-toxic to the skin, and can allow delivery of the active compound for systemic absorption into the blood stream via the skin. The carrier can take any number of forms such as creams and ointments, pastes, gels, and occlusive devices. The creams and ointments can be viscous liquid or semisolid emulsions of either the oil-in-water or water-in-oil type. Pastes comprised of absorptive powders dispersed in petroleum or hydrophilic petroleum containing the active compound can also be suitable. A variety of occlusive devices can be used to release the active compound into the blood stream, such as a semi-permeable membrane covering a reservoir containing the active compound with or without a carrier, or a matrix containing the active compound. Other occlusive devices are known in the literature.

Compounds described herein can be administered rectally or vaginally in the form of a conventional suppository. Suppository formulations can be made from traditional materials, including cocoa butter, with or without the addition of waxes to alter the suppository's melting point, and glycerin. Water-soluble suppository bases, such as polyethylene glycols of various molecular weights, can also be used.

Lipid formulations or nanocapsules can be used to introduce compounds of the present teachings into host cells either in vitro or in vivo. Lipid formulations and nanocapsules can be prepared by methods known in the art.

To increase the effectiveness of compounds of the present teachings, it can be desirable to combine a compound with other agents effective in the treatment of the target disease. For inflammatory diseases, other active compounds (i.e., other active ingredients or agents) effective in their treatment, and particularly in the treatment of asthma and arthritis, can be administered with active compounds of the present teachings. The other agents can be administered at the same time or at different times than the compounds disclosed herein.

Throughout the description, where compositions are described as having, including, or comprising specific components, or where processes are described as having, including, or comprising specific process steps, it is contemplated that compositions of the present teachings also consist essentially of, or consist of, the recited components, and that the processes of the present teachings also consist essentially of, or consist of, the recited processing steps.

In the application, where an element or component is said to be included in and/or selected from a list of recited elements or components, it should be understood that the element or component can be any one of the recited elements or components and can be selected from a group consisting of two or more of the recited elements or components. The use of the term “include” should be generally understood as open-ended and non-limiting unless specifically stated otherwise.

The use of the singular herein includes the plural (and vice versa) unless specifically stated otherwise. In addition, where the use of the term “about” is before a quantitative value, the present teachings also include the specific quantitative value itself, unless specifically stated otherwise.

It should be understood that the order of steps or order for performing certain actions is immaterial so long as the present teachings remain operable. Moreover, two or more steps or actions may be conducted simultaneously.

As used herein, a “natural amino acid” refers to an amino acid normally occurring in natural proteins, e.g., L-α-amino acids. Examples of natural amino acids include glycine, alanine, valine, leucine, isoleucine, serine, threonine, cysteine, methionine, aspartic acid, asparagine, glutamic acid, glutamine, arginine, lysine, pyrrolysine, hydroxylysine, histidine, phenylalanine, tyrosine, tryptophan, proline, and 4-hydroxyproline.

As used herein, a “non-natural amino acid” refers to an amino acid that is not normally found in proteins. For example, a non-natural amino acid can refer to an epimer of a natural L-amino acid, i.e., an amino acid having the D-configuration; β-amino acids; an α-amino acid where the amino acid side chain of a natural amino acid has been shortened by one or two methylene groups or lengthened by up to 10 carbon atoms such as an α-amino alkanoic acid with 5 and up to and including 10 carbon atoms in a linear chain; an unsubstituted or substituted aromatic amino acid such as phenylglycine or a substituted phenylalanine; a cyclic amino acid other than the natural cyclic amino acids; and boron analogues where a backbone methylene group is replaced by a boron group, e.g., —BHR′—, where R′ is a side chain of a natural or non-natural amino acid. Examples of non-natural amino acids include β-alanine, taurine, α-aminobutyric acid, γ-aminoisobutyric acid, β-aminoisobutyricacid, homocysteine, homoserine, cysteinesulfinic acid, cysteic acid, felinine, isovalthine, 2,3-diaminosuccinic acid, γ-hydroxyglutamic acid, α-aminoadipic acid, α,ε-diaminopimelic acid, α,β-diaminopropionic acid, α,γ-diaminobutyric acid, ornithine, citulline, homocitrulline, saccharopine, azetidine-2-carboxylic acid, 3-hydroyproline, pipecolic acid, 5-hydroxytryptophan, 3,4-dihydroxyphenylalanine, monoiodotyrosine, 3,5-diiodotyrosine, 3,5,3′-triiodothyronine, thyroxine, and azaserine. A “non-natural amino acid” may also refer to a further derivatised natural or non-natural amino acid. For example, derivatisation may occur at the N- or C-terminus, i.e. at the amino or the carboxylic acid terminus, or on the amino acid substituent on the alpha carbon opposing the alpha-hydrogen. Examples of such chemical substituents include halogen, C₁-C₈ alkyl, trihalo(C₁-C₈)alkyl, C₁-C₈ acyl, thiol, sulfonic acid, sulfuric acid, sulfonate, sulfonamide, ester, amide, amine, amidine, phosphonic acid, phosphonate, boronic acid, and boronic ester. As used herein, an “N-linked natural amino acid” refers to a natural amino acid where its basic amino group is lacking an amine hydrogen, which is replaced by a covalent bond to another chemical entity. As used herein, an “N-linked non-natural amino acid” refers to a non-natural amino acid where the basic amino group lacks an amine hydrogen, and which is replaced by a covalent bond to another chemical entity.

As used herein, “free carboxyl” refers to a carboxylic acid group, C(O)OH, e.g., a free carboxyl natural amino acid refers to a natural amino acid having a carboxylic acid group at a terminal position. As used herein, “carboxyl-protected” refers to carboxylic acid group that is protected or blocked to prevent undesirable side reactions occurring with the carboxylic acid group. A carboxyl-protected molecule can be converted to a free carboxyl molecule under the appropriate conditions. The protection of amino and carboxylic acid groups is described in McOmie, Protecting Groups in Organic Chemistry, Plenum Press, NY, 1973, and Greene and Wuts, Protecting Groups in Organic Synthesis, for example page 41, 4nd. Ed., John Wiley & Sons, NY, 2006. Examples of carboxy protecting groups include C₁-C₆ alkyl groups such as methyl, ethyl, t-butyl and t-amyl; aryl(C₁-C₄)alkyl groups such as benzyl, 4-nitrobenzyl, 4-methoxybenzyl, 3,4-dimethoxybenzyl, 2,4-dimethoxybenzyl, 2,4,6-trimethoxybenzyl, 2,4,6-trimethylbenzyl, benzhydryl and trityl; silyl groups such as trimethylsilyl and t-butyldimethylsilyl; and allyl groups such as allyl and 1-(trimethylsilylmethyl)prop-1-en-3-yl. Examples of amine protecting groups (PG) include acyl groups, such as groups of formula RCO in which R represents C₁-C₆ alkyl, C₃-C₁₀ cycloalkyl, phenyl C₁-C₆ alkyl, phenyl, C₁-C₆ alkoxy, phenyl C₁-C₆ alkoxy, or a C₃-C₁₀ cycloalkoxy, wherein a phenyl group may be optionally substituted, for example by one or two of halogen, C₁-C₄alkyl and C₁-C₄ alkoxy.

As used herein, the “tricyclic core” of compounds of formula I refers to:

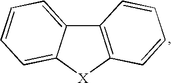

where X is as defined herein.

As used herein, “halo” or “halogen” refers to fluoro, chloro, bromo, and iodo.

As used herein, “oxo” refers to a double-bonded oxygen (i.e. “═O”).

As used herein, “alkyl” refers to a straight-chain or branched saturated hydrocarbon group. In some embodiments, an alkyl group can have from 1 to 10 carbon atoms (e.g, from 1 to 6 carbon atoms). Examples of alkyl groups include methyl (Me), ethyl (Et), propyl (e.g., n-propyl and isopropyl), butyl (e.g., n-butyl, isobutyl, s-butyl, t-butyl), pentyl (e.g., n-pentyl, isopentyl, neopentyl), and the like. In some embodiments, alkyl groups can be substituted with up to four substituents independently selected from —Z—R⁹ or —Z—R¹² groups, where R⁹, R¹², and Z are as defined herein. A lower alkyl group typically has up to 4 carbon atoms. Examples of lower alkyl groups include methyl, ethyl, propyl (e.g., n-propyl and isopropyl), and butyl groups (e.g., n-butyl, isobutyl, s-butyl, t-butyl).

As used herein, “alkenyl” refers to a straight-chain or branched alkyl group having one or more carbon-carbon double bonds. In some embodiments, an alkenyl group can have from 2 to 10 carbon atoms (e.g., from 2 to 6 carbon atoms). Examples of alkenyl groups include ethenyl, propenyl, butenyl, pentenyl, hexenyl, butadienyl, pentadienyl, hexadienyl groups, and the like. The one or more carbon-carbon double bonds can be internal (such as in 2-butene) or terminal (such as in 1-butene). In some embodiments, alkenyl groups can be substituted with up to four substituents independently selected from —Z—R⁹ or —Z—R¹² groups, where R⁹, R¹², and Z are as defined herein.

As used herein, “alkynyl” refers to a straight-chain or branched alkyl group having one or more carbon-carbon triple bonds. In some embodiments, an alkynyl group can have from 2 to 10 carbon atoms (e.g., from 2 to 6 carbon atoms). Examples of alkynyl groups include ethynyl, propynyl, butynyl, pentynyl, and the like. The one or more carbon-carbon triple bonds can be internal (such as in 2-butyne) or terminal (such as in 1-butyne). In some embodiments, alkynyl groups can be substituted with up to four substituents independently selected from —Z—R⁹ or —Z—R¹² groups, where R⁹, R¹², and Z are as defined herein.

As used herein, “alkoxy” refers to an —O-alkyl group. In some embodiments, an alkoxy group can have from 1 to 10 carbon atoms (e.g., from 1 to 6 carbon atoms). Examples of alkoxy groups include methoxy, ethoxy, propoxy (e.g., n-propoxy and isopropoxy), t-butoxy, and the like.

As used herein, “alkylthio” refers to an —S-alkyl group. In some embodiments, an alkylthio group can have from 1 to 10 carbon atoms (e.g., from 1 to 6 carbon atoms). Examples of alkylthio groups include methylthio, ethylthio, propylthio (e.g., n-propylthio and isopropylthio), t-butylthio, and the like.

As used herein, “acyl” refers to an —C(O)-alkyl group. In some embodiments, the alkyl group in an acyl group can have from 1 to 10 carbon atoms (e.g., from 1 to 6 carbon atoms). Examples of acyl groups include —C(O)CH₃, —C(O)CH₂CH₃, and the like.

As used herein, “haloalkyl” refers to an alkyl group having one or more halogen substituents. In some embodiments, a haloalkyl group can have from 1 to 10 carbon atoms (e.g., from 1 to 6 carbon atoms). Examples of haloalkyl groups include CF₃, C₂F₅, CHF₂, CH₂F, CCl₃, CHCl₂, CH₂C₁, C₂Cl₅, and the like. Perhaloalkyl groups, i.e., alkyl groups wherein all of the hydrogen atoms are replaced with halogen atoms (e.g., CF₃ and C₂F₅), are included within the definition of “haloalkyl.”

As used herein, “cycloalkyl” refers to a non-aromatic carbocyclic group that may be optionally fused to an aromatic moiety such as aryl or heteroaryl. The carbocyclic group may include cyclized alkyl, alkenyl, and alkynyl groups. A cycloalkyl group can be monocyclic (e.g., cyclohexyl) or polycyclic (e.g., containing fused, bridged, and/or spiro ring systems), wherein the carbon atoms are located inside or outside of the ring system. A cycloalkyl group, as a whole, can have from 3 to 14 ring atoms (e.g., from 3 to 8 carbon atoms for a monocyclic cycloalkyl group and from 7 to 14 carbon atoms for a polycyclic cycloalkyl group). Any suitable ring position of the cycloalkyl group can be covalently linked to the defined chemical structure. Examples of cycloalkyl groups include cyclopropyl, cyclopropylmethyl, cyclobutyl, cyclopentyl, cyclohexyl, cyclohexylmethyl, cyclohexylethyl, cycloheptyl, cyclopentenyl, cyclohexenyl, cyclohexadienyl, cycloheptatrienyl, norbornyl, norpinyl, norcaryl, adamantyl, and spiro[4.5]decanyl, as well as their homologs, isomers, and the like. In some embodiments, cycloalkyl groups can be substituted with up to four substituents independently selected from —Z—R⁹ or —Z—R¹² groups, where R⁹, R¹², and Z are as defined herein. For example, cycloalkyl groups can include 1-3 “oxo” groups, wherein an “oxo” group is where two R⁹ or R¹² groups attached to a single carbon atom may be replaced by the “oxo” group at the carbon atom.

As used herein, “heteroatom” refers to an atom of any element other than carbon or hydrogen and includes, for example, nitrogen, oxygen, sulfur, phosphorus, and selenium.

As used herein, “cycloheteroalkyl” refers to a non-aromatic cycloalkyl group that contains at least one (e.g., one, two, three, four or five ring heteratoms) ring heteroatom selected from O, N and S, and optionally contains one or more (e.g., one, two, or three) double or triple bonds. A cycloheteroalkyl group, as a whole, can have, for example, from 3 to 14 ring atoms and contains from 1 to 5 ring heteroatoms (e.g., from 3-6 ring atoms for a monocyclic cycloheteroalkyl group and from 7 to 14 ring atoms for a polycyclic cycloheteroalkyl group), and may be partially aromatic. One or more N or S atoms in a cycloheteroalkyl ring may be oxidized (e.g., morpholine N-oxide, thiomorpholine S-oxide, thiomorpholine S,S-dioxide). In some embodiments, nitrogen atoms of cycloheteroalkyl groups can bear a substituent, for example, a —Z—R⁹ group or a —Z—R¹² group, where R⁹, R¹², and Z are as defined herein. Cycloheteroalkyl groups can also contain one or more oxo groups, such as phthalimidyl, piperidonyl, oxazolidinonyl, 2,4(1H,3H)-dioxo-pyrimidinyl, pyridin-2(1H)-onyl, 1,3-oxazinane-2-one, morpholin-2-one, morpholin-3-one and the like. Examples of cycloheteroalkyl groups include, among others, morpholinyl, thiomorpholinyl, pyranyl, imidazolidinyl, imidazolinyl, oxazolidinyl, pyrazolidinyl, pyrazolinyl, pyrrolidinyl, pyrrolinyl, tetrahydrofuranyl, tetrahydrothienyl, piperidinyl, piperazinyl, and the like. In some embodiments, cycloheteroalkyl groups can be optionally substituted with up to four substituents independently selected from —Z—R⁹ or —Z—R¹² groups, where R⁹, R¹², and Z are as defined herein. In some embodiments, cycloheteroalkyl groups may be optionally fused to 1-2 cycloalkyl, cycloheteroalkyl, aryl or heteroaryl rings, for example, dihydrobenzofuran, dihydrobenzothiophene, indoline, benzo-oxazinone.

As used herein, “aryl” refers to an aromatic monocyclic hydrocarbon ring system or a polycyclic ring system where at least one of the rings present in the ring system is an aromatic hydrocarbon ring and any other aromatic rings present in the ring system include only hydrocarbons. An aryl group can have from 6 to 14 carbon atoms in its ring system, which can include multiple fused rings. In some embodiments, a polycyclic aryl group can have from 8 to 14 carbon atoms. Any suitable ring position of the aryl group can be covalently linked to the defined chemical structure. In some embodiments, an aryl group can have only aromatic carbocyclic rings e.g., phenyl, 1-naphthyl, 2-naphthyl, anthracenyl, phenanthrenyl groups, and the like. In other embodiments, an aryl group can be a polycyclic ring system in which at least one aromatic carbocyclic ring is fused (i.e., having a bond in common with) to one or more cycloalkyl or cycloheteroalkyl rings. Examples of such aryl groups include, among others, benzo derivatives of cyclopentane (i.e., an indanyl group, which is a 5,6-bicyclic cycloalkyl/aromatic ring system), cyclohexane (i.e., a tetrahydronaphthyl group, which is a 6,6-bicyclic cycloalkyl/aromatic ring system), imidazoline (i.e., a benzimidazolinyl group, which is a 5,6-bicyclic cycloheteroalkyl/aromatic ring system), and pyran (i.e., a chromenyl group, which is a 6,6-bicyclic cycloheteroalkyl/aromatic ring system). Other examples of aryl groups include 2,4-dihydro-1H-benzo[d][1,3]oxazinyl, benzodioxanyl, benzodioxolyl, chromanyl, indolinyl groups, and the like. In some embodiments, aryl groups optionally contain up to four substituents independently selected from —Z—R⁹ or —Z—R¹² groups, where R⁹, R¹², and Z are as defined herein.

As used herein, “heteroaryl” refers to an aromatic monocyclic ring system containing at least 1 ring heteroatom selected from oxygen (O), nitrogen (N) and sulfur (S) or a polycyclic ring system where at least one of the rings present in the ring system is aromatic and contains at least 1 ring heteroatom. A heteroaryl group, as a whole, can have, for example, from 5 to 14 ring atoms and contain 1-4 ring heteroatoms. Heteroaryl groups include monocyclic heteroaryl rings fused to one or more aromatic carbocyclic rings, non-aromatic carbocyclic rings, and non-aromatic cycloheteroalkyl rings. The heteroaryl group can be attached to the defined chemical structure at any heteroatom or carbon atom that results in a stable structure. Generally, heteroaryl rings do not contain O—O, S—S, or S—O bonds. However, one or more N or S atoms in a heteroaryl group can be oxidized (e.g., pyridine N-oxide, thiophene S-oxide, thiophene S,S-dioxide). Examples of heteroaryl groups include, for example, the 5-membered monocyclic and 5-6 bicyclic ring systems shown below:

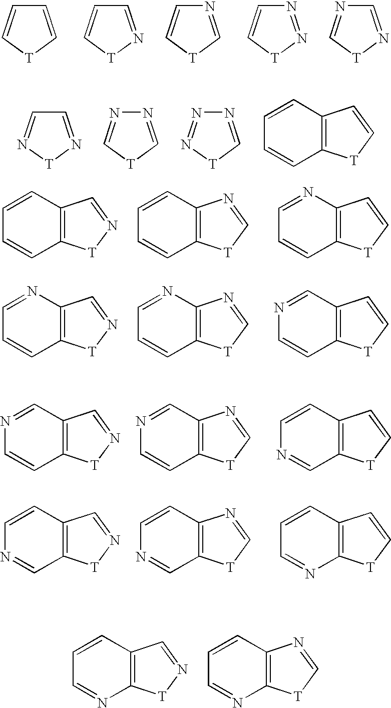

wherein T is O, S, NH, N—Z—R⁹, or N—Z—R¹², and R⁹, R¹², and Z are as defined herein. Examples of such heteroaryl rings include pyrrolyl, furyl, thienyl, pyridyl, pyrimidyl, pyridazinyl, pyrazinyl, triazolyl, tetrazolyl, pyrazolyl, imidazolyl, isothiazolyl, thiazolyl, thiadiazolyl, isoxazolyl, oxazolyl, oxadiazolyl, indolyl, isoindolyl, benzofuryl, benzothienyl, quinolyl, 2-methylquinolyl, isoquinolyl, quinoxalyl, quinazolyl, benzotriazolyl, benzimidazolyl, benzothiazolyl, benzisothiazolyl, benzisoxazolyl, benzoxadiazolyl, benzoxazolyl, cinnolinyl, 1H-indazolyl, 2H-indazolyl, indolizinyl, isobenzofuyl, naphthyridinyl, phthalazinyl, pteridinyl, purinyl, oxazolopyridinyl, thiazolopyridinyl, imidazopyridinyl, furopyridinyl, thienopyridinyl, pyridopyrimidinyl, pyridopyrazinyl, pyridopyridazinyl, thienothiazolyl, thienoxazolyl, thienoimidazolyl groups, and the like. Further examples of heteroaryl groups include 4,5,6,7-tetrahydroindolyl, tetrahydroquinolinyl, benzothienopyridinyl, benzofuropyridinyl groups, and the like. In some embodiments, heteroaryl groups can be substituted with up to four substituents independently selected from —Z—R⁹ or —Z—R¹² groups, wherein R⁹, R¹², and Z are as defined herein.

The compounds of the present teachings can include a “divalent group” defined herein as a linking group capable of forming a covalent bond with two other moieties. For example, compounds described herein can include a divalent C₁₋₁₀ alkyl group, such as, for example, a methylene group.

At various places in the present specification, substituents of compounds are disclosed in groups or in ranges. It is specifically intended that the description include each and every individual subcombination of the members of such groups and ranges. For example, the term “C₁₋₁₀ alkyl” is specifically intended to individually disclose C₁, C₂, C₃, C₄, C₅, C₆, C₇, C₈, C₉, C₁₀, C₁-C₁₀, C₁-C₉, C₁-C₈, C₁-C₇, C₁-C₆, C₁-C₅, C₁-C₄, C₁-C₃, C₁-C₂, C₂-C₁₀, C₂-C₉, C₂-C₈, C₂-C₇, C₂-C₆, C₂-C₅, C₂-C₄, C₂-C₃, C₃-C₁₀, C₃-C₉, C₃-C₈, C₃-C₇, C₃-C₆, C₃-C₅, C₃-C₄, C₄-C₁₀, C₄-C₉, C₄-C₈, C₄-C₇, C₄-C₆, C₄-C₅, C₅-C₁₀, C₅-C₉, C₅-C₈, C₅-C₇, C₅-C₆, C₆-C₁₀, C₆-C₉, C₆-C₈, C₆-C₇, C₇-C₁₀, C₇-C₉, C₇-C₈, C₈-C₁₀, C₈-C₉, and C₉-C₁₀ alkyl. By way of other examples, the term “5-14 membered heteroaryl group” is specifically intended to individually disclose a heteroaryl group having 5, 6, 7, 8, 9, 10, 11, 12, 13, 14, 5-14, 5-13, 5-12, 5-11, 5-10, 5-9, 5-8, 5-7, 5-6, 6-14, 6-13, 6-12, 6-11, 6-10, 6-9, 6-8, 6-7, 7-14, 7-13, 7-12, 7-11, 7-10, 7-9, 7-8, 8-14, 8-13, 8-12, 8-11, 8-10, 8-9, 9-14, 9-13, 9-12, 9-11, 9-10, 10-14, 10-13, 10-12, 10-11, 11-14, 11-13, 11-12, 12-14, 12-13, or 13-14 ring atoms; and the phrase “optionally substituted with 1-4 substituents” is specifically intended to individually disclose a chemical group that can include 0, 1, 2, 3, 4, 0-4,0-3, 0-2,0-1, 1-4, 1-3, 1-2, 2-4, 2-3, and 3-4 substituents. It is to be understood that substitution includes cyclic moieties such as cycloalkyl, cycloalkenyl, cycloheteroalkyl, aryl and heteroaryl wherein the cyclic moiety may be fused to a parent ring, where appropriate. Examples where the parent ring is an aryl ring include benzocycloalkyl, benzocycloalkenyl, benzocycloheteroalkyl, benzoaryl and benzoheteroaryl.

A chiral center is commonly, a carbon atom that contains four different groups attached to it. Compounds described herein can contain a chiral center with some of the compounds containing one or more asymmetric atoms or centers, giving rise to optical isomers (enantiomers) and diastereomers. The present teachings and compounds disclosed herein include such optical isomers (enantiomers) and diastereomers (geometric isomers), as well as the racemic and resolved, enantiomerically pure stereoisomers, as well as other mixtures of the R and S stereoisomers and pharmaceutically acceptable salts thereof. Optical isomers can be obtained in pure form by standard procedures known to those skilled in the art, which include diastereomeric salt formation and separation, kinetic resolution, and asymmetric synthesis. The present teachings also encompass cis and trans isomers of compounds containing alkenyl moieties (e.g., alkenes and imines). It is also understood that the present teachings encompass all possible regioisomers, and mixtures thereof, which can be obtained in pure form by standard separation procedures known to those skilled in the art, and include column chromatography, thin-layer chromatography, and high-performance liquid chromatography.

The compounds of the present teachings can be prepared in accordance with the procedures described below, from commercially available starting materials, compounds known in the literature, or readily prepared intermediates, by employing standard synthetic methods and procedures known to those skilled in the art. Standard synthetic methods and procedures for the preparation of organic molecules and functional group transformations and manipulations can be readily obtained from the relevant scientific literature or from standard textbooks in the field. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvent used, but such conditions can be determined by one skilled in the art by routine optimization procedures. Those skilled in the art of organic synthesis will recognize that the nature and order of the synthetic steps presented may be varied for the purpose of optimizing the formation of the compounds described herein.

The processes described herein can be monitored according to any suitable method known in the art. For example, product formation can be monitored by spectroscopic means, such as nuclear magnetic resonance spectroscopy (e.g., ¹H or ¹³C), infrared spectroscopy, spectrophotometry (e.g., UV-visible), or mass spectrometry, and/or by chromatography such as high performance liquid chromatograpy (HPLC) or thin layer chromatography.

Preparation of Compounds can Involve the Protection and Deprotection of Various chemical groups. The need for protection and deprotection and the selection of appropriate protecting groups can be readily determined by one skilled in the art. The chemistry of protecting groups can be found, for example, in Greene, et al., Protective Groups in Organic Synthesis, 4th Ed., Wiley & Sons, 2006, the entire disclosure of which is incorporated by reference herein for all purposes.

The reactions of the processes described herein can be carried out in suitable solvents which can be readily selected by one skilled in the art of organic synthesis.

Suitable solvents typically are substantially nonreactive with the reactants, intermediates, and/or products at the temperatures at which the reactions are carried out, i.e., temperatures that can range from the solvent's freezing temperature to the solvent's boiling temperature. A given reaction can be carried out in one solvent or a mixture of more than one solvent. Depending on the particular reaction step, suitable solvents for a particular reaction step can be selected.

The following examples illustrate various synthetic routes which can be used to prepare compounds of formula I.

### Example 1

### (S)-2-(8-(furan-3-yl)dibenzo[b,d]furan-3-sulfonamido)-3-methylbutanoic acid (Compound 7)

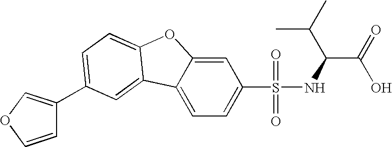

### Step 1: Preparation of 8-bromodibenzo[b,d]furan-3-sulfonyl chloride

Dibenzo[b,d]furan-3-sulfonyl chloride (5.3 g, 20 mmol, 1.0 eq.) was mixed with acetic acid (glacial, 120 mL) and bromine (10 mL, 10 eq.) and the resulting mixture was heated at 70° C. for 4 hours. The excess bromine was removed by bubbling nitrogen through the reaction mixture and trapped with saturated sodium sulfite (Na₂SO₃) solution. After cooled to room temperature, the mixture was filtered to produce 8-bromodibenzo[b,d]furan-3-sulfonyl chloride (5.4 g) as a light brown solid.

### Step 2: Preparation of (S)-methyl 2-(8-bromodibenzo[b,d]furan-3-sulfonamido)-3-methylbutanoate

8-Bromodibenzo[b,d]furan-3-sulfonyl chloride (3.46 g, 10 mmol) and (S)-methyl 2-amino-3-methylbutanoate hydrochloride (1.1 eq.) were mixed in 30 mL of methylene chloride (DCM), to which N,N-diisopropylethylamine (3.84 mL, 2.2 eq.) was added. The mixture was stirred at room temperature for 5 hours and the crude product was purified by silica gel column chromatography to produce (S)-methyl 2-(8-bromodibenzo[b,d]furan-3-sulfonamido)-3-methylbutanoate (4.7 g) as a white solid.

### Step 3: Preparation of methyl N-{[8-(3-furyl)dibenzo[b,d]furan-3-yl]sulfonyl}-L-valinate

(S)-Methyl 2-(8-bromodibenzo[b,d]furan-3-sulfonamido)-3-methylbutanoate 240 mg, 0.5 mmol), K₂CO₃ (242 mg, 3.5 eq.), 3-furanboronic acid (140 mg, 1.25 mmol), and palladium tetrakis(triphenylphosphine) (Pd(PPh₃)₄, 60 mg) were mixed in 3 mL of dimethoxyethane (DME) and 0.5 mL of water. The mixture was deoxygenated with nitrogen and stirred at 85° C. for 4 hours. Brine was added to the reaction and the resulting mixture was extracted with ethyl acetate (EtOAc). Removal of the solvent gave crude product, which was purified by column chromatography to produce methyl N-{[8-(3-furyl)dibenzo[b,d]furan-3-yl]sulfonyl}-L-valinate (200 mg) as a white solid.

### Step 4: Preparation of (S)-2-(8-(furan-3-yl)dibenzo[b,d]furan-3-sulfonamido)-3-methylbutanoic acid

(S)-Methyl 2-(8-(furan-3-yl)dibenzo[b,d]furan-3-sulfonamido)-3-methylbutanoate (200 mg) was dissolved in 4 mL of tetrahydrofuran (THF). Lithium hydroxide (LiOH, 200 mg) was added and the resulting suspension was heated at the reflux temperature for 6 hours. Acidic aqueous work-up afforded (S)-2-(8-(furan-3-yl)dibenzo[b,d]furan-3-sulfonamido)-3-methylbutanoic acid (165 mg) as a white powder. ¹H NMR (CDCl₃): δ 0.85 (d, J=6.6 Hz, 3H), 0.96 (d, J=6.9 Hz, 3H), 2.08 (m, 1H), 3.74 (dd, J=9.4, 4.4 Hz, 1H), 5.47 (d, J=9.4 Hz, 1H), 6.76 (dd, J=1.9, 0.6 Hz, 1H), 7.50 (dd, J=1.6, 1.6 Hz, 1H), 7.61 (dd, J=8.2, 1.6 Hz, 1H), 7.83 (dd, J=1.3, 1.3 Hz, 1H), 7.88 (dd, J=8.5, 1.6 Hz, 1H), 7.95 (d, J=1.3 Hz, 1H), 8.14 (d, J=8.2 Hz, 1H), 8.17 (d, J=7.8 Hz, 1H), 8.32 (d, J=1.3 Hz, 1H). High-resolution mass spectroscopy (HRMS, ESI-FTMS): calculated for C₂₁H₁₉NO₆S+H⁺: 414.10059. found: 414.1006.

### Example 1A

### (2S)-3-methyl-2-(8-(1-(2-methylbutyl)-1H-pyrazol-4-yl)dibenzo[b,d]furan-3-sulfonamido)butanoic acid (Compound 157)

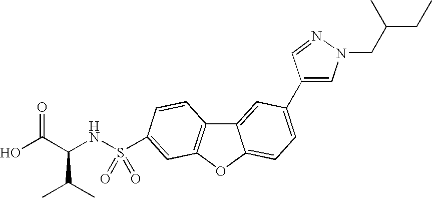

The title compound was prepared by the procedures described in Example 1, using 1-(2-methylbutyl)-4-(4,4,5,5-tetramethyl-1,3,2-dioxaborolan-2-yl)-1H-pyrazole-1-(2-morpholinoethyl)-1H-pyrazol-4-ylboronate instead of 3-furanboronic acid. The compound was obtained as an off-white solid. ¹H NMR (400 MHz, MeOD) δ ppm 0.91 (d, J=6.82 Hz, 3H), 0.96-1.03 (m, 9H), 1.59-1.70 (m, 1H), 1.79-1.88 (m, 2H), 2.03-2.15 (m, 1H), 3.78 (d, J=5.31 Hz, 1H), 4.18-4.25 (m, 2H), 7.59-7.65 (m, 1H), 7.68-7.73 (m, 1H), 7.83-7.90 (m, 3H), 8.07-8.16 (m, 3H). HRMS (ESI-FTMS): calcd for C₂₅H₂₉N₃O₅S+H⁺, 484.19007. found: 484.19134.

### Example 1B

### (S)-3-methyl-2-(8-(1-(2-morpholinoethyl)-1H-pyrazol-4-yl)dibenzo[b,d]furan-3-sulfonamido)butanoic acid (Compound 158)

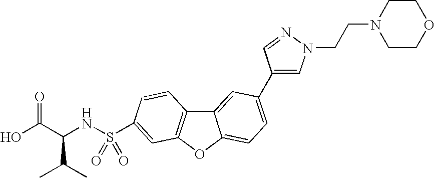

The title compound was prepared by the procedures described in Example 1, using 1-(2-morpholinoethyl)-1H-pyrazol-4-ylboronic acid instead of 3-furanboronic acid. The compound was obtained as an off-white solid. ¹H NMR (400 MHz, DMSO-d₆) δ ppm 0.83 (d, J=6.57 Hz, 3H), 0.92 (d, J=6.57 Hz, 3H), 1.97-2.11 (m, 1H), 2.42-2.51 (m, 4H), 2.82 (t, J=6.69 Hz, 2H), 3.34-3.43 (m, 1H), 3.59-3.65 (m, 4H), 4.29 (t, J=6.69 Hz, 2H), 7.65-7.69 (m, 1H), 7.72-7.78 (m, 1H), 7.82 (dd, J=7.96, 1.39 Hz, 1H), 7.90 (s, 1H), 8.01-8.05 (m, 1H), 8.14 (s, 1H), 8.21 (d, J=8.59 Hz, 1H), 8.30-8.34 (m, 1H). HRMS (ESI-FTMS): calcd for C₂₆H₃₀N₄O₆S+H⁺, 527.19588. found: 527.19814.

### Example 1C

### (S)-2-(8-(1-isobutyl-1H-pyrazol-4-yl)dibenzo[b,d]furan-3-sulfonamido)-3-methylbutanoic acid (Compound 159)

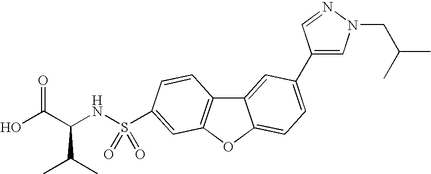

The title compound was prepared by the procedures described in Example 1, using 1-isobutyl-4-(4,4,5,5-tetramethyl-1,3,2-dioxaborolan-2-yl)-1H-pyrazole instead of 3-furan boronic acid. The compound was obtained as an off-white solid. ¹H NMR (400 MHz, MeOD) δ ppm 0.91 (d, J=6.82 Hz, 3H), 0.96-1.04 (m, 9H), 2.05-2.15 (m, 1H), 2.20-2.34 (m, 1H), 3.78 (d, J=5.31 Hz, 1H), 4.00 (d, J=7.33 Hz, 2H), 7.60-7.74 (m, 2H), 7.79-7.92 (m, 3H), 8.06-8.16 (m, 3H). HRMS (ESI-FTMS): calcd for C₂₄H₂₇N₃O₅S+H⁺, 470.17442. found: 470.17594.

### Example 1D

### (S)-3-methyl-2-(8-(1,3,5-trimethyl-1H-pyrazol-4-yl)dibenzo[b,d]furan-3-sulfonamido)butanoic acid (Compound 160)

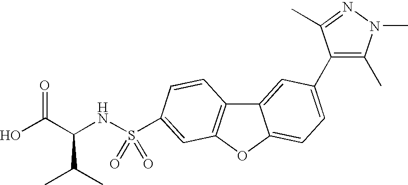

The title compound was prepared by the procedures described in Example 1, using 1,3,5-trimethyl-4-(4,4,5,5-tetramethyl-1,3,2-dioxaborolan-2-yl)-1H-pyrazole instead of 3-furanboronic acid. The compound was obtained as an off-white solid. ¹H NMR (400 MHz, MeOD) δ ppm 0.92 (d, J=6.82 Hz, 3H), 1.00 (d, J=6.82 Hz, 3H), 2.02-2.17 (m, 1H), 2.26 (s, 3H), 2.31 (s, 3H), 3.78 (d, J=5.05 Hz, 1H), 3.84 (s, 3H), 7.43 (dd, J=8.59, 1.77 Hz, 1H), 7.69 (d, J=8.59 Hz, 1H), 7.83-7.91 (m, 2H), 8.11 (dd, J=4.67, 3.16 Hz, 2H). HRMS (ESI-FTMS): calcd for C₂₃H₂₅N₃O₅S+H⁺, 456.15877. found: 456.16006.

### Example 1E

### (S)-3-methyl-2-(8-(5-methyl-3-phenylisoxazol-4-yl)dibenzo[b,d]furan-3-sulfonamido)butanoic acid (Compound 161)

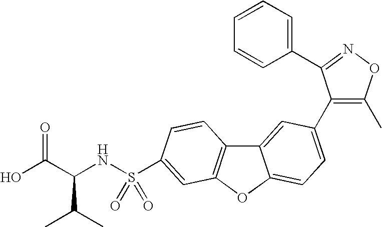

The title compound was prepared by the procedures described in Example 1, using 5-methyl-3-phenylisoxazol-4-ylboronic acid instead of 3-furanboronic acid. The compound was obtained as an off-white solid. ¹H NMR (400 MHz, MeOD) δ ppm 0.91 (d, J=6.82 Hz, 3H), 0.99 (d, J=6.82 Hz, 3H), 2.03-2.16 (m, 1H), 2.52 (s, 3H), 3.76 (d, J=5.05 Hz, 1H), 7.27-7.45 (m, 7H), 7.65 (d, J=8.59 Hz, 1H), 7.82-7.91 (m, 2H), 8.00-8.07 (m, 1H), 8.10-8.15 (m, 1H). HRMS (ESI-FTMS): calcd for C₂₇H₂₄N₂O₆S+H⁺, 505.14278. found: 505.1448.

### Example 1F

### (S)-3-methyl-2-(8-(5-methyl-1-phenyl-1H-pyrazol-4-yl)dibenzo[b,d]furan-3-sulfonamido)butanoic acid (Compound 162)

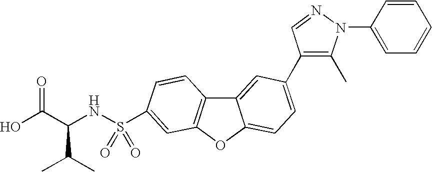

The title compound was prepared by the procedures described in Example 1, using 5-methyl-1-phenyl-4-(4,4,5,5-tetramethyl-1,3,2-dioxaborolan-2-yl)-1H-pyrazole instead of 3-furanboronic acid. The compound was obtained as an off-white solid. ¹H NMR (400 MHz, MeOD) δ ppm 0.91 (d, J=6.82 Hz, 3H), 0.97 (d, J=6.82 Hz, 3H), 2.03-2.14 (m, 1H), 2.49 (s, 3H), 3.74 (d, J=5.56 Hz, 1H), 7.45-7.60 (m, 5H), 7.64-7.69 (m, 1H), 7.70-7.75 (m, 1H), 7.89 (dd, J=8.34, 1.52 Hz, 1H), 8.12 (dd, J=9.60, 1.26 Hz, 2H), 8.18 (d, J=8.08 Hz, 1H). HRMS: calcd for C₂₇H₂₅N₃O₅S+H⁺, 504.15877. found: 504.16076.

### Example 1G

### (S)-3-methyl-2-(8-(4-methyl-2-phenylthiazol-5-yl)dibenzo[b,d]furan-3-sulfonamido)butanoic acid (Compound 163)

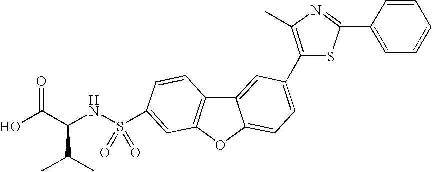

The title compound was prepared by the procedures described in Example 1, using 4-methyl-2-phenyl-5-(4,4,5,5-tetramethyl-1,3,2-dioxaborolan-2-yl)thiazole instead of 3-furanboronic acid. The compound was obtained as an off-white solid. ¹H NMR (400 MHz, MeOD) δ ppm 0.91 (d, J=6.82 Hz, 3H), 0.99 (d, J=6.82 Hz, 3H), 2.06-2.17 (m, 1H), 2.59 (s, 3H), 3.80 (d, J=5.05 Hz, 1H), 7.45-7.52 (m, 3H), 7.66-7.75 (m, 2H), 7.87-7.97 (m, 3H), 8.11-8.17 (m, 3H). HRMS: calcd for C₂₇H₂₄N₂O₅S₂+H⁺, 521.11994. found: 521.12182.

### Example 1H

### (S)-3-methyl-2-(8-(4-methyl-2-(4-(trifluoromethyl)phenyl)thiazol-5-yl)dibenzo[b,d]furan-3-sulfonamido)butanoic acid (Compound 164)

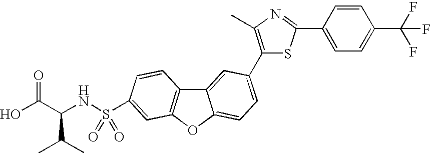

The title compound was prepared by the procedures described in Example 1, using 4-methyl-5-(4,4,5,5-tetramethyl-1,3,2-dioxaborolan-2-yl)-2-(4-(trifluoromethyl)phenyl)thiazole instead of 3-furanboronic acid. The compound was obtained as an off-white solid. ¹H NMR (400 MHz, MeOD) δ ppm 0.92 (d, J=6.82 Hz, 3H), 1.00 (d, J=6.82 Hz, 3H), 2.01-2.21 (m, 1H), 2.65 (s, 3H), 3.75 (d, J=5.05 Hz, 1H), 7.56-7.63 (m, 1H), 7.67-7.83 (m, 4H), 8.03 (dd, J=8.59, 2.02 Hz, 1H), 8.07-8.20 (m, 3H), 8.52-8.60 (m, 1H). HRMS (ESI-FTMS): calcd for C₂₈H₂₃F₃N₂O₅S₂+H⁺, 589.10732. found: 589.10815.

The following compounds in Table 2 were prepared using procedures analogous to those described above for the preparation of (S)-2-(8-(furan-3-yl)dibenzo[b,d]furan-3-sulfonamido)-3-methylbutanoic acid.

TABLE 2

| Compd | | | |
|---|---|---|---|
| No. | NMR | HRMS | MS |
| 5 | — | 444.0936 | — |
| 28 | — | 430.0774 | — |
| 29 | — | 430.0771 | — |
| 30 | — | — | 442.1 |
| 31 | — | — | 458.1 |
| 32 | — | 472.0879 | — |
| 33 | — | 444.0926 | — |
| 34 | — | 464.0381 | — |
| 35 | — | 456.1835 | — |
| 37 | ¹H NMR (DMSO-d6): δ 0.81 (d, J = 6.6 Hz, 3 H), 0.84 (d, J = 6.6 Hz, | — | 414.1 |
| | 3 H), 1.95 (m, 1H), 3.59 (m, 1H), 6.64 (dd, J = 3.2 and 1.6 Hz, | | |
| | 1H), 7.03 (dd, J = 3.2 and 0.9 Hz, 1H), 7.87-7.79 (m, 3 H), 7.97 (dd, | | |
| | J = 8.8 and 1.9 Hz, 1H), 8.07 (d, J = 1.3 Hz, 1H), 8.11 (m br, 1H), | | |
| | 8.40 (d, J = 8.2 Hz, 1H), 8.58 (d, J = 1.3 Hz, 1H), and 12.48 (s br, | | |
| | 1H). | | |
| 59 | — | 536.1016 | — |
| 66 | — | 388.12014 | — |
| 67 | — | 388.12018 | — |
| 69 | — | 414.13601 | — |
| 70 | — | — | 429.3 |
| 72 | — | 428.11603 | — |
| 89 | — | — | 458.1 |
| 94 | — | 448.06254 | — |
| 152 | — | 426.1129 | — |
| 153 | — | 456.1237 | — |

### Example 1I

### (S)-2-(8-(benzo[d][1,3]dioxol-5-yl)dibenzo[b,d]furan-3-sulfonamido)-3-methylbutanoic acid (Compound 308)

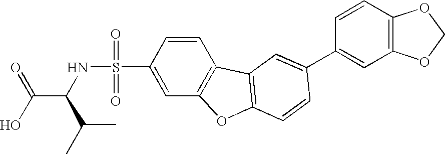

The title compound was prepared by the procedures described in Example 1, using benzo[d][1,3]dioxol-5-ylboronic acid instead of 3-furanboronic acid. The compound was obtained as a white solid (92%). ¹H NMR (DMSO-d6): 12.48 (s br, 1H); 8.52 (d, J=1.3 Hz, 1H); 8.37 (d, J=8.2 Hz, 1H); 8.11 (m, 1H); 8.07 (d, J=1.3 Hz, 1H); 7.86 (dd, J=8.8, 1.9 Hz, 1H); 7.82 (m, 1H); 7.81 (d, J=8.8 Hz, 1H); 7.38 (d, J=1.9 Hz, 1H); 7.27 (dd, J=8.2, 1.9 Hz, 1H); 7.05 (d, J=8.2 Hz, 1H). MS (ES⁻): 466.1.

### Example 1J

### (S)-3-methyl-2-(8-phenyldibenzo[b,d]furan-3-sulfonamido)butanoic acid (Compound 309)

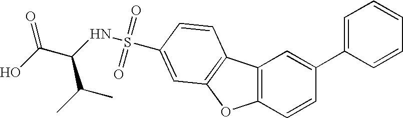

The title compound was prepared by the procedures described in Example 1, using phenylboronic acid instead of 3-furanboronic acid. The compound was obtained as a white solid. ¹H NMR (CDCl₃): 8.18 (m, 1H); 8.08 (m, 2H); 7.88-7.76 (m, 2H); 7.72-7.62 (m, 3H); 7.69 (m, 2H); 7.39 (m, 2H); 5.11 (d, J=10.1, 1H); 3.87 (dd, J=10.1, 4.7 Hz, 1H); 2.07 (m, 1H); 0.97 (d, J=6.9 Hz, 3H); 0.86 (d, J=6.9 Hz, 3H).

### Example 1K

### (S)-2-(8-(4-methoxyphenyl)dibenzo[b,d]furan-3-sulfonamido)-3-methyl butanoic acid (Compound 310)

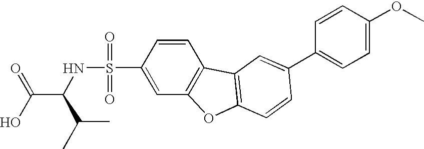

The title compound was prepared by the procedures described in Example 1, using 4-methoxyphenylboronic acid instead of 3-furanboronic acid. The compound was obtained as a white solid (88%). ¹H NMR (DMSO-d6): 12.48 (s br, 1H); 8.51 (d, J=1.3 Hz, 1H); 8.38 (d, J=8.2 HZ, 1H); 8.11 (m, 1H); 8.07 (d, J=1.3 Hz, 1H); 7.89-7.79 (m, 3H); 7.73 (d, J=8.8 Hz, 2H); 7.08 (d, J=8.8, 2H); 3.82 (s, 3H); 3.61 (m, 1H); 1.95 (m, 1H). MS (ES⁻): 452.1.

### Example 1L

### (S)-3-methyl-2-(8-(4-(trifluoromethyl)phenyl)dibenzo[b,d]furan-3-sulfonamido)butanoic acid (Compound 311)

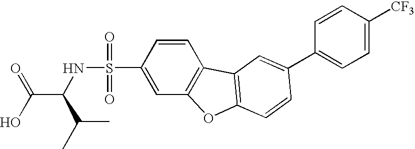

The title compound was prepared by the procedures described in Example 1, using 4-(trifluoromethyl)phenylboronic acid instead of 3-furanboronic acid. The compound was obtained as a white solid (80%). ¹H NMR (DMSO-d6): 12.47 (s br, 1H); 8.69 (d, J=1.6 Hz, 1H); 8.41 (d, J=8.2 HZ, 1H); 8.20-7.97 (m, 5H); 7.94-7.83 (m, 4H); 3.16 (m, 1H); 1.96 (m, 1H); 0.84 (d, J=6.9 Hz, 3H); 0.81 (d, J=6.9 Hz, 3H). MS (ES⁻): 490.1.

### Example 2

### (S)-2-(8-cyclopentyldibenzo[b,d]furan-3-sulfonamido)-3-methylbutanoic acid (Compound 71)

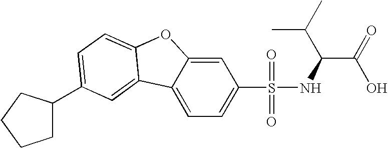

### Step 1: Preparation of (S)-methyl 2-(8-cyclopentyldibenzo[b,d]furan-3-sulfonamido)-3-methylbutanoate

(S)-Methyl 2-(8-cyclopentenyldibenzo[b,d]furan-3-sulfonamido)-3-methylbutanoate (170 mg, 40 mmol) and palladium on carbon (Pd/C, 100 mg) were mixed in 10 mL of methanol (MeOH). The reaction was carried out in a Parr® shaker at room temperature under 50 psi of hydrogen for 4 hours. The reaction mixture was filtered through a Celite® pad and the filtrate was concentrated to give the crude product, which was purified by column chromatography to produce (S)-methyl 2-(8-cyclopentyldibenzo[b,d]furan-3-sulfonamido)-3-methylbutanoate (125 mg) as a white solid.

### Step 2: Preparation of (S)-2-(8-cyclopentyldibenzo[b,d]furan-3-sulfonamido)-3-methylbutanoic acid

(S)-Methyl 2-(8-cyclopentyldibenzo[b,d]furan-3-sulfonamido)-3-methylbutanoate (120 mg) was dissolved in 1 mL of THF and to the resulting solution was added a LiOH solution (2 mL, 0.9 M). The reaction mixture was stirred at room temperature for 3 days, concentrated, and the resulting aqueous solution was acidified to pH of about 2. The mixture was filtered to produce (S)-2-(8-cyclopentyldibenzo[b,d]furan-3-sulfonamido)-3-methylbutanoic acid (106 mg) as a white solid. HRMS (ESI-FTMS): calculated for C₂₂H₂₅NO₅S+H⁺: 416.15262. found: 416.1519.

### Example 3

### (S)-3-methyl-2-(8-(pyridin-3-yl)dibenzo[b,d]furan-3-sulfonamido)butanoic acid (Compound 3)

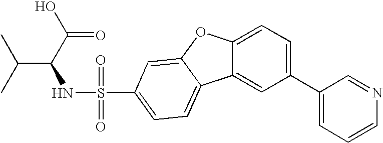

### Step 1: Preparation of 8-bromodibenzo[b,d]furan-3-sulfonyl chloride

Dibenzo[b,d]furan-3-sulfonyl chloride (5.3 g, 20 mmol, 1.0 eq.) was mixed with acetic acid (glacial, 120 mL) and bromine (10 mL, 10 eq.) and the resulting mixture was stirred at 70° C. for 4 hours. The excess bromine was removed by bubbling nitrogen through the reaction mixture and trapped with saturated Na₂SO₃ solution. After cooled to room temperature, the mixture was filtered to produce 8-bromodibenzo[b,d]furan-3-sulfonyl chloride (5.4 g) as a light brown solid.

### Step 2: Preparation of (S)-tert-butyl 2-(8-bromodibenzo[b,d]furan-3-sulfonamido)-3-methylbutanoate

8-Bromodibenzo[b,d]furan-3-sulfonyl chloride (3.46 g, 10 mmol) and (S)-t-butyl 2-amino-3-methylbutanoate hydrochloride (1.1 eq.) were mixed in 30 mL of DCM. N,N-Diisopropylethylamine (3.84 mL, 2.2 eq.) was added and the resulting mixture was stirred at room temperature for 5 hours. The crude product was purified by column chromatography to produce (S)-tert-butyl 2-(8-bromodibenzo[b,d]furan-3-sulfonamido)-3-methylbutanoate (4.7 g) as a white solid.

### Step 3: Preparation of (S)-tert-butyl 3-methyl-2-(8-(pyridin-3-yl)dibenzo[b,d]furan-3-sulfonamido)butanoate

(S)-Tert-butyl 2-(8-bromodibenzo[b,d]furan-3-sulfonamido)-3-methylbutanoate (240 mg, 0.5 mmol), K₂CO₃ (242 mg, 3.5 eq.), 3-pyridylboronic acid (1.25 mmol), and Pd(PPh₃)₄ (60 mg) were suspended in a mixture of 3 mL of DME and 0.5 mL of water. The reaction mixture was deoxygenated with nitrogen and stirred at 85° C. for 4 hours. Brine was added and the mixture was extracted with EtOAc. The combined organic layers were concentrated to give the crude product, which was purified by column chromatography to produce (S)-tert-butyl 3-methyl-2-(8-(pyridin-3-yl)dibenzo[b,d]furan-3-sulfonamido)butanoate (200 mg) as a white solid.

### Step 4: Preparation of (S)-3-methyl-2-(8-(pyridin-3-yl)dibenzo[b,d]furan-3-sulfonamido)butanoic acid

(S)-Tert-butyl 3-methyl-2-(8-(pyridin-3-yl)dibenzo[b,d]furan-3-sulfonamido) butanoate (200 mg) was dissolved in 4 mL of TFA/DCM (1:1) and the solution was stirred at room temperature for 4 hours. The resulting mixture was concentrated under vacuum and the residue was triturated in CH₃CN/water and dried by a freeze-dry process to produce (S)-3-Methyl-2-(8-(pyridin-3-yl)dibenzo[b,d]furan-3-sulfonamido)butanoic acid as a white solid. HRMS (ESI-FTMS): calculated for C₂₂H₂₀N₂O₅S+H⁺: 425.11657. found: 425.1177.

The following compounds in Table 3 were prepared using procedures analogous to those described above for the preparation of (S)-3-methyl-2-(8-(pyridin-3-yl)dibenzo[b,d]furan-3-sulfonamido)butanoic acid.

TABLE 3

| Compd | | | |
|---|---|---|---|
| No. | NMR | HRMS | MS |
| 1 | — | 523.154 | — |
| 4 | — | 523.1522 | — |
| 8 | — | 413.1169 | — |
| 9 | — | 443.1268 | — |
| 10 | — | 455.127 | — |
| 12 | ¹H NMR (DMSO-d6): δ 12.50 (s br, 1 H), 8.51 (d, J = 1.9 Hz, 1 H), | — | 478.1 |
| | 8.42 (d, J = 8.2 Hz, 1 H), 8.18-8.08 (m, 3 H), 7.98-7.91 (m, 3 H), 7.84 (m, 2 | | |
| | H), 7.48 (m, 2 H), 3.62 (dd, J = 9.4 and 6.0 Hz, 1 H), 1.96 (m, 1 H), | | |
| | 0.84 (d, J = 6.9 Hz, 3 H), and 0.81 (d, J = 6.9 Hz, 3 H). | | |
| 13 | ¹H NMR (DMSO-d6): δ 12.51 (s br, 1 H), 8.72 (d, J = 1.9 Hz, 1 H), | — | 478.1 |
| | 8.45 (d, J = 8.2 Hz, 1 H), 8.20-7.78 (m, 7 H), 7.46-7.34 (m, 3 H), 3.62 (dd, J = 9.4 | | |
| | and 6.0 Hz, 1 H), 1.96 (m, 1 H), 0.84 (d, J = 6.6 Hz, 3 H), and | | |
| | 0.82 (d, J = 6.9 Hz, 3 H). | | |
| 14 | ¹H NMR (DMSO-d6): δ 12.51 (s br, 1 H), 8.93 (dd, J = 4.4 and 1.9 Hz, | — | 473.1 |
| | 1 H), 8.77 (d, J = 1.9 Hz, 1 H), 8.49-8.40 (m, 3 H), 8.24 (dd, J = 8.8 | | |
| | and 2.2 Hz, 1 H), 8.19-8.08 (m, 4 H), 7.94 (d, J = 9.1 Hz, 1 H), | | |
| | 7.87 (dd, J = 8.2 and 1.3 Hz, 1 H), 7.60 (dd, J = 8.2 and 4.1 Hz, 1 H), and | | |
| | 3.63 (m, 1 H). | | |
| 16 | ¹H NMR (DMSO-d6): δ 12.49 (s br, 1 H), 8.89 (d, J = 1.6 Hz, 1 H), | — | 423.1 |
| | 8.83 (d, J = 6.3 HZ, 2 H), 8.42 (d, J = 8.2 Hz, 1 H), 8.20-8.10 (m, 5 H), | | |
| | 7.98 (d, J = 8.8 Hz, 1 H), 7.89 (dd, J = 8.2 and 1.6 Hz, 1 H), 3.63 (dd, J = 9.4 | | |
| | and 6.0 Hz, 1 H), and 1.96 (m, 1 H). | | |
| 17 | ¹H NMR (CDCl₃): δ 8.07-7.99 (m, 3 H), 7.82 (dd, J = 8.2 and 1.3 Hz, 1 | — | 442.1 |
| | H), 7.69 (dd, J = 8.8 and 1.9 Hz, 1 H), 7.54 (d, J = 8.5 Hz, 1 H), | | |
| | 7.10 (d, J = 3.8 Hz, 1 H), 6.72 (m, 1 H), 5.56 (d, J = 9.4 Hz, 1 H), 3.72 (dd, J = 9.4 | | |
| | and 4.7 Hz, 1 H), 2.48 (s, 3 H), 2.05 (m, 1 H), 0.95 (d, J = 6.9 Hz, | | |
| | 3 H), and 0.83 (d, J = 6.9 Hz, 3 H). | | |
| 18 | — | 428.1275 | — |
| 19 | — | 442.1437 | — |
| 20 | — | 484.191 | — |
| 21 | — | 456.1589 | — |
| 22 | — | 504.1592 | — |
| 23 | — | 414.1119 | — |
| 64 | — | — | 457.1 |
| 111 | — | — | 473.3 |
| 112 | — | — | 472.2 |

### Example 4

### (R)-2-(7-(furan-2-yl)dibenzo[b,d]furan-2-sulfonamido)-3-methylbutanoic acid (Compound 98)

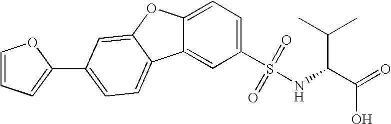

### Step 1: Preparation of 3-nitrodibenzo[b,d]furan

Dibenzofuran (50 g, fine powder) was mixed with 400 mL of trifluoroacetic acid (TFA) and the resulting suspension was cooled in an ethanol-ice bath before the addition of HNO₃ (11.7 mL, >90%) over 10 minutes. The reaction mixture was warmed to room temperature and stirred for 2 hours. After filtration, the resulting solid was triturated with methanol and dried under vacuum (see, e.g., Keumi, T. et al. (1991), J. O. C. 56: 4671) to produce 3-nitrodibenzo[b,d]furan (45 g, 70% yield) as a solid.

### Step 2: Preparation of 7-nitrodibenzo[b,d]furan-2-sulfonic acid

To a round-bottom flask containing 3-nitrodibenzo[b,d]furan (21.4 g, 100 mmol) in 200 mL of chloroform was slowly added chlorosulfonic acid (15.2 g, 130 mmol) at 0° C. The resulting suspension was warmed to room temperature and stirred for 4 hours. The reaction mixture was cooled to 0° C. and 7-nitrodibenzo[b,d]furan-2-sulfonic acid (24.1 g, 81% yield) was obtained by filtration as a white solid.

### Step 3: Preparation of 7-nitrodibenzo[b,d]furan-2-sulfonyl chloride

7-Nitrodibenzo[b,d]furan-2-sulfonic acid (2.93 g, 10 mmol) was mixed with thionyl chloride (15 mL) and a few drops of dimethylformamide (DMF) were slowly added. After stirred at 80° C. for 24 hours, the reaction mixture was filtered and excess thionyl chloride in the filtrate was removed under reduced pressure. The crude product from the filtrate was triturated with ice water to provide 7-nitrodibenzo[b,d]furan-2-sulfonyl chloride (2.78 g, 89% yield) as an off-white solid.

### Step 4: Preparation of (R)-methyl 3-methyl-2-(7-nitrodibenzo[b,d]furan-2-sulfonamido)butanoate

7-Nitrodibenzo[b,d]furan-2-sulfonyl chloride (570 mg, 1.83 mmol) and (R)-methyl 2-amino-3-methylbutanoate hydrochloride (334 mg, 2.0 mmol) were mixed with 5 mL of DCM. N,N-Diisopropylethylamine (520 mg, 4 mmol) was added slowly at 0° C. and the resulting mixture was stirred at room temperature for 4 hours. The crude product was purified by column chromatography to provide the (R)-valine sulfonamide (88% yield) as a white solid.

### Step 5: Preparation of (R)-methyl 2-(7-aminodibenzo[b,d]furan-2-sulfonamido)-3-methylbutanoate

(R)-Methyl 3-methyl-2-(7-nitrodibenzo[b,d]furan-2-sulfonamido)butanoate (480 mg) was mixed with Pd/C (100 mg, 10%) in 20 mL of MeOH. The reaction was carried out in a Parr® shaker at room temperature under hydrogen (50 psi) overnight. The reaction mixture was filtered through a Celite® pad and MeOH was removed to produce (R)-methyl 2-(7-aminodibenzo[b,d]furan-2-sulfonamido)-3-methylbutanoate (430 mg, quantitative yield) as an off-white solid.

The t-butyl ester analog, as well as the (S)-isomer analog, were prepared similarly using the corresponding amino acid analog at step 4.

### Step 6: Preparation of (R)-methyl 2-(7-iododibenzo[b,d]furan-2-sulfonamido)-3-methylbutanoate

(R)-Methyl 2-(7-aminodibenzo[b,d]furan-2-sulfonamido)-3-methylbutanoate (2.165 g, 5.75 mmol) was mixed with hydrochloric acid (12 mL, 18%) and the resulting solution was cooled to 0° C. An aqueous solution of sodium nitrite (9 mL, 1.0 M) was slowly added and the reaction mixture was stirred for 20 minutes, followed by very slow addition of a sodium iodide solution (948 mg, 6.32 mmol, in 3 mL of water). The reaction mixture was stirred for 20 minutes, water was added, and the precipitate was filtered to produce (R)-methyl 2-(7-iododibenzo[b,d]furan-2-sulfonamido)-3-methylbutanoate (71% yield) as a dark brown solid.

### Step 7: Preparation of (R)-methyl 2-(7-(furan-2-yl) dibenzo[b,d]furan-2-sulfonamido)-3-methylbutanoate

(R)-Methyl 2-(7-iododibenzo[b,d]furan-2-sulfonamido)-3-methylbutanoate (200 mg, 0.41 mmol) was mixed with 2-(furan-2-yl)-4,4,5,5-tetramethyl-1,3,2-dioxaborolane (238 mg, 1.23 mmol), Pd(PPh₃)₄ (24 mg, 0.02 mmol), and K₂CO₃ (283 mg, 2.05 mmol) in 2 mL of DME and 0.5 mL of water. The reaction mixture was heated to 80° C. for 3 hours, and was diluted with ethyl acetate and water. The organic layer was separated and concentrated to give the crude product, which was purified by a preparative HPLC to yield (R)-methyl 2-(7-(furan-2-yl) dibenzo[b,d]furan-2-sulfonamido)-3-methylbutanoate (52% yield).

### Step 8: Preparation of (R)-2-(7-(furan-2-yl) dibenzo[b,d]furan-2-sulfonamido)-3-methylbutanoic acid

(R)-Methyl 2-(7-(furan-2-yl)dibenzo[b,d]furan-2-sulfonamido)-3-methylbutanoate (90 mg, 0.21 mmol) was dissolved in a mixture of THF, MeOH, and water (2 mL) and lithium hydroxide (5 eq.) was added. The resulting mixture was stirred overnight and water was added. The pH of the solution was adjusted to between 4 and 5 and the resulting precipitate was filtered to produce (R)-2-(7-(furan-2-yl)dibenzo[b,d]furan-2-sulfonamido)-3-methylbutanoic acid (58% yield) as a white solid. ¹H NMR (400 MHz, MeOD): δ 0.91 (d, J=7.07 Hz, 3H), 1.00 (d, J=6.82 Hz, 3H), 2.05-2.16 (m, 1H), 3.77 (d, J=5.05 Hz, 1H), 6.82-6.84 (m, 1H), 7.56 (t, J=1.64 Hz, 1H), 7.59 (dd, J=8.08, 1.52 Hz, 1H), 7.66 (dd, J=8.59, 0.51 Hz, 1H), 7.73 (s, 2H), 7.89-7.91 (m, 1H), 7.96 (dd, J=8.59, 2.02 Hz, 1H), 8.01 (dd, J=8.08, 0.51 Hz, 1H), 8.01 (dd, J=8.08, 0.51 Hz, 1H), 8.47-8.49 (m, 1H). HRMS (ESI-FTMS): calculated for C₂₁H₁₉NO₆S+H⁺: 414.10059. found: 414.10071.

The following compounds were prepared by the procedure described in Example 4 for the preparation of (S)-2-(7-(furan-2-yl)dibenzo[b,d]furan-2-sulfonamido)-3-methylbutanoic acid.

### Example 4A

### (S)-2-(7-(4-bromo-5-ethylthiophen-2-yl)dibenzo[b,d]furan-3-sulfonamido)-3-methylbutanoic acid (Compound 165)

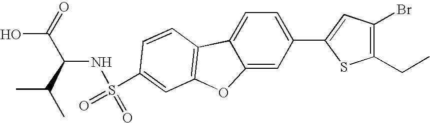

The title compound was prepared by the procedures described in Example 4, using (S)-methyl 2-(7-iododibenzo[b,d]furan-3-sulfonamido)-3-methylbutanoate (an intermediate in the preparation of Example 8). The compound was obtained as a white solid in 100% yield. ¹H NMR (400 MHz, CHLOROFORM-d) δ ppm 0.84 (d, J=6.82 Hz, 3H), 0.95 (d, J=6.57 Hz, 3H), 1.30-1.37 (m, J=7.58, 7.58 Hz, 4H), 2.85 (q, J=7.41 Hz, 1H), 5.12-5.24 (m, 1H), 7.24 (s, 1H), 7.57 (dd, J=8.21, 1.39 Hz, 1H), 7.75 (d, J=1.01 Hz, 1H), 7.82 (dd, J=8.08, 1.52 Hz, 1H), 7.95 (d, J=8.08 Hz, 1H), 8.00 (d, J=8.08 Hz, 1H), 8.05 (d, J=1.26 Hz, 1H). HRMS (ESI-FTMS): calcd for C₂₃H₂₂BrNO₅S₂+H⁺, 536.01955. found: 536.0192.

### Example 4B

### (S)-2-(7-(2′,5-diethyl-2,3% bithiophen-5′-yl)dibenzo[b,d]furan-3-sulfonamido)-3-methylbutanoic acid (Compound 166)

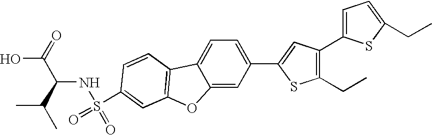

The title compound was isolated as a by-product (20% yield) in the preparation of (S)-2-(7-(4-bromo-5-ethylthiophen-2-yl)dibenzo[b,d]furan-3-sulfonamido)-3-methylbutanoic acid (compound 165). The compound was obtained as a white solid. ¹H NMR (400 MHz, CHLOROFORM-d) δ ppm 0.79 (d, J=7.07 Hz, 3H), 0.91 (d, J=6.82 Hz, 3H), 1.32-1.43 (m, 6H), 1.94-2.06 (m, J=3.79 Hz, 1H), 2.88 (q, 2H), 3.03 (q, J=7.58 Hz, 2H), 3.78 (dd, J=9.98, 4.67 Hz, 1H), 5.19 (d, J=10.10 Hz, 1H), 6.75-6.80 (m, 1H), 6.95 (d, J=3.54 Hz, 1H), 7.41 (s, 1H), 7.62 (dd, J=8.08, 1.52 Hz, 1H), 7.76-7.82 (m, 2H), 7.92 (d, J=8.08 Hz, 1H), 7.97 (d, J=8.08 Hz, 1H), 8.03 (d, J=1.01 Hz, 1H). HRMS (ESI-FTMS): calcd for C₂₉H₂₉NO₅S₃+H⁺, 568.12806. found: 568.1281.

### Example 4C

### (R)-3-methyl-2-(7-(pyrimidin-5-yl)dibenzo[b,d]furan-2-sulfonamido) butanoic acid (Compound 167)

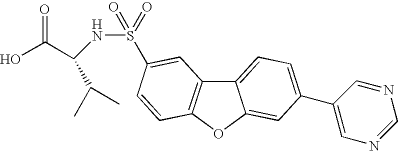

The title compound was prepared by the procedures described in Example 4, using pyrimidin-5-ylboronic acid instead of 2-(furan-2-yl)-4,4,5,5-tetramethyl-1,3,2-dioxaborolane. The compound was obtained as an off-white solid. ¹H NMR (400 MHz, MeOD) δ ppm 0.92 (d, J=6.82 Hz, 3H), 1.01 (d, J=6.57 Hz, 3H), 2.06-2.16 (m, 1H), 3.71 (d, J=4.80 Hz, 1H), 7.69-7.77 (m, 2H), 7.92-7.96 (m, 1H), 8.05 (dd, J=8.59, 2.02 Hz, 1H), 8.24 (d, J=7.83 Hz, 1H), 8.59 (d, J=2.02 Hz, 1H), 9.13 (s, 2H), 9.21 (s, 1H). HRMS (ESI-FTMS): calcd for C₂₁H₁₉N₃O₅S+H⁺, 426.11182. found: 426.11074.

### Example 4D

### (R)-2-(7-(2-methoxypyrimidin-5-yl)dibenzo[b,d]furan-2-sulfonamido)-3-methylbutanoic acid (Compound 168)

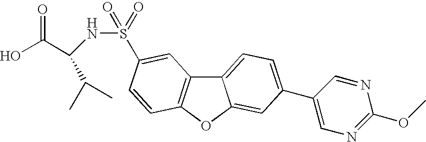

The title compound was prepared by the procedures described in Example 4, using 2-methoxypyrimidin-5-ylboronic acid instead of 2-(furan-2-yl)-4,4,5,5-tetramethyl-1,3,2-dioxaborolane. The compound was obtained as an off-white solid. ¹H NMR (400 MHz, MeOD) δ ppm 0.89 (d, J=6.82 Hz, 3H), 0.94 (d, J=6.82 Hz, 3H), 1.90-2.13 (m, 1H), 3.71 (d, J=5.81 Hz, 1H), 7.69-7.87 (m, 2H), 7.95-8.09 (m, 2H), 8.30 (d, J=8.08 Hz, 1H), 8.62 (d, J=2.02 Hz, 1H), 9.00 (s, 2H). HRMS (ESI-FTMS): calcd for C₂₂H₂₁N₃O₆S+H⁺, 456.12238. found: 456.12374.

### Example 4E

### (R)-2-(7-(2,4-dimethylthiazol-5-yl)dibenzo[b,d]furan-2-sulfonamido)-3-methylbutanoic acid (Compound 169)

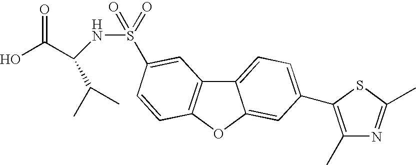

The title compound was prepared by the procedures described in Example 4, using 2,4-dimethyl-5-(4,4,5,5-tetramethyl-1,3,2-dioxaborolan-2-yl)thiazole instead of 2-(furan-2-yl)-4,4,5,5-tetramethyl-1,3,2-dioxaborolane. The compound was obtained as an off-white solid. ¹H NMR (400 MHz, MeOD) δ ppm 0.93 (d, J=6.82 Hz, 3H), 0.99 (d, J=6.82 Hz, 3H), 1.98-2.15 (m, 1H), 2.51 (s, 3H), 2.72 (s, 3H), 3.76 (d, J=5.31 Hz, 1H), 7.50 (dd, J=8.08, 1.52 Hz, 1H), 7.67-7.75 (m, 2H), 8.01 (dd, J=8.84, 2.02 Hz, 1H), 8.12 (d, J=8.08 Hz, 1H), 8.55 (d, J=1.77 Hz, 1H). HRMS (ESI-FTMS): calcd for C₂₂H₂₂N₂O₅S₂+H⁺, 459.10429. found: 459.10432.

### Example 4F

### (2R)-3-methyl-2-(7-(1-(2-methylbutyl)-1H-pyrazol-4-yl)dibenzo[b,d]furan-2-sulfonamido)butanoic acid (Compound 170)

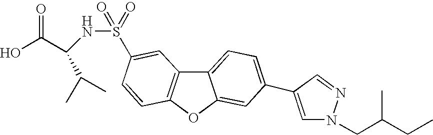

The title compound was prepared by the procedures described in Example 4, using 1-(2-methylbutyl)-4-(4,4,5,5-tetramethyl-1,3,2-dioxaborolan-2-yl)-1H-pyrazole instead of 2-(furan-2-yl)-4,4,5,5-tetramethyl-1,3,2-dioxaborolane. The compound was obtained as an off-white solid. ¹H NMR (400 MHz, MeOD) δ ppm 0.91 (d, J=6.82 Hz, 3H), 0.96-1.04 (m, 9H), 1.58-1.69 (m, 1H), 1.78-1.88 (m, 2H), 2.04-2.15 (m, 1H), 3.77 (d, J=5.05 Hz, 1H), 4.17-4.25 (m, 2H), 7.57 (dd, J=8.08, 1.26 Hz, 1H), 7.65 (d, J=8.84 Hz, 1H), 7.72 (d, J=0.76 Hz, 1H), 7.86 (d, J=10.36 Hz, 2H), 7.94 (dd, J=8.72, 1.89 Hz, 1H), 8.00 (d, J=8.08 Hz, 1H), 8.47 (d, J=2.02 Hz, 1H). HRMS (ESI-FTMS): calcd for C₂₅H₂₉N₃O₅S+H⁺, 484.19007. found: 484.19146.

### Example 4G

### (R)-3-methyl-2-(7-(1-propyl-1H-pyrazol-4-yl)dibenzo[b,d]furan-2-sulfonamido)butanoic acid (Compound 171)

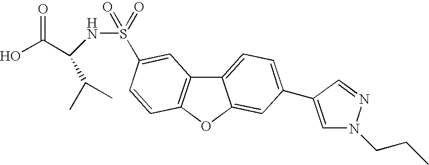

The title compound was prepared by the procedures described in Example 4, using 1-(2-methylbutyl)-4-(4,4,5,5-tetramethyl-1,3,2-dioxaborolan-2-yl)-1H-pyrazole instead of 2-(furan-2-yl)-4,4,5,5-tetramethyl-1,3,2-dioxaborolane. The compound was obtained as an off-white solid. ¹H NMR (400 MHz, MeOD) δ ppm 0.95-1.03 (m, 9H), 1.88-2.00 (m, 2H), 2.01-2.18 (m, 1H), 3.77 (d, J=5.31 Hz, 1H), 4.17 (t, J=7.07 Hz, 2H), 7.59 (dd, J=8.21, 1.39 Hz, 1H), 7.66 (d, J=8.84 Hz, 1H), 7.74 (d, J=1.52 Hz, 1H), 7.89 (s, 2H), 7.95 (dd, J=8.72, 1.89 Hz, 1H), 8.01 (d, J=8.08 Hz, 1H), 8.47 (d, J=2.02 Hz, 1H). HRMS (ESI-FTMS): calcd for C₂₃H₂₅N₃O₅S+H⁺, 456.15877. found: 456.1601.

### Example 4H

### (R)-3-methyl-2-(7-(1-(2-morpholinoethyl)-1H-pyrazol-4-yl)dibenzo [b,d ]furan-2-sulfonamido)butanoic acid (Compound 172)

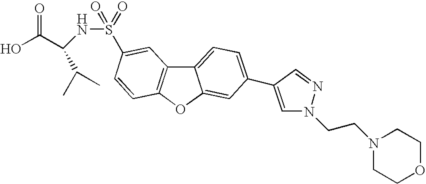

The title compound was prepared by the procedures described in Example 4, using 4-(2-(4-(4,4,5,5-tetramethyl-1,3,2-dioxaborolan-2-yl)-1H-pyrazol-1-yl)ethyl) morpholine instead of 2-(furan-2-yl)-4,4,5,5-tetramethyl-1,3,2-dioxaborolane. The compound was obtained as an off-white solid. ¹H NMR (400 MHz, MeOD) δ ppm 0.94 (d, J=6.82 Hz, 3H), 1.02 (d, J=6.82 Hz, 3H), 1.93-2.22 (m, 1H), 2.51-2.62 (m, 4H), 2.93 (t, J=6.57 Hz, 2H), 3.63-3.79 (m, 5H), 4.36 (t, J=6.57 Hz, 2H), 7.61 (d, J=1.52 Hz, 1H), 7.69 (d, J=8.59 Hz, 1H), 7.78 (d, J=1.01 Hz, 1H), 7.93 (s, 1H), 7.99 (dd, J=8.59, 2.02 Hz, 1H), 8.03-8.08 (m, 2H), 8.25 (dd, J=9.09, 7.07 Hz, 1H), 8.51 (d, J=1.77 Hz, 1H). HRMS (ESI-FTMS): calcd for C₂₆H₃₀N₄O₆S+H⁺, 527.19588. found: 527.19749.

### Example 4I

### (R)-2-(7-(1-isobutyl-1H-pyrazol-4-yl)dibenzo[b,d]furan-2-sulfonamido)-3-methylbutanoic acid (Compound 173)

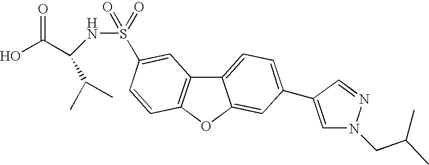

The title compound was prepared by the procedures described in Example 4, using 1-isobutyl-4-(4,4,5,5-tetramethyl-1,3,2-dioxaborolan-2-yl)-1H-pyrazole instead of 2-(furan-2-yl)-4,4,5,5-tetramethyl-1,3,2-dioxaborolane. The compound was obtained as an off-white solid. ¹H NMR (400 MHz, MeOD) δ ppm 0.91 (d, J=6.82 Hz, 3H), 0.99 (t, J=6.06 Hz, 9H), 2.03-2.17 (m, 1H), 2.19-2.36 (m, 1H), 3.77 (d, J=5.05 Hz, 1H), 4.00 (d, J=7.07 Hz, 2H), 7.58 (dd, J=8.08, 1.26 Hz, 1H), 7.65 (d, J=9.09 Hz, 1H), 7.73 (s, 1H), 7.87 (d, J=13.14 Hz, 2H), 7.95 (dd, J=8.72, 1.89 Hz, 1H), 8.01 (d, J=8.08 Hz, 1H), 8.47 (d, J=1.77 Hz, 1H). HRMS (ESI-FTMS): calcd for C₂₄H₂₇N₃O₅S+H⁺, 470.17442. found: 470.17607.

### Example 4J

### (R)-3-methyl-2-(7-(1,3,5-trimethyl-1H-pyrazol-4-yl)dibenzo[b,d]furan-2-sulfonamido)butanoic acid (Compound 174)

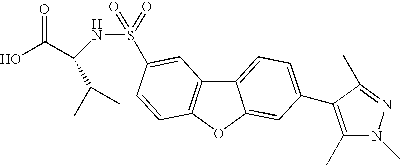

The title compound was prepared by the procedures described in Example 4, using 1,3,5-trimethyl-4-(4,4,5,5-tetramethyl-1,3,2-dioxaborolan-2-yl)-1H-pyrazole instead of 2-(furan-2-yl)-4,4,5,5-tetramethyl-1,3,2-dioxaborolane. The compound was obtained as an off-white solid. ¹H NMR (400 MHz, MeOD) δ ppm 0.91 (d, J=6.82 Hz, 3H), 0.99 (d, J=6.82 Hz, 3H), 2.00-2.19 (m, 1H), 2.27 (s, 3H), 2.32 (s, 3H), 3.77 (d, J=5.05 Hz, 1H), 3.81 (s, 3H), 7.31 (dd, J=7.96, 1.39 Hz, 1H), 7.48 (d, J=0.51 Hz, 1H), 7.67 (d, J=8.59 Hz, 1H), 7.97 (dd, J=8.59, 2.02 Hz, 1H), 8.06 (d, J=8.08 Hz, 1H), 8.51 (d, J=1.52 Hz, 1H). HRMS (ESI-FTMS): calcd for C₂₃H₂₅N₃O₅S+H⁺, 456.15877. found: 456.16019.

### Example 4K

### (R)-2-(7-(1-benzyl-1H-pyrazol-4-yl)dibenzo[b,d]furan-2-sulfonamido)-3-methylbutanoic acid (Compound 175)

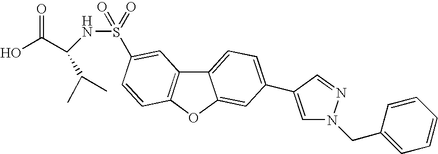

The title compound was prepared by the procedures described in Example 4, using 1-benzyl-4-(4,4,5,5-tetramethyl-1,3,2-dioxaborolan-2-yl)-1H-pyrazole instead of 2-(furan-2-yl)-4,4,5,5-tetramethyl-1,3,2-dioxaborolane. The compound was obtained as an off-white solid. ¹H NMR (400 MHz, MeOD) δ ppm 0.91 (d, J=7.07 Hz, 3H), 0.99 (d, J=6.82 Hz, 3H), 1.97-2.17 (m, 1H), 3.76 (d, J=5.05 Hz, 1H), 5.39 (s, 2H), 7.24-7.45 (m, 5H), 7.52-7.61 (m, 1H), 7.61-7.76 (m, 2H), 7.84-8.05 (m, 4H), 8.47 (d, J=1.77 Hz, 1H). HRMS (ESI-FTMS): calcd for C₂₇H₂₅N₃O₅S+H⁺, 504.15877. found: 504.16076.

### Example 4L

### (R)-3-methyl-2-(7-(4-methyl-2-phenylthiazol-5-yl)dibenzo[b,d]furan-2-sulfonamido)butanoic acid (Compound 176)

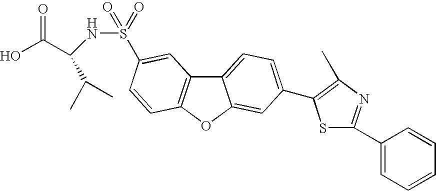

The title compound was prepared by the procedures described in Example 4, using 4-methyl-2-phenyl-5-(4,4,5,5-tetramethyl-1,3,2-dioxaborolan-2-yl)thiazole instead of 2-(furan-2-yl)-4,4,5,5-tetramethyl-1,3,2-dioxaborolane. The compound was obtained as an off-white solid. ¹H NMR (400 MHz, MeOD) δ ppm 0.89 (d, J=6.82 Hz, 3H), 0.99 (d, J=6.82 Hz, 3H), 1.94-2.21 (m, 1H), 2.63 (s, 3H), 3.66 (d, J=4.55 Hz, 1H), 7.44-7.52 (m, 3H), 7.56 (dd, J=8.08, 1.26 Hz, 1H), 7.63-7.78 (m, 2H), 7.87-8.03 (m, 3H), 8.10 (d, J=8.08 Hz, 1H), 8.55 (d, J=1.52 Hz, 1H). MS (LC-ESIMS) calcd for C₂₇H₂₄N₂O₅S₂−H⁺: 519.1. found 518.9.

### Example 4M

### (R)-3-methyl-2-(7-(4-methyl-2-(4-(trifluoromethyl)phenyl)thiazol-5-yl)dibenzo[b,d]furan-2-sulfonamido)butanoic acid (Compound 177)

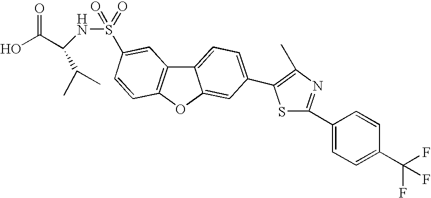

The title compound was prepared by the procedures described in Example 4, using 4-methyl-5-(4,4,5,5-tetramethyl-1,3,2-dioxaborolan-2-yl)-2-(4-(trifluoromethyl)phenyl) thiazole instead of 2-(furan-2-yl)-4,4,5,5-tetramethyl-1,3,2-dioxaborolane. The compound was obtained as an off-white solid. ¹H NMR (400 MHz, MeOD) δ ppm 0.92 (d, J=6.82 Hz, 3H), 1.00 (d, J=6.82 Hz, 3H), 1.97-2.19 (m, 1H), 2.62 (s, 3H), 3.78 (d, J=5.31 Hz, 2H), 7.69-7.78 (m, 4H), 7.91 (dd, J=8.21, 1.64 Hz, 1H), 8.10 (d, J=8.08 Hz, 2H), 8.13-8.19 (m, 3H). HRMS (ESI-FTMS): calcd for C₂₈H₂₃F₃N₂O₅S₂+H⁺, 589.10732. found: 589.10832.

The following compounds in Table 4 were prepared following procedures analogous to those described above for the preparation of (R)-2-(7-(furan-2-yl) dibenzo[b,d]furan-2-sulfonamido)-3-methylbutanoic acid.

TABLE 4

| Compd | | | |
|---|---|---|---|
| No. | NMR | HRMS | MS |
| 96 | ¹H NMR (400 MHz, MeOD): δ 1.12 (d, J = 6.82 Hz, 3 H), 1.21 (d, J = 6.82 Hz, | 414.10156 | — |
| | 3 H), 2.28 (s, 1 H), 3.83 (d, J = 4.80 Hz, 1 H), 7.78 (s, 2 H), | | |
| | 7.92 (d, J = 8.59 Hz, 1 H), 8.00 (s, 2 H), 8.19 (s, 2 H), 8.34 (d, J = 8.08 Hz, | | |
| | 1 H), and 8.75 (d, J = 2.02 Hz, 1 H). | | |
| 97 | ¹H NMR (400 MHz, MeOD): δ 0.91 (d, J = 6.82 Hz, 3 H), 0.98 (d, J = 6.82 Hz, | 428.06287 | — |
| | 3 H), 1.97-2.12 (m, 1 H), 3.71 (d, J = 5.56 Hz, 1 H), | | |
| | 7.50-7.55 (m, 1 H), 7.55-7.59 (m, 1 H), 7.70 (dd, J = 8.72 and 0.63 Hz, 1 H), | | |
| | 7.74-7.79 (m, 2 H), 7.91-7.95 (m, 1 H), 7.98 (dd, J = 8.84 and 2.02 Hz, | | |
| | 1 H), 8.11 (dd, J = 8.08 and 0.76 Hz, 1 H), and 8.53 (dd, J = 2.02 and | | |
| | 0.51 Hz, 1 H). | | |
| 99 | ¹H NMR (400 MHz, MeOD): δ 0.90 (d, J = 6.82 Hz, 3 H), 0.99 (d, J = 6.82 Hz, | 444.09316 | — |
| | 3 H), 0.99 (d, J = 6.82 Hz, 2 H), 2.31-2.35 (m, 3 H), 3.78 (d, J = 5.05 Hz, | | |
| | 1 H), 7.09-7.13 (m, 1 H), 7.32 (d, J = 3.28 Hz, 1 H), | | |
| | 7.47 (dd, J = 8.08 and 1.52 Hz, 1 H), 7.62-7.65 (m, 1 H), 7.66-7.71 (m, 2 H), | | |
| | 7.97 (dd, J = 8.59 and 2.02 Hz, 1 H), 8.02-8.07 (m, 1 H), and | | |
| | 8.49-8.53 (m, 1 H). | | |
| 100 | ¹H NMR (400 MHz, MeOD): δ 0.91 (d, J = 6.82 Hz, 3 H), 1.00 (d, J = 6.82 Hz, | 480.09455 | — |
| | 3 H), 2.06-2.17 (m, 1 H), 3.78 (d, J = 5.05 Hz, 1 H), | | |
| | 7.33-7.42 (m, 2 H), 7.66-7.74 (m, 2 H), 7.78-7.91 (m, 3 H), 7.94-8.02 (m, 2 H), | | |
| | 8.07 (dd, J = 8.08 and 0.51 Hz, 1 H), and 8.50-8.53 (m, 1 H). | | |
| 101 | ¹H NMR (400 MHz, MeOD): δ 0.93 (d, J = 6.82 Hz, 3 H), 1.00 (d, J = 6.82 Hz, | 459.07786 | — |
| | 3 H), 2.06-2.14 (m, 1 H), 3.77 (d, J = 5.31 Hz, 1 H), 7.57 (d, J = 8.34 Hz, | | |
| | 1 H), 7.70 (dd, J = 8.08 and 1.52 Hz, 1 H), 7.74 (d, J = 8.59 Hz, | | |
| | 1 H), 7.90 (d, J = 1.52 Hz, 1 H), 8.03 (dd, J = 8.84 and 2.02 Hz, 1 | | |
| | H), 8.13 (dd, J = 8.34 and 2.53 Hz, 1 H), 8.20 (d, J = 8.08 Hz, 1 H), | | |
| | 8.57 (d, J = 2.02 Hz, 1 H), and 8.70 (d, J = 2.53 Hz, 1 H). | | |
| 102 | ¹H NMR (400 MHz, MeOD): δ 0.92 (d, J = 6.82 Hz, 3 H), 1.00 (d, J = 6.82 Hz, | 455.12739 | — |
| | 3 H), 2.05-2.16 (m, 1 H), 3.78 (d, J = 5.31 Hz, 1 H), | | |
| | 6.93-6.98 (m, 1 H), 7.53 (s, 2 H), 7.63 (dd, J = 8.08 and 1.52 Hz, 1 H), | | |
| | 7.68-7.72 (m, 1 H), 7.80-7.82 (m, 1 H), 7.97-8.03 (m, 2 H), 8.12 (d, J = 8.08 Hz, | | |
| | 1 H), 8.46-8.49 (m, 1 H), and 8.52-8.55 (m, 1 H). | | |
| 103 | ¹H NMR (400 MHz, MeOD): δ 1.13 (d, J = 6.82 Hz, 3 H), 1.20 (d, J = 6.82 Hz, | 414.11197 | — |
| | 3 H), 2.21-2.33 (m, 1 H), 3.94 (d, J = 5.56 Hz, 1 H), 7.92 (d, J = 8.84 Hz, | | |
| | 2 H), 8.10 (s, 1 H), 8.18 (dd, J = 8.72 and 1.89 Hz, 1 H), | | |
| | 8.28-8.35 (m, 3 H), and 8.73 (d, J = 1.52 Hz, 1 H). | | |
| 104 | ¹H NMR (400 MHz, MeOD): δ 0.90 (d, J = 6.82 Hz, 3 H), 0.99 (d, J = 6.82 Hz, | 456.18402 | — |
| | 3 H), 1.16-1.45 (m, 5 H), 1.65-1.92 (m, 5 H), 2.03-2.15 (m, 1 | | |
| | H), 2.14-2.28 (m, 1 H), 3.74 (d, J = 5.05 Hz, 1 H), 6.29-6.40 (m, 1 H), | | |
| | 6.45-6.54 (m, 1 H), 7.39-7.45 (m, J = 8.08, 1.26 Hz, 1 H), | | |
| | 7.55-7.66 (m, 2 H), 7.88-7.97 (m, 2 H), and 8.40-8.47 (m, 1 H). | | |
| 105 | ¹H NMR (400 MHz, MeOD): δ 0.90 (d, J = 6.82 Hz, 3 H), 1.01 (d, J = 6.82 Hz, | 472.08897 | — |
| | 3 H), 1.99-2.15 (m, 1 H), 2.61 (s, 3 H), 3.63 (d, J = 5.05 Hz, 1 | | |
| | H), 7.59 (d, J = 4.04 Hz, 1 H), 7.68-7.74 (m, 1 H), 7.79 (dd, J = 8.08 | | |
| | and 1.52 Hz, 1 H), 7.86 (d, J = 4.04 Hz, 1 H), 7.96-8.06 (m, 2 H), | | |
| | 8.14 (d, J = 8.08 Hz, 1 H), and 8.53-8.58 (m, 1 H). | | |
| 115 | ¹H NMR (400 MHz, MeOD): δ 0.91 (d, J = 6.57 Hz, 3 H), 0.97 (d, J = 6.82 Hz, | 486.13347 | — |
| | 3 H), 1.98-2.11 (m, 1 H), 3.57 (s, 3 H), 3.71 (d, J = 5.56 Hz, 1 | | |
| | H), 4.01 (s, 3 H), 7.55 (dd, J = 8.08 and 1.52 Hz, 1 H), 7.72 (dd, J = 8.59 | | |
| | and 0.51 Hz, 1 H), 7.80 (d, J = 1.01 Hz, 1 H), 8.00 (dd, J = 8.72 | | |
| | and 1.89 Hz, 1 H), 8.09-8.17 (m, 2 H), and 8.55-8.56 (m, 1 H). | | |
| 116 | ¹H NMR (400 MHz, MeOD): δ 0.89 (d, J = 6.82 Hz, 3 H), 0.98 (d, J = 6.82 Hz, | 413.1169 | — |
| | 3 H), 1.94-2.13 (m, 1 H), 3.63 (d, J = 5.31 Hz, 1 H), 6.20 (dd, | | |
| | J = 3.54 and 2.53 Hz, 1 H), 6.63 (dd, J = 3.54 and 1.52 Hz, 1 H), | | |
| | 6.86 (dd, J = 2.78 and 1.52 Hz, 1 H), 7.62-7.70 (m, 2 H), 7.79 (d, J = 0.76 Hz, | | |
| | 1 H), 7.92 (dd, J = 8.72 and 1.89 Hz, 1 H), 8.00-8.05 (m, 1 H), and | | |
| | 8.42-8.49 (m, 1 H). | | |
| 117 | ¹H NMR (400 MHz, MeOD): δ 0.92 (d, J = 6.82 Hz, 3 H), 0.98 (d, J = 6.82 Hz, | 428.12793 | — |
| | 3 H), 1.97-2.14 (m, 1 H), 3.73 (d, J = 5.81 Hz, 1 H), 7.72 (dd, | | |
| | J = 7.96 and 1.39 Hz, 1 H), 7.78 (d, J = 8.59 Hz, 1 H), 7.90-7.95 (m, 1 | | |
| | H), 7.97-8.04 (m, 2 H), 8.13-8.21 (m, 2 H), and 8.54-8.58 (m, 1 H). | | |
| 118 | ¹ H NMR (400 MHz, MeOD): δ 0.95 (d, J = 6.82 Hz, 3 H), 1.00 (d, J = 6.82 Hz, | 428.06263 | — |
| | 3 H), 2.03-2.15 (m, 1 H), 3.76 (d, J = 5.81 Hz, 1 H), | | |
| | 7.21-7.27 (m, 1 H), 7.58 (dd, J = 5.05 and 1.26 Hz, 1 H), 7.65-7.69 (m, 1 H), | | |
| | 7.80-7.88 (m, 2 H), 8.01-8.09 (m, 2 H), 8.21-8.26 (m, 1 H), and | | |
| | 8.59-8.67 (m, 1 H). | | |
| 119 | ¹H NMR (400 MHz, MeOD): δ 0.92 (d, J = 6.82 Hz, 3 H), 0.98 (d, J = 6.82 Hz, | 462.1008 | — |
| | 3 H), 1.97-2.14 (m, 1 H), 3.74 (d, J = 5.56 Hz, 1 H), | | |
| | 7.21-7.43 (m, 4 H), 7.54-7.70 (m, 3 H), 7.72-7.79 (m, 1 H), 7.96-8.07 (m, 2 H), | | |
| | 8.16-8.25 (m, 2 H), and 8.56-8.62 (m, 1 H). | | |
| 120 | ¹H NMR (400 MHz, MeOD): δ 0.91 (d, J = 6.82 Hz, 3 H), 0.98 (d, J = 6.82 Hz, | 490.09294 | — |
| | 3 H), 1.99-2.12 (m, 1 H), 3.70 (d, J = 5.56 Hz, 1 H), | | |
| | 7.71-7.84 (m, 4 H), 7.90-7.98 (m, 2 H), 7.97-8.04 (m, 2 H), 8.18-8.26 (m, 1 H), | | |
| | and 8.55-8.63 (m, 1 H). | | |
| 121 | ¹H NMR (400 MHz, MeOD): δ 0.92 (d, J = 6.82 Hz, 3 H), 0.98 (d, J = 6.82 Hz, | 477.14873 | — |
| | 3 H), 1.98-2.10 (m, 1 H), 3.73 (d, J = 5.56 Hz, 1 H), 3.83 (s, 3 | | |
| | H), 6.60-6.66 (m, 1 H), 7.04-7.14 (m, 1 H), 7.18-7.27 (m, 1 H), 7.44 (d, | | |
| | J = 7.58 Hz, 1 H), 7.55-7.62 (m, 2 H), 7.65 (dd, J = 8.08 and 1.52 Hz, 1 | | |
| | H), 7.72-7.78 (m, 1 H), 7.83-7.86 (m, 1 H), 7.97-8.06 (m, 1 H), and | | |
| | 8.18-8.27 (m, 1 H). | | |
| 122 | ¹H NMR (400 MHz, MeOD): δ 0.92 (d, J = 6.82 Hz, 3 H), 0.98 (d, J = 6.82 Hz, | 479.10709 | — |
| | 3 H), 1.98-2.12 (m, 1 H), 3.74 (d, J = 6.06 Hz, 1 H), | | |
| | 6.85-6.94 (m, 1 H), 6.94-6.98 (m, 1 H), 7.22 (dd, J = 9.73 and 2.40 Hz, 1 H), | | |
| | 7.33-7.42 (m, 1 H), 7.73 (d, J = 8.59 Hz, 1 H), 7.99 (dd, J = 8.59 and | | |
| | 2.02 Hz, 1 H), 8.04-8.08 (m, 1 H), 8.15 (d, J = 8.34 Hz, 1 H), and | | |
| | 8.53-8.57 (m, 1 H). | | |
| 144 | — | 507.99051 | — |
| 145 | — | 443.12839 | — |
| 146 | — | 446.08511 | — |

### Example 5

### (S)-2-(8-(5-chlorofuran-2-yl)dibenzo[b,d]furan-3-sulfonamido)-3-methyl butanoic acid (Compound 73)

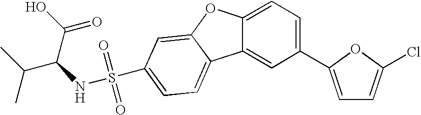

### Step 1: Preparation of (S)-methyl 2-(8-(5-chlorofuran-2-yl)dibenzo[b,d]furan-3-sulfonamido)-3-methylbutanoate

(S)-Methyl 2-(8-(furan-2-yl)dibenzo[b,d]furan-3-sulfonamido)-3-methylbutanoate (353 mg, 0.826 mmol) and N-chlorosuccinimide (NCS, 132 mg, 1.2 eq.) were mixed in 3.0 mL of methylene chloride and a catalytical amount of TFA was added. The mixture was stirred at room temperature until no starting material was left according to liquid chromatography-mass spectrometry (LC-MS). Dimethylsulfoxide (DMSO, 0.5 mL) was added and the clear solution was stirred at room temperature for 1 hour. Brine was added; and the organic layer was separated, washed with water/brine, and was concentrated to yield the crude product as a brown solid which was purified by column chromatography to give (S)-methyl 2-(8-(5-chlorofuran-2-yl)dibenzo[b,d]furan-3-sulfonamido)-3-methylbutanoate (270 mg) as a white solid.

### Step 2: Preparation of (S)-2-(8-(5-chlorofuran-2-yl)dibenzo[b,d]furan-3-sulfonamido)-3-methylbutanoic acid

Following the procedures for methyl ester hydrolysis described in Example 4 (Step 8), (S)-methyl 2-(8-(5-chlorofuran-2-yl)dibenzo[b,d]furan-3-sulfonamido)-3-methylbutanoate was treated with LiOH solution to produce (S)-2-(8-(5-chlorofuran-2-yl)dibenzo[b,d]furan-3-sulfonamido)-3-methylbutanoic acid as a white power. ¹H NMR (400 MHz, DMSO-d₆): δ 0.83 (m, 6H), 1.91-2.01 (m, 1H), 3.62 (dd, J=9.47 and 5.94 Hz, 1H), 6.67 (d, J=3.54 Hz, 1H), 7.14 (d, J=3.54 Hz, 1H), 7.82-7.86 (m, 1H), 7.86 (d, J=8.08 Hz, 1H), 7.93-7.98 (m, 1H), 8.08 (d, J=1.52 Hz, 1H), 8.20 (d, J=9.60 Hz, 1H), 8.43 (d, J=8.08 Hz, 1H), 8.58 (d, J=1.77 Hz, 1H), and 12.55 (s, 1H). HRMS (ESI-FTMS): calculated for C₂₁H₁₈ClNO₆S+H⁺: 448.06161. found: 448.06236.

The following compounds were prepared by the procedure as described in Example 5 for the preparation of (S)-2-(8-(5-chlorofuran-2-yl)dibenzo[b,d]furan-3-sulfon amido)-3-methylbutanoic acid.

### Example 5A

### (R)-2-(8-(5-chlorofuran-2-yl)dibenzo[b,d]furan-3-sulfonamido)-3-methylbutanoic acid (Compound 178)

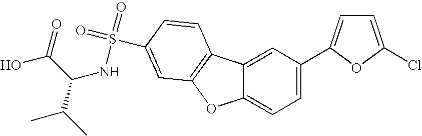

The title compound was prepared by the procedure described in Example 5, using the corresponding (R)-isomer. The compound was obtained as a white solid in 90% yield. ¹H NMR (400 MHz, DMSO-d₆) δ ppm 0.83 (m, J=6H) 1.96 (dd, J=12.88, 6.82 Hz, 1H) 3.62 (dd, J=9.47, 5.94 Hz, 1H) 6.66 (d, J=3.54 Hz, 1H) 7.13 (d, J=3.54 Hz, 1H) 7.81-7.89 (m, 2H) 7.92-7.99 (m, 1H) 8.08 (d, J=1.01 Hz, 1H) 8.16 (d, J=9.60 Hz, 1H) 8.43 (d, J=8.08 Hz, 1H) 8.57 (d, J=1.26 Hz, 1H). HRMS (ESI-FTMS): calcd for C₂₁H₁₈ClNO₆S+H⁺, 448.06161. found: 448.06132.

### Example 5B

### (S)-2-(8-(2-chlorothiazol-5-yl)dibenzo[b,d]furan-3-sulfonamido)-3-methylbutanoic acid (Compound 179)

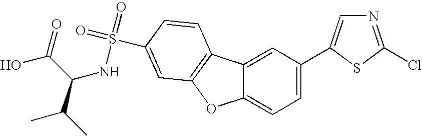

The title compound was prepared by the procedure described in Example 5, using the corresponding (S)-methyl 3-methyl-2-(8-(thiazol-5-yl)dibenzo[b,d]furan-3-sulfonamido)butanoate. The final compound was obtained as a white solid in 100% yield. ¹H NMR (400 MHz, DMSO-d₆) δ ppm 0.82 (m, 6H), 1.95 (d, J=6.32 Hz, 1H), 3.59 (s, 1H), 7.78-7.92 (m, 2H), 7.92-7.97 (m, 1H), 8.11 (d, J=1.52 Hz, 1H), 8.44 (d, J=8.34 Hz, 1 H), 8.57 (d, J=1.77 Hz, 1H), 9.22 (s, 1H). HRMS (ESI-FTMS): calcd for C₂₀H₁₇ClN₂O₆S₂+H⁺, 465.03402. found: 465.03486.

### Example 5C

### (S)-2-(8-(2-chlorothiazol-4-yl)dibenzo[b,d]furan-3-sulfonamido)-3-methylbutanoic acid (Compound 180)

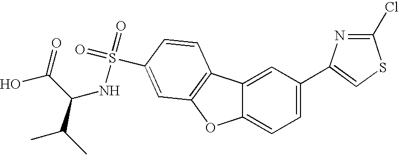

The title compound was prepared by the procedures described in Example 5, using (S)-methyl 3-methyl-2-(8-(thiazol-4-yl)dibenzo[b,d]furan-3-sulfonamido)butanoate. The final compound was obtained as a white solid in 100% yield. HRMS (ESI-FTMS): calcd for C₂₀H₁₇ClN₂O₆S₂+H⁺, 465.03402. found: 465.03475.

### Example 5D

### (S)-2-(7-(2-chlorothiazol-5-yl)dibenzo[b,d]furan-3-sulfonamido)-3-methylbutanoic acid (Compound 181)

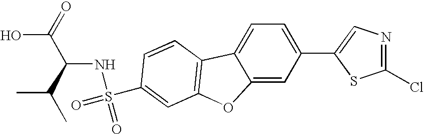

The title compound was prepared by the procedures described in Example 5, using (S)-methyl 3-methyl-2-(7-(thiazol-5-yl)dibenzo[b,d]furan-3-sulfonamido)butanoate. The final compound was obtained as a white solid in 60% yield. ¹H NMR (400 MHz, MeOD) δ ppm 0.82 (d, 3H), 0.88 (d, J=6.82 Hz, 3H), 1.88-2.03 (m, J=12.51, 6.69 Hz, 1H), 3.64 (d, J=5.56 Hz, 1H), 7.63 (dd, J=8.34, 1.52 Hz, 1H), 7.81 (dd, J=8.08, 1.52 Hz, 1H), 7.93 (d, J=1.01 Hz, 1H), 8.02 (d, J=1.01 Hz, 1H), 8.13 (d, J=8.34 Hz, 3H), 8.93 (s, 1H). HRMS (ESI-FTMS): calcd for C₂₀H₁₇ClN₂O₅S₂+H⁺, 465.03402. found: 465.0351.

### Example 5E

### (S)-2-(7-(5-chlorofuran-2-yl)dibenzo[b,d]thiophene-3-sulfonamido)-3-methylbutanoic acid (Compound 182)

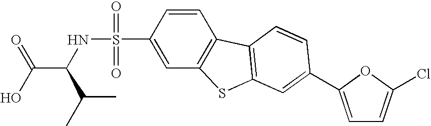

The title compound was prepared by the procedures described in Example 5, using (S)-2-(7-(furan-2-yl)dibenzo[b,d]thiophene-3-sulfonamido)-3-methylbutanoic acid (Compound 193). The final compound was obtained as a white solid. ¹H NMR (300 MHz, DMSO-d₆) δppm 12.52 (br. s., 1H), 8.48-8.58 (m, 2H), 8.47 (d, J=1.5 Hz, 1H), 8.42 (d, J=1.2 Hz, 1H), 8.10 (d, J=10.0 Hz, 1H), 7.90 (dd, J=3.4, 1.6 Hz, 1H), 7.87 (dd, J=3.5, 1.8 Hz, 1H), 7.26 (d, J=3.2 Hz, 1H), 6.70 (d, J=3.5 Hz, 1H), 3.61 (dd, J=8.8, 6.2 Hz, 1H), 1.88-2.04 (m, 1H), 0.84 (d, J=6.7 Hz, 3H), 0.81 (d, J=6.7 Hz, 3H). ESIMS (m/z) 463.95 (MH⁺).

The following compounds in Table 5 were prepared following procedures analogous to those described above for the preparation of (S)-2-(8-(5-chlorofuran-2-yl)dibenzo[b,d]furan-3-sulfonamido)-3-methylbutanoic acid.

TABLE 5

| Compd No. | HRMS |
|---|---|
| 74 | 464.03938 |
| 75 | 497.99929 |
| 84 | 464.03836 |
| 85 | 497.99944 |
| 86 | 531.95936 |
| 95 | 482.02357 |
| 134 | 482.02196 |

### Example 6

### (S)-2-(8-(methoxyethynyl)dibenzo[b,d]furan-3-sulfonamido)-3-methyl butanoic acid (Compound 38)

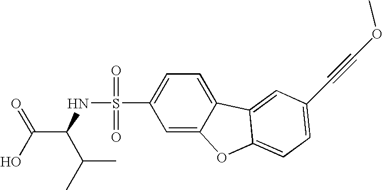

### Step 1: Preparation of (S)-tert-butyl 2-(8-(3-methoxyprop-1-ynyl)dibenzo[b,d]furan-3-sulfonamido)-3-methylbutanoate

(S)-Tert-butyl 2-(8-bromodibenzo[b,d]furan-3-sulfonamido)-3-methylbutanoate (482 mg, 1 mmol) and CuI (6.5 mg, 0.035 mmol) were dissolved in a mixture of 14 mL of acetonitrile and 6 mL of triethylamine (TEA). The solution was deoxygenated by bubbling nitrogen through for 10 minutes and palladium catalyst (0.035 mmol) was added, followed by 3-methoxyprop-1-yne (1.5 mmol). The mixture was heated at 90° C. until no starting material was left according to LC-MS. It was concentrated and the residue was partitioned between a mixture of 15 mL of methylene chloride (DCM) and 20 mL of water. The aqueous phase was extracted twice with DCM (15 mL×2) and the combined organic layers were dried over sodium sulfate (Na₂SO₄) and concentrated in vacuum to provide a residue, which was purified by silica gel column chromatography to give (S)-tert-butyl 2-(8-(3-methoxyprop-1-ynyl)dibenzo[b,d]furan-3-sulfonamido)-3-methylbutanoate.

### Step 2: Preparation of (S)-2-(8-(methoxyethynyl)dibenzo[b,d]furan-3-sulfonamido)-3-methylbutanoic acid

(S)-tert-Butyl 2-(8-(3-methoxyprop-1-ynyl)dibenzo[b,d]furan-3-sulfonamido)-3-methyl butanoate was treated with 5 mL of TFA in methylene chloride (30%) at room temperature for 4 hours. Concentration of the reaction mixture under reduced pressure afforded (S)-2-(8-(methoxyethynyl)dibenzo[b,d]furan-3-sulfonamido)-3-methylbutanoic acid as a white powder. ¹H NMR (DMSO-d6): δ 0.80 (d, J=6.6 Hz, 3H), 0.84 (d, J=6.6 Hz, 3H), 1.95 (m, 1H), 3.37 (s, 3H), 3.62 (dd, J=9.4, 6.0 Hz, 1H), 4.38 (s, 2H), 7.63 (dd, J=8.2, 1.6 Hz, 1H), 7.88 (dd, J=8.5, 1.6 Hz, 1H), 8.11 (d, J=9.4 Hz, 1H), 8.27 (d, J=1.6 Hz, 1H), 8.46 (d, J=8.2 Hz, 1H), 8.49 (d, J=1.6 Hz, 1H), 8.54 (d, J=8.5 Hz, 1H), 12.49 (s br, 1H). MS (IS, [M+H]⁺): 416.1.

The following compounds in Table 6 were prepared following procedures analogous to those described above for the preparation of (S)-2-(8-(methoxyethynyl)dibenzo[b,d]furan-3-sulfonamido)-3-methylbutanoic acid.

TABLE 6

| Compd | | | |
|---|---|---|---|
| No. | NMR | HRMS | MS |
| 2 | — | 429.1495 | — |
| 6 | — | — | 430.1 |
| 15 | — | — | 452.14 |
| 39 | ¹H NMR (DMSO-d6): δ 0.80 (d, J = 6.9 Hz, 3 H), 0.83 (d, J = 6.9 Hz, | — | 441.2 |
| | 3 H), 1.29 (t, J = 7.2 Hz, 6 H), 1.95 (m, 1 H), 3.31 (m, 4 H), 3.62 (dd, | | |
| | J = 9.4 and 6.3 Hz, 1 H), 4.43 (s, 2 H), 7.78 (dd, J = 8.5 and 1.6 Hz, | | |
| | 1 H), 7.85 (dd, J = 8.8 and 1.6 Hz, 1 H), 7.85 (d, J = 8.5, 1 H), | | |
| | 8.10 (d, J = 1.3 Hz, 1 H), 8.17 (d, J = 9.4 Hz, 1 H), 8.36 (d, J = 8.2 Hz, 1 | | |
| | H), and 8.49 (d, J = 1.6 Hz, 1 H). | | |

### Example 7

### (S)-2-(8-(3-((dimethylamino)methyl)furan-2-yl)dibenzo[b,d]furan-3-sulfonamido)-3-methylbutanoic acid (Compound 48)

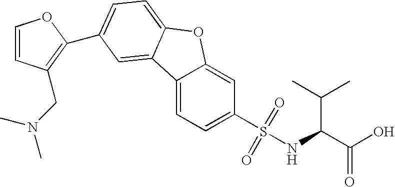

(S)-2-(8-(3-Formylfuran-2-yl)dibenzo[b,d]furan-3-sulfonamido)-3-methylbutanoic acid (220 mg, 0.5 mmol) was dissolved in dimethylformamide (DMF) and dimethyl amine (5 mL, 2.0 M in methanol, 10 mmol) and sodium cyanoborohydride (NaCNBH₃, 630 mg, 10 mmol) were added. The mixture was stirred at room temperature for 3 hours, water was added, and the reaction mixture was purified by a preparative HPLC to produce (S)-2-(8-(3-((dimethylamino)methyl)furan-2-yl)dibenzo[b,d]furan-3-sulfonamido)-3-methylbutanoic acid as a white solid. HRMS (ESI-FTMS): calculated for C₂₄H₂₆N₂O₆S+H⁺, 471.15843. found: 471.1608.

The following compounds in Table 7 were prepared using procedures analogous to those described above for the preparation of (S)-2-(8-(3-((dimethylamino)methyl)furan-2-yl)dibenzo[b,d]furan-3-sulfonamido)-3-methylbutanoic acid.

TABLE 7

| Compd No. | HRMS | MS |
|---|---|---|
| 49 | 487.1379 | — |
| 50 | 501.1491 | — |
| 68 | | 432.3 |

### Example 8

### (S)-3-methyl-2-(7-(5-methylfuran-2-yl)dibenzo[b,d]furan-3-sulfonamido) butanoic acid (Compound 135)

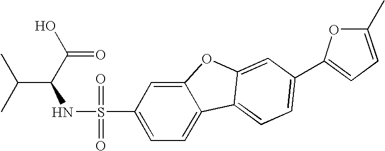

### Step 1: Preparation of 8-bromodibenzo[b,d]furan-3-sulfonyl chloride

Dibenzo[b,d]furan-3-sulfonyl chloride (5.3 g, 20 mmol, 1.0 eq.) was mixed with acetic acid (glacial, 120 mL) and bromine (10 mL, 10 eq.). The mixture was stirred at 70° C. for 4 hours. The excess bromine was removed by bubbling nitrogen through the reaction mixture and trapped with saturated Na₂SO₃ solution. The resulting solution was cooled down to room temperature and filtered to produce 8-bromodibenzo[b,d]furan-3-sulfonyl chloride (5.4 g) as a light brown solid.

### Step 2: Preparation of (S)-methyl 2-(8-bromodibenzo[b,d]furan-3-sulfonamido)-3-methylbutanoate

8-Bromodibenzo[b,d]furan-3-sulfonyl chloride (3.46 g, 10 mmol) and (S)-methyl 2-amino-3-methylbutanoate hydrochloride (1.1 eq.) were mixed in 30 mL of DCM and N,N-diisopropylethylamine (3.84 mL, 2.2 eq.) was added. The mixture was stirred at room temperature for 5 hours, concentrated, and purified by column chromatography to produce (S)-methyl 2-(8-bromodibenzo[b,d]furan-3-sulfonamido)-3-methylbutanoate (4.7 g) as a white solid.

### Step 3: Preparation of (S)-methyl 2-(8-bromo-7-nitrodibenzo[b,d]furan-3-sulfonamido)-3-methylbutanoate

A mixture of (S)-methyl 2-(8-bromodibenzo[b,d]furan-3-sulfonamido)-3-methyl butanoate (724 mg, 1.6 mmol) and nitric acid (HNO₃, 0.27 g, 4.2 mmol) in 15 mL of TFA and 1 mL of DCM was stirred at room temperature for 5 hours. The solvents were removed under vacuum and the crude product was purified by column chromatography to produce (S)-methyl 2-(8-bromo-7-nitrodibenzo[b,d]furan-3-sulfonamido)-3-methylbutanoate (625 mg) as a yellow solid.

### Step 4: Preparation of (S)-methyl 2-(7-aminodibenzo[b,d]furan-3-sulfonamido)-3-methylbutanoate

(S)-Methyl 2-(8-bromo-7-nitrodibenzo[b,d]furan-3-sulfonamido)-3-methylbutanoate (11.56 g, 23.8 mmol) was mixed with 200 mL of MeOH and Pd/C (700 mg) was added. The reaction was carried out in a Parr® shaker at room temperature under hydrogen (50 psi) overnight. The reaction mixture was filtered through a Celite® pad and concentrated to produce (S)-methyl 2-(7-aminodibenzo[b,d]furan-3-sulfonamido)-3-methylbutanoate (8.92 g) as a grey solid.

### Step 5: Preparation of (S)-methyl 2-(7-iododibenzo[b,d]furan-3-sulfonamido)-3-methylbutanoate

(S)-Methyl 2-(7-aminodibenzo[b,d]furan-3-sulfonamido)-3-methylbutanoate (3.72 g, 9.9 mmol) and HCl (3.5 mL) in 12 mL of H₂O and 50 mL of acetic acid were cooled to 0° C. A NaNO₂ solution (2 M, 7.5 mL) was added dropwise, followed by the addition of NaI (11.87 g, 80 mmol). The mixture was slowly warmed to room temperature, stirred for 3 hours, and filtered to provide the crude product, which was purified by column chromatography to produce (S)-methyl 2-(7-iododibenzo[b,d]furan-3-sulfonamido)-3-methylbutanoate (3.94 g) as a grey solid.

### Step 6: Preparation of (S)-methyl 3-methyl-2-(7-(5-methylfuran-2-yl)dibenzo[b,d]furan-3-sulfonamido)butanoate

(S)-Methyl 2-(7-iododibenzo[b,d]furan-3-sulfonamido)-3-methylbutanoate (200 mg, 0.41 mmol), 5-methylfuran-2-boronic acid pinacol ester (214 mg, 2.5 mmol), Pd(PPh₃)₄ (40 mg), and K₂CO₃ (227 mg, 1.6 mmol) were mixed in 2 mL of DME and 0.5 mL of water. The resulting mixture was deoxygenated with nitrogen flow for 5 minutes and was irradiated under microwave at 120° C. for 15 minutes. The crude product was purified by column chromatography to produce (S)-methyl 3-methyl-2-(7-(5-methylfuran-2-yl)dibenzo[b,d]furan-3-sulfonamido)butanoate (170 mg) as a white solid.

### Step 7: Preparation of (S)-3-methyl-2-(7-(5-methylfuran-2-yl)dibenzo[b,d]furan-3-sulfonamido)butanoic acid

(S)-Methyl 3-methyl-2-(7-(5-methylfuran-2-yl)dibenzo[b,d]furan-3-sulfonamido) butanoate (168 mg) was dissolved in 2 mL of THF, LiOH solution (0.9 M, 2 mL) was added, and the resulting mixture was stirred at room temperature for 3 days. THF was removed under vacuum and the remaining aqueous solution was acidified to pH ˜2. The mixture was filtered to give (S)-3-methyl-2-(7-(5-methylfuran-2-yl)dibenzo[b,d]furan-3-sulfonamido) butanoic acid (162 mg) as a white solid. HRMS (ESI-FTMS): calculated for C₂₂H₂₁NO₆S+H⁺: 428.11624. found: 428.11669.

The following compounds in Table 8 were prepared using procedures analogous to those described above for the preparation of (S)-3-methyl-2-(7-(5-methylfuran-2-yl)dibenzo[b,d]furan-3-sulfonamido)butanoic acid.

TABLE 8

| Compd No. | HRMS |
|---|---|
| 78 | 414.10065 |
| 79 | 414.10041 |
| 80 | 448.06376 |
| 81 | 430.07789 |
| 136 | 480.09414 |
| 137 | 431.07391 |
| 142 | 444.09395 |
| 143 | 464.11675 |

### Example 9

### (S)-2-(8-(N-isopropylcarbamimidoyl)dibenzo[b,d]furan-3-sulfonamido)-3-methylbutanoic acid (Compound 76)

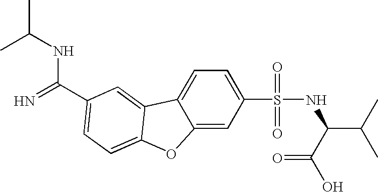

### Step 1: Preparation of (S)-methyl 2-(8-cyanodibenzo[b,d]furan-3-sulfonamido)-3-methylbutanoate

(S)-Methyl 2-(8-bromodibenzo[b,d]furan-3-sulfonamido)-3-methylbutanoate (1.0 g, 2.27 mmol), zinc cyanide (ZnCN₂, 293 mg, 2.5 mmol), and Pd(PPh₃)₄ (79 mg, 0.07 mmol) were dissolved in 20 mL of N-methylpyrrolidone (NMP) in a 20-mL microwave vial. The solution was deoxygenated by bubbling nitrogen for 5 minutes and was irradiated with microwave at 100° C. until no starting material was left according to LC-MS. Water was added to the reaction mixture and the precipitate was filtered to give the crude product, which was precipitated from methylene chloride/hexane solution. The precipitate was filtered to give (S)-methyl 2-(8-cyanodibenzo[b,d]furan-3-sulfonamido)-3-methylbutanoate as a white solid.

### Step 2: Preparation of (S)-methyl 2-(8-(imino(methoxy)methyl)dibenzo[b,d]furan-3-sulfonamido)-3-methylbutanoate

(S)-Methyl 2-(8-cyanodibenzo[b,d]furan-3-sulfonamido)-3-methylbutanoate (57 mg) was dissolved in 1 mL of dry MeOH and 1 mL of THF and gaseous HCl was bubbled at 0° C. for 15 minutes. The mixture was stirred overnight. The solvent was removed and the residue triturated with diethyl ether and filtered to produce (S)-methyl 2-(8-(imino(methoxy)methyl)dibenzo[b,d]furan-3-sulfonamido)-3-methylbutanoate (47 mg) as a white solid.

### Step 3: Preparation of (S)-methyl 2-(8-(N-isopropylcarbamimidoyl)dibenzo[b,d]furan-3-sulfonamido)-3-methylbutanoate

(S)-Methyl 2-(8-(imino(methoxy)methyl)dibenzo[b,d]furan-3-sulfonamido)-3-methy Ibutanoate (0.38 g, 0.83 mmol) was suspended in 10 mL of dry THF and, after addition of isopropyl amine (0.4 mL, 4.18 mmol), the mixture was heated at 70° C. overnight. The reaction mixture was concentrated and the residue was purified by a neutral alumina column chromatography to produce (S)-methyl 2-(8-(N-isopropylcarbamimidoyl)dibenzo[b,d]furan-3-sulfonamido)-3-methylbutanoate (260 mg, 70% yield).

### Step 4: Preparation of (S)-2-(8-(N-isopropylcarbamimidoyl)dibenzo[b,d]furan-3-sulfonamido)-3-methylbutanoic acid

(S)-Methyl 2-(8-(N-isopropylcarbamimidoyl)dibenzo[b,d]furan-3-sulfonamido)-3-methylbutanoate (60 mg, 0.13 mmol) was dissolved in 0.3 mL of glacial acetic acid and 1.2 mL of concentrated HCl in a sealed tube and the solution was heated at 65° C. for 24 hours. The reaction mixture was concentrated and the solid was washed with diethyl ether and dried to produce (S)-2-(8-(N-isopropylcarbamimidoyl)dibenzo[b,d]furan-3-sulfonamido)-3-methylbutanoic acid as a hydrochloride salt. ¹H NMR (DMSO-d6): δ 0.78 (d, J=6.9 Hz, 3H), 0.91 (d, J=6.9 Hz, 3H), 1.31 (d br, J=5.5 Hz, 6H), 2.04 (m, 1H), 3.02 (m, 1H), 4.05 (m, 1H), 8.02-7.77 (m, 3H), 8.12 (s, 1H), 8.31 (m, 1H), 8.75 (s, 1H), 9.35 (s br, 2H). MS (ES, [M+H]⁺): 432.2.

The following compounds in Table 9 were prepared using procedures analogous to those described above for the preparation of (S)-2-(8-(N-isopropylcarbamimidoyl)dibenzo b,d]furan-3-sulfonamido)-3-methylbutanoic acid.

TABLE 9

| Compd | | |
|---|---|---|
| No. | NMR | MS |
| 77 | ¹H NMR (DMSO-d6): δ0.82 (d, J = 6.9 Hz, 3 H), 0.93 (d, J = 6.9 Hz, 3 H), | 416.2 |
| | 2.05 (m, 1 H), 3.15 (d, J = 4.0 Hz, 1 H), 3.77 (s, 4 H), 7.58 (d, J = 8.8 Hz, 1 H), | |
| | 7.81 (dd, J = 8.1 and 1.5 Hz, 1 H), 7.95 (dd, J = 8.8 and 1.3 Hz, 1 H), 8.05 (d, J = 1.3 Hz, | |
| | 1 H), 8.15 (d, J = 8.1 Hz, 1 H), and 8.55 (s br, 1 H). | |
| 83 | ¹H NMR (DMSO-d6): δ0.81 (d, J = 6.9 Hz, 3 H), 0.85 (d, J = 6.9 Hz, 3 H), | 417.0 |
| | 1.96 (m, 1 H), 3.55 (d, J = 5.7 Hz, 1 H), 4.03 (t, J = 9.7 Hz, 2 H), 4.49 (t, J = 9.7 Hz, 2 | |
| | H), 7.83 (dd, J = 8.0 and 1.3 Hz, 1 H), 7.87 (d, J = 8.8 Hz, 1 H), 8.09 (d, J = 1.6 Hz, | |
| | 1 H), 8.15 (dd, J = 8.9 and 1.8 Hz, 1 H), 8.47 (d, J = 8.0 Hz, 1 H), and | |
| | 8.77 (d, J = 1.6 Hz, 1 H). | |
| 87 | ¹H NMR (DMSO-d6): δ0.77 (d, J = 6.9 Hz, 3 H), 0.88 (d, J = 6.9 Hz, 3 H), | 466.0 |
| | 2.00 (m, 1 H), 2.46 (m, 1 H), 3.09 (m, 1 H), 6.37 (s br, 2 H), 6.97 (m, 3 H), 7.34 (m, 2 | |
| | H), 7.81 (d br, J = 8.0 Hz, 2 H), 8.08 (s br, 1 H), 8.24 (s br, 1 H), 8.33 (d, J = 8.0 Hz, | |
| | 1 H), and 8.84 (s br, 1 H). | |
| 88 | ¹H NMR (DMSO-d6): δ0.79 (d, J = 6.9 Hz, 3 H), 0.91 (d, J = 6.9 Hz, 3 H), | 480.1 |
| | 2.04 (m, 1 H), 3.11 (m, 1 H), 3.27 (m, 1 H), 4.63 (s, br, 2 H), 7.52-7.27 (m, 6 H), | |
| | 7.73 (d, J = 9.0 Hz, 1 H), 7.79 (d, J = 7.9 Hz, 1 H), 8.00 (d br, J = 6.8 Hz, 1 H), | |
| | 8.06 (s, 1 H), 8.20 (d br, J = 8.5 Hz, 1 H), and 8.66 (s br, 1 H). | |
| 106 | ¹H NMR (DMSO-d6): δ0.80 (d, J = 6.9 Hz, 3 H), 0.83 (d, J = 6.9 Hz, 3 H), 1.12 (t, | 446.2 |
| | J = 7.1 Hz, 3 H), 1.32 (t, J = 7.1 Hz, 3 H), 1.96 (m, 1 H), 3.31 (q, J = 7.1 Hz, 2 H), | |
| | 3.63 (dd, J = 9.3 and 3.4 Hz, 1 H), 3.69 (q, J = 7.1 Hz, 2 H), 7.83 (dd, J = 8.3 | |
| | and 1.8 Hz, 1 H), 7.90 (dd, J = 8.3 and 1.6 Hz, 1 H), 8.04 (d, J = 8.6 Hz, 1 H), | |
| | 8.16 (d, J = 1.6 Hz, 1 H), 8.22 (d, J = 9.4 Hz, 1 H), 8.42 (d, J = 8.1 Hz, 1 H), | |
| | 8.57 (d, J = 1.6 Hz, 1 H), 9.11 (s br, 1 H), and 9.44 (s br, 1 H). | |
| 107 | ¹H NMR (DMSO-d6): δ0.81 (d, J = 6.9 Hz, 3 H), 0.84 (d, J = 6.9 Hz, 3 H), 1.96 (m, | 433.1 |
| | 1 H), 3.63 (dd, J = 8.1 and 8.1 Hz, 2 H), 3.67 (dd, J = 9.3 and 3.4 Hz, 1 H), | |
| | 4.49 (dd, J = 8.1 and 8.1 Hz, 2 H), 7.87 (dd, J = 8.2 and 1.6 Hz, 1 H), 7.94 (d, J = 8.7 Hz, | |
| | 1 H), 8.12 (d, J = 1.0 Hz, 1 H), 8.14 (dd, J = 8.7 and 1.9 Hz, 1 H), 8.19 (d, J = 9.3 Hz, | |
| | 1 H), 8.49 (d, J = 8.2 Hz, 1 H), and 8.78 (d, J = 1.6 Hz, 1 H). | |
| 108 | ¹H NMR (DMSO-d6): δ0.81 (d, J = 6.9 Hz, 3 H), 0.84 (d, J = 6.9 Hz, 3 H), 1.96 (m, | 420.2 |
| | 1 H), 3.62 (dd, J = 9.4 and 6.0 Hz, 1 H), 3.86 (s, 3 H), 7.98-7.85 (m, 3 H), | |
| | 8.12 (d, J = 1.2 Hz, 1 H), 8.18 (d, J = 9.5 Hz, 1 H), 8.37 (d, J = 8.2 Hz, 1 H), and | |
| | 8.62 (d, J = 1.0 Hz, 1 H). | |
| 109 | ¹H NMR (DMSO-d6): δ0.80 (d, J = 6.9 Hz, 3 H), 0.83 (d, J = 6.9 Hz, 3 H), 1.12 (t, | 446.3 |
| | J = 6.9 Hz, 3 H), 1.29 (t, J = 7.4 Hz, 3 H), 1.96 (m, 1 H), 3.24 (m, 2 H), 3.47 (m, 2 | |
| | H), 3.63 (dd, J = 9.3 and 6.0 Hz, 1 H), 7.84 (dd, J = 8.5 and 2.0 Hz, 1 H), | |
| | 7.90 (dd, J = 8.2 and 1.5 Hz, 1 H), 8.04 (d, J = 8.5 Hz, 1 H), 8.16 (d, J = 1.5 Hz, 1 | |
| | H), 8.21 (d, J = 9.2 Hz, 1 H), 8.42 (d, J = 8.5 Hz, 1 H), 8.57 (d, J = 2.0 Hz, 1 H), | |
| | 9.25 (t br, 1 H), and 9.70 (t br, 1 H). | |
| 110 | ¹H NMR (DMSO-d6): δ0.80 (d, J = 6.9 Hz, 3 H), 0.83 (d, J = 6.9 Hz, 3 H), 1.19 (d, | 445.93 |
| | J = 6.6 Hz, 6 H), 1.97 (m, 1 H), 3.13 (s, 3 H), 3.63 (dd, J = 9.3 and 5.8 Hz, 1 H), | |
| | 3.82 (m, 1 H), 7.84 (dd, J = 8.2 and 1.6 Hz, 1 H), 7.91 (dd, J = 8.2 and 1.8 Hz, 1 | |
| | H), 8.06 (d, J = 8.7 Hz, 1 H), 8.17 (m, 1 H), 8.22 (d, J = 9.5 Hz, 1 H), 8.42 (d, J = 8.5 Hz, | |
| | 1 H), 8.55 (d, J = 1.5 Hz, 1 H), 9.02 (s br, 1 H), 9.40 (s br, 1 H), and | |
| | 12.50 (s br, 1 H). | |
| 127 | ¹H NMR (DMSO-d6 + trifluoroacetic acid (TFA)): δ0.80 (d, J = 6.7 Hz, 3 H), | 444.1 |
| | 0.82 (d, J = 7.0 Hz, 3 H), 1.81-2.01 (m, 4 H), 2.02-2.17 (m, 1 H), 3.49 (t, J = 6.7 Hz, 2 | |
| | H), 3.56-3.68 (m, 3 H), 7.88 (dd, J = 3.1 and 1.6 Hz, 1 H), 7.91 (dd, J = 2.6 and | |
| | 1.8 Hz, 1 H), 8.03 (d, J = 8.5 Hz, 1 H), 8.16 (d, J = 1.2 Hz, 1 H), 8.21 (d, J = 9.4 Hz, | |
| | 1 H), 8.41 (d, J = 8.2 Hz, 1 H), 8.60 (d, J = 1.5 Hz, 1 H), 8.93 (s, 1 H), and | |
| | 9.40 (s, 1 H). | |
| 128 | ¹H NMR (DMSO-d6): δ0.81 (d, J = 7.2 Hz, 3 H), 0.83 (d, J = 7.2 Hz, 3 H), 1.31 (t, | 418.1 |
| | J = 7.3 Hz, 3 H), 1.87-2.05 (m, 1 H), 3.42-3.55 (m, 2 H), 3.64 (dd, J = 9.5 and | |
| | 6.0 Hz, 1 H), 7.92 (dd, J = 8.2 and 1.5 Hz, 1 H), 7.97 (dd, J = 8.8 and 2.0 Hz, 1 | |
| | H), 8.06 (d, J = 8.5 Hz, 1 H), 8.17 (d, J = 1.2 Hz, 1 H), 8.22 (d, J = 9.4 Hz, 1 H), | |
| | 8.41 (d, J = 8.5 Hz, 1 H), 8.70 (d, J = 1.5 Hz, 1 H), 9.07 (br. s., 1 H), 9.53 (br. s., | |
| | 1 H), and 9.85 (t, J = 5.0 Hz, 1 H). | |

### Example 10

### Preparation of (S)-3-methyl-2-(8-(5-methyl-1,2,4-oxadiazol-3-yl)dibenzo [b,d]furan-3-sulfonamido)butanoic acid (Compound 91)

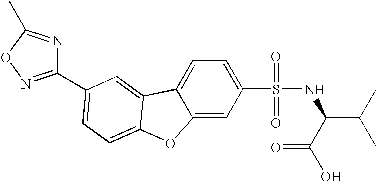

### Step 1: Preparation of (S)-methyl 2-(8-cyanodibenzo[b,d]furan-3-sulfonamido)-3-methylbutanoate

(S)-Methyl 2-(8-bromodibenzo[b,d]furan-3-sulfonamido)-3-methylbutanoate (1.0 g, 2.27 mmol), zinc cyanide (293 mg, 2.5 mmol), and Pd(PPh₃)₄ (79 mg, 0.07 mmol) were dissolved in 20 mL of NMP in a 20-mL microwave vial. The solution was deoxygenated for 5 minutes and was irradiated with microwave at 100° C. until no starting material was left according to LC-MS. Water was added to the reaction mixture and the precipitate was filtered to give the crude product, which was precipitated from methylene chloride/hexane solution, upon filtration, to give (S)-methyl 2-(8-cyanodibenzo[b,d]furan-3-sulfonamido)-3-methylbutanoate as a white solid.

### Step 2: Preparation of (S)-methyl 2-(8-(N-hydroxycarbamimidoyl)dibenzo[b,d]furan-3-sulfonamido)-3-methylbutanoate

(S)-Methyl 2-(8-cyanodibenzo[b,d]furan-3-sulfonamido)-3-methylbutanoate (500 mg, 1.29 mmol) was dissolved in 20 mL of DMF in a 100-mL round-bottom flask and hydroxylamine hydrochloride (448 mg, 6.45 mmol) and triethylamine (2.7 mL, 19.4 mmol) were added. The reaction was stirred at room temperature overnight, diluted with water, and the resulting mixture was filtered to produce (S)-methyl 2-(8-(N-hydroxycarbamimidoyl) dibenzo[b,d]furan-3-sulfonamido)-3-methylbutanoate (460 mg, 85% yield) as a white solid.

### Step 3: Preparation of (S)-methyl 3-methyl-2-(8-(5-methyl-1,2,4-oxadiazol-3-yl)dibenzo[b,d]furan-3-sulfonamido)butanoate

(S)-Methyl 2-(8-(N-hydroxycarbamimidoyl)dibenzo[b,d]furan-3-sulfonamido)-3-methylbutanoate (15 mg, 0.24 mmol) was dissolved in 0.3 mL of acetic acid and the resulting solution was cooled to 0° C. Acetic anhydride (0.3 mL) was added and the reaction mixture was stirred at 0° C. for 30 minutes, heated at 92° C. for 4 hours, and concentrated. The residue was diluted with 1.0 mL of water, stirred for 10 minutes, and filtered to produce (S)-methyl-3-methyl-2-(8-(5-methyl-1,2,4-oxadiazol-3-yl)dibenzo[b,d]furan-3-sulfonamido) butanoate (13 mg, 85% yield).

### Step 4: Preparation of (S)-3-methyl-2-(8-(5-methyl-1,2,4-oxadiazol-3-yl)dibenzo [b,d]furan-3-sulfonamido)butanoic acid

(S)-Methyl-3-methyl-2-(8-(5-methyl-1,2,4-oxadiazol-3-yl)dibenzo[b,d]furan-3-sulfonamido)butanoate (13 mg, 0.03 mmol) was suspended in a mixture of 0.5 mL of concentrated hydrochloric acid and 0.5 mL of acetic acid. The reaction mixture was heated to 90° C. for two hours and cooled to room temperature. Water was added and the resulting solid was filtered to produce (S)-3-methyl-2-(8-(5-methyl-1,2,4-oxadiazol-3-yl)dibenzo [b,d]furan-3-sulfonamido)butanoic acid (10 mg, 81%) as a white solid. MS (ESI, [M−H]⁻): 428.11.

### Example 10A

### (R)-3-methyl-2-(8-(5-(trifluoromethyl)-1,2,4-oxadiazol-3-yl)dibenzo[b,d]furan-3-sulfonamido)butanoic acid (Compound 185)

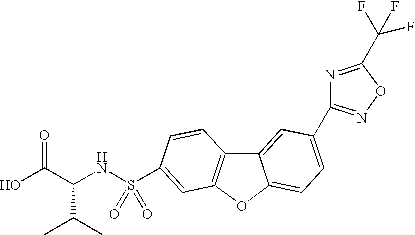

The title compound was prepared by the procedures described in Example 10, using L-Valine instead of D-Valine and 2,2,2-trifluoroacetic anhydride and TFA were used instead of acetic anhydride and acetic acid. The compound was obtained as an off-white solid. ¹H NMR (400 MHz, MeOD) δ ppm 1.01 (d, J=6.82 Hz, 3H), 1.07 (d, J=6.82 Hz, 3H), 2.10-2.20 (m, 1H), 3.84 (d, J=5.56 Hz, 1H), 7.94-8.06 (m, 2H), 8.23-8.27 (m, 1H), 8.39-8.50 (m, 2H), 8.99-9.05 (m, 1H). HRMS (ESI-FTMS): calcd for C₂₀H₁₆F₃N₃O₆S+H⁺, 484.07847. found: 484.07811.

The following compounds in Table 10 were prepared using procedures analogous to those described above for the preparation of (S)-3-methyl-2-(8-(5-methyl-1,2,4-oxadiazol-3-yl)dibenzo[b,d]furan-3-sulfonamido)butanoic acid.

TABLE 10

| Compd | | |
|---|---|---|
| No. | NMR | MS |
| 92 | ¹H NMR (MeOD): δ0.91 (d, J = 6.82 Hz, 3 H), 0.97 (d, J = 6.82 Hz, 3 H), 1.99-2.12 (m, 1 | 481.92 |
| | H), 3.74 (d, J = 5.81 Hz, 1 H), 7.84-7.96 (m, 2 H), 8.11-8.18 (m, 1 H), 8.29-8.39 (m, 2 H), | |
| | and 8.88-8.96 (m, 1 H). | |
| 93 | — | 416.1 |
| 113 | ¹H NMR (DMSO-d6): δ8.92 (d, J = 1.77 Hz, 1 H), 8.55 (d, J = 8.59 Hz, 1 H), 8.26 (dd, J = 8.72 | 470.3 |
| | and 1.89 Hz, 1 H), 8.11 (d, J = 1.52 Hz, 1 H), 7.97 (d, J = 8.59 Hz, 1 H), 7.84 (dd, J = 8.08 | |
| | and 1.52 Hz, 1 H), 1.85-2.02 (m, 1 H), 1.49 (s, 9 H), and 0.82 (dd, J = 19.71 and 6.82 Hz, | |
| | 6 H). | |
| 114 | ¹H NMR (DMSO-d6): δ8.93 (d, J = 1.26 Hz, 1 H), 8.56 (d, J = 8.08 Hz, 1 H), 8.26 (dd, J = 8.84 | 458 |
| | and 1.77 Hz, 1 H), 8.12 (d, J = 1.26 Hz, 1 H), 7.98 (d, J = 8.84 Hz, 1 H), 7.85 (dd, J = 8.21 | |
| | and 1.64 Hz, 1 H), 1.90-2.01 (m, 1 H), 1.42 (dd, 6 H), and 0.83 (dd, 6 H). | |
| 123 | ¹H NMR (DMSO-d6): δ8.94 (d, J = 1.77 Hz, 1 H), 8.55 (d, J = 8.08 Hz, 1 H), 8.26 (dd, J = 8.59 | 444.21 |
| | and 1.77 Hz, 1 H), 8.12 (d, J = 1.52 Hz, 1 H), 7.98 (d, J = 8.84 Hz, 1 H), 7.85 (dd, J = 8.21 | |
| | and 1.39 Hz, 1 H), 3.55-3.66 (m, 1 H), 3.07 (q, J = 7.58 Hz, 2 H), 1.89-2.02 (m, 1 H), | |
| | 1.39 (t, J = 7.58 Hz, 3 H), and 0.83 (dd, J = 14.40 and 6.82 Hz, 6 H). | |

### Example 11

### (S)-2-(8-(N-hydroxycarbamimidoyl)dibenzo[b,d]furan-3-sulfonamido)-3-methylbutanoic acid (Compound 82)

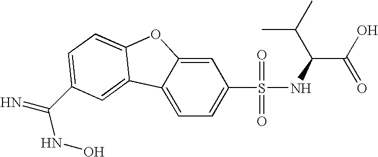

(S)-2-(8-(N-Hydroxycarbamimidoyl)dibenzo[b,d]furan-3-sulfonamido)-3-methyl butanoic acid was obtained as a white powder by acid hydrolysis of (S)-methyl 2-(8-(N-hydroxycarbamimidoyl)dibenzo[b,d]furan-3-sulfonamido)-3-methylbutanoate. MS (ES, [M+H]⁺): 406.1.

### Example 12

### (S)-3-methyl-2-(8-(2-methyl-2H-tetrazol-5-yl)dibenzo[b,d]furan-3-sulfonamido)butanoic acid (Compound 132)

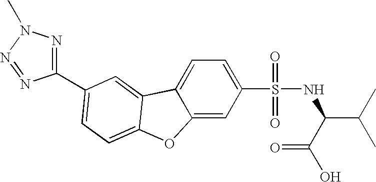

### Step 1: Preparation of (S)-tert-butyl 2-(8-cyanodibenzo[b,d]furan-3-sulfonamido)-3-methylbutanoate

(S)-Tert-butyl 2-(8-bromodibenzo[b,d]furan-3-sulfonamido)-3-methylbutanoate (5 g, 0.01 mol, 1 eq.) and zinc cyanide (3.04 g, 0.026 mol, 2.5 eq.) were mixed in 50 mL of dimethylacetamide (DMA). The solution was deoxygenated with nitrogen for 15 minutes and Pd(PPh₃)₄ (700 mg, 0.62 mmol, 0.06 eq.) was added. The reaction mixture was heated at 120° C. for 2 hours, diluted with water, and the resulting solution was extracted with ethyl acetate. The combined ethyl acetate fractions were washed with water, brine, dried over anhydrous Na₂SO₄, and concentrated to provide a yellow liquid, which was purified by silica gel column chromatography to produce (S)-tert-butyl 2-(8-cyanodibenzo[b,d]furan-3-sulfonamido)-3-methylbutanoate (2.74 g, 62% yield).

### Step 2: Preparation of (S)-tert-butyl 2-(8-(2H-tetrazol-5-yl)dibenzo[b,d]furan-3-sulfonamido)-3-methylbutanoate

(S)-Tert-butyl 2-(8-(2H-tetrazol-5-yl)dibenzo[b,d]furan-3-sulfonamido)-3-methyl butanoate was prepared following a literature procedure described for similar compounds (see, e.g., Synthesis, 1999: 1004).

### Step 3: Preparation of (S)-tert-butyl 3-methyl-2-(8-(2-methyl-2H-tetrazol-5-yl)dibenzo[b,d]furan-3-sulfonamido)butanoate

(S)-Tert-butyl 2-(8-(2H-tetrazol-5-yl)dibenzo[b,d]furan-3-sulfonamido)-3-methyl butanoate (180 mg) was dissolved in 2 mL of acetonitrile (CH₃CN) and, after addition of MeI (50 mg, 22 μL), the resulting mixture was stirred at room temperature overnight. The solvent was removed, 2 mL of TFA/DCM (30%) was added, and the resulting mixture was stirred at room temperature until no starting material was left. The crude product was purified by a preparative HPLC to produce (S)-tert-butyl 3-methyl-2-(8-(2-methyl-2H-tetrazol-5-yl)dibenzo[b,d]furan-3-sulfonamido)butanoate. MS (ES, [M+H]⁺): 430.15.

### Example 13

### (S)-2-(8-(2H-tetrazol-5-yl)dibenzo[b,d]furan-3-sulfonamido)-3-methyl butanoic acid (Compound 130)

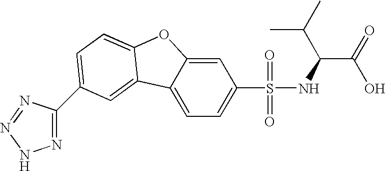

(S)-2-(8-(2H-Tetrazol-5-yl)dibenzo[b,d]furan-3-sulfonamido)-3-methylbutanoic acid was obtained by treating (S)-tert-butyl 2-(8-(2H-tetrazol-5-yl)dibenzo[b,d]furan-3-sulfonamido)-3-methylbutanoate with 2 mL of TFA/DCM (30%). The crude product was purified by a preparative HPLC to produce (S)-2-(8-(2H-tetrazol-5-yl)dibenzo[b,d]furan-3-sulfonamido)-3-methylbutanoic acid. MS (ES, [M+H]⁺): 416.07.

### Example 14

### (S)-2-(7-(5-isopropyl-1,2,4-oxadiazol-3-yl)dibenzo[b,d]furan-3-sulfonamido)-3-methylbutanoic acid (Compound 154)

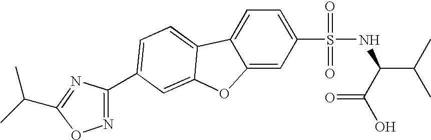

### Step 1: Preparation of 8-bromodibenzo[b,d]furan-3-sulfonyl chloride

Dibenzo[b,d]furan-3-sulfonyl chloride (5.3 g, 20 mmol, 1.0 eq.) was mixed with acetic acid (glacial, 120 mL) and bromine (10 mL, 10 eq.) and the mixture was stirred at 70° C. for 4 hours. The excess bromine was removed by bubbling nitrogen through the reaction mixture and trapped with saturated Na₂SO₃ solution. The resulting solution was cooled to room temperature and filtered to give 8-bromodibenzo[b,d]furan-3-sulfonyl chloride (5.4 g) as a light brown solid.

### Step 2: Preparation of (S)-methyl 2-(8-bromodibenzo[b,d]furan-3-sulfonamido)-3-methylbutanoate

8-Bromodibenzo[b,d]furan-3-sulfonyl chloride (3.46 g, 10 mmol) and (S)-methyl 2-amino-3-methylbutanoate hydrochloride (1.1 eq.) was mixed in 30 mL of DCM, N,N-diisopropylethylamine (3.84 mL, 2.2 eq.) was added, and the resulting mixture was stirred at room temperature for 5 hours. The crude product mixture was purified by column chromatography to produce (S)-methyl 2-(8-bromodibenzo[b,d]furan-3-sulfonamido)-3-methylbutanoate (4.7 g) as a white solid.

### Step 3: Preparation of (S)-methyl 2-(8-bromo-7-nitrodibenzo[b,d]furan-3-sulfonamido)-3-methylbutanoate

(S)-Methyl 2-(8-bromodibenzo[b,d]furan-3-sulfonamido)-3-methylbutanoate (724 mg, 1.6 mmol) and HNO₃ (0.27 g, 4.2 mmol) was dissolved in a mixture of 15 mL of TFA and 1 mL of DCM and the resulting solution were stirred at room temperature for 5 hours. The solvents were removed to provide the crude product, which was purified by column chromatography to produce (S)-methyl 2-(8-bromo-7-nitrodibenzo[b,d]furan-3-sulfonamido)-3-methylbutanoate (625 mg) as a yellow solid.

### Step 4: Preparation of (S)-methyl 2-(7-aminodibenzo[b,d]furan-3-sulfonamido)-3-methylbutanoate

(S)-Methyl 2-(8-bromo-7-nitrodibenzo[b,d]furan-3-sulfonamido)-3-methylbutanoate (11.56 g, 23.8 mmol) was mixed with Pd/C (700 mg) in 200 mL of MeOH and the reaction was carried out in a Parr® shaker at room temperature under hydrogen (50 psi) overnight. The reaction mixture was filtered through a Celite® pad and the filtrate was concentrated to produce (S)-methyl 2-(7-aminodibenzo[b,d]furan-3-sulfonamido)-3-methylbutanoate (8.92 g) as a grey solid.

### Step 5: Preparation of (S)-methyl 2-(7-iododibenzo[b,d]furan-3-sulfonamido)-3-methylbutanoate

(S)-Methyl 2-(7-aminodibenzo[b,d]furan-3-sulfonamido)-3-methylbutanoate (3.72 g, 9.9 mmol) was dissolved in a mixture of 3.5 mL of HCl, 12 mL of H₂O, and 50 mL of acetic acid and a NaNO₂ solution (2 M, 7.5 mL) was added dropwise at 0° C., followed by the addition of NaI (11.87 g, 80 mmol). The mixture was slowly warmed to room temperature, stirred for 3 h, and filtered. The resulting solid was washed with water and purified by column chromatography to produce (S)-methyl 2-(7-iododibenzo[b,d]furan-3-sulfonamido)-3-methylbutanoate (3.94 g) as a grey solid.

### Step 6: Preparation of (S)-methyl 2-(7-cyanodibenzo[b,d]furan-3-sulfonamido)-3-methylbutanoate

(S)-Methyl 2-(7-iododibenzo[b,d]furan-3-sulfonamido)-3-methylbutanoate (1.02 g, 2.1 mmol), CuCN (0.28 g, 3.1 mmol), and Pd(PPh₃)₄ (130 mg) were dissolved in 8 mL of NMP; and the resulting solution was deoxygenated with nitrogen for 5 minutes and was irradiated with microwave at 120° C. for 20 minutes. The reaction mixture was purified by column chromatography to produce (S)-methyl 2-(7-cyanodibenzo[b,d]furan-3-sulfonamido)-3-methylbutanoate (670 mg) as a white solid.

### Step 7: Preparation of (S)-methyl 2-(7-(N-hydroxycarbamimidoyl)dibenzo[b,d]furan-3-sulfonamido)-3-methylbutanoate

A solution of (S)-methyl 2-(7-cyanodibenzo[b,d]furan-3-sulfonamido)-3-methyl butanoate (120 mg, 0.31 mmol), hydroxylamine hydrochloride (324 mg, 4.6 mmol), and triethyl amine (629 mg, 6.2 mmol) in 2 mL of DMF was stirred at room temperature for 6 hours and the crude product was purified by a preparative HPLC to produce (S)-methyl 2-(7-(N-hydroxycarbamimidoyl)dibenzo[b,d]furan-3-sulfonamido)-3-methylbutanoate (123 mg) as a white solid.

### Step 8: Preparation of (S)-methyl 2-(7-(5-isopropyl-1,2,4-oxadiazol-3-yl)dibenzo [b,d]furan-3-sulfonamido)-3-methylbutanoate

(S)-Methyl 2-(7-(N-hydroxycarbamimidoyl)dibenzo[b,d]furan-3-sulfonamido)-3-methyl butanoate (60 mg, 0.14 mmol) was suspended in 2 mL of isobutyric acid and the resulting mixture was cooled to 0° C. Isobutyric anhydride (360 mg, 2.3 mmol) was added dropwise and the reaction mixture was slowly heated to 90° C. and stirred for 3 hours. The crude product was purified by a preparative HPLC to produce (S)-methyl 2-(7-(5-isopropyl-1,2,4-oxadiazol-3-yl)dibenzo[b,d]furan-3-sulfonamido)-3-methylbutanoate (59 mg) as a white solid.

### Step 9: Preparation of (S)-2-(7-(5-isopropyl-1,2,4-oxadiazol-3-yl)dibenzo[b,d]furan-3-sulfonamido)-3-methylbutanoic acid

(S)-Methyl 2-(7-(5-isopropyl-1,2,4-oxadiazol-3-yl)dibenzo[b,d]furan-3-sulfonamido)-3-methylbutanoate (59 mg) was dissolved in 1 mL of THF and a LiOH solution (1 mL, 0.9 M) was added. The reaction mixture was stirred at room temperature for 3 days, concentrated, and the remaining aqueous solution was acidified to pH ˜2. The mixture was filtered and the filtrate was concentrated to produce (S)-2-(7-(5-isopropyl-1,2,4-oxadiazol-3-yl) dibenzo[b,d]furan-3-sulfonamido)-3-methylbutanoic acid (46 mg) as a white solid. MS (LC-MS, [M+H]⁺): 456.32.

### Example 14A

### (S)-2-(7-(N-hydroxycarbamimidoyl)dibenzo[b,d]furan-3-sulfonamido)-3-methylbutanoic acid (Compound 186)

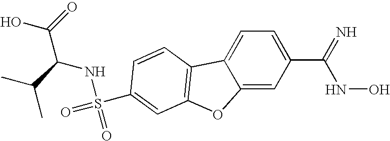

The title compound was prepared by acid hydrolysis (6 N HCl, 80° C., 4 hours in acetic acid) of the intermediate (S)-methyl 2-(7-(N-hydroxycarbamimidoyl)dibenzo[b,d]furan-3-sulfonamido)-3-methylbutanoate (an intermediate after step 7 in the preparation of Example 14). The final product was obtained as a white solid in 30% yield. ¹H NMR (400 MHz, MeOD) δ ppm 1.17 (d, J=6.82 Hz, 3H), 1.25 (d, J=6.82 Hz, 3H), 2.28-2.36 (m, 1H), 3.96 (d, J=5.56 Hz, 1H), 8.03 (dd, J=8.08, 1.26 Hz, 1H), 8.15 (dd, J=8.21, 1.64 Hz, 1H), 8.22 (s, 1H), 8.37 (d, J=1.01 Hz, 1H), 8.42 (d, J=8.34 Hz, 1H), 8.48 (d, J=8.08 Hz, 1H). HRMS (ESI-FTMS): calcd for C₁₈H₁₉N₃O₆S+H⁺, 406.10673. found: 406.10709.

### Example 14B

### (S)-2-(7-(5-cyclopropyl-1,2,4-oxadiazol-3-yl)dibenzo[b,d]furan-3-sulfonamido)-3-methylbutanoic acid (Compound 187)

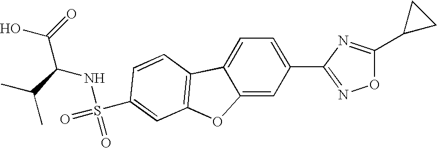

The title compound was prepared by the procedures described in Example 14, using cyclopropanecarbonyl chloride instead of isobutyric anhydride and isobutyric acid. The reaction was carried out in dichloromethane in the presence of aqueous sodium bicarbonate. The product was obtained as a white solid in 90% yield. ¹H NMR (400 MHz, MeOD) δ ppm 1.24 (d, J=6.82 Hz, 3H), 1.30 (d, J=6.82 Hz, 3H), 1.57-1.70 (m, 4H), 2.31-2.47 (m, 1H), 2.62-2.73 (m, 1H), 4.06 (d, 1H), 8.23 (dd, J=8.08, 1.52 Hz, 1H), 8.39-8.49 (m, 2H), 8.54-8.62 (m, 3H). HRMS (ESI-FTMS): calcd for C₂₂H₂₁N₃O₆S+H⁺, 456.12238. found: 456.12296.

### Example 14C

### (S)-2-(7-(5-(4-fluorophenyl)-1,2,4-oxadiazol-3-yl)dibenzo[b,d]furan-3-sulfonamido)-3-methylbutanoic acid (Compound 188)

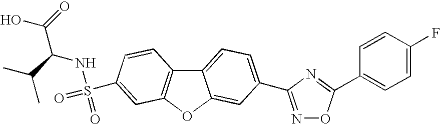

The title compound was prepared by the procedures described in Example 14, using 4-fluorobenzoyl chloride instead of isobutyric anhydride and isobutyric acid. The reaction was carried out in dichloromethane in the presence of aqueous sodium bicarbonate. The final product was obtained as a white solid in 40% yield. ¹H NMR (400 MHz, MeOD) δ ppm 1.13 (d, J=6.32 Hz, 3H), 1.21 (d, J=6.32 Hz, 3H), 2.26-2.34 (m, 1H), 3.95 (s, 1H), 7.55-7.66 (m, 2H), 8.08-8.17 (m, 1H), 8.37 (s, 1H), 8.43-8.58 (m, 5H), 8.64 (s, 1H). HRMS (ESI-FTMS): calcd for C₂₅H₂₀FN₃O₆S+H⁺, 510.11296. found: 510.11472.

The following compounds in Table 11 were prepared using procedures analogous to those described above for the preparation of (S)-2-(7-(5-isopropyl-1,2,4-oxadiazol-3-yl)dibenzo[b,d]furan-3-sulfonamido)-3-methylbutanoic acid.

TABLE 11

| Compd No. | MS |
|---|---|
| 155 | 470.33 |
| 156 | 428.27 |

### Example 15

### (R)-3-methyl-2-(7-(5-methyl-1,2,4-oxadiazol-3-yl)dibenzo[b,d]furan-2-sulfonamido)butanoic acid (Compound 139)

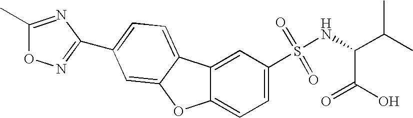

### Step 1: Preparation of 3-nitrodibenzo[b,d]furan

Dibenzofuran (50 g, fine powder) was mixed with 400 mL of TFA and the resulting suspension was cooled in an ethanol-ice bath. Fuming HNO₃ (11.7 mL, >90%) was added drop-wise over 10 minutes and the reaction mixture was warmed to room temperature and stirred for two hours. After filtration, the solid was triturated with methanol and dried under vacuum to produce 3-nitrodibenzo[b,d]furan (45 g, 70% yield) as a white solid.

### Step 2: Preparation of 7-nitrodibenzo[b,d]furan-2-sulfonic acid

To a round-bottom flask containing 3-nitrodibenzo[b,d]furan (21.4 g, 100 mmol) in 200 mL of chloroform was slowly added chlorosulfonic acid (15.2 g, 130 mmol) at 0° C. The resulting suspension was warmed to room temperature and stirred for 4 hours. The reaction mixture was cooled to 0° C. and filtered to produce 7-nitrodibenzo[b,d]furan-2-sulfonic acid (24.1 g, 81% yield) as a white solid.

### Step 3: Preparation of 7-nitrodibenzo[b,d]furan-2-sulfonyl chloride

7-Nitrodibenzo[b,d]furan-2-sulfonic acid (2.93 g, 10 mmol) was mixed with thionyl chloride (15 mL) and DMF (2 drops) was added slowly. The resulting mixture was stirred at 80° C. for 24 hours, cooled to room temperature, filtered, and the excess thionyl chloride in the filtrate was removed under reduced pressure. The crude product was triturated with ice-water to produce 7-nitrodibenzo[b,d]furan-2-sulfonyl chloride (2.78 g, 89% yield) as an off-white solid.

### Step 4: Preparation of (R)-methyl 3-methyl-2-(7-nitrodibenzo[b,d]furan-2-sulfonamido)butanoate

7-Nitrodibenzo[b,d]furan-2-sulfonyl chloride (570 mg, 1.83 mmol) and (R)-methyl 2-amino-3-methylbutanoate hydrochloride (334 mg, 2.0 mmol) were mixed in 5 mL of DCM and N,N-diisopropylethylamine (520 mg, 4 mmol) was added slowly at 0° C. The reaction mixture was warmed to room temperature and stirred for 4 hours. The crude product was purified by column chromatography to produce (R)-methyl 3-methyl-2-(7-nitrodibenzo[b,d]furan-2-sulfonamido)butanoate (0.658 g, 88% yield) as a white solid.

### Step 5: Preparation of (R)-methyl 2-(7-aminodibenzo[b,d]furan-2-sulfonamido)-3-methylbutanoate

(R)-Methyl 3-methyl-2-(7-nitrodibenzo[b,d]furan-2-sulfonamido)butanoate (480 mg) was dissolved in 20 mL of MeOH and Pd/C (100 mg, 10%) was added. The reaction was carried out in a Parr® shaker at room temperature under hydrogen (50 psi) overnight. The reaction mixture was filtered through a Celite® pad and the filtrate was concentrated to produce (R)-methyl 2-(7-aminodibenzo[b,d]furan-2-sulfonamido)-3-methylbutanoate (430 mg, quantitative yield) as an off-white solid.

The t-butyl ester analog, as well as the (S)-isomer analog, were prepared similarly using the corresponding amino acid analog at step 4.

### Step 6: Preparation of (R)-methyl 2-(7-iododibenzo[b,d]furan-2-sulfonamido)-3-methylbutanoate

(R)-Methyl 2-(7-aminodibenzo[b,d]furan-2-sulfonamido)-3-methylbutanoate (2.165 g, 5.75 mmol) was mixed with 12 mL of hydrochloric acid (18%), a NaNO₂ solution (9 mL, 1.0 M) was added at 0° C., and the resulting mixture was stirred at 0° C. for 20 minutes. A solution of sodium iodide (0.948 g, 6.32 mmol, in 3 mL of water) was added very slowly and the reaction mixture was stirred for 20 minutes. Upon addition of water, the resulting solid was filtered to give (R)-methyl 2-(7-iododibenzo[b,d]furan-2-sulfonamido)-3-methylbutanoate (71% yield) as a dark brown solid.

### Step 7: Preparation of (R)-methyl 2-(7-cyanodibenzo[b,d]furan-2-sulfonamido)-3-methylbutanoate

(R)-Methyl 2-(7-iododibenzo[b,d]furan-2-sulfonamido)-3-methylbutanoate (1.0 g, 2.27 mmol), zinc cyanide (0.293 g, 2.5 mmol), and Pd(PPh₃)₄ (79 mg, 0.07 mmol) were dissolved in 20 mL of NMP in a 20-mL microwave vial. The solution was deoxygenated for 5 minutes and was irradiated with microwave at 100° C. until no starting material was left according to LC-MS. Upon completion, water was added to the reaction mixture and the precipitate was filtered to give the crude product, which was re-precipitated from DCM/hexane to produce (R)-methyl 2-(7-cyanodibenzo[b,d]furan-2-sulfonamido)-3-methylbutanoate as a white solid.

### Step 8: Preparation of (R)-methyl 2-(7-(N-hydroxycarbamimidoyl)dibenzo[b,d]furan-2-sulfonamido)-3-methylbutanoate

(R)-Methyl 2-(7-cyanodibenzo[b,d]furan-2-sulfonamido)-3-methylbutanoate (5.0 g, 12.9 mmol) was dissolved in 200 mL of DMF in a 500-mL round-bottom flask, to which were added hydroxylamine hydrochloride (4.483 g, 64.5 mmol) and triethylamine (27 mL, 194 mmol). The reaction mixture was stirred at room temperature overnight and filtered after addition of water to produce (R)-methyl 2-(7-(N-hydroxycarbamimidoyl) dibenzo[b,d]furan-2-sulfonamido)-3-methylbutanoate (4.60 g, 85%) as a white solid.

### Step 9: Preparation of (R)-methyl 3-methyl-2-(7-(5-methyl-1,2,4-oxadiazol-3-yl) dibenzo[b,d]furan-2-sulfonamido)butanoate

(R)-Methyl 2-(7-(N-hydroxycarbamimidoyl)dibenzo[b,d]furan-2-sulfonamido)-3-methylbutanoate (100 mg, 0.24 mmol) was dissolved in 2 mL of acetic acid and acetic anhydride (10 eq.) was added. The reaction mixture was stirred at room temperature for 30 minutes and heated at 90° C. for 2 hours. After the solution was cooled to room temperature, 3 mL of water was added and the resulting mixture was filtered to give (R)-methyl 3-methyl-2-(7-(5-methyl-1,2,4-oxadiazol-3-yl)dibenzo[b,d]furan-2-sulfonamido)butanoate (115 mg, 90% yield) as a white solid.

### Step 10: Preparation of (R)-3-methyl-2-(7-(5-methyl-1,2,4-oxadiazol-3-yl)dibenzo [b,d]furan-2-sulfonamido)butanoic acid

(R)-Methyl 3-methyl-2-(7-(5-methyl-1,2,4-oxadiazol-3-yl)dibenzo[b,d]furan-2-sulfonamido)butanoate (90 mg, 0.21 mmol) was dissolved in 2 mL of THF/MeOH/water and a LiOH (5 eq.) solution was added. The reaction was stirred overnight, water was added, and pH of the solution was adjusted to between 4 and 5 with diluted hydrochloric acid. The precipitate was filtered to produce (R)-3-methyl-2-(7-(5-methyl-1,2,4-oxadiazol-3-yl) dibenzo[b,d]furan-2-sulfonamido)butanoic acid (72 mg, 80% yield) as a white solid.

### Compound 15A

### (R)-3-methyl-2-(7-(5-neopentyl-1,2,4-oxadiazol-3-yl)dibenzo[b,d]furan-2-sulfonamido)butanoic acid (Compound 189)

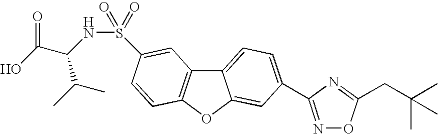

The title compound was prepared by the procedures described in Example 15, using 3,3-dimethylbutanoyl chloride instead of acetic anhydride and acetic acid. The compound was obtained as an off-white solid. ¹H NMR (400 MHz, DMSO-d₆) δ ppm 0.80 (d, J=6.82 Hz, 3H), 0.85 (d, J=6.57 Hz, 3H), 1.07 (s, 9H), 1.87-2.02 (m, 1H), 2.97 (s, 2H), 3.45-3.59 (m, 1H), 7.91-8.04 (m, 2H), 8.12 (dd, J=8.08, 1.26 Hz, 1H), 8.33 (d, J=1.26 Hz, 1H), 8.50 (d, J=7.83 Hz, 1H), 8.69 (d, J=2.02 Hz, 1H). HRMS (ESI-FTMS): calcd for C₂₄H₂₇N₃O₆S+H⁺, 486.16933. found: 486.17016.

### Compound 15B

### (R)-2-(7-(5-cyclopentyl-1,2,4-oxadiazol-3-yl)dibenzo[b,d]furan-2-sulfonamido)-3-methylbutanoic acid (Compound 190)

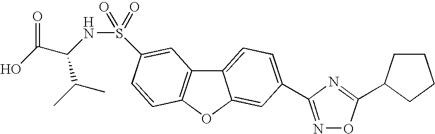

The title compound was prepared by the procedures described in Example 15, using cyclopentylcarbonylchloride instead of acetic anhydride and acetic acid. The compound was obtained as an off-white solid. ¹H NMR (400 MHz, DMSO-d₆) δ ppm 0.79 (d, J=6.57 Hz, 3H), 1.64-1.85 (m, 4H), 1.87-2.07 (m, 3H), 2.08-2.24 (m, 2H), 3.44-3.60 (m, 2H), 7.86-8.04 (m, 2H), 8.10 (dd, J=8.21, 1.39 Hz, 1H), 8.31 (s, 1H), 8.49 (d, J=8.34 Hz, 1H), 8.69 (d, J=1.77 Hz, 1H). HRMS (ESI-FTMS): calcd for C₂₄H₂₆N₃O₆S+H⁺, 484.15368. found: 484.15444.

### Compound 15C

### (R)-2-(7-(5-(cyclopentylmethyl)-1,2,4-oxadiazol-3-yl)dibenzo[b,d] furan-2-sulfonamido)-3-methylbutanoic acid (Compound 191)

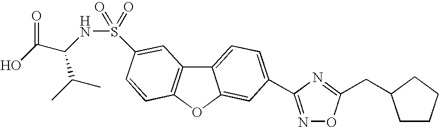

The title compound was prepared by the procedures described in Example 15, using 2-cyclopentylacetyl chloride instead of acetic anhydride and acetic acid. The compound was obtained as an off-white solid. ¹H NMR (400 MHz, DMSO-d₆) δ ppm 0.80 (d, J=6.82 Hz, 3H), 0.84 (d, J=6.82 Hz, 3H), 1.22-1.36 (m, 2H), 1.51-1.72 (m, 4H), 1.78-1.91 (m, 2H), 1.91-2.01 (m, 1H), 2.34-2.43 (m, 1H), 3.06 (d, J=7.33 Hz, 2H), 3.58-3.70 (m, 1H), 7.91-8.03 (m, 2H), 8.12 (dd, J=8.08, 1.26 Hz, 1H), 8.28-8.34 (m, 1H), 8.50 (d, J=8.08 Hz, 1H), 8.69 (d, J=2.02 Hz, 1H). HRMS (ESI-FTMS): calcd for C₂₅H₂₇N₃O₆S+H⁺, 498.16933. found: 498.16902.

### Compound 15D

### (R)-2-(7-(5-cyclohexyl-1,2,4-oxadiazol-3-yl)dibenzo[b,d]furan-2-sulfonamido)-3-methylbutanoic acid (Compound 192)

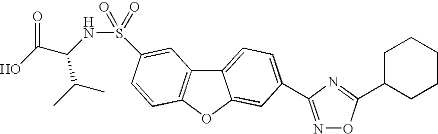

The title compound was prepared by the procedures described in Example 15, using cyclohexylcarbonylchloride instead of acetic anhydride and acetic acid. The compound was obtained as an off-white solid. ¹H NMR (400 MHz, DMSO-d₆) δ ppm 0.81 (d, J=6.82 Hz, 3H), 0.84 (d, J=6.82 Hz, 3H), 1.26-1.51 (m, 3H), 1.58-1.74 (m, 3H), 1.74-1.85 (m, 2H), 1.90-2.02 (m, 1H), 2.06-2.17 (m, 2H), 3.08-3.23 (m, 1H), 3.57-3.66 (m, 1H), 7.90-8.05 (m, 2H), 8.04-8.16 (m, 2H), 8.31 (s, 1H), 8.49 (d, J=8.08 Hz, 1H), 8.69 (d, J=2.02 Hz, 1H). HRMS (ESI-FTMS): calcd for C₂₅H₂₇N₃O₆S+H⁺, 498.16933. found: 498.16966.

The following compounds in Table 12 were prepared following procedures analogous to those described above for the preparation of (R)-3-methyl-2-(7-(5-methyl-1,2,4-oxadiazol-3-yl)dibenzo[b,d]furan-2-sulfonamido)butanoic acid.

TABLE 12

| Compd | | | |
|---|---|---|---|
| No. | NMR | HRMS | MS |
| 133 | ¹H NMR (DMSO-d6): δ8.70 (d, J = 1.77 Hz, 1 H), 8.51 (d, J = 8.08 Hz, | — | 472.21 |
| | 1 H), 8.32 (s, 1 H), 8.12 (dd, J = 8.08 and 1.52 Hz, 1 H), | | |
| | 7.93-8.04 (m, 2 H), 3.62 (dd, J = 9.22 and 6.19 Hz, 1 H), 1.90-2.02 (m, 1 | | |
| | H), 1.49 (s, 9 H), and 0.82 (dd, 6 H). | | |
| 140 | ¹H NMR (DMSO-d6): δ8.70 (d, J = 1.77 Hz, 1 H), 8.51 (d, J = 8.08 Hz, | — | 458.2 |
| | 1 H), 8.32 (s, 1 H), 8.08-8.17 (m, 2 H), 7.93-8.04 (m, 2 H), | | |
| | 3.58-3.67 (m, 1 H), 3.36-3.46 (m, 1 H), 1.90-2.03 (m, 1 H), 1.42 (d, | | |
| | 6 H), and 0.82 (dd, 6 H). | | |
| 141 | ¹H NMR (DMSO-d6): δ8.74 (d, J = 1.26 Hz, 1 H), 8.58 (d, J = 8.08 Hz, | — | 484.1 |
| | 1 H), 8.43 (s, 1 H), 8.11-8.22 (m, 2 H), 7.95-8.06 (m, 2 H), | | |
| | 3.59-3.67 (m, 1 H), 1.92-2.01 (m, 1 H), and 0.82 (dd, 6 H). | | |
| 147 | — | 456.12362 | — |
| 148 | — | 470.13951 | — |
| 149 | — | 472.15539 | — |
| 150 | — | 492.12397 | — |

### Example 16

### (S)-3-methyl-2-(8-(pyridin-3-yl)dibenzo[b,d] thiophene-3-sulfonamido) butanoic acid (Compound 11)

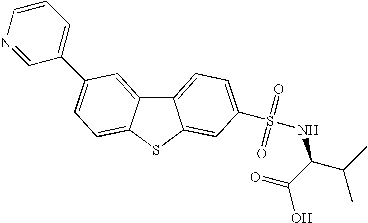

### Step 1: Preparation of dibenzo[b,d]thiophene-3-sulfonyl chloride

5-(Trifluoromethyl)-5H-dibenzo[b,d]thiophenium-3-sulfonate (200 mg) was mixed with 10 mL of thionyl chloride (SOCl₂) and a few drops DMF was added. The mixture was stirred at 80° C. for 24 hours, the excess SOCl₂ was removed under vacuum, and the residue was triturated with ice-cold water followed by filtration to produce dibenzo[b,d]thiophene-3-sulfonyl chloride (150 mg) as a white solid.

### Step 2: Preparation of 8-bromodibenzo[b,d]thiophene-3-sulfonyl chloride

Dibenzo[b,d]thiophene-3-sulfonyl chloride (10.0 g, 35.5 mmol) was mixed with acetic acid (glacial, 55 mL) and bromine (17.0 g, 3 eq.) and the mixture was stirred at 70° C. for 4 hours. The excess bromine was removed by bubbling nitrogen through the reaction mixture and the resulting solid was collected by filtration and washed with acetic acid to produce 8-bromodibenzo[b,d]thiophene-3-sulfonyl chloride (10.1 g) as a light brown solid.

### Step 3: Preparation of (S)-tert-butyl 2-(8-bromodibenzo[b,d]thiophene-3-sulfonamido)-3-methylbutanoate

8-Bromodibenzo[b,d]thiophene-3-sulfonyl chloride (7.2 g, 20 mmol) and (S)-tert-butyl 2-amino-3-methylbutanoate hydrochloride (4.6 g, 22 mmol) were mixed with 50 mL of DCM and N,N-diisopropylethylamine (7.68 mL, 44 mmol) was added. The mixture was stirred at room temperature overnight and was concentrated to give the crude product, which was purified by column chromatography to produce (S)-tert-butyl 2-(8-bromodibenzo [b,d]thiophene-3-sulfonamido)-3-methylbutanoate (9.4 g) as a white solid.

### Step 4: Preparation of (5)-tert-butyl 3-methyl-2-(8-(pyridin-3-yl)dibenzo[b,d]thiophene-3-sulfonamido)butanoate

(S)-Tert-butyl 2-(8-bromodibenzo[b,d]thiophene-3-sulfonamido)-3-methylbutanoate (366 mg, 0.73 mmol) were mixed with K₂CO₃ (355 mg, 3.5 eq.), pyridin-3-ylboronic acid (226 mg, 1.84 mmol), and Pd(Ph₃)₄ (80 mg) in a mixture of 3 mL of DME and 0.5 mL of water. The reaction mixture was deoxygenated with nitrogen and stirred at 85° C. for 4 hours. Brine was added and the mixture was extracted with EtOAc. The combined EtOAc layers were concentrated to give the crude product, which was purified by column chromatography to produce (S)-tert-butyl 3-methyl-2-(8-(pyridin-3-yl)dibenzo[b,d]thiophene-3-sulfonamido) butanoate (237 mg) as a white solid.

### Step 5: Preparation of (S)-3-methyl-2-(8-(pyridin-3-yl)dibenzo[b,d]thiophene-3-sulfonamido)butanoic acid

(S)-Tert-butyl 3-methyl-2-(8-(pyridin-3-yl)dibenzo[b,d]thiophene-3-sulfonamido) butanoate (189 mg) was dissolved in a mixture of 3 mL of DCM and 3 mL of TFA and the resulting solution was stirred at room temperature for 4 hours. The reaction mixture was concentrated and the residue was triturated in ether/hexane followed by filtration to produce (S)-3-methyl-2-(8-(pyridin-3-yl)dibenzo[b,d]thiophene-3-sulfonamido)butanoic acid (210 mg) as a white solid. HRMS (ESI-FTMS): calculated for C₂₂H₂₀N₂O₄S₂+H⁺: 441.09372. found: 441.0934.

The following compounds in Table 13 were prepared using procedures analogous to those described above for the preparation of (S)-3-methyl-2-(8-(pyridin-3-yl) dibenzo[b,d] thiophene-3-sulfonamido)butanoic acid.

TABLE 13

| Compd No. | HRMS | MS |
|---|---|---|
| 24 | 460.0716 | — |
| 25 | 430.0788 | — |
| 26 | 446.0557 | — |
| 27 | — | 459.1 |
| 40 | 444.1054 | — |
| 41 | 458.1214 | — |
| 42 | 472.1368 | — |
| 43 | 500.1688 | — |
| 44 | 520.1373 | — |
| 45 | 430.0901 | — |
| 46 | 496.0726 | — |
| 47 | 488.0664 | — |
| 51 | — | 478.37 |
| 52 | — | 478.36 |
| 53 | 430.0779 | — |
| 54 | 629.1107 | — |
| 55 | 471.1056 | — |
| 56 | 441.0936 | — |
| 57 | 429.0949 | — |
| 58 | 472.1607 | — |
| 61 | 552.0834 | — |
| 62 | 460.0703 | — |
| 63 | 475.0547 | — |

### Example 17

### (S)-2-(7-(furan-3-yl)dibenzo[b,d]thiophene-3-sulfonamido)-3-methylbutanoic acid (Compound 60)

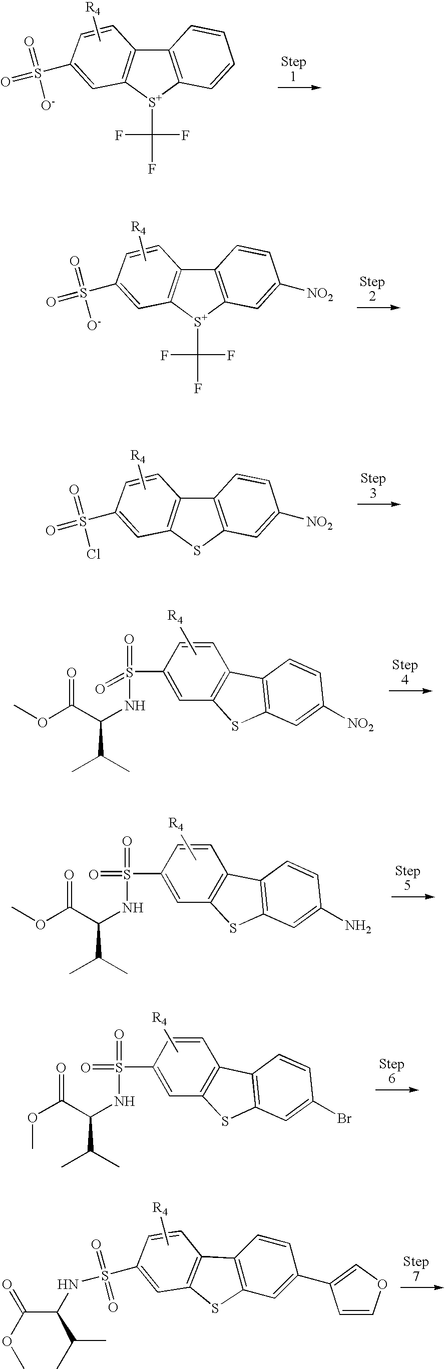

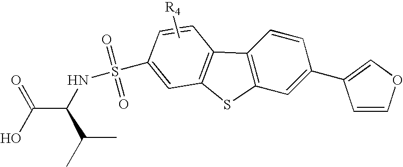

### Step 1: Preparation of 7-nitro-5-(trifluoromethyl)-5H-dibenzo[b,d] thiophenium-3-sulfonate

5-(Trifluoromethyl)-5H-dibenzo[b,d]thiophenium-3-sulfonate (5.0 g) was added portion-wise to a mixture of 3.3 mL oleum (30%) and 1.7 mL of HNO₃ (90%) and the resulting mixture was stirred at room temperature overnight. After slow addition of the mixture above to 250 mL of cold diethyl ether, the resulting solid was collected by filtration to produce 7-nitro-5-(trifluoromethyl)-5H-dibenzo[b,d]thiophenium-3-sulfonate (5.37 g, 95% yield).

### Step 2: Preparation of 7-nitrodibenzo[b,d]thiophene-3-sulfonyl chloride

7-Nitro-5-(trifluoromethyl)-5H-dibenzo[b,d]thiophenium-3-sulfonate (5 g) was dissolved in 35 mL of thionyl chloride and a few drops of DMF were added. The resulting mixture was heated at 80° C. for 24 hours, the excess of thionyl chloride was removed under reduced pressure, and the residue triturated twice with DCM to produce 7-nitrodibenzo[b,d]thiophene-3-sulfonyl chloride in quantitative yield.

### Step 3: Preparation of (S)-methyl 3-methyl-2-(7-nitrodibenzo[b,d]thiophene-3-sulfonamido)butanoate

Following the procedure described in step 2 for the preparation (S)-2-(8-(furan-3-yl)dibenzo[b,d]furan-3-sulfonamido)-3-methylbutanoic acid, (S)-methyl 3-methyl-2-(7-nitrodibenzo[b,d]thiophene-3-sulfonamido) butanoate (95% yield) was obtained as a white solid. ¹H NMR (DMSO-d6): δ 0.85 (d, J=6.9 Hz, 3H), 0.87 (d, J=6.9 Hz, 3H), 1.99 (m, 1H), 3.35 (s, 3H), 3.74 (d, J=6.3 Hz, 1H), 7.96 (dd, J=8.5, 1.9 Hz, 1H), 7.99 (s br, 1H), 8.35 (dd, J=8.8, 2.2 Hz, 1H), 8.55 (dd, J=1.6, 0.6 Hz, 1H), 8.66 (d, J=8.2 Hz, 1H), 8.67 (d, J=8.8 Hz, 1H), 9.05 (d, J=1.9 Hz, 1H).

### Step 4: Preparation of (S)-methyl 2-(7-aminodibenzo[b,d]thiophene-3-sulfonamido)-3-methylbutanoate

(S)-Methyl 3-methyl-2-(7-nitrodibenzo[b,d]thiophene-3-sulfonamido) butanoate (1 g, 3 mmol) and ammonium formate (5 g) were dissolved in 40 mL of MeOH. Pd/C (150 mg, 10% w/w) was added and the mixture was stirred at the reflux temperature overnight. Upon completion according to TLC, the reaction mixture was filtered through a Celite® plug, concentrated, and the residue partitioned between NaHCO₃ (1.0 M) and EtOAc. The organic layer was separated, dried over Na₂SO₄, and concentrated to give the crude product, which was purified by column chromatography to produce (S)-methyl 2-(7-aminodibenzo[b,d]thiophene-3-sulfonamido)-3-methylbutanoate (440 mg). ¹H NMR (CDCl₃): δ 0.85 (d, J=6.9 Hz, 3H), 0.89 (d, J=6.9 Hz, 3H), 2.05-1.89 (m, 1H), 2.13 (s br, 2H), 3.28 (s, 3H), 3.73 (dd, J=10.1, 5.4 Hz, 1H), 5.60 (d, J=10.1 Hz, 1H), 6.84 (dd, J=8.5, 2.2 Hz, 1H), 7.10 (d, J=1.9, 1H), 7.76 (dd, J=8.2, 1.6 Hz, 1H), 7.90 (d, J=8.5 Hz, 1H), 7.97 (d, J=8.5 Hz, 1H), 8.18 (d, J=1.6 Hz, 1H).

### Step 5: Preparation of (S)-methyl 2-(7-bromodibenzo[b,d]thiophene-3-sulfonamido)-3-methylbutanoate

(S)-Methyl 2-(7-aminodibenzo[b,d]thiophene-3-sulfonamido)-3-methylbutanoate (400 mg, 1.02 mmol) was dissolved in 20 mL of acetonitrile and CuBr (700 mg, 5 mmol) was added followed by slow addition of isoamylnitrite (600 mg, 5 mmol). The resulting mixture was stirred at room temperature for 30 minutes, diluted with 50 mL of EtOAc, and washed with diluted ammonia. The organic phase was separated, dried over Na₂SO₄, and concentrated to provide the crude product, which was purified by column chromatography to produce (S)-methyl 2-(7-bromodibenzo[b,d]thiophene-3-sulfonamido)-3-methylbutanoate (200 mg, 45% yield) as a pale yellow solid. ¹H NMR (CDCl₃): δ 0.89 (d, J=6.9 Hz, 3H), 0.96 (d, J=6.6 Hz, 3H), 2.13-1.95 (m, 1H), 3.33 (s, 3H), 3.82 (dd, J=10.1, 5.03 Hz, 1H), 5.15 (d, J=10.1 Hz, 1H), 7.64 (dd, J=8.8, 1.9 Hz, 1H), 7.89 (dd, J=8.3, 1.6, 1H), 8.05 (d, J=1.9 Hz, 1H), 8.06 (d, J=8.3 Hz, 1H), 8.21 (d, J=8.5 Hz, 1H), 8.34 (d, J=1.6 Hz, 1 H).

### Step 6: Preparation of (S)-2-(7-(furan-3-yl)dibenzo[b,d]thiophene-3-sulfonamido)-3-methylbutanoic acid

Following the procedures described above for the preparation of (S)-2-(8-(furan-3-yl)dibenzo[b,d]furan-3-sulfonamido)-3-methylbutanoic acid, (S)-2-(7-(furan-3-yl)dibenzo [b,d]thiophene-3-sulfonamido)-3-methylbutanoic acid was prepared by a Suzuki reaction of (S)-methyl 2-(7-bromodibenzo[b,d]thiophene-3-sulfonamido)-3-methylbutanoate with 3-furanboronic acid followed by hydrolysis of the methyl ester under basic condition. ¹H NMR (CDCl₃): δ 0.85 (d, J=6.6 Hz, 3H), 0.96 (d, J=6.9 Hz, 3H), 2.08 (m, 1H), 3.74 (dd, J=9.4, 4.4 Hz, 1H), 5.47 (d, J=9.4 Hz, 1H), 6.76 (dd, J=1.9, 0.6 Hz, 1H), 7.50 (dd, J=1.6, 1.6 Hz, 1H), 7.61 (dd, J=8.2, 1.6 Hz, 1H), 7.83 (dd, J=1.3, 1.3 Hz, 1H), 7.88 (dd, J=8.5, 1.6 Hz, 1H), 7.95 (d, J=1.3 Hz, 1H), 8.14 (d, J=8.2 Hz, 1H), 8.17 (d, J=7.8 Hz, 1H), 8.32 (d, J=1.3 Hz, 1H). MS (ESI, [M+H]⁺): 430.0.

### Example 17A

### (S)-2-(7-(furan-2-yl)dibenzo[b,d]thiophene-3-sulfonamido)-3-methyl butanoic acid (Compound 193)

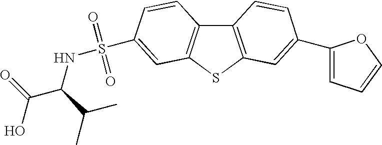

The title compound was prepared by the procedures described in Example 17, using 2-furanboronic acid instead of 3-furanboronic acid. The compound was obtained as a white solid. ¹H NMR (300 MHz, DMSO-d₆) δ ppm 12.51 (br. s., 1H), 8.38-8.57 (m, 4H), 8.09 (d, J=10.0 Hz, 1H), 7.92 (dd, J=8.4, 1.6 Hz, 1H), 7.87 (dd, J=8.4, 1.6 Hz, 1H), 7.84 (d, J=1.8 Hz, 1H), 7.16 (d, J=3.2 Hz, 1H), 6.68 (dd, J=3.4, 1.9 Hz, 1H), 3.52-3.76 (m, 1H), 1.89-2.03 (m, 1H), 0.85 (d, J=6.7 Hz, 3H), 0.81 (d, J=7.0 Hz, 3H). ESIMS (m/z) 430.11 (MH⁺).

### Example 17B

### (R)-2-(7-(furan-2-yl)dibenzo[b,d]thiophene-3-sulfonamido)-3-methyl butanoic acid (Compound 129)

The title compound was prepared following the procedures described in Example 17A, using D-valine instead of the L-valine at the early stage of the preparation (Ref. Step 3, Example 17). The compound was obtained as a white solid. MS (ESI, [M+H]⁺): 430.0.

### Example 17C

### (S)-3-methyl-2-(7-phenyldibenzo[b,d]thiophene-3-sulfonamido)butanoic acid (Compound 313)

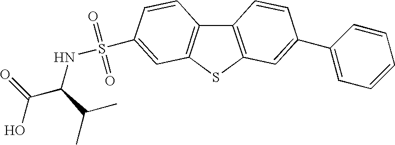

The title compound was prepared by the procedures described in Example 17, using phenylboronic acid instead of 3-furanboronic acid. The compound was obtained as a white solid. ¹H NMR (CDCl3): 0.87 (d, J=6.9 Hz, 3H); 0.98 (d, J=6.9 Hz, 3H); 2.09 (m, 1H); 3.88 (dd, J=9.8, 4.7 Hz, 1H); 5.12 (d, J=9.8 Hz, 1H); 7.41 (dd, J=7.6, 7.6 Hz, 1H); 7.50 (dd, J=7.6, 7.6 Hz, 2H); 7.69 (d, J=7.6 Hz, 2H); 7.76 (dd, J=8.2, 1.6 Hz, 1H); 7.90 (dd, J=8.5, 1.9 Hz, 1H); 8.10 (d, J=1.6 Hz, 1H); 8.24 (d, J=8.5 Hz, 1H); 8.25 (d, J=8.2 Hz, 1H); 8.37 (d, J=1.9 Hz, 1H). MS (ES⁻): 492.1.

### Example 18

### (S)-2-(7-(3-methoxyprop-1-ynyl)dibenzo[b,d]thiophene-3-sulfonamido)-3-methylbutanoic acid (Compound 65)

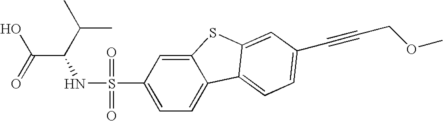

Following the procedures described above for the preparation of (S)-tert-butyl 2-(8-(3-methoxyprop-1-ynyl)dibenzo[b,d]furan-3-sulfonamido)-3-methylbutanoate, (S)-2-(7-(3-methoxyprop-1-ynyl)dibenzo[b,d]thiophene-3-sulfonamido)-3-methylbutanoic acid (36% overall yield) was prepared using (S)-methyl 2-(7-bromodibenzo[b,d]thiophene-3-sulfonamido)-3-methylbutanoate and 3-methoxyprop-1-yne. ¹H NMR (DMSO-d₆): δ 0.80 (d, J=6.6 Hz, 3H), 0.84 (d, J=6.6 Hz, 3H), 1.95 (m, 1H), 3.37 (s, 3H), 3.62 (dd, J=9.4, 6.0 Hz, 1H), 4.38 (s, 2H), 7.63 (dd, J=8.2, 1.6 Hz, 1H), 7.88 (dd, J=8.5, 1.6 Hz, 1H), 8.11 (d, J=9.4 Hz, 1H), 8.27 (d, J=1.6 Hz, 1H), 8.46 (d, J=8.2 Hz, 1H), 8.49 (d, J=1.6 Hz, 1H), 8.54 (d, J=8.5 Hz, 1H), 12.49 (s br, 1H). MS (ESI, [M+H]⁺): 432.0.

### Example 19

### (R)-2-(7-(3-methoxyprop-1-ynyl)dibenzo[b,d]thiophene-3-sulfonamido)-3-methylbutanoic acid (Compound 90)

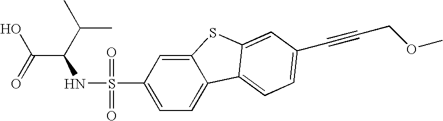

Following the procedures described above for the preparation of (S)-tert-butyl 2-(8-(3-methoxyprop-1-ynyl)dibenzo[b,d]furan-3-sulfonamido)-3-methylbutanoate, (R)-2-(7-(3-methoxyprop-1-ynyl)dibenzo[b,d]thiophene-3-sulfonamido)-3-methylbutanoic acid was prepared using (R)-methyl 2-(7-bromodibenzo[b,d]thiophene-3-sulfonamido)-3-methyl butanoate and 3-methoxyprop-1-yne. MS (ESI, [M+H]⁺): 432.0.

### Example 19A

### (R)-3-methyl-2-(7-phenyldibenzo[b,d]thiophene-3-sulfonamido)butanoic acid. (Compound 314)

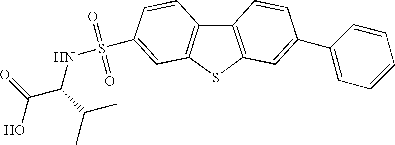

The title compound was prepared following the procedures described in Example 19, using phenylboronic acid instead of 3-methoxyprop-1-yne. The compound was obtained as a white solid. ¹H NMR (CDCl3): 0.87 (d, J=6.9 Hz, 3H); 0.98 (d, J=6.9 Hz, 3H); 2.09 (m, 1H); 3.88 (dd, J=9.8, 4.7 Hz, 1H); 5.12 (d, J=9.8 Hz, 1H); 7.41 (dd, J=7.6, 7.6 Hz, 1H); 7.50 (dd, J=7.6, 7.6 Hz, 2H); 7.69 (d, J=7.6 Hz, 2H); 7.76 (dd, J=8.2, 1.6 Hz, 1H); 7.90 (dd, J=8.5, 1.9 Hz, 1H); 8.10 (d, J=1.6 Hz, 1H); 8.24 (d, J=8.5 Hz, 1H); 8.25 (d, J=8.2 Hz, 1H); 8.37 (d, J=1.9 Hz, 1H). MS (ES⁻): 492.1.

### Example 20

### (S)-3-methyl-2-(8-(thiazol-2-yl)dibenzo[b,d]furan-3-sulfonamido)butanoic acid (Compound 36)

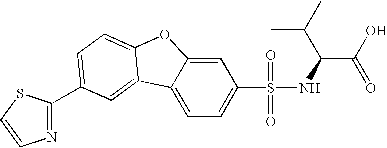

### Step 1: Preparation of 8-bromodibenzo[b,d]furan-3-sulfonyl chloride

Dibenzo[b,d]furan-3-sulfonyl chloride (5.3 g, 20 mmol, 1.0 eq.) was mixed with acetic acid (glacial, 120 mL) and bromine (10 mL, 10 eq.) and the mixture was stirred at 70° C. for 4 hours. The excess bromine was removed by bubbling nitrogen through the reaction mixture and trapped with saturated Na₂SO₃ solution. The resulting solution was cooled to room temperature and filtered to produce 8-bromodibenzo[b,d]furan-3-sulfonyl chloride (5.4 g, 78% yield) as a light brown solid.

### Step 2: Preparation of (S)-tert-butyl 2-(8-bromodibenzo[b,d]furan-3-sulfonamido)-3-methylbutanoate

8-Bromodibenzo[b,d]furan-3-sulfonyl chloride (3.46 g, 10 mmol) and (S)-t-butyl 2-amino-3-methylbutanoate hydrochloride (1.1 eq.) were mixed in 30 mL of DCM and N,N-diisopropylethylamine (3.84 mL, 2.2 eq.) was added. The resulting mixture was stirred at room temperature for 5 hours, concentrated, and the crude product was purified by column chromatography to produce (S)-tert-butyl 2-(8-bromodibenzo[b,d]furan-3-sulfonamido)-3-methylbutanoate (4.7 g, 97.5% yield) as a white solid.

### Step 3: Preparation of (S)-tert-butyl 3-methyl-2-(8-(4,4,5,5-tetramethyl-1,3,2-dioxaborolan-2-yl)dibenzo[b,d]furan-3-sulfonamido)butanoate

(S)-Tert-butyl 2-(8-bromodibenzo[b,d]furan-3-sulfonamido)-3-methylbutanoate (2 g, 4.15 mmol), CH₃COOK (1.22 g, 12.45 mmol), [1,1′-Bis(diphenylphosphino)ferrocene] dichloropalladium(II) (PdCl₂-dppf₂, 170 mg), and bis-pinacolate diboron (3.16 g, 12.45 mmol) were dissolved in 40 mL of DMSO and the mixture was stirred at 90° C. for 2 hours. The reaction was monitored by a LC-MS, and, after completion of the reaction, the mixture was cooled at room temperature, 150 mL of water was added, and the mixture was extracted with two 100 mL-portions of DCM. The combined organic phases were dried over Na₂SO₄, concentrated, and the residue was purified by column chromatography to produce (S)-tert-butyl 3-methyl-2-(8-(4,4,5,5-tetramethyl-1,3,2-dioxaborolan-2-yl)dibenzo[b,d]furan-3-sulfonamido)butanoate (2.10 g, 95% yield).

### Step 4: Preparation of (S)-tert-butyl 3-methyl-2-(8-(thiazol-2-yl)dibenzo[b,d]furan-3-sulfonamido)butanoate

(S)-Tert-butyl 3-methyl-2-(8-(4,4,5,5-tetramethyl-1,3,2-dioxaborolan-2-yl)dibenzo[b,d]furan-3-sulfonamido)butanoate (4.15 g, 7.85 mmol), bromothiazole (2.83 g, 17.26 mmol), K₂CO₃ (2.7 g, 19.6 mmol), and Pd(PPh₃)₄ (800 mg) were dissolved in a mixture of 70 mL of DME and 10 mL of water. After deoxygenated by bubbling nitrogen through for 20 minutes, the solution was heated at 85° C. until no starting material was left according to LC-MS. The reaction mixture was cooled to room temperature before the addition of 100 mL of brine and 100 mL of EtOAc. The organic phase was separated and the aqueous layer was extracted with two 100 mL-portions of EtOAc. The combined organic layers were dried over Na₂SO₄, concentrated, and the residue was purified by column chromatography to produce (S)-tert-butyl 3-methyl-2-(8-(thiazol-2-yl)dibenzo[b,d]furan-3-sulfonamido)butanoate (2.53 g, 66% yield).

### Step 5: Preparation of (S)-3-methyl-2-(8-(thiazol-2-yl)dibenzo[b,d]furan-3-sulfonamido)butanoic acid

(S)-Tert-butyl 3-methyl-2-(8-(thiazol-2-yl)dibenzo[b,d]furan-3-sulfonamido)butanoate (2.53 g, 5.2 mmol) was dissolved in 40 mL of TFA in DCM (30%). The reaction solution was stirred overnight, concentrated, and the residue was purified by reverse phase flash chromatography (C-18 Silica) to produce (S)-3-methyl-2-(8-(thiazol-2-yl)dibenzo[b,d]furan-3-sulfonamido)butanoic acid (1.83 g, 82% yield) as a white powder. ¹H NMR (DMSO-d6): δ 0.87 (d, J=6.6 Hz, 3H), 0.89 (d, J=6.6 Hz, 3H), 2.00 (m, 1H), 3.67 (d, J=6.0 Hz, 1H), 7.74 (d, J=3.2 Hz, 1H), 7.86 (dd, J=8.5, 0.6 Hz, 1H), 7.88 (dd, J=8.2, 1.6 Hz, 1H), 7.95 (d, J=3.5 Hz, 1H), 8.11 (dd, J=1.6, 0.6 Hz, 1H), 8.21 (dd, J=8.8, 1.9 Hz, 1H), 8.43 (d, J=8.2 Hz, 1H), 8.81 (dd, J=1.9, 0.6 Hz, 1H). MS (ES, [M+H]⁺): 431.2.

The following compounds were prepared by the procedures as described in Example 20 for the preparation of (S)-3-methyl-2-(8-(thiazol-2-yl)dibenzo[b,d]furan-3-sulfonamido)butanoic acid.

### Example 20A

### (S)-2-(8-(benzo[d]oxazol-2-yl)dibenzo[b,d]furan-3-sulfonamido)-3-methyl butanoic acid (Compound 194)

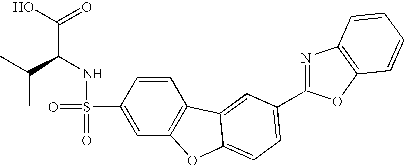

The title compound was prepared by the procedures described in Example 20, using 2-chlorobenzo[d]oxazole instead of 2-bromothiazole. The compound was obtained as a white solid in 100% yield. ¹H NMR (400 MHz, MeOD) δ ppm 1.06-1.16 (m, 3H), 1.17-1.24 (m, 3H), 3.33-3.37 (m, 1H), 3.68-3.72 (m, 1H), 7.63-7.68 (m, 2H), 7.92-7.97 (m, 1H), 7.97-8.02 (m, 1H), 8.10 (d, J=8.59 Hz, 1H), 8.18 (s, 1H), 8.38 (s, 1H), 8.56 (d, J=8.08 Hz, 1H), 8.72 (dd, J=8.84, 1.77 Hz, 1H), 9.24 (d, J=1.52 Hz, 1H). HRMS (ESI-FTMS): calcd for C₂₄H₂₀N₂O₆S+H⁺, 465.11148. found: 465.11293.

### Example 20B

### (S)-2-(2,2′-bidibenzo[b,d]furan-7-sulfonamido)-3-methylbutanoic acid (Compound 195)

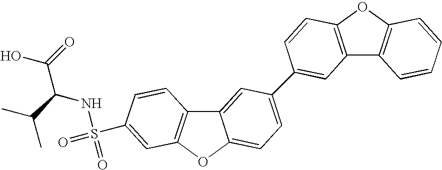

The title compound was prepared by the procedures described in Example 20, using 2-bromodibenzo[b,d]furan instead of 2-bromothiazole. The compound was obtained as a white solid in 95% yield. ¹H NMR (400 MHz, MeOD) δ ppm 1.14 (d, J=6.82 Hz, 3H), 1.21 (d, J=6.82 Hz, 3H), 2.25-2.33 (m, 1H), 3.91 (d, 1H), 7.57-7.65 (m, 1H), 7.69-7.78 (m, 1H), 7.84 (d, J=8.34 Hz, 1H), 7.91 (d, J=8.59 Hz, 1H), 7.98 (d, J=8.59 Hz, 1H), 8.07 (dd, J=8.59, 1.77 Hz, 1H), 8.12 (dd, J=8.08, 1.52 Hz, 1H), 8.17 (dd, J=8.59, 2.02 Hz, 1 H), 8.33 (d, J=1.01 Hz, 1H), 8.36 (d, 1H), 8.49 (d, J=8.08 Hz, 1H), 8.59 (d, J=1.52 Hz, 1H), 8.67 (d, J=1.52 Hz, 1H). HRMS (ESI-FTMS): calcd for C₂₉H₂₃NO₆S+H⁺, 514.13189. found: 514.13185.

### Example 20C

### (S)-2-(8-(5-ethylthiophen-2-yl)dibenzo[b,d]furan-3-sulfonamido)-3-methylbutanoic acid (Compound 196)

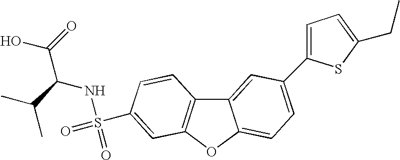

The title compound was prepared by the procedures described in Example 20, using 2-bromo-5-ethylthiazole instead of 2-bromothiazole. The compound was obtained as a white solid in 45% yield. ¹H NMR (400 MHz, MeOD) δ ppm 0.94 (d, J=6.82 Hz, 3H), 1.00 (d, J=6.82 Hz, 3H), 1.37 (t, J=7.58 Hz, 3H), 2.01-2.14 (m, 1H), 2.86-2.96 (m, 2H), 3.75 (d, J=5.56 Hz, 1H), 7.27 (d, J=3.54 Hz, 1H), 7.64 (d, J=8.59 Hz, 1H), 7.81 (dd, J=8.72, 1.89 Hz, 1H), 7.89 (dd, J=8.34, 1.52 Hz, 1H), 8.09 (d, J=1.01 Hz, 1H), 8.22 (d, J=8.08 Hz, 1H), 8.29 (d, J=1.26 Hz, 1H). HRMS (ESI-FTMS): calcd for C₂₃H₂₃NO₅S₂+H⁺, 458.10904. found: 458.10998.

### Example 20D

### (S)-3-methyl-2-(8-(5-propylthiophen-2-yl)dibenzo[b,d]furan-3-sulfonamido)butanoic acid (Compound 197)

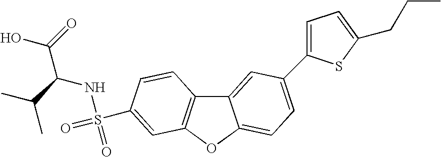

The title compound was prepared by the procedures described in Example 20, using 2-bromo-5-propylthiazole instead of 2-bromothiazole. The compound was obtained as a white solid in 50% yield. ¹H NMR (400 MHz, MeOD) δ ppm 0.82 (d, J=6.82 Hz, 3H), 0.88 (d, J=6.82 Hz, 3H), 0.93 (t, J=7.33 Hz, 3H), 1.57-1.72 (m, 2H), 1.96 (dd, J=12.51, 6.69 Hz, 1H), 2.74 (t, J=7.45 Hz, 2H), 3.63 (d, J=5.56 Hz, 1H), 6.72 (d, J=3.54 Hz, 1H), 7.17 (d, J=3.54 Hz, 1H), 7.54 (d, J=8.84 Hz, 1H), 7.71 (dd, J=8.72, 1.89 Hz, 1H), 7.77 (dd, J=8.08, 1.52 Hz, 1H), 7.98 (d, J=1.01 Hz, 1H), 8.13 (d, J=8.08 Hz, 1H), 8.20 (d, J=2.02 Hz, 1H). HRMS (ESI-FTMS): calcd for C₂₄H₂₅NO₅S₂+H⁺, 472.12469. found: 472.12692.

### Example 20E

### (S)-2-(8-(5-tert-butylfuran-2-yl)dibenzo[b,d]furan-3-sulfonamido)-3-methylbutanoic acid (Compound 198)

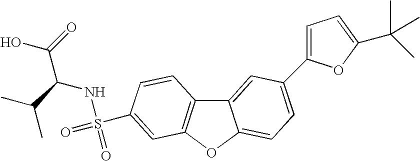

The title compound was prepared by the procedures described in Example 20, using 2-bromo-5-tert-butylthiazole instead of 2-bromothiazole. The compound was obtained as a white solid in 50% yield. ¹H NMR (400 MHz, MeOD) δ ppm 1.13 (d, J=6.82 Hz, 3H), 1.20 (d, J=6.57 Hz, 3H), 1.61 (s, 9H), 2.22-2.31 (m, 1H), 3.94 (d, J=5.56 Hz, 1H), 6.36 (d, J=3.28 Hz, 1H), 6.93 (d, J=3.28 Hz, 1H), 7.88 (d, J=8.59 Hz, 1H), 8.06-8.10 (m, 1H), 8.11 (dd, J=3.16, 1.64 Hz, 1H), 8.30 (d, J=1.01 Hz, 1H), 8.46 (d, J=8.08 Hz, 1H), 8.57 (d, J=1.77 Hz, 1H). HRMS (ESI-FTMS): calcd for C₂₅H₂₇NO₆S+H⁺, 470.16318. found: 470.16531.

### Example 20F

### (S)-3-methyl-2-(8-(5-(5-methyl-1,2,4-oxadiazol-3-yl)thiophen-2-yl)dibenzo [b,d]furan-3-sulfonamido)butanoic acid (Compound 199)

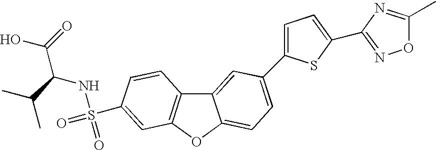

The title compound was prepared by the procedures described in Example 20, using 3-(2-bromothiazol-5-yl)-5-methyl-1,2,4-oxadiazole instead of 2-bromothiazole. The compound was obtained as a white solid in 40% yield. ¹H NMR (400 MHz, DMSO-d₆) δ ppm 0.80 (d, J=6.82 Hz, 3H), 0.85 (d, J=6.82 Hz, 3H), 1.95 (d, J=6.57 Hz, 1H), 2.67 (s, 3H), 3.50 (s, 1H), 7.75 (d, J=3.79 Hz, 1H), 7.81-7.85 (m, 1H), 7.88 (d, J=8.59 Hz, 2H), 8.02 (dd, J=8.59, 2.02 Hz, 1H), 8.09 (s, 1H), 8.42 (d, J=8.08 Hz, 1H), 8.71 (d, J=1.77 Hz, 1H). HRMS (ESI-FTMS): calcd for C₂₄H₂₁N₃O₆S₂+H⁺, 512.09445. found: 512.09393.

### Example 20G

### (S)-2-(8-(5-chloro-4-(trifluoromethyl)thiazol-2-yl)dibenzo[b,d]furan-3-sulfonamido)-3-methylbutanoic acid (Compound 200)

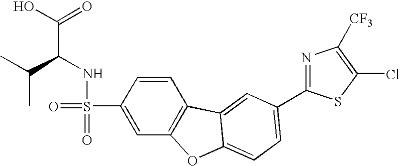

The title compound was prepared by the procedures described in Example 20, using 5-chloro-2-fluoro-4-(trifluoromethyl)thiazole instead of 2-bromothiazole. The compound was obtained as a white solid in 40% yield. ¹H NMR (400 MHz, DMSO-d₆) δ ppm 0.82 (m, 6H), 1.87-2.02 (m, 1H), 3.57 (s, 1H), 7.87 (dd, J=8.08, 1.52 Hz, 1H), 7.97 (d, J=8.84 Hz, 1H), 8.12 (d, J=1.52 Hz, 1H), 8.19 (dd, J=8.59, 2.02 Hz, 1H), 8.53 (d, J=8.08 Hz, 1H), 8.91 (d, J=2.02 Hz, 1H). HRMS (ESI-FTMS): calcd for C₂₁H₁₆ClF₃N₂O₆S₂+H⁺, 533.02140. found: 533.02113.

### Example 20H

### (S)-2-(8-(2,4-dimethylthiazol-5-yl)dibenzo[b,d]furan-3-sulfonamido)-3-methylbutanoic acid (Compound 201)

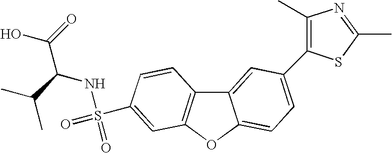

The title compound was prepared by the procedures described in Example 20, using 5-bromo-2,4-dimethylthiazole instead of 2-bromothiazole. The compound was obtained as a white solid in 60% yield. ¹H NMR (400 MHz, DMSO-d₆) δ ppm 0.82 (m, 6H), 1.95 (dd, J=13.01, 6.69 Hz, 1H), 2.43 (s, 3H), 2.66 (s, 3H), 3.59 (s, 1H), 7.67 (dd, J=8.59, 2.02 Hz, 1H), 7.80-7.91 (m, 2H), 8.09 (d, J=1.01 Hz, 1H), 8.35 (d, J=1.26 Hz, 1H), 8.41 (d, J=8.34 Hz, 1H). HRMS (ESI-FTMS): calcd for C₂₂H₂₂N₂O₆S₂+H⁺, 459.10429. found: 459.10506.

### Example 20I

### (S)-3-methyl-2-(8-(2-methylthiazol-5-yl)dibenzo[b,d]furan-3-sulfonamido)butanoic acid (Compound 202)

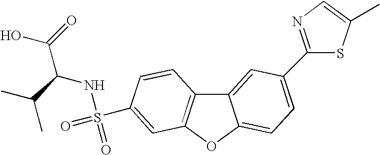

The title compound was prepared by the procedures described in Example 20, using 2-bromo-5-methylthiazole instead of 2-bromothiazole. The compound was obtained as a white solid in 90% yield. ¹H NMR (400 MHz, MeOD) δ ppm 1.14 (d, J=6.82 Hz, 3H), 1.20 (d, J=6.82 Hz, 3H), 2.22-2.32 (m, 1H), 2.78 (d, J=1.01 Hz, 3H), 3.96 (d, J=5.81 Hz, 1H), 5.70 (s, 1H), 7.79 (d, J=1.26 Hz, 1H), 7.97 (d, J=8.59 Hz, 1H), 8.13 (dd, J=8.08, 1.52 Hz, 1H), 8.31-8.36 (m, 2H), 8.49 (d, J=8.84 Hz, 1H), 8.85 (d, J=2.02 Hz, 1H). HRMS (ESI-FTMS): calcd for C₂₂H₂₂N₂O₆S₂+H⁺, 459.10429. found: 459.10506.

### Example 20J

### (S)-2-(8-(6-chlorobenzo[d]thiazol-2-yl)dibenzo[b,d]furan-3-sulfonamido)-3-methylbutanoic acid (Compound 203)

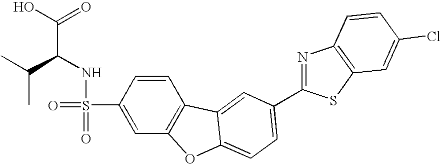

The title compound was prepared by the procedures described in Example 20, using 2,6-dichlorobenzo[d]thiazole instead of 2-bromothiazole. The compound was obtained as a white solid in 88% yield. ¹H NMR (400 MHz, DMSO-d₆) δ ppm 0.84 (m, 6H), 1.96 (s, 1H), 3.63 (d, J=9.35 Hz, 1H), 7.62 (dd, J=8.72, 2.15 Hz, 1H), 7.89 (dd, J=8.08, 1.52 Hz, 1H), 8.00 (d, J=8.34 Hz, 1H), 8.05-8.16 (m, 2H), 8.18 (s, 1H), 8.33-8.44 (m, 2H), 8.58 (d, J=8.34 Hz, 1H), 9.06 (d, J=1.77 Hz, 1H). HRMS (ESI-FTMS): calcd for C₂₄H₁₉ClN₂O₆S₂+H+, 515.04967. found: 515.05179.

### Example 20K

### (S)-2-(8-(2-isobutyl-4-methylthiazol-5-yl)dibenzo[b,d]furan-3-sulfonamido)-3-methylbutanoic acid (Compound 204)

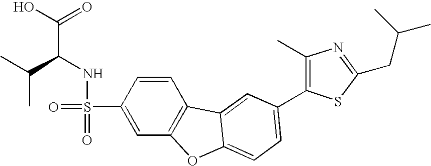

The title compound was prepared by the procedures described in Example 20, using 5-bromo-2-isobutyl-4-methylthiazole instead of 2-bromothiazole. The compound was obtained as a white solid in 71% yield. ¹H NMR (400 MHz, MeOD) δ ppm 1.13 (d, J=6.82 Hz, 3H), 1.20 (d, J=6.82 Hz, 3H), 1.26 (d, J=6.57 Hz, 6H), 2.22-2.40 (m, 2H), 2.69 (s, 3H), 3.10 (d, J=7.07 Hz, 2H), 3.93 (s, 1H), 7.85-7.91 (m, 1H), 7.94-7.99 (m, 1H), 8.11 (dd, J=8.08, 1.52 Hz, 1H), 8.33 (d, J=1.26 Hz, 1H), 8.43 (d, J=1.77 Hz, 1H), 8.46 (d, J=8.08 Hz, 1H). HRMS (ESI-FTMS): calcd for C₂₅H₂₈N₂O₅S₂+H⁺, 501.15124. found: 501.15186.

### Example 20L

### (S)-3-methyl-2-(8-(5-phenyl-3-(trifluoromethyl)-1H-pyrazol-4-yl)dibenzo [b,d]furan-3-sulfonamido)butanoic acid (Compound 206)

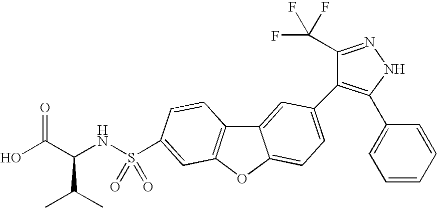

The title compound was prepared by the procedures described in Example 20, using 4-bromo-5-phenyl-3-(trifluoromethyl)-1H-pyrazole instead of 2-bromothiazole. The compound was obtained as an off-white solid. ¹H NMR (400 MHz, MeOD) δ ppm 1.12 (d, J=6.82 Hz, 3H), 1.20 (d, J=6.57 Hz, 3H), 2.20-2.33 (m, 1H), 3.88 (d, J=5.31 Hz, 1H), 7.50-7.59 (m, 5H), 7.65-7.71 (m, 1H), 7.89 (d, J=8.59 Hz, 1H), 8.04-8.09 (m, 1H), 8.22-8.25 (m, 1H), 8.30-8.36 (m, 2H). HRMS (ESI-FTMS): calcd for C₂₇H₂₂F₃N₃O₅S+H⁺, 558.13050. found: 558.13073.

### Example 20M

### (S)-2-(8-(5-(1H-tetrazol-5-yl)thiophen-2-yl)dibenzo[b,d]furan-3-sulfonamido)-3-methylbutanoic acid (Compound 207)

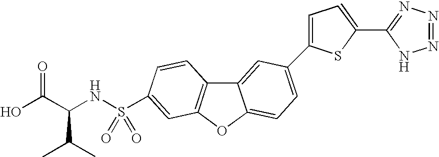

The title compound was prepared by the procedures described in Example 20, using 5-(5-bromothiophen-2-yl)-1H-tetrazole instead of 2-bromothiazole. The compound was obtained as an off-white solid. ¹H NMR (400 MHz, MeOD) δ ppm 0.93 (d, J=6.82 Hz, 3H), 0.99 (d, J=6.82 Hz, 3H), 2.02-2.13 (m, 1H), 3.76 (d, J=5.56 Hz, 1H), 7.56 (d, J=3.79 Hz, 1H), 7.70 (d, J=8.84 Hz, 1H), 7.76 (d, J=4.04 Hz, 1H), 7.88-7.93 (m, 2H), 8.08-8.11 (m, 1H), 8.23 (d, J=8.34 Hz, 1H). HRMS (ESI-FTMS): calcd for C₂₂H₁₉N₅O₅S₂+H⁺, 498.09004. found: 498.09028.

### Example 20N

### (S)-2-(8-(6-methoxybenzo[d]thiazol-2-yl)dibenzo[b,d]furan-3-sulfonamido)-3-methylbutanoic acid (Compound 208)

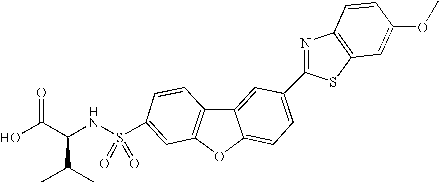

The title compound was prepared by the procedures described in Example 20, using 2-chloro-6-methoxybenzo[d]thiazole instead of 2-bromothiazole. The compound was obtained as an off-white solid. ¹H NMR (400 MHz, MeOD) δ ppm 0.93 (d, J=6.82 Hz, 3H), 1.00 (d, J=6.82 Hz, 3H), 2.03-2.13 (m, 1H), 3.75 (d, J=5.56 Hz, 1H), 3.92 (s, 3H), 7.15 (dd, J=8.97, 2.65 Hz, 1H), 7.52 (d, J=2.53 Hz, 1H), 7.73 (s, 2H), 7.79 (dd, J=8.59, 0.51 Hz, 1H), 7.91-7.96 (m, 2H), 8.14 (dd, J=1.52, 0.51 Hz, 1H), 8.24-8.28 (m, 2H), 8.76 (dd, J=1.89, 0.63 Hz, 1H). HRMS (ESI-FTMS): calcd for C₂₅H₂₂N₂O₆S₂+H⁺, 511.09920. found: 511.09909.

### Example 20O

### (S)-2-(8-(6-fluorobenzo[d]thiazol-2-yl)dibenzo[b,d]furan-3-sulfonamido)-3-methylbutanoic acid (Compound 209)

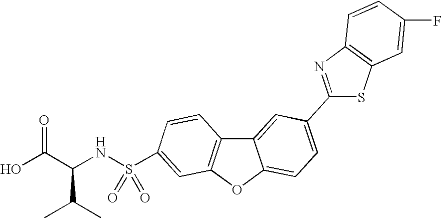

The title compound was prepared by the procedures described in Example 20, using 2-chloro-6-fluorobenzo[d]thiazole instead of 2-bromothiazole. The compound was obtained as an off-white solid. ¹H NMR (400 MHz, MeOD) δ ppm 0.93 (d, J=6.57 Hz, 3H), 1.00 (d, J=6.82 Hz, 3H), 2.03-2.13 (m, 1H), 3.77 (d, J=5.31 Hz, 1H), 7.29-7.32 (m, 1H), 7.71-7.82 (m, 2H), 7.92-7.96 (m, 1H), 8.01-8.06 (m, 1H), 8.14-8.16 (m, 1H), 8.24-8.32 (m, 2H), 8.78-8.81 (m, 1H). HRMS (ESI-FTMS): calcd for C₂₄H₁₉FN₂O₅S₂+H⁺, 499.07922. found: 99.07901.

### Example 20P

### (S)-3-methyl-2-(8-(6-methylbenzo[d]thiazol-2-yl)dibenzo[b,d]furan-3-sulfonamido)butanoic acid (Compound 210)

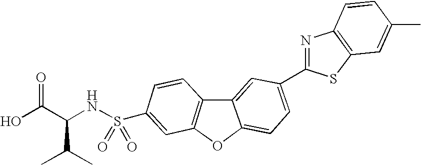

The title compound was prepared by the procedures described in Example 20, using 2-chloro-6-methylbenzo[d]thiazole instead of 2-bromothiazole. The compound was obtained as an off-white solid. ¹H NMR (400 MHz, MeOD) δ ppm 0.93 (d, J=6.82 Hz, 3H), 1.00 (d, J=6.57 Hz, 3H), 2.06-2.16 (m, 1H), 2.54 (s, 3H), 3.77 (d, J=5.05 Hz, 1H), 7.35-7.40 (m, 1H), 7.76-7.79 (m, 2H), 7.91-7.97 (m, 2H), 8.15 (d, J=1.01 Hz, 1H), 8.22-8.30 (m, 2H), 8.74-8.78 (m, 1H). HRMS (ESI-FTMS): calcd for C₂₅H₂₂N₂O₅S₂+H⁺, 495.10429. found: 495.10413.

### Example 20Q

### (S)-2-(8-(5-(isoxazol-5-yl)thiophen-2-yl)dibenzo[b,d]furan-3-sulfonamido)-3-methylbutanoic acid (Compound 211)

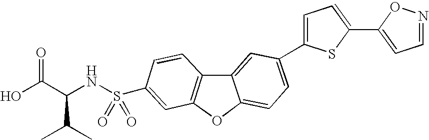

The title compound was prepared by the procedures described in Example 20, using 5-(5-bromothiophen-2-yl)isoxazole instead of 2-bromothiazole. The compound was obtained as an off-white solid. ¹H NMR (400 MHz, MeOD) δ ppm 0.90-0.97 (m, 6H), 2.00-2.10 (m, 1H), 3.79 (d, J=6.32 Hz, 1H), 7.54 (d, J=4.04 Hz, 1H), 7.58 (s, 3H), 7.73 (d, J=8.59 Hz, 1H), 7.86-7.95 (m, 3H), 8.10-8.12 (m, 1H), 8.20 (d, J=8.08 Hz, 1H), 8.40 (d, J=1.52 Hz, 1H). MS (ESI-FTMS) m/z 497.08356.

### Example 20R

### (S)-3-methyl-2-(8-(5-((4-methylpiperazin-1-yl)methyl)thiazol-2-yl)dibenzo [b,d]furan-3-sulfonamido)butanoic acid (Compound 212)

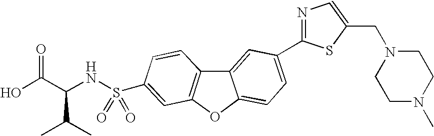

The title compound was prepared by the procedures described in Example 20, using 2-chloro-5-((4-methylpiperazin-1-yl)methyl)thiazole instead of 2-bromothiazole. The compound was obtained as an off-white solid. ¹H NMR (400 MHz, MeOD) δ ppm 0.89 (d, J=7.07 Hz, 3H), 1.01 (d, J=6.82 Hz, 3H), 2.07-2.20 (m, 1H), 2.40 (s, 3H), 2.43-2.58 (m, 4H), 2.62-2.73 (m, 4H), 3.58-3.65 (m, 2H), 7.58 (s, 1H), 7.70 (d, J=8.34 Hz, 1H), 7.88-7.95 (m, 1H), 8.03-8.10 (m, 1H), 8.12 (s, 1H), 8.17 (d, J=8.34 Hz, 1H), 8.54-8.59 (m, 1H). HRMS (ESI-FTMS): calcd for C₂₆H₃₀N₄O₅S₂+H⁺, 543.17304. found: 543.17434.

### Example 20S

### (S)-2-(8-(5-(((cyclopropylmethyl)(propyl)amino)methyl)thiazol-2-yl)dibenzo[b,d]furan-3-sulfonamido)-3-methylbutanoic acid (Compound 213)

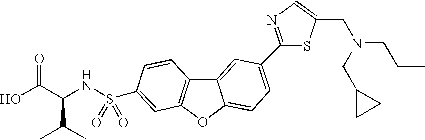

The title compound was prepared by the procedures described in Example 20, using N-((2-chlorothiazol-5-yl)methyl)-N-(cyclopropylmethyl)propan-1-amine instead of 2-bromothiazole. The compound was obtained as an off-white solid. ¹H NMR (400 MHz, MeOD) δ ppm 0.24-0.33 (m, 2H), 0.62-0.71 (m, 2H), 0.89-1.01 (m, 7H), 1.08 (d, J=6.82 Hz, 3H), 1.55-1.71 (m, 2H), 2.07-2.20 (m, 1H), 2.58-2.82 (m, 4H), 3.69-3.90 (m, 3H), 7.57 (s, 1H), 7.64 (d, J=8.84 Hz, 1H), 7.91-8.01 (m, 2H), 8.09-8.18 (m, 2H), 8.32-8.36 (m, 1H). HRMS (ESI-FTMS): calcd for C₂₈H₃₃N₃O₅S₂+H⁺, 556.19344. found: 556.19443.

### Example 20T

### (S)-2-(8-(5-((1H-pyrazol-1-yl)methyl)thiazol-2-yl)dibenzo[b,d]furan-3-sulfonamido)-3-methylbutanoic acid (Compound 214)

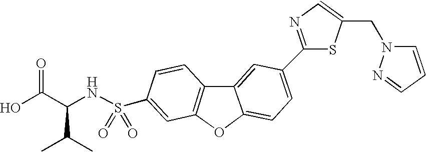

The title compound was prepared by the procedures described in Example 20, using 5-((1H-pyrazol-1-yl)methyl)-2-chlorothiazole instead of 2-bromothiazole. ¹H NMR (400 MHz, MeOD) δ ppm 0.92 (d, J=6.82 Hz, 3H), 0.99 (d, J=6.57 Hz, 3H), 2.01-2.08 (m, 1H), 4.24-4.31 (m, 1H), 5.64 (s, 2H), 6.32-6.39 (m, 1H), 7.51-7.60 (m, 2H), 7.68-7.76 (m, 3H), 7.83 (s, 1H), 7.88-7.94 (m, 1H), 8.07-8.14 (m, 2H), 8.21 (d, J=7.83 Hz, 1H). HRMS (ESI-FTMS): calcd for C₂₄H₂₂N₄O₅S₂+H⁺, 511.11044. found: 511.11086.

### Example 20U

### (S)-2-(8-(5-(hydroxymethyl)thiazol-2-yl)dibenzo[b,d]furan-3-sulfonamido)-3-methylbutanoic acid (Compound 215)

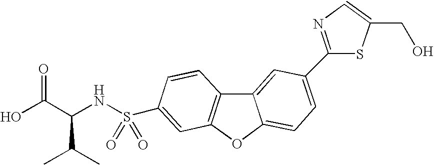

The title compound was prepared by the procedures described in Example 20, using (2-chlorothiazol-5-yl)methyl acetate instead of 2-bromothiazole. The compound was obtained as an off-white solid. ¹H NMR (400 MHz, MeOD) δ ppm 0.93 (d, J=6.82 Hz, 3H), 0.99 (d, J=6.82 Hz, 3H), 2.05-2.14 (m, 1H), 3.77 (d, J=5.31 Hz, 1H), 4.86 (d, J=0.76 Hz, 2H), 7.68-7.74 (m, 2H), 7.91 (dd, J=8.08, 1.52 Hz, 1H), 8.08-8.15 (m, 2H), 8.21 (d, J=8.08 Hz, 1H), 8.60 (d, J=1.77 Hz, 1H). HRMS (ESI-FTMS): calcd for C₂₁H₂₀N₂O₆S₂+H⁺, 461.08355. found: 461.08399.

### Example 20V

### (S)-2-(8-(5-(isoxazol-3-yl)thiophen-2-yl)dibenzo[b,d]furan-3-sulfonamido)-3-methylbutanoic acid (Compound 216)

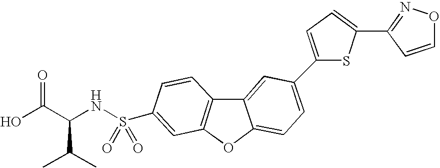

The title compound was prepared by the procedures described in Example 20, using 3-(5-bromothiophen-2-yl)isoxazole instead of 2-bromothiazole. The compound was obtained as an off-white solid. ¹H NMR (400 MHz, MeOD) δ ppm 0.91 (d, J=6.82 Hz, 3H), 1.00 (d, J=6.82 Hz, 3H), 2.06-2.16 (m, 1H), 2.67 (s, 1H), 3.71 (d, J=5.31 Hz, 1H), 7.57 (d, J=4.04 Hz, 1H), 7.72 (d, J=8.59 Hz, 1H), 7.88-7.96 (m, 3H), 8.12 (d, J=1.52 Hz, 1H), 8.17-8.24 (m, 2H), 8.43 (d, J=1.52 Hz, 1H). MS (LC-ESIMS) m/z 497.2 (MH⁺).

### Example 20W

### (S)-2-(8-(4-bromothiazol-2-yl)dibenzo[b,d]furan-3-sulfonamido)-3-methylbutanoic acid (Compound 217)

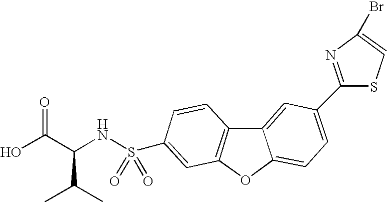

The title compound was prepared by the procedures described in Example 20, using 2,4-dibromothiazole instead of 2-bromothiazole. The compound was obtained as an off-white solid. ¹H NMR (400 MHz, MeOD) δ ppm 0.93 (d, J=6.82 Hz, 3H), 0.99 (d, J=6.82 Hz, 3H), 2.05-2.15 (m, 1H), 3.78 (d, J=5.31 Hz, 1H), 7.47 (s, 1H), 7.64-7.76 (m, 1H), 7.88-7.95 (m, 1H), 8.07-8.16 (m, 2H), 8.20 (d, J=8.08 Hz, 1H), 8.64 (d, J=2.02 Hz, 1H). HRMS (ESI-FTMS): calcd for C₂₀H₁₇BrN₂O₅S₂+H⁺, 508.98350. found: 508.98535.

### Example 20X

### (S)-2-(8-(4-fluorobenzo[d]thiazol-2-yl)dibenzo[b,d]furan-3-sulfonamido)-3-methylbutanoic acid (Compound 218)

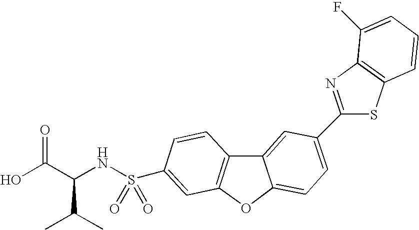

The title compound was prepared by the procedures described in Example 20, using 2-bromo-4-fluorobenzo[d]thiazole instead of 2-bromothiazole. The compound was obtained as an off-white solid. ¹H NMR (400 MHz, MeOD) δ ppm 0.92 (d, J=6.82 Hz, 3H), 0.92 (d, 4H), 1.00 (d, J=6.82 Hz, 3H), 2.05-2.17 (m, 1H), 3.79 (d, J=5.31 Hz, 1H), 7.20-7.31 (m, 1H), 7.37-7.47 (m, 1H), 7.71-7.82 (m, 2H), 7.89-7.98 (m, 1H), 8.15 (d, J=1.52 Hz, 1 H), 8.23 (d, J=8.34 Hz, 1H), 8.31 (dd, J=8.59, 2.02 Hz, 1H), 8.80-8.88 (m, 1H). HRMS (ESI-FTMS): calcd for C₂₄H₁₉FN₂O₅S₂+H⁺, 499.07922. found: 499.08045.

### Example 20Y

### (S)-2-(8-(5-fluorobenzo[d]thiazol-2-yl)dibenzo[b,d]furan-3-sulfonamido)-3-methylbutanoic acid (Compound 219)

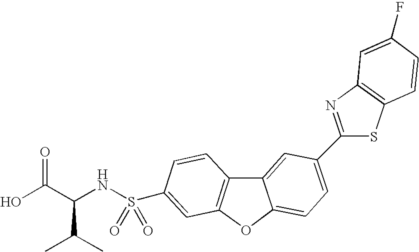

The title compound was prepared by the procedures described in Example 20, using 2-bromo-5-fluorobenzo[d]thiazole instead of 2-bromothiazole. The compound was obtained as an off-white solid. ¹H NMR (400 MHz, MeOD) δ ppm 0.92 (d, J=6.82 Hz, 3H), 1.01 (d, J=6.82 Hz, 3H), 1.99-2.22 (m, 1H), 3.78 (d, J=5.05 Hz, 1H), 7.15-7.28 (m, 1H), 7.69-7.82 (m, 2H), 7.88-8.03 (m, 2H), 8.15 (d, J=1.52 Hz, 1H), 8.21 (d, J=8.08 Hz, 1H), 8.27 (dd, J=8.72, 1.89 Hz, 1H), 8.76 (d, J=2.02 Hz, 1H). HRMS (ESI-FTMS): calcd for C₂₄H₁₉FN₂O₅S₂+H⁺, 499.07922. found: 499.08056.

### Example 20Z

### (S)-2-(8-(5,6-difluorobenzo[d]thiazol-2-yl)dibenzo[b,d]furan-3-sulfonamido)-3-methylbutanoic acid (Compound 220)

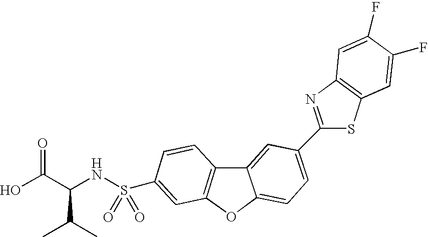

The title compound was prepared by the procedures described in Example 20, using 2-bromo-5,6-difluorobenzo[d]thiazole instead of 2-bromothiazole. The compound was obtained as an off-white solid. ¹H NMR (400 MHz, MeOD) δ ppm 0.92 (d, J=6.82 Hz, 3H), 1.00 (d, J=6.82 Hz, 3H), 2.04-2.19 (m, 1H), 3.80 (d, J=5.31 Hz, 1H), 7.72-7.98 (m, 4 H), 8.15 (s, 1H), 8.18-8.31 (m, 2H), 8.74 (d, J=1.77 Hz, 1H). HRMS (ESI-FTMS): calcd for C₂₄H₁₈F₂N₂O₅S₂+H⁺, 517.06979. found: 517.07054.

### Example 20AA

### (S)-3-methyl-2-(8-(6-(trifluoromethoxy)benzo[d]thiazol-2-yl)dibenzo [b,d]furan-3-sulfonamido)butanoic acid (Compound 221)

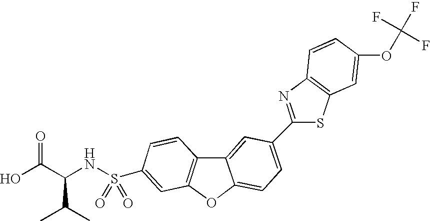

The title compound was prepared by the procedures described in Example 20, using 2-bromo-6-trifluoromethoxybenzo[d]thiazole instead of 2-bromothiazole. The compound was obtained as an off-white solid. ¹H NMR (400 MHz, MeOD) δ ppm 0.93 (d, J=6.82 Hz, 3H), 1.00 (d, J=6.82 Hz, 3H), 1.98-2.22 (m, 1H), 3.79 (d, J=5.31 Hz, 1H), 7.44 (d, J=9.85 Hz, 1H), 7.80 (d, J=8.59 Hz, 1H), 7.87-7.98 (m, 2H), 8.10 (d, J=8.84 Hz, 1H), 8.15 (s, 1H), 8.24 (d, J=8.34 Hz, 1H), 8.30 (dd, J=8.72, 1.89 Hz, 1H), 8.79 (d, J=1.77 Hz, 1H). HRMS (ESI-FTMS): calcd for C₂₅H₁₉F₃N₂O₆S₂+H⁺, 565.07094. found: 565.07111.

### Example 20AB

### (S)-3-methyl-2-(8-(4,5,6-trifluorobenzo[d]thiazol-2-yl)dibenzo[b,d] furan-3-sulfonamido)butanoic acid (Compound 222)

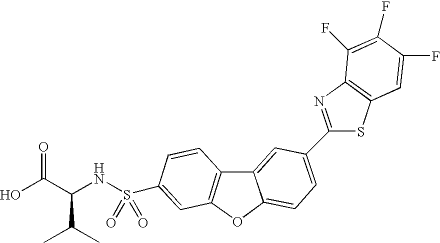

The title compound was prepared by the procedures described in Example 20, using 2-bromo-4,5,6-trifluorobenzo[d]thiazole instead of 2-bromothiazole. The compound was obtained as an off-white solid. ¹H NMR (400 MHz, MeOD) δ ppm 0.92 (d, J=7.07 Hz, 3H), 0.99 (d, J=6.82 Hz, 3H), 2.02-2.16 (m, 1H), 3.75 (d, J=5.31 Hz, 1H), 7.78-7.88 (m, 3H), 7.90-7.98 (m, 1H), 8.10-8.20 (m, 1H), 8.29-8.42 (m, 2H), 8.90-8.93 (m, 1H). HRMS (ESI-FTMS): calcd for C₂₄H₁₇F₃N₂O₅S₂+H⁺, 535.06037. found: 535.0601.

### Example 20AC

### (S)-2-(8-(4-methoxybenzo[d]thiazol-2-yl)dibenzo[b,d]furan-3-sulfonamido)-3-methylbutanoic acid (Compound 223)

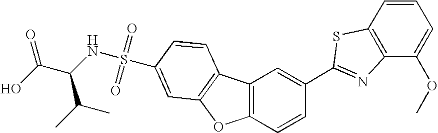

The title compound was prepared by the procedures described in Example 20, using 2-bromo-4-methoxybenzo[d]thiazole instead of 2-bromothiazole. The compound was obtained as a white solid. 1H NMR (300 MHz, DMSO-d₆) δ ppm 12.52 (br. s., 1H), 9.01 (d, J=1.5 Hz, 1H), 8.59 (d, J=8.2 Hz, 1H), 8.34 (dd, J=8.7, 1.9 Hz, 1H), 8.18 (d, J=9.7 Hz, 1H), 8.12 (d, J=1.2 Hz, 1H), 7.97 (d, J=8.5 Hz, 1H), 7.88 (dd, J=8.2, 1.8 Hz, 1H), 7.72 (d, J=7.3 Hz, 1H), 7.44 (t, J=8.1 Hz, 1H), 7.12 (d, J=7.3 Hz, 1H), 4.03 (s, 3H), 3.64 (dd, J=9.5, 6.0 Hz, 1H), 1.82-2.06 (m, J=13.3, 6.9, 6.9, 6.7 Hz, 1H), 0.85 (d, J=6.7 Hz, 3H), 0.82 (d, J=6.7 Hz, 3H). ESIMS (m/z) 511.17 (MH⁺).

### Example 20AD

### (S)-2-(8-(5-chlorothiazol-2-yl)dibenzo[b,d]furan-3-sulfonamido)-3-methylbutanoic acid (Compound 224)

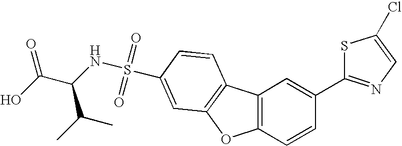

The title compound was prepared by the procedures described in Example 20, using 2-bromo-5-chlorothiazole instead of 2-bromothiazole. The compound was obtained as a white solid. 1H NMR (300 MHz, DMSO-d₆) d ppm 12.50 (s, 1H), 8.84 (d, J=1.5 Hz, 1H), 8.49 (d, J=7.9 Hz, 1H), 8.18 (d, J=9.7 Hz, 1H), 8.17 (dd, J=8.8, 2.1 Hz, 1H), 8.11 (d, J=1.2 Hz, 1H), 8.01 (s, 1H), 7.93 (d, J=8.8 Hz, 1H), 7.87 (dd, J=8.2, 1.5 Hz, 1H), 3.52-3.73 (m, 1H), 1.88-2.04 (m, J=13.2, 6.7, 6.6, 6.6 Hz, 1H), 0.85 (d, J=6.7 Hz, 3H), 0.82 (d, J=7.0 Hz, 3H). ESIMS (m/z) 465.14 (MH+).

### Example 20AE

### (S)-2-(8-(5-methoxybenzo[d]thiazol-2-yl)dibenzo[b,d]furan-3-sulfonamido)-3-methylbutanoic acid (Compound 225)

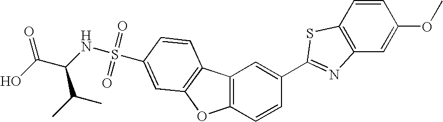

The title compound was prepared by the procedures described in Example 20, using 2-bromo-5-methoxybenzo[d]thiazole instead of 2-bromothiazole. The compound was obtained as a white solid. 1H NMR (300 MHz, DMSO-d₆) δ ppm 12.51 (br. s., 1H), 9.01 (d, J=1.5 Hz, 1H), 8.56 (d, J=8.2 Hz, 1H), 8.34 (dd, J=8.5, 1.8 Hz, 1H), 8.18 (d, J=8.5 Hz, 1 H), 8.10-8.15 (m, 1H), 8.06 (d, J=8.8 Hz, 1H), 7.98 (d, J=8.8 Hz, 1H), 7.88 (dd, J=8.2, 1.2 Hz, 1H), 7.63 (d, J=2.3 Hz, 1H), 7.13 (dd, J=8.8, 2.3 Hz, 1H), 3.90 (s, 3H), 3.63 (dd, J=8.9, 5.7 Hz, 1H), 1.80-2.11 (m, 1H), 0.85 (d, J=7.0 Hz, 3H), 0.82 (d, J=7.0 Hz, 3H). ESIMS (m/z) 511.17 (MH⁺).

The following compounds in Table 14 were prepared using procedures analogous to those described above for the preparation of (S)-3-methyl-2-(8-(thiazol-2-yl)dibenzo[b,d]furan-3-sulfonamido)butanoic acid.

TABLE 14

| Compd No. | HRMS |
|---|---|
| 124 | 448.06898 |
| 125 | 693.15962 |
| 126 | 499.06041 |
| 131 | 498.06565 |
| 151 | 481.0895 |

### Example 21

### (S)-2-(7-(benzo[d]thiazol-2-yl)dibenzo[b,d]furan-3-sulfonamido)-3-methyl butanoic acid (Compound 226)

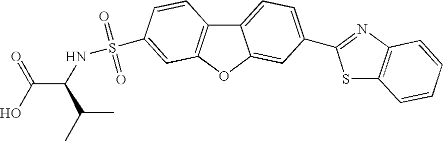

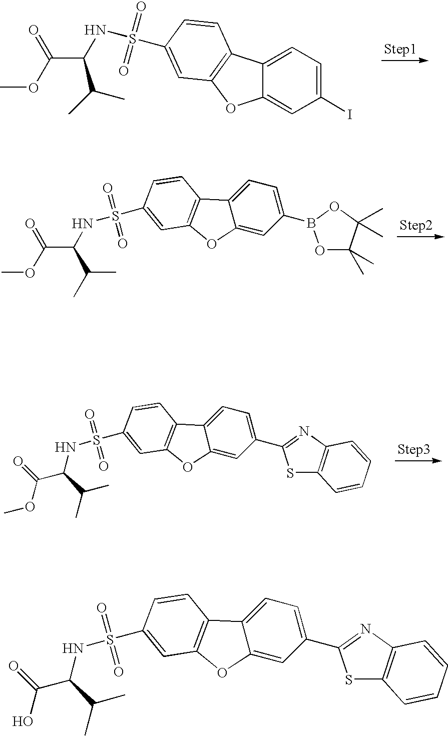

### Step 1: Preparation of (S)-methyl 3-methyl-2-(7-(4,4,5,5-tetramethyl-1,3,2-dioxaborolan-2-yl)dibenzo[b,d]furan-3-sulfonamido)butanoate

(S)-Methyl 2-(7-iododibenzo[b,d]furan-3-sulfonamido)-3-methylbutanoate (an intermediate in the preparation of Example 8) (1.026 g, 2.10 mmol), CH₃COOK (0.62 g, 6.31 mmol), PdCl₂-dppf₂ (90 mg), and bis-pinacolate diboron (1.61 g, 6.33 mmol) were mixed in DMSO (20 ml) and the resulting mixture was stirred at 90° C. for 2 h. The reaction was monitored by LC-MS. After completion of the reaction, the mixture was cooled to room temperature, water (100 ml) was added and the mixture was extracted with DCM (100 ml×2). The organic phases were combined and dried over Na₂SO₄ and concentrated. The residue was purified by silica gel column chromatography to afford the desired product (S)-methyl 3-methyl-2-(7-(4,4,5,5-tetramethyl-1,3,2-dioxaborolan-2-yl)dibenzo[b,d]furan-3-sulfonamido)butanoate (1.02 g, 100% yield) as a white solid.

### Step 2: Preparation of (S)-methyl 2-(7-(benzo[d]thiazol-2-yl)dibenzo[b,d]furan-3-sulfonamido)-3-methylbutanoate

(S)-Methyl 3-methyl-2-(7-(4,4,5,5-tetramethyl-1,3,2-dioxaborolan-2-yl)dibenzo [b,d]furan-3-sulfonamido)butanoate (216 mg, 0.44 mmol), 2-bromo benzo[d]thiazole (190 mg, 0.89 mmol), Pd(PPh₃)₄ (40 mg), K₂CO₃ (123 mg, 0.89 mmol), 2 mL of DME, and 0.5 mL of water were mixed and deoxygenated with nitrogen gas for 10 min. The mixture was stirred in a microwave oven at 120° C. for 15 min, and then purified by flash column chromatography to provide 142 mg of (S)-methyl 2-(7-(benzo[d]thiazol-2-yl)dibenzo[b,d]furan-3-sulfonamido)-3-methylbutanoate as a white solid.

### Step 3: Preparation of (S)-2-(7-(benzo[d]thiazol-2-yl)dibenzo[b,d]furan-3-sulfonamido)-3-methylbutanoic acid

A solution of (S)-methyl 2-(7-(benzo[d]thiazol-2-yl)dibenzo[b,d]furan-3-sulfonamido)-3-methylbutanoate (90 mg) in 0.5 mL of THF was treated with LiOH solution (0.9 M, 0.5 mL) and stirred at room temperature for 3 days. The THF was removed under reduced pressure and the aqueous solution was acidified to pH ˜2. The mixture was filtered and the solid was collected and dried in the air, providing (S)-2-(7-(benzo[d]thiazol-2-yl)dibenzo[b,d]furan-3-sulfonamido)-3-methylbutanoic acid as a white solid (88 mg, 92% yield). ¹H NMR (400 MHz, DMSO-d₆) δ ppm 0.81 (d, J=6.82 Hz, 3H), 0.85 (d, J=6.82 Hz, 3H), 1.97 (d, J=5.81 Hz, 1H), 3.58 (s, 1H), 7.46-7.55 (m, 1H), 7.55-7.64 (m, 1H), 7.87 (dd, J=8.08, 1.52 Hz, 1H), 8.08-8.16 (m, 2H), 8.19-8.27 (m, 2H), 8.44 (t, J=8.21 Hz, 2H), 8.49 (s, 1H). HRMS (ESI-FTMS): calcd for C₂₄H₂₀N₂O₅S₂+H⁺, 481.08864. found: 481.0887.

### Example 21A

### (S)-2-(7-(benzo[d]oxazol-2-yl)dibenzo[b,d]furan-3-sulfonamido)-3-methylbutanoic acid (Compound 227)

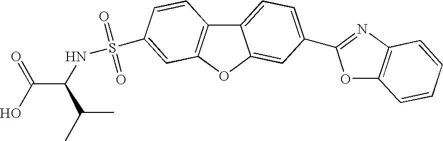

The title compound was prepared by the procedures described in Example 21, using 2-chlorobenzo[d]oxazole instead of 2-bromobenzo[d]thiazole. The compound was obtained as a white solid in 61% yield. ¹H NMR (400 MHz, MeOD) δ ppm 0.95 (d, J=6.82 Hz, 3H), 1.01 (d, J=6.82 Hz, 3H), 2.09 (d, J=6.57 Hz, 1H), 3.77 (d, J=5.56 Hz, 1H), 7.42-7.52 (m, 2H), 7.75 (dd, J=6.44, 2.15 Hz, 1H), 7.78-7.83 (m, 1H), 7.95 (dd, J=8.34, 1.52 Hz, 1H), 8.18 (d, J=1.01 Hz, 1H), 8.30 (d, J=8.34 Hz, 1H), 8.34-8.38 (m, 2H), 8.53 (s, 1H). HRMS (ESI-FTMS): calcd for C₂₄H₂₀N₂O₆S+H⁺, 465.11148. found: 465.11037.

### Example 21A

### (S)-3-methyl-2-(7-(5-(5-methyl-1,2,4-oxadiazol-3-yl)thiazol-2-yl)dibenzo [b,d]furan-3-sulfonamido)butanoic acid (Compound 228)

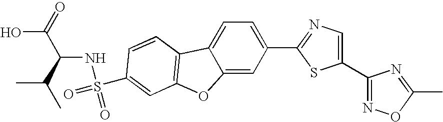

The title compound was prepared by the procedures described in Example 21, using 3-(2-bromothiazol-5-yl)-5-methyl-1,2,4-oxadiazole instead of 2-bromobenzo[d]thiazole. The compound was obtained as a white solid in 85% yield. ¹H NMR (400 MHz, DMSO-d₆) δ ppm 0.78-0.83 (m, 3H), 0.83-0.87 (m, 3H), 1.24 (s, 2H), 2.30-2.36 (m, 1H), 2.67 (s, 3H), 3.52-3.62 (m, 1H), 7.80-7.91 (m, 4H), 8.08 (s, 1H), 8.24 (d, J=1.01 Hz, 1H), 8.34 (dd, J=11.24, 8.21 Hz, 2H). HRMS (ESI-FTMS): calcd for C₂₄H₂₁N₃O₆S₂+H⁺, 512.09445. found: 512.09398.

### Example 21B

### (S)-2-(7-(5-ethylthiophen-2-yl)dibenzo[b,d]furan-3-sulfonamido)-3-methylbutanoic acid (Compound 229)

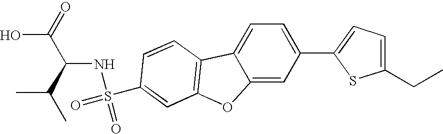

The title compound was prepared by the procedures described in Example 21, using 2-bromo-5-ethylthiophene instead of 2-bromobenzo[d]thiazole. The compound was obtained as a white solid in 100% yield. ¹H NMR (400 MHz, MeOD) δ ppm 0.80 (d, J=6.82 Hz, 3H), 0.88 (d, J=6.82 Hz, 3H), 1.26 (t, J=7.58 Hz, 3H), 1.88-2.01 (m, 1H), 2.72-2.87 (m, 2H), 3.57 (d, J=5.56 Hz, 1H), 6.72-6.78 (m, 1H), 7.27 (d, J=3.54 Hz, 1H), 7.58 (dd, J=8.08, 1.52 Hz, 1H), 7.72-7.78 (m, 2H), 7.93-8.00 (m, 2H), 8.04 (d, J=8.84 Hz, 1H). HRMS (ESI-FTMS): calcd for C₂₃H₂₃NO₅S₂+H⁺, 458.10904. found: 458.1090.

### Example 21C

### (S)-2-(7-(2,4-dimethylthiazol-5-yl)dibenzo[b,d]furan-3-sulfonamido)-3-methylbutanoic acid (Compound 230)

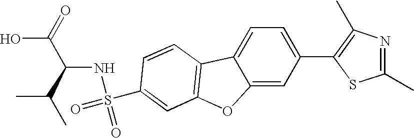

The title compound was prepared by the procedures described in Example 21, using 5-bromo-2,4-dimethylthiazole instead of 2-bromobenzo[d]thiazole. The compound was obtained as a white solid in 100% yield. ¹H NMR (400 MHz, MeOD) δ ppm 1.12 (d, J=6.82 Hz, 3H), 1.20 (d, J=6.57 Hz, 3H), 2.27 (s, 1H), 2.71 (s, 3H), 2.92 (s, 3H), 3.80-3.92 (m, 1H), 7.74 (dd, J=8.21, 1.39 Hz, 1H), 7.97 (s, 1H), 8.11 (dd, J=8.08, 1.52 Hz, 1H), 8.32 (s, 1H), 8.41 (t, J=8.59 Hz, 2H). HRMS (ESI-FTMS): calcd for C₂₂H₂₂N₂O₅S₂+H⁺, 459.10429. found: 459.10494.

### Example 21D

### (S)-2-(7-(5-tert-butylfuran-2-yl)dibenzo[b,d]furan-3-sulfonamido)-3-methylbutanoic acid (Compound 231)

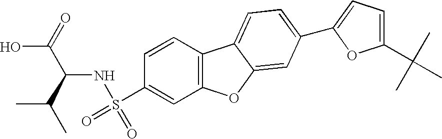

The title compound was prepared by the procedures described in Example 21, using 2-bromo-5-tert-butylfuran instead of 2-bromobenzo[d]thiazole. The compound was obtained as a white solid in 100% yield. ¹H NMR (400 MHz, DMSO-d₆) δ ppm 0.76-0.88 (m, 6H), 1.34 (s, 9H), 1.89-2.01 (m, 1H), 3.57 (s, 1H), 4.03 (s, 1H), 6.26 (d, J=3.54 Hz, 1H), 7.05 (d, J=3.28 Hz, 1H), 7.79 (t, J=1.52 Hz, 1H), 7.81 (t, J=1.52 Hz, 1H), 8.03 (d, 1H), 8.05 (t, J=1.64 Hz, 1H), 8.25 (d, J=8.08 Hz, 1H), 8.29 (d, J=8.08 Hz, 1H). HRMS (ESI-FTMS): calcd for C₂₅H₂₇NO₆S+H⁺, 470.16318. found: 470.163.

### Example 21E

### (S)-3-methyl-2-(7-(5-propyl)thiophen-2-yl)dibenzo[b,d]furan-3-sulfonamido)butanoic acid (Compound 232)

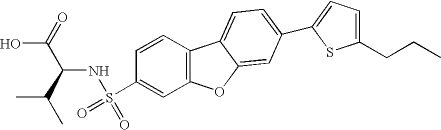

The title compound was prepared by the procedures described in Example 21, using 2-bromo-5-propylthiophene instead of 2-bromobenzo[d]thiazole. The compound was obtained as a white solid in 100% yield. ¹H NMR (400 MHz, DMSO-d₆) δ ppm 0.77-0.87 (m, J=13.14, 6.82 Hz, 6H), 0.97 (t, J=7.45 Hz, 3H), 1.59-1.74 (m, 2H), 1.89-2.00 (m, J=6.06 Hz, 1H), 2.07 (s, 1H), 2.81 (t, J=7.58 Hz, 2H), 3.55-3.66 (m, 1H), 6.92 (d, J=3.54 Hz, 1H), 7.56 (d, J=3.54 Hz, 1H), 7.71 (dd, J=8.21, 1.64 Hz, 1H), 7.81 (dd, J=8.21, 1.64 Hz, 1H), 8.03 (dd, J=8.21, 1.14 Hz, 2H), 8.16 (dd, 1H), 8.23 (d, J=8.08 Hz, 1H), 8.30 (d, J=8.08 Hz, 1H). HRMS (ESI-FTMS): calcd for C₂₄H₂₅NO₅S₂+H⁺, 472.12469. found: 472.12456.

### Example 21F

### (S)-2-(7-(5-chloro-4-(trifluoromethyl)thiazol-2-yl)dibenzo[b,d]furan-3-sulfonamido)-3-methylbutanoic acid (Compound 233)

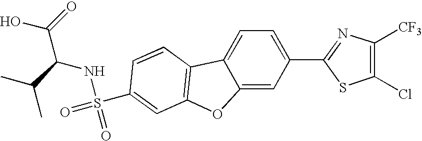

The title compound was prepared by the procedures described in Example 21, using 5-chloro-2-fluoro-4-(trifluoromethyl)thiazole instead of 2-bromobenzo[d]thiazole. The compound was obtained as a white solid in 100% yield. ¹H NMR (400 MHz, MeOD) δ ppm 1.14 (d, J=6.82 Hz, 3H), 1.20 (d, J=6.82 Hz, 3H), 2.20-2.33 (m, J=6.82, 5.81 Hz, 1H), 3.96 (d, J=5.56 Hz, 1H), 8.13 (dd, J=8.08, 1.52 Hz, 1H), 8.23 (dd, J=8.08, 1.52 Hz, 1H), 8.35 (d, J=1.52 Hz, 1H), 8.42-8.50 (m, 3H). HRMS (ESI-FTMS): calcd for C₂₁H₁₆ClF₃N₂O₅S₂+H⁺, 533.02140. found: 533.02178.

### Example 21G

### (S)-3-methyl-2-(7-(5-methylthiazol-2-yl)dibenzo[b,d]furan-3-sulfonamido)butanoic acid (Compound 234)

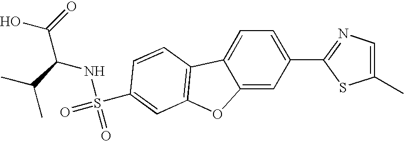

The title compound was prepared by the procedures described in Example 21, using 2-bromo-5-methylthiazole instead of 2-bromobenzo[d]thiazole. The compound was obtained as a white solid in 100% yield. ¹H NMR (400 MHz, DMSO-d₆) δ ppm 0.83 (dd, J=13.77, 6.69 Hz, 6H), 1.88-2.01 (m, 1H), 2.52-2.57 (m, J=1.01 Hz, 3H), 3.58 (s, 1H), 7.69 (d, J=1.01 Hz, 1H), 7.84 (dd, J=8.21, 1.64 Hz, 1H), 8.00 (dd, J=8.08, 1.52 Hz, 1H), 8.08 (d, J=1.01 Hz, 1H), 8.25 (d, J=1.01 Hz, 1H), 8.35 (dd, J=10.36, 8.08 Hz, 2H). HRMS (ESI-FTMS): calcd for C₂₁H₂₀N₂O₅S₂+H⁺, 445.08864. found: 445.08932.

### Example 21H

### (S)-2-(7-(2-isobutyl-4-methylthiazol-5-yl)dibenzo[b,d]furan-3-sulfonamido)-3-methylbutanoic acid (Compound 235)

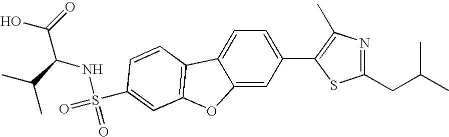

The title compound was prepared by the procedures described in Example 21, using 5-bromo-2-isobutyl-4-methylthiazole instead of 2-bromobenzo[d]thiazole. The compound was obtained as a white solid in 100% yield. ¹H NMR (400 MHz, MeOD) δ ppm 1.13 (d, J=6.82 Hz, 3H), 1.20 (d, J=6.82 Hz, 3H), 1.26 (d, J=6.57 Hz, 6H), 2.18-2.43 (m, J=7.07 Hz, 2H), 2.74 (s, 3H), 3.10 (d, J=7.33 Hz, 2H), 3.86-3.97 (m, 1H), 7.76 (dd, J=8.21, 1.39 Hz, 1H), 7.99 (d, J=1.52 Hz, 1H), 8.11 (dd, J=8.21, 1.39 Hz, 1H), 8.32 (d, J=1.01 Hz, 1H), 8.41 (t, J=8.34 Hz, 2H). HRMS (ESI-FTMS): calcd for C₂₅H₂₈N₂O₅S₂+H⁺, 501.15124. found: 501.15233.

### Example 21I

### (S)-3-methyl-2-(7-(6-(trifluoromethyl)benzo[d]thiazol-2-yl)dibenzo [b,d]furan-3-sulfonamido)butanoic acid (Compound 236)

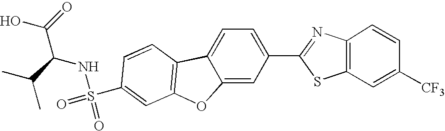

The title compound was prepared by the procedures described in Example 21, using 2-bromo-6-(trifluoromethyl)benzo[d]thiazole instead of 2-bromobenzo[d]thiazole. The compound was obtained as a white solid in 100% yield. ¹H NMR (400 MHz, MeOD) δ ppm 1.14 (d, J=6.57 Hz, 3H), 1.21 (d, J=6.82 Hz, 3H), 2.30 (s, 1H), 3.96 (d, J=5.56 Hz, 1H), 8.05 (dd, 1H), 8.15 (dd, J=8.08, 1.52 Hz, 1H), 8.38 (d, J=1.01 Hz, 1H), 8.43-8.46 (m, 1H), 8.47 (d, J=1.52 Hz, 1H), 8.49 (d, J=8.34 Hz, 1H), 8.51-8.55 (m, 1H), 8.66-8.69 (m, 1H), 8.70 (s, 1H). HRMS (ESI-FTMS): calcd for C₂₅H₁₉F₃N₂O₅S₂+H⁺, 549.07602; found: 549.07735.

### Example 21J

### (S)-2-(7-(6-fluorobenzo[d]thiazol-2-yl)dibenzo[b,d]furan-3-sulfonamido)-3-methylbutanoic acid (Compound 237)

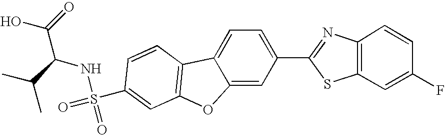

The title compound was prepared by the procedures described in Example 21, using 2-bromo-6-fluorobenzo[d]thiazole instead of 2-bromobenzo[d]thiazole. The compound was obtained as a white solid in 100% yield. ¹H NMR (400 MHz, MeOD) δ ppm 1.13 (d, J=6.82 Hz, 3H), 1.21 (d, J=6.82 Hz, 3H), 2.29 (d, J=5.56 Hz, 1H), 3.91 (d, J=5.56 Hz, 1H), 7.53-7.62 (m, 1H), 7.79 (s, 1H), 7.81-7.91 (m, 1H), 8.04 (dd, J=8.34, 2.78 Hz, 1H), 8.14 (dd, J=8.08, 1.52 Hz, 1H), 8.29 (dd, J=9.09, 4.80 Hz, 1H), 8.49 (dd, J=10.99, 8.21 Hz, 2H), 8.62 (s, 1H). HRMS (ESI-FTMS): calcd for C₂₄H₁₉FN₂O₅S₂+H⁺, 499.07922. found: 499.07982.

### Example 22

### (R)-3-methyl-2-(7-(thiazol-2-yl)dibenzo[b,d]furan-2-sulfonamido)butanoic acid (Compound 291)

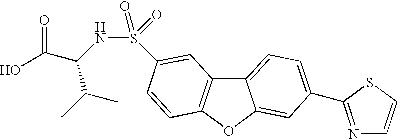

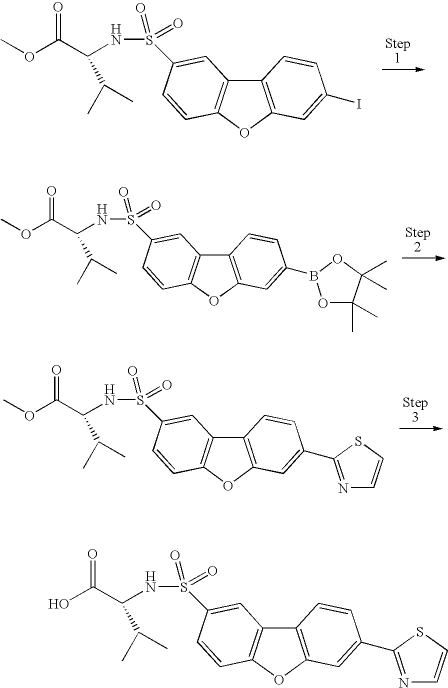

### Step 1: Preparation of (R)-methyl 3-methyl-2-(7-(4,4,5,5-tetramethyl-1,3,2-dioxaborolan-2-yl)dibenzo[b,d]furan-2-sulfonamido)butanoate

A mixture of (R)-methyl 2-(7-iododibenzo[b,d]furan-2-sulfonamido)-3-methylbutanoate (5000 mg, 10.25 mmol) (an intermediate synthesized in Step 6 of Example 4), 4,4,4′,4′,5,5,5′,5′-octamethyl-2,2′-bi(1,3,2-dioxaborolane) (2858 mg, 11.25 mmol), PdCl₂(dppf).CH₂Cl₂ (250 mg, 0.30 mmol), KOAc (3020 mg, 23.8 mmol) and DMSO (40 ml) was heated at 80° C. for 5 hours. After cooling to RT, the mixture was poured into ethyl acetate and water, the organic layer was separated, concentrated under reduced pressure, and the crude residue purified by column chromatography to provide (R)-methyl 3-methyl-2-(7-(4,4,5,5-tetramethyl-1,3,2-dioxaborolan-2-yl)dibenzo[b,d]furan-2-sulfonamido)butanoate as a white solid (4.2 g).

### Step 2: Preparation of (R)-methyl 3-methyl-2-(7-(thiazol-2-yl)dibenzo[b,d]furan-2-sulfonamido)butanoate

A mixture of (R)-methyl 3-methyl-2-(7-(4,4,5,5-tetramethyl-1,3,2-dioxaborolan-2-yl)dibenzo[b,d]furan-2-sulfonamido)butanoate (100 mg, 0.2 mmol), 2-bromothiazole (35 uL, 0.4 mmol), PdCl₂(dppf).CH₂Cl₂ (17 mg, 0.02 mmol), K₃PO₄ (2 M solution in water) (0.6 mL, 1.2 mmol) and DMF (4 ml) was heated at 80° C. for 3 hours. After cooling to RT, the mixture was poured into ethyl acetate and water, the organic layer was separated, concentrated under reduced pressure, and the crude residue was purified by preparative HPLC to yield (R)-methyl 3-methyl-2-(7-(thiazol-2-yl)dibenzo[b,d]furan-2-sulfonamido)butanoate (53 mg).

### Step 3: Preparation of (R)-3-methyl-2-(7-(thiazol-2-yl)dibenzo[b,d]furan-2-sulfonamido)butanoic acid

A solution of (R)-methyl 3-methyl-2-(7-(thiazol-2-yl)dibenzo[b,d]furan-2-sulfonamido)butanoate (40.7 mg, 0.09 mmol) in THF/MeOH/water (2 mL) was treated with LiOH (5 equivalents), and the reaction was stirred overnight at RT. Following the addition of water, the pH of the solution was adjusted to between 4-5, and the precipitate obtained was then filtered to yield (R)-3-methyl-2-(7-(thiazol-2-yl)dibenzo[b,d]furan-2-sulfonamido)butanoic acid as a white solid (21.6 mg). ¹H NMR (400 MHz, DMSO-d₆) δ ppm 0.81 (d, J=6.57 Hz, 3H), 0.84 (d, J=6.82 Hz, 3H), 1.88-2.02 (m, 1H), 3.56-3.65 (m, 1H), 7.89 (d, J=3.28 Hz, 1H), 7.90-7.95 (m, 1H), 7.95-8.03 (m, 2H), 8.07 (dd, J=8.08, 1.52 Hz, 2H), 8.33 (d, J=1.01 Hz, 1H), 8.43 (d, J=8.08 Hz, 1H), 8.66 (d, J=2.02 Hz, 1H). HRMS (ESI-FTMS): calcd for C₂₀H₁₈N₂O₅S₂+H⁺: 431.07299. found: 431.07384.

### Example 22A

### (R)-2-(7-(5-ethylthiophen-2-yl)dibenzo[b,d]furan-2-sulfonamido)-3-methylbutanoic acid (Compound 239)

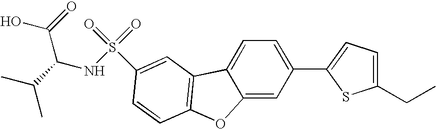

The title compound was prepared by the procedures described in Example 22, using 2-bromo-5-ethylthiophene instead of 2-bromobenzo[d]thiazole. The compound was obtained as a white solid in 100% yield. ¹H NMR (400 MHz, CHLOROFORM-d) δ ppm 0.88 (d, 3H), 0.97 (d, J=6.82 Hz, 3H), 1.37 (t, J=7.45 Hz, 3H), 2.00-2.09 (m, 1H), 2.84-2.94 (m, 2H), 3.35 (s, 3H), 3.83 (dd, J=10.23, 5.18 Hz, 1H), 5.17 (d, J=10.11 Hz, 1H), 6.78-6.87 (m, 1H), 7.25 (d, J=3.54 Hz, 1H), 7.61-7.66 (m, 2H), 7.77 (s, 1H), 7.88-7.95 (m, 2H), 8.43 (d, J=1.52 Hz, 1H). HRMS (ESI-FTMS): calcd for C₂₃H₂₃NO₅S₂+H⁺, 458.10904. found: 458.11102.

### Example 22B

### (R)-2-(7-(5-tert-butylfuran-2-yl)dibenzo[b,d]furan-2-sulfonamido)-3-methylbutanoic acid (Compound 240)

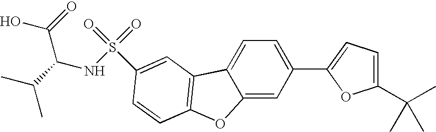

The title compound was prepared by the procedures described in Example 22, using 2-bromo-5-tert-buthylthiophene instead of 2-bromobenzo[d]thiazole. The compound was obtained as a white solid in 100% yield. ¹H NMR (400 MHz, MeOD) δ ppm 0.93 (d, J=6.82 Hz, 3H), 1.00 (d, J=6.82 Hz, 3H), 1.41 (s, 9H), 1.99-2.18 (m, 1H), 3.73 (d, J=5.31 Hz, 1H), 6.19 (d, J=3.28 Hz, 1H), 6.82 (d, J=3.28 Hz, 1H), 7.66-7.81 (m, 2H), 7.89 (s, 1H), 7.99 (dd, J=8.72, 1.89 Hz, 1H), 8.11 (d, J=8.34 Hz, 1H), 8.54 (d, J=1.77 Hz, 1H). HRMS (ESI-FTMS): calcd for C₂₅H₂₇NO₆S+H⁺, 470.16318. found: 470.164982.

### Example 22C

### (S)-2-(7-(5-tert-butylfuran-2-yl)dibenzo[b,d]furan-2-sulfonamido)-3-methylbutanoic acid (Compound 241)

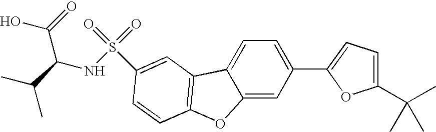

The title compound was prepared using the same procedures described in Example 22, using (S)-isomer. The compound was obtained as a white solid in 100% yield. ¹H NMR (400 MHz, MeOD) δ ppm 1.13 (d, J=6.82 Hz, 3H), 1.20 (d, J=6.82 Hz, 3H), 1.60 (s, 9H), 2.27 (dd, J=12.63, 6.82 Hz, 1H), 3.94 (d, J=5.56 Hz, 1H), 7.00 (d, J=3.54 Hz, 1H), 7.86-7.97 (m, 2H), 8.06 (d, J=1.26 Hz, 1H), 8.18 (dd, J=8.59, 2.02 Hz, 1H), 8.28 (d, J=8.34 Hz, 1H), 8.72 (d, J=1.77 Hz, 1H). HRMS (ESI-FTMS): calcd for C₂₅H₂₇NO₆S+H⁺, 470.16318. found: 470.16513.

### Example 22D

### (S)-2-(7-(5-ethylthiophen-2-yl)dibenzo[b,d]furan-2-sulfonamido)-3-methylbutanoic acid (Compound 242)

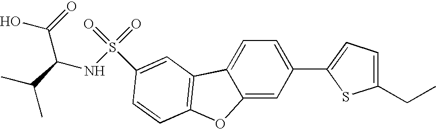

The title compound was prepared using the same procedures described in Example 22, using (S)-isomer. The compound was obtained as a white solid in 100% yield. ¹H NMR (400 MHz, MeOD) δ ppm 1.13 (d, J=6.82 Hz, 3H), 1.20 (d, J=6.82 Hz, 3H), 1.57 (t, J=7.58 Hz, 3H), 2.27 (dd, J=12.76, 6.44 Hz, 1H), 3.11 (q, J=7.66 Hz, 2H), 3.91 (d, J=5.56 Hz, 1H), 7.06 (d, J=3.54 Hz, 1H), 7.56 (d, J=3.54 Hz, 1H), 7.90 (t, 2H), 8.04 (s, 1H), 8.19 (dd, J=8.59, 2.02 Hz, 1H), 8.29 (d, J=8.08 Hz, 1H), 8.74 (d, J=1.77 Hz, 1H). HRMS (ESI-FTMS): calcd for C₂₃H₂₃NO₅S₂+H⁺, 458.10904. found: 458.11081.

### Example 22E

### (R)-3-methyl-2-(7-(5-propyl)thiophen-2-yl)dibenzo[b,d]furan-2-sulfonamido)butanoic acid (Compound 243)

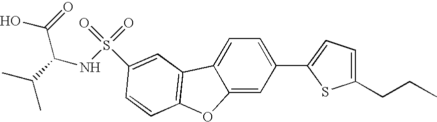

The title compound was prepared by the procedures described in Example 22, using 2-bromo-5-propylthiophene instead of 2-bromobenzo[d]thiazole. The compound was obtained as a white solid in 100% yield. ¹H NMR (400 MHz, MeOD) δ ppm 1.14 (d, J=6.82 Hz, 3H), 1.16-1.28 (m, 6H), 1.89-2.04 (m, 2H), 2.27 (d, J=6.57 Hz, 1H), 3.06 (t, J=7.45 Hz, 2H), 3.94 (d, J=5.56 Hz, 1H), 6.96-7.07 (m, 1H), 7.56 (d, J=3.54 Hz, 1H), 7.85-7.96 (m, 2H), 8.05 (s, 1H), 8.19 (dd, J=8.72, 1.90 Hz, 1H), 8.29 (d, J=8.08 Hz, 1H), 8.74 (d, J=2.02 Hz, 1H). HRMS (ESI-FTMS): calcd for C₂₄H₂₅NO₅S₂+H⁺, 472.12469. found: 472.12707.

### Example 22F

### (R)-2-(7-(2-isobutylthiazol-5-yl)dibenzo[b,d]furan-2-sulfonamido)-3-methylbutanoic acid (Compound 244)

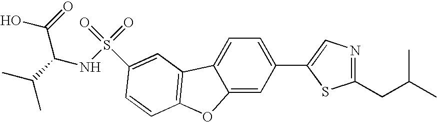

The title compound was prepared by the procedures described in Example 22, using 5-bromo-2-isobutylthiazole instead of 2-bromobenzo[d]thiazole. The compound was obtained as a white solid in 50% yield. ¹H NMR (400 MHz, DMSO-d₆) δ ppm 0.73 (d, J=6.82 Hz, 3H), 0.77 (d, J=6.82 Hz, 3H), 0.92 (d, J=6.57 Hz, 6H), 1.89 (dd, J=12.76, 6.69 Hz, 1H), 1.95-2.07 (m, 1H), 2.83 (d, J=7.07 Hz, 2H), 3.51 (s, 1H), 7.66 (dd, J=8.21, 1.64 Hz, 1H), 7.78-7.84 (m, 1H), 7.84-7.90 (m, 1H), 8.01 (d, J=1.26 Hz, 1H), 8.20 (s, 1H), 8.27 (d, J=8.34 Hz, 1H), 8.54 (d, J=1.52 Hz, 1H). HRMS (ESI-FTMS): calcd for C₂₄H₂₆N₂O₅S₂+H⁺, 487.13559. found: 487.13647.

### Example 22G

### (R)-2-(7-(2-isobutyl-4-methylthiazol-5-yl)dibenzo[b,d]furan-2-sulfonamido)-3-methylbutanoic acid (Compound 245)

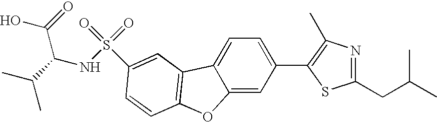

The title compound was prepared by the procedures described in Example 22, using 5-bromo-2-isobutyl-4-methylthiazole instead of 2-bromobenzo[d]thiazole. The compound was obtained as a white solid in 100% yield. ¹H NMR (400 MHz, MeOD) δ ppm 0.81 (d, J=6.82 Hz, 3H), 0.89 (d, J=6.82 Hz, 3H), 0.94 (d, J=6.57 Hz, 6H), 1.92-2.09 (m, 1H), 2.42 (d, 3H), 2.78 (d, 1H), 2.78 (d, J=7.33 Hz, 2H), 3.59 (d, J=5.31 Hz, 1H), 7.43 (dd, J=7.83, 1.52 Hz, 1H), 7.59-7.70 (m, 2H), 7.92 (dd, J=8.84, 2.02 Hz, 1H), 8.08 (d, J=8.08 Hz, 1H), 8.48 (d, J=1.26 Hz, 1H). HRMS (ESI-FTMS): calcd for C₂₅H₂₈N₂O₅S₂+H⁺, 501.15124. found: 501.1516.

### Example 22H

### (S)-3-methyl-2-(7-(5-propyl)thiophen-2-yl)dibenzo[b,d]furan-2-sulfonamido)butanoic acid (Compound 246)

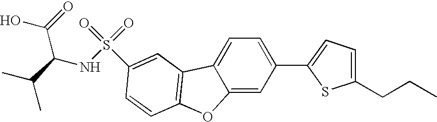

The title compound was prepared using the same procedures described in preparation of Example 22, using (S)-isomer. The compound was obtained as a white solid in 100% yield. ¹H NMR (400 MHz, MeOD) δ ppm 0.92 (d, J=6.82 Hz, 3H), 0.97-1.09 (m, 6H), 1.70-1.88 (m, 2H), 1.99-2.14 (m, 1H), 2.86 (t, J=7.20 Hz, 2H), 3.64 (d, J=5.05 Hz, 1H), 6.86 (d, J=3.54 Hz, 1H), 7.37 (d, J=3.79 Hz, 1H), 7.71 (t, 2H), 7.86 (s, 1H), 7.99 (dd, J=8.72, 1.89 Hz, 1H), 8.11 (d, J=7.58 Hz, 1H), 8.37 (s, 1H), 8.54 (d, J=1.52 Hz, 1H). HRMS (ESI-FTMS): calcd for C₂₄H₂₅NO₅S₂+H⁺, 472.12469. found: 472.12673.

### Example 22I

### (S)-2-(7-(2-isobutyl-4-methylthiazol-5-yl)dibenzo[b,d]furan-2-sulfonamido)-3-methylbutanoic acid (Compound 247)

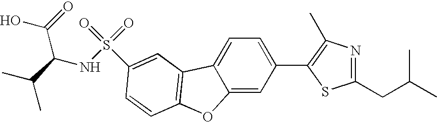

The title compound was prepared using the same procedures described in preparation of Example 22, using (S)-isomer. The compound was obtained as a white solid in 100% yield. ¹H NMR (400 MHz, MeOD) δ ppm 0.82 (d, J=6.82 Hz, 3H), 0.88 (d, J=6.82 Hz, 3H), 0.91-0.96 (m, 6H), 1.88-2.08 (m, 1H), 2.36-2.45 (m, 2H), 2.78 (d, J=7.33 Hz, 1H), 3.65 (d, J=5.56 Hz, 1H), 7.42 (dd, J=7.96, 1.39 Hz, 1H), 7.64 (dd, J=4.80, 3.79 Hz, 2H), 7.92 (dd, J=8.72, 1.89 Hz, 1H), 8.06 (d, J=8.08 Hz, 1H), 8.48 (d, J=1.26 Hz, 1H). HRMS (ESI-FTMS): calcd for C₂₅H₂₈N₂O₅S₂+H⁺, 501.15124. found: 501.15111.

### Example 22J

### (R)-3-methyl-2-(7-(5-methyl-1,3,4-thiadiazol-2-yl)dibenzo[b,d]furan-2-sulfonamido)butanoic acid (Compound 292)

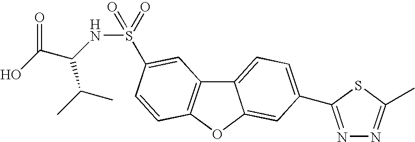

The title compound was prepared by the procedures described in Example 22, using 2-bromo-5-methyl-1,3,4-thiadiazole instead of 2-bromothiazole. The compound was obtained as an off-white solid. ¹H NMR (400 MHz, DMSO-d₆) δ ppm 0.81 (d, J=6.82 Hz, 3H), 0.84 (d, J=6.82 Hz, 3H), 1.84-2.04 (m, 1H), 2.82 (s, 3H), 3.53-3.67 (m, 1H), 7.90-8.14 (m, 4H), 8.36 (d, J=1.52 Hz, 1H), 8.48 (d, J=8.08 Hz, 1H), 8.69 (d, J=2.02 Hz, 1H). HRMS (ESI-FTMS): calcd for C₂₀H₁₉N₃O₅S₂+H⁺: 446.08389. found: 446.08487.

### Example 22K

### (R)-2-(7-(benzo[d]thiazol-2-yl)dibenzo[b,d]furan-2-sulfonamido)-3-methylbutanoic acid (Compound 293)

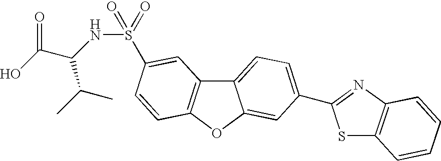

The title compound was prepared by the procedures described in Example 22, using 2-bromo-benzo[d]thiazole instead of 2-bromothiazole. The compound was obtained as an off-white solid. ¹H NMR (400 MHz, DMSO-d₆) δ ppm 0.79 (d, J=6.82 Hz, 3H), 0.86 (d, J=6.82 Hz, 3H), 2.30-2.37 (m, 1H), 2.63-2.69 (m, 1H), 7.47-7.64 (m, 3H), 7.92-8.02 (m, 2H), 8.12 (d, J=8.08 Hz, 1H), 8.21 (dd, 2H), 8.47-8.54 (m, 2H), 8.70 (d, 1H). HRMS (ESI-FTMS): calcd for C₂₄H₂₀N₂O₅S₂+H⁺: 481.08864. found: 481.08877.

### Example 23

### (S)-2-(8-(2-isobutylthiazol-5-yl)dibenzo[b,d]furan-3-sulfonamido)-3-methylbutanoic acid (Compound 248)

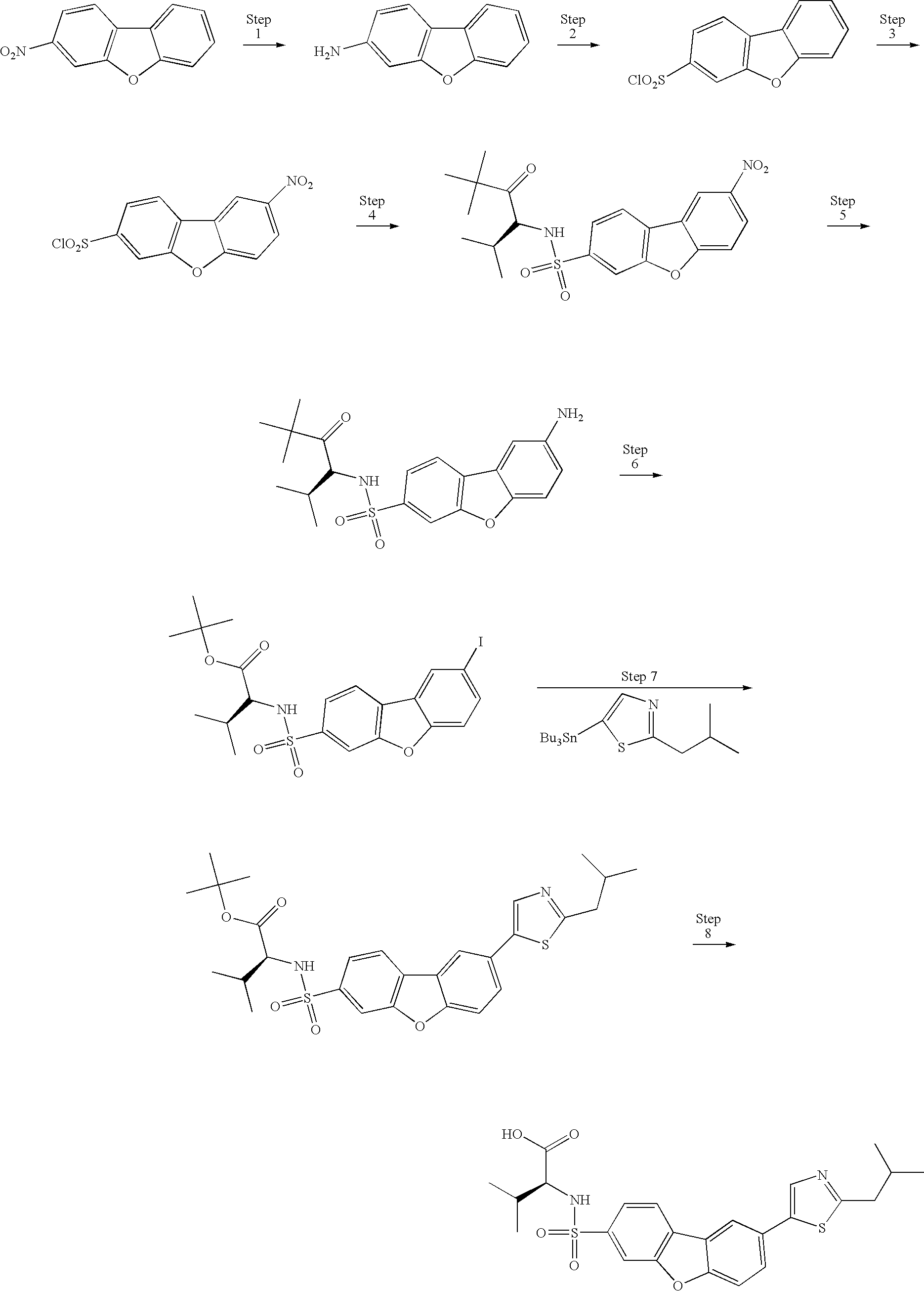

### Step 1: Preparation of dibenzo[b,d]furan-3-amine

3-Nitrodibenzo[b,d]furan (an intermediate in the preparation of Example 4) (2.13 g, 10 mmole) was mixed with 20 mL of MeOH and 0.5 g of 10% Pd/C (wt/wt), and the reaction was shaken with a Parr shaker at room temperature under an atmosphere of hydrogen (50 psi) overnight. The reaction mixture was filtered through Celite® and the filtrate was concentrated to give 1.80 g of pure dibenzo[b,d]furan-3-amine as an off-white solid in a 98% yield.

### Step 2: Preparation of dibenzo[b,d]furan-3-sulfonyl chloride

A mixture of dibenzo[b,d]furan-3-amine (6 g, 32.4 mmol), glacial acetic acid (AcOH, 60 mL) and concentrated hydrochloric acid (HCl, 60 mL) was added slowly to sodium nitrite (NaNO₂) (2.68 g, 38.8 mmol) in 20 mL of H₂O at −20° C. to give a yellow suspension. The suspension was stirred at −20° C. for 30 minutes, then was treated with a mixture of sulfur dioxide (30 mL) in 40 mL of 50% AcOH and dihydrate of copper (I) chloride (CuCl₂.2H₂O, 11.5 g, 676.2 mmol) at −23° C. The mixture was slowly warmed to room temperature and stirred for 21 hours. Once the disappearance of the starting material was confirmed by thin layer chromatography (TLC), the reaction mixture was quenched with water, was extracted with ethyl acetate (EtOAc, 3×50 mL), and the combined organic layers were washed with a saturated solution of sodium bicarbonate and brine. The organic layers were dried over sodium sulfate and the solvent was removed under reduced pressure to obtain 4.44 g of the desired dibenzo[b,d]furan-3-sulfonyl chloride as a white solid in a 51% yield.

### Step 3: Preparation of 8-nitrodibenzo[b,d]furan-3-sulfonyl chloride

A solution of dibenzo[b,d]furan-3-sulfonyl chloride (10.64 g, 40 mmol) in CH₂Cl₂ (60 mL) was treated with TFA (100 mL) and nitric acid (HNO₃, 10.6 g, 168 mmol), which were added dropwise. The mixture was stirred at room temperature for 6 hours and monitored by ¹H NMR, and the desired product precipitated out of the reaction mixture. While the solvent CH₂Cl₂ was being removed under reduced pressure, more precipitation occurred in the remaining TFA. More TFA (60 mL) was added to the reaction mixture for digestion before filtration. The filter cake was washed with cold water to provide 10.11 g of 8-nitrodibenzo[b,d]furan-3-sulfonyl chloride as a yellow solid in a 78% yield.

### Step 4: Preparation of (S)-tert-butyl 3-methyl-2-(8-nitrodibenzo[b,d]furan-3-sulfonamido)butanoate

L-Valine t-butyl ester (HCl salt, 14.98 g, 71.4 mmol) and di-isopropylethylamine (20 g, 24.9 mL) were mixed in CH₂Cl₂ (250 mL), and 8-nitrodibenzo[b,d]furan-3-sulfonyl chloride from Step 3 (22.26 g, 71.4 mmol) was added slowly portion-wise at 0° C. Upon completion of the addition, the ice bath was removed and the reaction was allowed to warm up to room temperature for 2 hours while being monitored by TLC. Water (200 mL) was added to the reaction flask, and CH₂Cl₂ was removed under reduced pressure with continuous stirring. The desired product precipitated out as a white solid in the aqueous media after complete removal of CH₂Cl₂. The suspension was filtered, and the filter cake was washed with water and dried to give 30.4 g of (S)-tert-butyl 3-methyl-2-(8-nitrodibenzo[b,d]furan-3-sulfonamido)butanoate in a 94% yield.

### Step 5: Preparation of (S)-tert-butyl 2-(8-aminodibenzo[b,d]furan-3-sulfonamido)-3-methylbutanoate

(S)-tert-Butyl 3-methyl-2-(8-nitrodibenzo[b,d]furan-3-sulfonamido) butanoate (6.12 g) in MeOH (150 mL) and 0.6 g of 10% Pd/C (50% water) were reacted in a Parr shaker apparatus under an atmosphere of hydrogen (50 psi) for 6 hours. The suspension was filtered through Celite® and the filtrate concentrated under reduced pressure to afford 5.70 g of (S)-tert-butyl 2-(8-aminodibenzo[b,d]furan-3-sulfonamido)-3-methylbutanoate as a white solid in a 98% yield.

### Step 6: Preparation of (S)-tert-butyl 2-(8-iododibenzo[b,d]furan-3-sulfonamido)-3-methylbutanoate

(S)-tert-Butyl 2-(8-aminodibenzo[b,d]furan-3-sulfonamido)-3-methylbutanoate (3.83 g, 9.2 mmol) was mixed with hydrochloric acid (3.5 ml), water (12 ml) and acetic acid (50 ml), and the solution was cooled to 0° C. An aqueous solution of sodium nitrite (2 M, 6.85 mL) was slowly added and the reaction mixture was stirred for 20 min, followed by very slow addition of sodium iodide solution (6.8 g, 45 mmol, in 20 ml of water). The reaction mixture was stirred for another 20 min and then was allowed to slowly warm up to room temperature. More water was added to the reaction mixture and the precipitate was filtered to produce (S)-tert-butyl 2-(8-iododibenzo[b,d]furan-3-sulfonamido)-3-methylbutanoate as a brown solid in 50% yield.

### Step 7: Preparation of (S)-tert-butyl 2-(8-(2-isobutylthiazol-5-yl)dibenzo[b,d]furan-3-sulfonamido)-3-methylbutanoate

A mixture of (S)-tert-butyl 2-(8-iododibenzo[b,d]furan-3-sulfonamido)-3-methylbutanoate (207 mg, 0.39 mmol), 2-isobutyl-5-(tributylstannyl)thiazole (336 mg, 0.78 mmol), Pd(PPh₃)₄ (60 mg), K₂CO₃ (215 mg, 1.56 mmol), and 2 mL of DME was stirred at 120° C. for 6 hours. The reaction mixture was purified by column chromatography. 110 mg of (S)-tert-butyl 2-(8-(2-isobutylthiazol-5-yl)dibenzo[b,d]furan-3-sulfonamido)-3-methylbutanoate was obtained as white solid (52%).

### Step 8: Preparation of (S)-tert-butyl 2-(8-(2-isobutylthiazol-5-yl)dibenzo[b,d]furan-3-sulfonamido)-3-methylbutanoate

(S)-tert-butyl 2-(8-(2-isobutylthiazol-5-yl)dibenzo[b,d]furan-3-sulfonamido)-3-methyl butanoate (100 mg) was dissolved in 30% TFA in DCM (2 ml), and the solution was stirred overnight. The solvents were removed under reduced pressure and the residue was triturated in CH₃CN/water and then freeze-dried to give 90 mg of (S)-tert-butyl 2-(8-(2-isobutylthiazol-5-yl)dibenzo[b,d]furan-3-sulfonamido)-3-methylbutanoate as an off-white solid (100% yield). ¹H NMR (400 MHz, DMSO-d₆) δ ppm 0.83 (dd, J=12.25, 6.69 Hz, 6H), 0.99 (d, J=6.57 Hz, 6H), 1.95 (d, J=6.57 Hz, 1H), 2.04-2.16 (m, 1H), 2.90 (d, J=7.33 Hz, 2H), 3.61 (dd, J=9.47, 5.94 Hz, 1H), 7.74 (dd, J=8.08, 1.52 Hz, 1H), 7.83 (dd, J=8.34, 1.52 Hz, 1H), 8.07 (d, J=1.52 Hz, 1H), 8.11 (d, J=1.26 Hz, 1H), 8.15 (d, J=9.35 Hz, 1H), 8.25-8.31 (m, 2H), 8.33 (d, J=8.34 Hz, 1H). HRMS (ESI-FTMS): calcd for C₂₄H₂₆N₂O₅S₂+H⁺, 487.13559. found: 487.13618.

The following compounds were prepared by the procedures as described in Example 23 for the preparation of (S)-tert-butyl 2-(8-(2-isobutylthiazol-5-yl)dibenzo[b,d]furan-3-sulfonamido)-3-methylbutanoate.

### Example 23A

### (S)-2-(7-(2-isobutylthiazol-5-yl)dibenzo[b,d]furan-3-sulfonamido)-3-methylbutanoic acid (Compound 249)

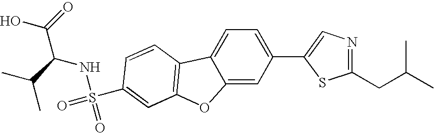

The title compound was prepared by the procedures described in Example 23, but started from (S)-methyl 2-(7-iodo-dibenzo[b,d]furan-3-sulfonamido)-3-methylbutanoate (an intermediate in preparation of Example 8). The compound was obtained as a white solid in 100% yield. ¹H NMR (400 MHz, DMSO-d₆) δ ppm 0.83 (dd, J=12.25, 6.69 Hz, 6H), 0.99 (d, J=6.57 Hz, 6H), 1.95 (d, J=6.57 Hz, 1H), 2.04-2.16 (m, 1H), 2.90 (d, J=7.33 Hz, 2H), 3.61 (dd, J=9.47, 5.94 Hz, 1H), 7.74 (dd, J=8.08, 1.52 Hz, 1H), 7.83 (dd, J=8.34, 1.52 Hz, 1H), 8.07 (d, J=1.52 Hz, 1H), 8.11 (d, J=1.26 Hz, 1H), 8.15 (d, J=9.35 Hz, 1H), 8.25-8.31 (m, 2H), 8.33 (d, J=8.34 Hz, 1H). HRMS (ESI-FTMS): calcd for C₂₄H₂₆N₂O₅S₂+H⁺, 487.13559. found: 487.13633.

### Example 24

### (S)-2-(7-(1H-tetrazol-5-yl)dibenzo[b,d]furan-3-sulfonamido)-3-methyl utanoic acid (Compound 250)

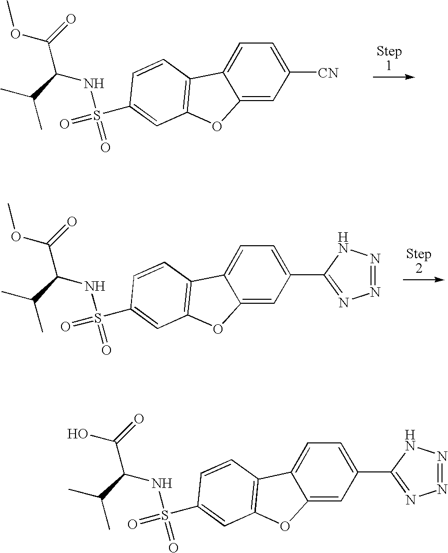

### Step 1: Preparation of (S)-methyl-2-(7-(1H-tetrazol-5-yl)dibenzo[b,d]furan-3-sulfonamido)-3-methylbutanoate

(S)-Methyl-2-(7-(1H-tetrazol-5-yl)dibenzo[b,d]furan-3-sulfonamido)-3-methyl butanoate was prepared using (S)-methyl 2-(7-cyanodibenzo[b,d]furan-3-sulfonamido)-3-methylbutanoate as the starting material and following literature procedure described for similar compounds (see e.g., Synthesis, 1999: 1004).

### Step 2: Preparation of (S)-2-(7-(1H-tetrazol-5-yl)dibenzo[b,d]furan-3-sulfonamido)-3-methylbutanoic acid

A solution of (S)-methyl 2-(7-(1H-tetrazol-5-yl)dibenzo[b,d]furan-3-sulfonamido)-3-methylbutanoate (100 mg) in THF/MeOH/water (2 mL) was treated with lithium hydroxide (5 equivalents), and the reaction was stirred overnight. After diluting with water, the pH of the solution was adjusted to between 4-5 and the precipitate obtained was then filtered to yield (S)methyl-2-(7-(1H-tetrazol-5-yl)dibenzo[b,d]furan-3-sulfonamido)-3-methylbutanoate as a white solid (95% yield). ¹H NMR (400 MHz, MeOD) δ ppm 1.14 (d, J=6.82 Hz, 3H), 1.20 (d, J=6.82 Hz, 3H), 2.28 (d, J=5.81 Hz, 1H), 3.97 (d, J=5.81 Hz, 1H), 8.14 (dd, J=8.08, 1.52 Hz, 1H), 8.32-8.40 (m, 2H), 8.49 (d, J=8.84 Hz, 1H), 8.53-8.58 (m, 2H). HRMS (ESI-FTMS): calcd for C₁₈H₁₇N₅O₅S+H⁺, 416.10232. found: 416.10226.

### Example 25

### 2-(8-(thiazol-2-yl)dibenzo[b,d]furan-3-sulfonamido)acetic acid (Compound 251)

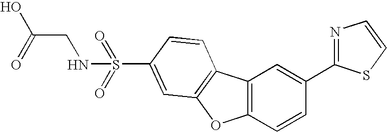

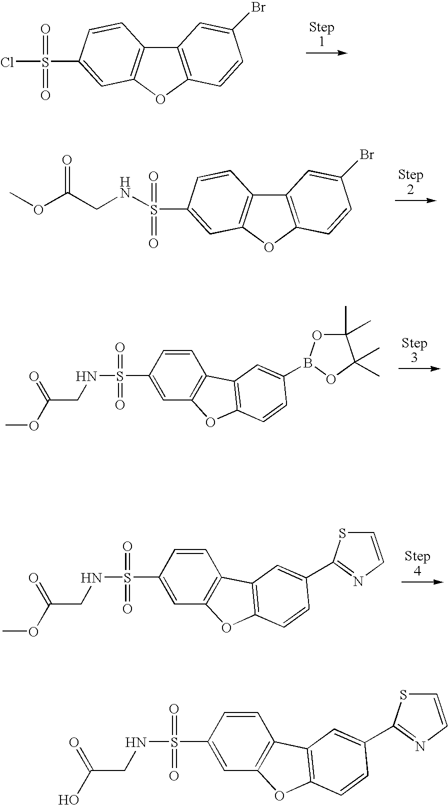

### Step 1: Preparation of methyl 2-(8-bromodibenzo[b,d]furan-3-sulfonamido)acetate

A solution of 8-bromodibenzo[b,d]furan-3-sulfonyl chloride (0.34 g, 1.0 mmol) (the intermediate of example 1) and methyl glycinate hydrochloride (1.1 eq.) in methylene chloride (DCM) (5 mL) was treated with N,N-diisopropylethylamine (0.38 mL, 2.2 eq.), and the mixture was stirred at room temperature for 2 hours. The crude reaction mixture was purified by silica gel column chromatography to produce methyl 2-(8-bromodibenzo[b,d]furan-3-sulfonamido)acetate (0.35 g) as a white solid.

### Step 2: methyl 2-(8-(4,4,5,5-tetramethyl-1,3,2-dioxaborolan-2-yl)dibenzo[b,d]furan-3-sulfonamido)acetate

Methyl 2-(8-bromodibenzo[b,d]furan-3-sulfonamido)acetate (300 mg), KOAc (4.0 eq.), 4,4,4′,4′,5,5,5′,5′-octamethyl-2,2′-bi(1,3,2-dioxaborolane) (1.1 equiv.), and Pd(dppf₂)Cl₂ (20 mg) were mixed in 3 mL of DMSO, and the mixture was deoxygenated with nitrogen, then was stirred at 120° C. for 4 hours. Brine was added to the reaction and the resulting mixture was extracted with ethyl acetate (EtOAc), the organic layers were concentrated under reduced pressure, and the crude residue was purified by flash column chromatography to provide methyl 2-(8-(4,4,5,5-tetramethyl-1,3,2-dioxaborolan-2-yl)dibenzo[b,d]furan-3-sulfonamido)acetate (197 mg) as a white solid.

### Step 3: methyl 2-(8-(thiazol-2-yl)dibenzo[b,d]furan-3-sulfonamido)acetate

Methyl-2-(8-(4,4,5,5-tetramethyl-1,3,2-dioxaborolan-2-yl)dibenzo[b,d]furan-3-sulfonamido)acetate (100 mg), 2-bromothiazole (1.2 equiv.), and Pd(dppf₂)Cl₂ (20 mg) were mixed in 2 mL of DMF and 0.3 mL of 2 M aqueous solution of potassium phosphate. The mixture was deoxygenated with nitrogen and stirred at 80° C. for 4 hours, then water was added and the precipitate was filtered and purified using preparative HPLC to give methyl 2-(8-(thiazol-2-yl)dibenzo[b,d]furan-3-sulfonamido)acetate (67 mg) as a white solid.

### Step 4: Preparation of 2-(8-(thiazol-2-yl)dibenzo[b,d]furan-3-sulfonamido)acetic acid

A solution of methyl 2-(8-(thiazol-2-yl)dibenzo[b,d]furan-3-sulfonamido)acetate (67 mg) in THF (2 mL) and water (2 mL) was treated with lithium hydroxide (LiOH, 100 mg) and the resulting mixture was stirred at RT overnight. The organic solvent was removed and the residue was diluted with water (2 mL) and acidified with 1 N HCl to pH ˜4. The precipitate was filtered to provide 2-(8-(thiazol-2-yl)dibenzo[b,d]furan-3-sulfonamido)acetic acid (50 mg) as a white solid. ¹H NMR (400 MHz, DMSO-d₆) δ ppm 8.89 (d, J=1.52 Hz, 1H), 8.53 (d, J=8.34 Hz, 1H), 8.23 (dd, J=8.72, 1.64 Hz, 1H), 8.15 (s, 1H), 7.97 (d, J=3.28 Hz, 1H), 7.85-7.94 (m, 1H), 7.83 (d, J=3.03 Hz, 1H), 7.45-7.60 (m, 2H), 3.59 (s, 2H). MS calcd for C₁₇H₁₂N₂O₅S₂+H⁺: 389.21. found: 389.1.

### Example 26

### (S)-2-(8-(5-tert-butyl-1,2,4-oxadiazol-3-yl)dibenzo[b,d]furan-3-sulfonamido)-4-methylpentanoic acid (Compound 252)

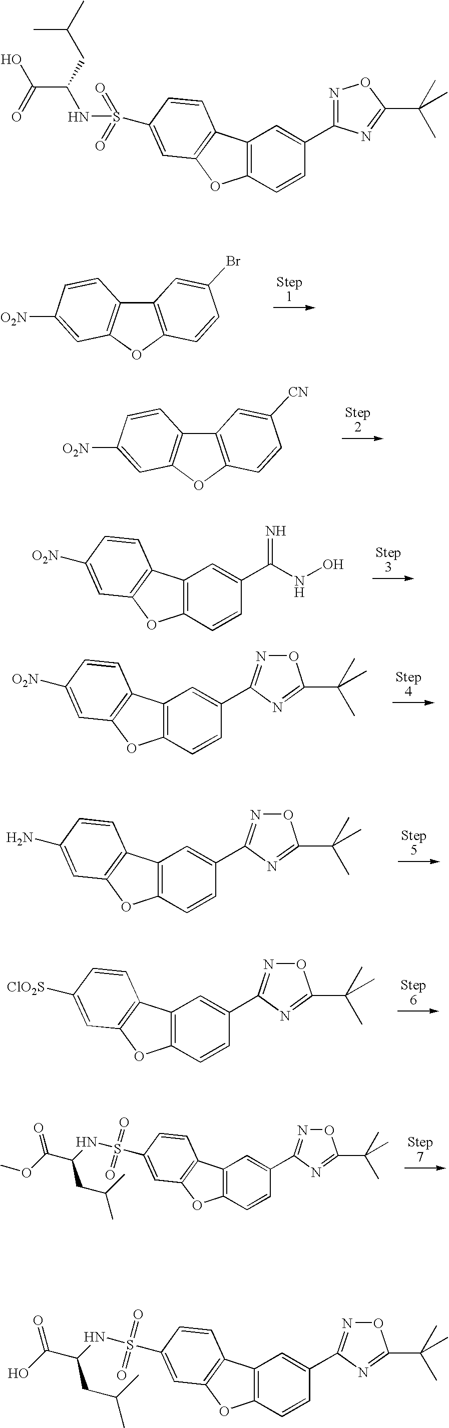

### Step 1: Preparation of 7-nitrodibenzo[b,d]furan-2-carbonitrile

2-Bromo-7-nitrodibenzo[b,d]furan (0.29 g, 1 mmol), zinc cyanide (2.0 equiv.), and Pd(PPh₃)₄ (20 mg) were dissolved in 2 mL of NMP in a 5 mL microwave vial. The solution was deoxygenated for 5 minutes and then irradiated with microwaves at 120° C. for 30 min. Upon completion, water was added to the reaction mixture and the precipitate was filtered to give the product, 7-nitrodibenzo[b,d]furan-2-carbonitrile (0.25 g) as a white solid.

### Step 2: Preparation of N-hydroxy-7-nitrodibenzo[b,d]furan-2-carboximidamide

A solution of 7-nitrodibenzo[b,d]furan-2-carbonitrile (0.25 g) in DMF (5 mL) was treated with hydroxylamine hydrochloride (2.0 equiv.) and triethylamine (3.0 equiv.), and the reaction mixture was stirred at room temperature overnight. The addition of water caused the formation of a precipitate, and the mixture was filtered to provide N-hydroxy-7-nitrodibenzo[b,d]furan-2-carboximidamide (0.27 g) as a white solid.

### Step 3: Preparation of 5-tert-butyl-3-(7-nitrodibenzo[b,d]furan-2-yl)-1,2,4-oxadiazole

A suspension of N-hydroxy-7-nitrodibenzo[b,d]furan-2-carboximidamide (270 mg) in CH₂Cl₂ (5 mL) was treated with 2,2,2-trimethylacetic anhydride (3 equiv.) and the reaction mixture was stirred at room temperature for 1 hour. The solvent was removed under reduced pressure, and the crude residue was dissolved in DMSO (2 mL) and heated at 90° C. overnight. After the reaction was cooled to room temperature, 3 mL of water was added and the resulting mixture was filtered to give 5-tert-butyl-3-(7-nitrodibenzo[b,d]furan-2-yl)-1,2,4-oxadiazole (0.34 g) as a white solid.

### Step 4: Preparation of 5-tert-butyl-3-(7-aminodibenzo[b,d]furan-2-yl)-1,2,4-oxadiazole

A solution of 5-tert-butyl-3-(7-nitrodibenzo[b,d]furan-2-yl)-1,2,4-oxadiazole (0.34 g) in MeOH (20 mL) was treated with 10% Pd/C (60 mg) and the reaction mixture was shaken using a Parr shaker apparatus at room temperature under an atmosphere of hydrogen (50 psi) overnight. The reaction mixture was filtered through a Celite pad and the filtrate was concentrated under reduced pressure to provide 5-tert-butyl-3-(7-aminodibenzo[b,d]furan-2-yl)-1,2,4-oxadiazole (0.26 g) as an off-white solid.

### Step 5: Preparation of 8-(5-tert-butyl-1,2,4-oxadiazol-3-yl)dibenzo[b,d]furan-3-sulfonyl chloride

A solution of 5-tert-butyl-3-(7-aminodibenzo[b,d]furan-2-yl)-1,2,4-oxadiazole (0.92 g, 3 mmol) in acetic acid (18 mL), water (15 mL) and hydrochloric acid (36%, 1.4 mL), was treated with aqueous NaNO₂ (1.5 mL, 5.5 M) at 0° C., and the resulting mixture was stirred at 0° C. for 1 h, then was poured into a mixture of copper (II) chloride (2 g), toluene (12 mL), and acetic acid (12 mL). After cooling with an ice-ethanol bath, sulfur dioxide was bubbled through the reaction mixture for one hour. The bath was then removed and mixture was stirred at RT for two hours. Upon addition of water a precipitate formed and was collected by filtration to provide a white solid, which was then suspended in 20 mL of acetic acid and water (1:2). The suspension was cooled to 0° C., chlorine was bubbled through for 90 min, and the mixture was then filtered to provide a white solid. The white solid was then treated with thionyl chloride (30 mL) and DMF (1 drop) and was stirred at 70° C. for 4 hours. Removal of the solvent under reduced pressure provided 8-(5-tert-butyl-1,2,4-oxadiazol-3-yl)dibenzo[b,d]furan-3-sulfonyl chloride (0.76 g) as a white solid.

### Step 6: Preparation of (S)-methyl 2-(8-(5-tert-butyl-1,2,4-oxadiazol-3-yl)dibenzo[b,d]furan-3-sulfonamido)-4-methylpentanoate

A solution of 8-bromodibenzo[b,d]furan-3-sulfonyl chloride (0.08 g) and L-leucine methyl ester hydrochloride (1.1 eq.) in CH₂Cl₂ (5 mL) was treated with aqueous Na₂CO₃ (2 mL, 2 M solution), and the mixture was stirred at room temperature for 2 hours. The organic solvent was removed under reduced pressure and the mixture was diluted with water and the resulting precipitate was collected via filtration to afford (S)-methyl 2-(8-(5-tert-butyl-1,2,4-oxadiazol-3-yl)dibenzo[b,d]furan-3-sulfonamido)-4-methylpentanoate (67 mg).

### Step 7: Preparation of (S)-2-(8-(5-tert-butyl-1,2,4-oxadiazol-3-yl)dibenzo[b,d]furan-3-sulfonamido)-4-methylpentanoic acid

A solution of (S)-methyl 2-(8-(5-tert-butyl-1,2,4-oxadiazol-3-yl)dibenzo[b,d]furan-3-sulfonamido)-4-methylpentanoate (67 mg) in THF (2 mL) and water (2 mL) was treated with LiOH (100 mg), and the resulting mixture was stirred at RT overnight. The organic solvent was removed and the residue was diluted with water (2 mL) and acidified with 1 N hydrochloric acid to pH ˜4. The resulting precipitate was collected via filtration to give (S)-2-(8-(5-tert-butyl-1,2,4-oxadiazol-3-yl)dibenzo[b,d]furan-3-sulfonamido)-4-methylpentanoic acid (40 mg) as a white solid after preparative HPLC purification. ¹H NMR (400 MHz, MeOD) δ ppm 9.05 (d, J=1.26 Hz, 1H), 8.48-8.55 (m, 2H), 8.34 (d, J=1.01 Hz, 1H), 8.13 (dd, J=8.08, 1.52 Hz, 1H), 7.92-8.05 (m, J=8.08 Hz, 1H), 4.10-4.19 (m, 1H), 1.74-1.77 (m, 9H), 1.22 (dd, 2H), 1.13 (d, J=6.6 Hz, 3H), 1.08 (d, J=6.56 Hz, 3H). MS calcd for C₂₄H₂₇N₃O₆S+H⁺) 486.16. found: 486.3.

### Example 26A

### (R)-2-(8-(5-tert-butyl-1,2,4-oxadiazol-3-yl)dibenzo[b,d]furan-3-sulfonamido)-4-methylpentanoic acid (Compound 253)

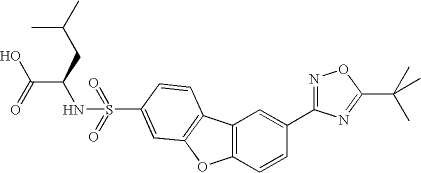

The title compound was prepared by the procedures described in Example 26, using D-leucine methyl ester hydrochloride instead of L-leucine methyl ester hydrochloride in step 6. The compound was obtained as an off-white solid. ¹H NMR (400 MHz, MeOD) δ ppm 9.04 (d, J=1.77 Hz, 1H), 8.48-8.55 (m, 2H), 8.34 (s, 1H), 8.10-8.18 (m, 1H), 8.02 (d, J=8.59 Hz, 1H), 4.11 (t, J=6.82 Hz, 1H), 1.92-2.10 (m, 2H), 1.72-1.82 (m, 9H), 1.14 (d, J=6.57 Hz, 3H), 1.09 (d, J=6.45 Hz, 3H). MS calcd for C₂₄H₂₇N₃O₆S+H⁺486.16. found: 486.3.

### Example 26B

### (S)-2-(8-(5-tert-butyl-1,2,4-oxadiazol-3-yl)dibenzo[b,d]furan-3-sulfonamido)-2-phenylacetic acid (Compound 254)

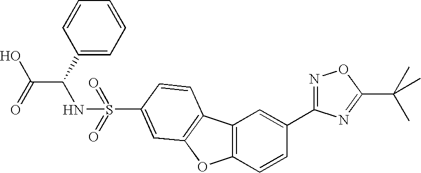

The title compound was prepared by the procedures described in Example 26, using methyl (S)-2-amino phenylacetate hydrochloride instead of L-leucine methyl ester hydrochloride in step 6. The compound was obtained as an off-white solid. ¹H NMR (400 MHz, MeOD) δ ppm 9.02 (d, J=1.26 Hz, 1H), 8.51 (dd, J=8.84, 1.77 Hz, 1H), 8.40 (d, J=8.34 Hz, 1H), 8.21 (d, J=1.01 Hz, 1H), 7.93-8.08 (m, 2H), 7.45-7.53 (m, 2H), 7.28-7.40 (m, 3H), 5.22 (s, 1H), 1.71-1.80 (m, 9H). MS calcd for C₂₆H₂₃N₃O₆S+H⁺: 506.13. found: 506.2.

### Example 26C

### (R)-2-(8-(5-tert-butyl-1,2,4-oxadiazol-3-yl)dibenzo[b,d]furan-3-sulfonamido)-2-phenylacetic acid (Compound 255)

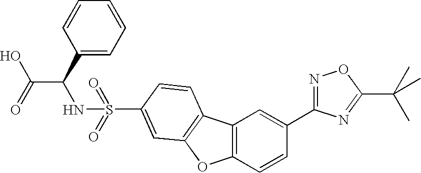

The title compound was prepared by the procedures described in Example 26, using methyl (R)-2-amino phenylacetate hydrochloride instead of L-leucine methyl ester hydrochloride in step 6. The compound was obtained as an off-white solid. ¹H NMR (400 MHz, MeOD) δ ppm 9.02 (d, J=1.77 Hz, 1H), 8.52 (dd, J=8.59, 1.77 Hz, 1H), 8.40 (d, J=8.34 Hz, 1H), 8.21 (d, J=1.01 Hz, 1H), 7.93-8.08 (m, 2H), 7.45-7.53 (m, 2H), 7.26-7.41 (m, 3H), 5.24 (s, 1H), 1.70-1.83 (m, 9H). MS calcd for C₂₆H₂₃N₃O₆S+H⁺: 506.13. found: 506.3.

### Example 26D

### (R)-2-(8-(5-tert-butyl-1,2,4-oxadiazol-3-yl)dibenzo[b,d]furan-3-sulfonamido)-3-(1H-indol-3-yl)propanoic acid (Compound 256)

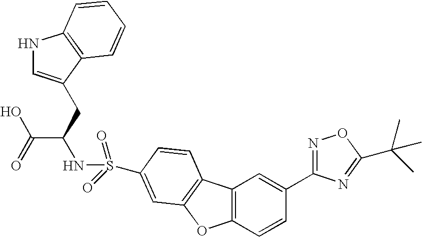

The title compound was prepared by the procedures described in Example 26, using D-tryptophan methyl ester hydrochloride instead of L-leucine methyl ester hydrochloride in step 6. The compound was obtained as an off-white solid. ¹H NMR (400 MHz, MeOD) δ ppm 8.96 (d, J=1.77 Hz, 1H), 8.53 (dd, J=8.59, 1.77 Hz, 1H), 8.03 (dd, J=31.33, 8.59 Hz, 2H), 7.87 (s, 1H), 7.71 (dd, J=8.21, 1.64 Hz, 1H), 7.49 (d, J=7.83 Hz, 1H), 7.20 (s, 1H), 6.91-7.05 (m, 2H), 6.83 (t, J=7.58 Hz, 1H), 4.35 (dd, J=9.60, 4.29 Hz, 1H), 3.43 (dd, J=14.53, 4.42 Hz, 1H), 3.15 (dd, J=14.53, 9.47 Hz, 1H), 1.76-1.81 (m, 9H). MS calcd for C₂₉H₂₆N₄O₆S+H⁺: 559.16. found: 559.2.

### Example 26E

### (S)-2-(8-(5-tert-butyl-1,2,4-oxadiazol-3-yl)dibenzo[b,d]furan-3-sulfonamido)-3,3-dimethylbutanoic acid (Compound 257)

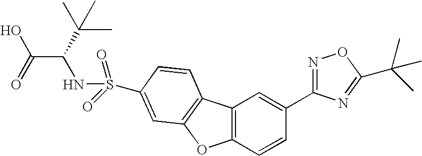

The title compound was prepared by the procedures described in Example 26, using L-t-leucine methyl ester hydrochloride instead of L-leucine methyl ester hydrochloride in step 6. The compound was obtained as an off-white solid. ¹H NMR (400 MHz, MeOD) δ ppm 9.05 (d, J=1.77 Hz, 1H), 8.47-8.55 (m, 2H), 8.35 (d, J=1.52 Hz, 1H), 8.13 (dd, J=8.21, 1.39 Hz, 1H), 8.02 (d, J=8.59 Hz, 1H), 3.75 (s, 1H), 1.75 (s, 9H), 1.22 (s, 9H). MS calcd for C₂₄H₂₇N₃O₆S−H, 484.16. found: 484.6.

### Example 26F

### (R)-2-(8-(5-tert-butyl-1,2,4-oxadiazol-3-yl)dibenzo[b,d]furan-3-sulfonamido)-3-methylbutanoic acid (Compound 258)

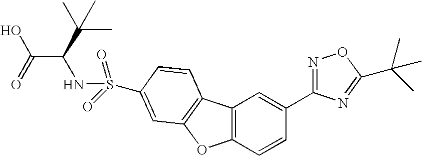

The title compound was prepared by the procedures described in Example 26, using D-valine methyl ester hydrochloride instead of L-leucine methyl ester hydrochloride in step 6. The compound was obtained as an off-white solid. ¹H NMR (400 MHz, DMSO-d₆) δ ppm 8.92 (d, J=1.77 Hz, 1H), 8.56 (d, J=8.08 Hz, 1H), 8.27 (dd, J=8.59, 1.77 Hz, 1H), 8.12 (d, J=1.01 Hz, 1H), 7.98 (d, J=8.59 Hz, 1H), 7.86 (dd, J=8.34, 1.52 Hz, 1H), 3.56-3.67 (m, 1H), 1.50 (s, 9H), 0.83 (dd, 6H). MS calcd for C₂₃H₂₆N₃O₆S+H⁺) 472.75. found: 472.3.

### Example 27

### (S)-2-(8-(5-cyclopropyl-1,2,4-oxadiazol-3-yl)dibenzo[b,d]furan-3-sulfonamido)-3-methylbutanoic acid (Compound 259)

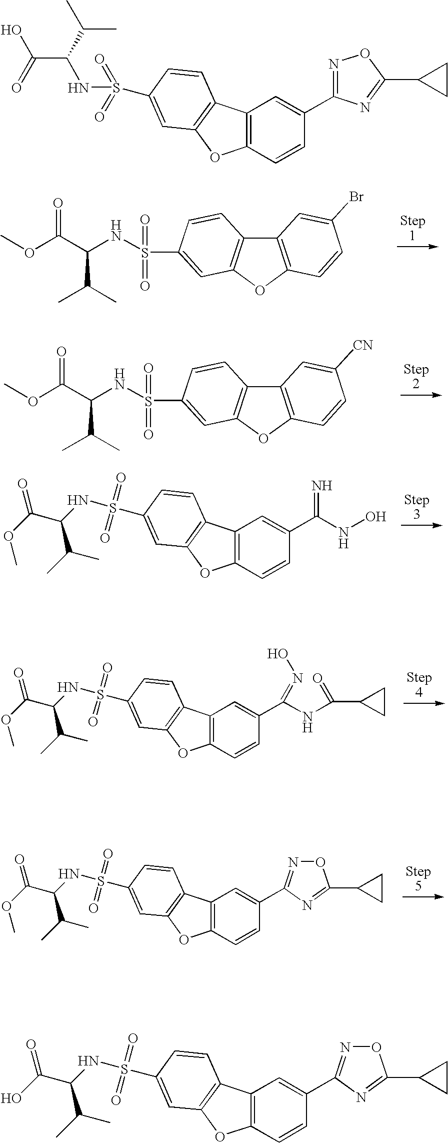

### Step 1: Preparation of (S)-methyl 2-(8-cyanodibenzo[b,d]furan-3-sulfonamido)-3-methylbutanoate

(S)-Methyl 2-(8-bromodibenzo[b,d]furan-3-sulfonamido)-3-methylbutanoate (the intermediate of example 10) (1.0 g, 2.27 mmol), zinc cyanide (293 mg, 2.5 mmol), and Pd(PPh₃)₄ (79 mg, 0.07 mmol) were dissolved in 20 mL of NMP in a 20-mL microwave vial. The solution was deoxygenated for 5 minutes and was irradiated with microwaves at 100° C. until no starting material was left according to LC-MS. Water was added to the reaction mixture and the precipitate was filtered to give the crude product, which was recrystallized from methylene chloride/hexane, then collected by filtration to provide (S)-methyl 2-(8-cyanodibenzo[b,d]furan-3-sulfonamido)-3-methylbutanoate as a white solid.

### Step 2: Preparation of (S)-methyl 2-(8-(N-hydroxycarbamimidoyl)dibenzo[b,d]furan-3-sulfonamido)-3-methylbutanoate

A solution of (S)-methyl 2-(8-cyanodibenzo[b,d]furan-3-sulfonamido)-3-methylbutanoate (500 mg, 1.29 mmol) in DMF (20 mL) was treated with hydroxylamine hydrochloride (448 mg, 6.45 mmol) and triethylamine (2.7 mL, 19.4 mmol), and the reaction was stirred at room temperature overnight. After diluting with water, the resulting precipitate was collected via filtration to provide (S)-methyl 2-(8-(N-hydroxycarbamimidoyl) dibenzo[b,d]furan-3-sulfonamido)-3-methylbutanoate (460 mg, 85% yield) as a white solid.

### Step 3: Preparation of (S)-methyl 2-(8-(N-(cyclopropanecarbonyl)-N % hydroxycarbamimidoyl)dibenzo[b,d]furan-3-sulfonamido)-3-methylbutanoate

A suspension of (S)-methyl 2-(8-(N-hydroxycarbamimidoyl)dibenzo[b,d]furan-3-sulfonamido)-3-methylbutanoate (100 mg, 0.24 mmol) CH₂Cl₂ (3 mL) was cooled to 0° C. and treated with cyclopropylcarbanyl chloride (0.1 mL), followed by aqueous saturated sodium bicarbonate solution (3 mL). The reaction mixture was stirred at rt for 2 hours, whereupon additional cyclopropylcarbanyl chloride (0.06 mL) was added. After 1 hour, the organic solvent was removed under reduced pressure and water was added. The resulting precipitate was collected via filtration to provide (S)-methyl 2-(8-(N-(cyclopropanecarbonyl)-N′-hydroxycarbamimidoyl)dibenzo[b,d]furan-3-sulfonamido)-3-methylbutanoate (120 mg, 100% yield).

### Step 4: Preparation of (S)-methyl 2-(8-(5-cyclopropyl-1,2,4-oxadiazol-3-yl)dibenzo [b,d]furan-3-sulfonamido)-3-methylbutanoate

A solution of (S)-methyl 2-(8-(N-(cyclopropanecarbonyl)-N′-hydroxy arbamimidoyl)dibenzo[b,d]furan-3-sulfonamido)-3-methylbutanoate (120 mg) in DMSO (2 mL) was heated at 90° C. overnight. After cooling to RT, water was added, and the resulting precipitate was collected via filtration to provide (S)-methyl 2-(8-(5-cyclopropyl-1,2,4-oxadiazol-3-yl)dibenzo[b,d]furan-3-sulfonamido)-3-methylbutanoate (105 mg) as a white solid.

### Step 5: Preparation of (S)-2-(8-(5-cyclopropyl-1,2,4-oxadiazol-3-yl)dibenzo[b,d]furan-3-sulfonamido)-3-methylbutanic acid

A suspension of (S)-methyl 2-(8-(5-cyclopropyl-1,2,4-oxadiazol-3-yl) dibenzo[b,d]furan-3-sulfonamido)-3-methylbutanoate (105 mg) in 1:1 THF:H₂O (2 mL) was treated with LiOH (5 equiv.) and the resulting mixture was stirred at RT overnight. The organic solvent was removed under reduced pressure and the residue was dissolved in water (2 mL) and acidified with 1 N hydrochloric acid to pH ˜4. The resulting precipitate was collected via filtration to provide (S)-2-(8-(5-cyclopropyl-1,2,4-oxadiazol-3-yl)dibenzo[b,d]furan-3-sulfonamido)-3-methylbutanic acid (92 mg) as a white solid. ¹H NMR (400 MHz, DMSO-d₆) δ ppm 8.89 (d, J=1.77 Hz, 1H), 8.53 (d, J=8.08 Hz, 1H), 8.22 (dd, J=8.59, 1.77 Hz, 1H), 8.12 (d, J=1.01 Hz, 1H), 7.96 (d, J=8.84 Hz, 1H), 7.86 (dd, J=8.21, 1.64 Hz, 1H), 3.62 (t, 1H), 1.97 (d, J=6.06 Hz, 1H), 1.29-1.37 (m, 2H), 1.21-1.28 (m, 2H), 0.83 (dd, J=12.25, 6.69 Hz, 6H). MS calcd for C₂₂H₂₁N₃O₆S+H⁺: 456.12. found: 456.2.

### Example 27A

### (S)-3-methyl-2-(8-(5-(tetrahydro-2H-pyran-4-yl)-1,2,4-oxadiazol-3-yl)dibenzo[b,d]furan-3-sulfonamido)butanoic acid (Compound 260)

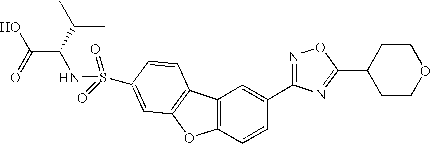

The title compound was prepared by the procedures described in Example 27, using tetrahydro-2H-pyran-4-carbonyl chloride instead of cyclopropylcarbonyl chloride in step 3. The compound was obtained as an off-white solid. ¹H NMR (400 MHz, DMSO-d₆) δ ppm 9.07 (d, J=1.77 Hz, 1H), 8.68 (d, J=8.34 Hz, 1H), 8.40 (dd, J=8.72, 1.89 Hz, 1H), 8.25 (d, J=1.52 Hz, 1H), 8.11 (d, J=9.09 Hz, 1H), 7.99 (dd, J=8.34, 1.52 Hz, 1H), 3.67 (dd, 1H), 3.40-3.47 (m, 8H), 0.95 (dd, 6H). MS calcd for C₂₄H₂₅N₃O₇S+H⁺: 500.14. found: 500.

### Example 27B

### (S)-3-methyl-2-(8-(5-neopentyl-1,2,4-oxadiazol-3-yl)dibenzo[b,d]furan-3-sulfonamido)butanoic acid (Compound 261)

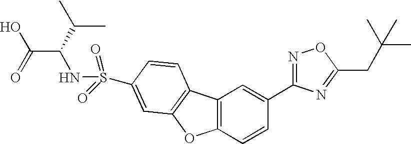

The title compound was prepared by the procedures described in Example 27, using t-butylacetyl chloride instead of cyclopropylcarbonyl chloride in step 3. The compound was obtained as an off-white solid. ¹H NMR (400 MHz, DMSO-d₆) δ ppm 9.05 (d, J=1.77 Hz, 1 H), 8.66 (d, J=8.08 Hz, 1H), 8.38 (dd, J=8.59, 1.77 Hz, 1H), 8.23 (d, J=1.26 Hz, 1H), 8.09 (d, J=8.59 Hz, 1H), 7.97 (dd, J=8.08, 1.52 Hz, 1H), 3.74 (dd, 1H), 3.08 (s, 2H), 2.02-2.14 (m, 1H), 1.19 (s, 9H), 0.94 (m, 6H). MS calcd for C₂₄H₂₇N₃O₆S+H⁺: 486.16. found: 486.3.

### Example 27C

### (S)-2-(8-(5-cyclobutyl-1,2,4-oxadiazol-3-yl)dibenzo[b,d]furan-3-sulfonamido)-3-methylbutanoic acid (Compound 262)

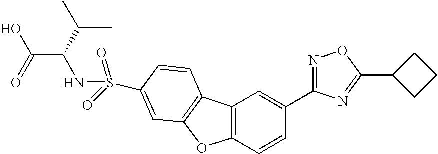

The title compound was prepared by the procedures described in Example 27, using cyclobutylcarbonyl chloride instead of cyclopropylcarbonyl chloride in step 3. The compound was obtained as an off-white solid. ¹H NMR (400 MHz, DMSO-d₆) δ ppm 9.00 (d, J=1.26 Hz, 1H), 8.61 (d, J=8.08 Hz, 1H), 8.32 (dd, J=8.59, 1.77 Hz, 1H), 8.18 (d, J=1.52 Hz, 1H), 8.03 (d, J=8.59 Hz, 1H), 7.91 (dd, J=8.08, 1.52 Hz, 1H), 4.03 (dd, 1H), 2.51 (dd, 1H), 2.12 (dd, 4H), 1.81 (dd, 1H), 0.87 (dd, 6H). MS calcd for C₂₃H₂₃N₃O₆S+H⁺: 470.13. found: 470.2.

### Example 27D

### (S)-2-(8-(5-cyclopentyl-1,2,4-oxadiazol-3-yl)dibenzo[b,d]furan-3-sulfonamido)-3-methylbutanoic acid (Compound 263)

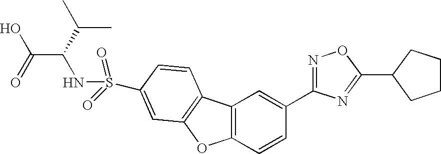

The title compound was prepared by the procedures described in Example 27, using cyclopentylcarbonyl chloride instead of cyclopropylcarbonyl chloride in step 3. The compound was obtained as an off-white solid. ¹H NMR (400 MHz, DMSO-d₆) δ ppm 8.97 (d, J=1.26 Hz, 1H), 8.58 (d, J=8.08 Hz, 1H), 8.30 (dd, J=8.59, 1.77 Hz, 1H), 8.16 (d, J=1.52 Hz, 1H), 8.01 (d, J=9.35 Hz, 1H), 7.86 (dd, J=8.21, 1.64 Hz, 1H), 3.60 (t, J=8.08 Hz, 1H), 1.68-2.31 (m, 11H), 0.87 (m, 6H). MS calcd for C₂₄H₂₅N₃O₆S+H⁺: 484.15. found: 484.3.

### Example 27E

### (S)-3-methyl-2-(8-(5-(thiophen-2-yl)-1,2,4-oxadiazol-3-yl)dibenzo[b,d] furan-3-sulfonamido)butanoic acid (Compound 264)

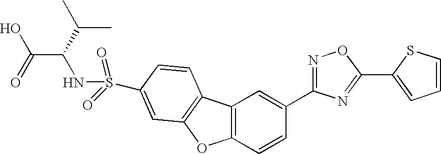

The title compound was prepared by the procedures described in Example 27, using 2-thiophenylcarbonyl chloride instead of cyclopropylcarbonyl chloride in step 3. The compound was obtained as an off-white solid. ¹H NMR (400 MHz, DMSO-d₆) δ ppm 9.00 (d, J=1.77 Hz, 1H), 8.59 (d, J=8.08 Hz, 1H), 8.33 (dd, J=8.84, 1.77 Hz, 1H), 8.10-8.19 (m, 3H), 8.01 (d, J=8.59 Hz, 1H), 7.87 (dd, J=8.08, 1.52 Hz, 1H), 7.41 (dd, J=5.05, 3.79 Hz, 1H), 3.62 (dd, 1H), 1.97 (m, 1H), 0.84 (m, 6H). MS calcd for C₂₃H₁₉N₃O₆S₂+H⁺: 498.07. found: 498.2.

### Example 27F

### (S)-3-methyl-2-(8-(5-phenyl-1,2,4-oxadiazol-3-yl)dibenzo[b,d]furan-3-sulfonamido)butanoic acid (Compound 265)

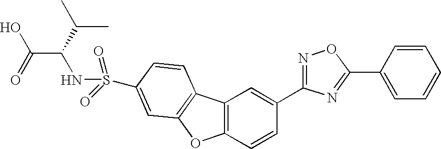

The title compound was prepared by the procedures described in Example 27, using benzoyl chloride instead of cyclopropylcarbonyl chloride in step 3. The compound was obtained as an off-white solid. ¹H NMR (400 MHz, DMSO-d₆) δ ppm 9.04 (d, J=1.77 Hz, 1H), 8.57 (d, J=8.08 Hz, 1H), 8.37 (dd, J=8.72, 1.89 Hz, 1H), 8.23-8.29 (m, 2H), 8.14 (d, J=1.01 Hz, 1H), 8.03 (d, J=8.59 Hz, 1H), 7.88 (dd, J=8.08, 1.52 Hz, 1H), 7.67-7.81 (m, 3H), 1.89-2.03 (m, 1H), 0.85 (m, 6H). MS calcd for C₂₅H₂₁N₃O₆S+H⁺: 492.12. found: 491.8.

### Example 27G

### (S)-2-(8-(5-benzyl-1,2,4-oxadiazol-3-yl)dibenzo[b,d]furan-3-sulfonamido)-3-methylbutanoic acid (Compound 266)

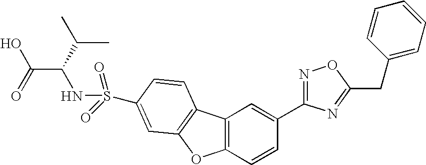

The title compound was prepared by the procedures described in Example 27, using phenylacetyl chloride instead of cyclopropylcarbonyl chloride in step 3. The compound was obtained as an off-white solid. ¹H NMR (400 MHz, DMSO-d₆) δ ppm 8.93 (d, J=1.26 Hz, 1H), 8.54 (d, J=8.34 Hz, 1H), 8.24 (dd, J=8.72, 1.90 Hz, 2H), 8.11 (s, 1H), 7.97 (d, J=8.84 Hz, 1H), 7.84 (dd, J=8.34, 1.26 Hz, 1H), 7.28-7.48 (m, 4H), 3.29-3.36 (m, 2H), 1.87-2.04 (m, 1H), 0.82 (dd, 6H). MS calcd for C₂₆H₂₃N₃O₆S+H⁺: 506.13. found: 506.2.

### Example 27H

### (S)-2-(8-(5-(methoxymethyl)-1,2,4-oxadiazol-3-yl)dibenzo[b,d]furan-3-sulfonamido)-3-methylbutanoic acid (Compound 267)

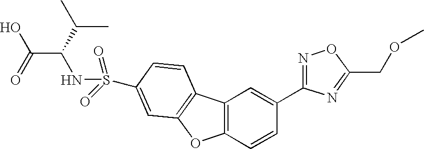

The title compound was prepared by the procedures described in Example 27, using 2-methoxyacetyl chloride instead of cyclopropylcarbonyl chloride in step 3. The compound was obtained as an off-white solid. ¹H NMR (400 MHz, DMSO-d₆) δ ppm 8.98 (s, 1H), 8.55 (d, J=8.59 Hz, 1H), 8.29 (d, J=10.36 Hz, 1H), 8.13 (s, 1H), 8.00 (d, J=8.59 Hz, 1H), 7.87 (s, 1H), 4.88 (s, 2H), 3.47 (s, 3H), 1.85-2.04 (m, 1H), 0.83 (m, 6H). MS calcd for C₂₁H₂₁N₃O₇S+H⁺: 460.11. found: 460.2.

### Example 27I

### (2S)-3-methyl-2-(8-(5-(tetrahydrofuran-3-yl)-1,2,4-oxadiazol-3-yl)dibenzo [b,d]furan-3-sulfonamido)butanoic acid (Compound 268)

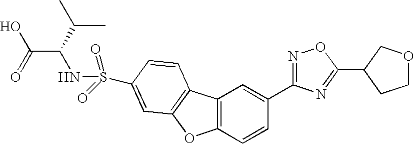

The title compound was prepared by the procedures described in Example 27, using tetrahydrofuran-3-carbonyl chloride instead of cyclopropylcarbonyl chloride in step 3. The compound was obtained as an off-white solid. ¹H NMR (400 MHz, DMSO-d₆) δ ppm 8.93 (d, J=1.26 Hz, 1H), 8.54 (d, J=8.34 Hz, 1H), 8.27 (dd, J=8.84, 1.77 Hz, 1H), 8.13 (d, J=1.26 Hz, 1H), 7.99 (d, J=8.59 Hz, 1H), 7.86 (dd, J=8.21, 1.39 Hz, 1H), 3.77-4.17 (m, 4H), 3.63 (d, J=5.81 Hz, 1H), 1.87-2.37 (m, 2H), 0.83 (m, 6H). MS calcd for C₂₃H₂₃N₃O₇S+H⁺: 486.13. found: 486.2.

### Example 27J

### (S)-2-(8-(5-(2,4-difluorophenyl)-1,2,4-oxadiazol-3-yl)dibenzo[b,d]furan-3-sulfonamido)-3-methylbutanoic acid (Compound 269)

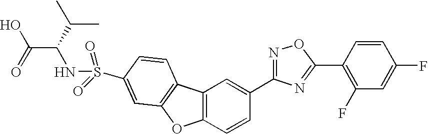

The title compound was prepared by the procedures described in Example 27, using 2,4-difluorobenzoyl chloride instead of cyclopropylcarbonyl chloride in step 3. The compound was obtained as an off-white solid. ¹H NMR (400 MHz, DMSO-d₆) δ ppm 9.03 (d, J=1.26 Hz, 1H), 8.57 (d, J=7.58 Hz, 1H), 8.32-8.43 (m, 2H), 8.14 (d, J=1.01 Hz, 1H), 8.03 (d, J=8.84 Hz, 1H), 7.87 (dd, J=8.21, 1.39 Hz, 1H), 7.70 (s, 1H), 7.46 (s, 1H), 1.87-2.04 (m, 1H), 0.75-0.91 (m, 6H). MS calcd for C₂₅H₁₉F₂N₃O₆S+H⁺: 528.1. found: 527.9.

### Example 27K

### (S)-2-(8-(5-(2,4-dichlorophenyl)-1,2,4-oxadiazol-3-yl)dibenzo[b,d]furan-3-sulfonamido)-3-methylbutanoic acid (Compound 270)

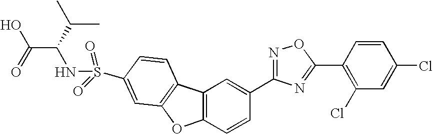

The title compound was prepared by the procedures described in Example 27, using 2,4-dichlorobenzoyl chloride instead of cyclopropylcarbonyl chloride in step 3. The compound was obtained as an off-white solid. ¹H NMR (400 MHz, DMSO-d₆) δ ppm 9.04 (d, J=1.77 Hz, 1H), 8.57 (d, J=8.08 Hz, 1H), 8.36 (dd, J=8.72, 1.89 Hz, 1H), 8.27 (d, J=8.34 Hz, 1H), 8.14 (d, J=1.01 Hz, 1H), 8.03 (dd, J=5.56, 3.28 Hz, 2H), 7.87 (dd, J=8.08, 1.52 Hz, 1H), 7.77 (dd, J=8.46, 2.15 Hz, 1H), 1.91-2.03 (m, 1H), 0.83 (dd, 6H). MS calcd for C₂₅H₁₉Cl₂N₃O₆S+H⁺: 560.04. found: 559.9.

### Example 27L

### (S)-3-methyl-2-(8-(5-(4-(trifluoromethyl)phenyl)-1,2,4-oxadiazol-3-yl)dibenzo[b,d]furan-3-sulfonamido)butanoic acid (Compound 271)

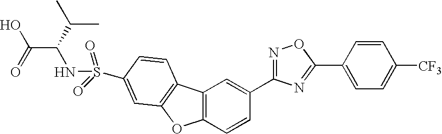

The title compound was prepared by the procedures described in Example 27, using 4-trifluoromethylbenzoyl chloride instead of cyclopropylcarbonyl chloride in step 3. The compound was obtained as an off-white solid. ¹H NMR (400 MHz, DMSO-d₆) δ ppm 9.03 (d, J=1.77 Hz, 1H), 8.54 (d, J=8.34 Hz, 1H), 8.47 (d, J=8.08 Hz, 2H), 8.38 (dd, J=8.72, 1.89 Hz, 1H), 8.13-8.17 (m, 1H), 8.09 (d, J=8.34 Hz, 2H), 8.03 (d, J=8.84 Hz, 1H), 7.89 (dd, J=8.08, 1.52 Hz, 1H), 1.92-2.04 (m, 1H), 0.83 (dd, 6H). MS calcd for C₂₆H₂₀F₃N₃O₆S+H⁺: 560.1. found: 559.5.

### Example 27M

### (S)-2-(8-(5-(4-fluorophenyl)-1,2,4-oxadiazol-3-yl)dibenzo[b,d]furan-3-sulfonamido)-3-methylbutanoic acid (Compound 272)

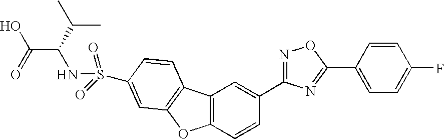

The title compound was prepared by the procedures described in Example 27, using 4-fluorobenzoyl chloride instead of cyclopropylcarbonyl chloride in step 3. The compound was obtained as an off-white solid. ¹H NMR (400 MHz, DMSO-d₆) δ ppm 9.00 (d, J=1.77 Hz, 1H), 8.53 (d, J=8.08 Hz, 1H), 8.28-8.39 (m, 3H), 8.14 (d, J=1.52 Hz, 1H), 8.02 (d, J=8.59 Hz, 1H), 7.88 (dd, J=8.21, 1.64 Hz, 1H), 7.50-7.61 (m, 2H), 1.90-2.05 (m, 1H), 0.82 (dd, 6H). MS calcd for C₂₅H₂₀N₃O₆S+H⁺: 510.11. found: 509.9.

### Example 27N

### (S)-7-(N-(1-carboxy-2-methylpropyl)sulfamoyl)dibenzo[b,d]furan-2-carboxylic acid (Compound 273)

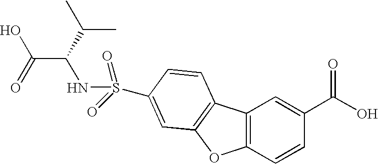

The title compound was obtained as a by-product of the preparation of (S)-2-(8-(5-(4-fluorophenyl)-1,2,4-oxadiazol-3-yl)dibenzo[b,d]furan-3-sulfonamido)-3-methylbutanoic acid (the proceeding compound). The compound was isolated as an off-white solid. ¹H NMR (400 MHz, DMSO-d₆) δ ppm 8.87 (d, J=1.26 Hz, 1H), 8.47 (d, J=7.83 Hz, 1H), 8.21 (dd, J=8.84, 1.77 Hz, 1H), 8.11 (d, J=1.01 Hz, 1H), 7.80-7.91 (m, 2H), 1.86-2.05 (m, 1H), 0.82 (dd, 6H). MS calcd for C₁₈H₁₇NO₇S−H, 390.07. found: 390.

### Example 28

### 2-(7-(5-tert-butyl-1,2,4-oxadiazol-3-yl)dibenzo[b,d]furan-2-sulfonamido) acetic acid (Compound 274)

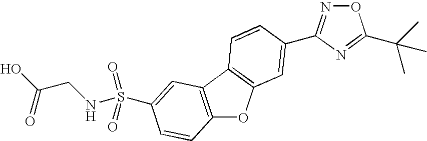

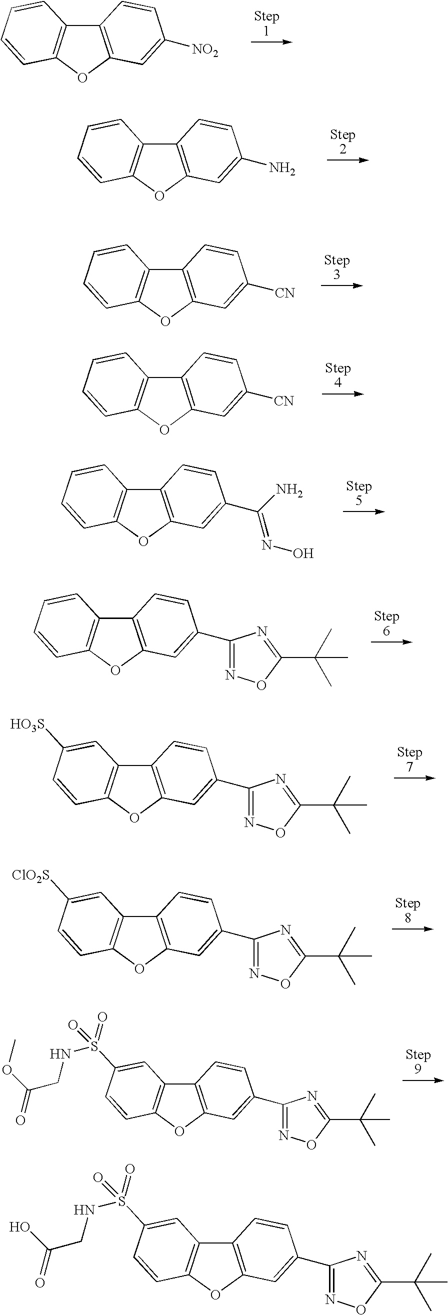

### Step 1: Preparation of dibenzo[b,d]furan-3-amine

3-Nitrodibenzofuran (7.5 g) (an intermediate of example 15) was suspended in 150 mL of MeOH and Pd/C (100 mg, 10% wt/wt) was added. The reaction was carried out in a Parr shaker at room temperature under an atmosphere of hydrogen (50 psi) overnight. The reaction mixture was filtered through a Celite pad and the filtrate was concentrated to produce dibenzo[b,d]furan-3-amine (7.0 g) as an off-white solid.

### Step 2: Preparation of 3-Iododibenzofuran

Dibenzo[b,d]furan-3-amine (4.0 g) was dissolved in hydrochloric acid (18%, 40 mL), and was treated with aqueous NaNO₂ (30 mL, 1 M, 1.5 equiv.) at 0° C. The resulting mixture was stirred at 0° C. for 0.5 hours, whereupon an aqueous sodium iodide (2M, 20 mL) was added. After stirring at RT for 4 hours, the mixture was treated with sodium sulfite and the precipitate was collected via filtration to provide 3-iododibenzofuran (5.6 g) as white solid.

### Step 3: Preparation of 3-cyanodibenzofuran

3-Iododibenzofuran (1.08 g), zinc cyanide (0.86 g, 2 equiv.), and Pd(PPh₃)₄ (48 mg) were dissolved in 15 mL of DMF in a round bottom flask. The solution was deoxygenated for 5 minutes and heated to 100° C. until no starting material was left according to TLC. Upon completion, water was added to the reaction mixture and the precipitate was filtered to give the crude product, which was re-precipitated from DCM/hexane to produce 3-cyanodibenzofuran (0.68 g) as a white solid.

### Step 4: Preparation of N′-hydroxydibenzo[b,d]furan-3-carboximidamide

A solution of 3-cyanodibenzofuran (2.65 g) in DMF (50 mL) was treated with hydroxylamine hydrochloride (2.5 equiv.) and triethylamine (2.5 equiv.), and the reaction was stirred at room temperature overnight. After the addition of water, the resulting precipitate was collected via filtration to provide N′-hydroxydibenzo[b,d]furan-3-carboximidamide (2.9 g) as a white solid.

### Step 5: Preparation of 5-tert-butyl-3-(dibenzo[b,d]furan-3-yl)-1,2,4-oxadiazole

N′-hydroxydibenzo[b,d]furan-3-carboximidamide (1.38 g) was mixed with 2,2,2-trimethylacetic acid (3.0 g) and 2,2,2-trimethylacetic anhydride (10 mL) was added. The reaction mixture was stirred at room temperature for 30 minutes and heated at 90° C. for 4 hours. After the solution was cooled to room temperature, 30 mL of water was added and the resulting mixture was filtered to give 5-tert-butyl-3-(dibenzo[b,d]furan-3-yl)-1,2,4-oxadiazole (2.1 g) as white solid.

### Step 6: Preparation of 7-(5-tert-butyl-1,2,4-oxadiazol-3-yl)dibenzo[b,d]furan-2-sulfonic acid

To a round-bottom flask containing 3-nitrodibenzo[b,d]furan (2 g) in 30 mL of chloroform was slowly added chlorosulfonic acid (2.0 equiv.) at 0° C. The resulting suspension was warmed to room temperature and stirred for 2 hours. The reaction mixture was cooled to 0° C. and filtered to produce 7-(5-tert-butyl-1,2,4-oxadiazol-3-yl)dibenzo[b,d]furan-2-sulfonic acid (2.67 g) as a white solid.

### Step 7: Preparation of 7-(5-tert-butyl-1,2,4-oxadiazol-3-yl)dibenzo[b,d]furan-2-sulfonyl chloride

7-(5-tert-Butyl-1,2,4-oxadiazol-3-yl)dibenzo[b,d]furan-2-sulfonic acid (2.67 g) was mixed with thionyl chloride (20 mL) and DMF (1 drop) was added slowly. The resulting mixture was stirred at 75° C. for 3 hours. The solvent was removed under reduced pressure and the crude residue was triturated with ice-water to produce 7-(5-tert-butyl-1,2,4-oxadiazol-3-yl)dibenzo[b,d]furan-2-sulfonyl chloride (2.7 g) as an off-white solid.

### Step 8: Preparation of methyl 2-(7-(5-tert-butyl-1,2,4-oxadiazol-3-yl)dibenzo[b,d]furan-2-sulfonamido)acetate

7-(5-tert-butyl-1,2,4-oxadiazol-3-yl)dibenzo[b,d]furan-2-sulfonyl chloride (0.10 g) and glycine methyl ester hydrochloride (1.1 eq.) were mixed in 5 mL of methylene chloride (DCM), to which a 2 M ageous solution of sodium carbonate (2 mL) was added. The mixture was stirred at room temperature for 2 hours and the organic solvent was removed under reduced pressure. The mixture was then diluted with water and the precipitate was collected via filtration to provide methyl 2-(7-(5-tert-butyl-1,2,4-oxadiazol-3-yl)dibenzo[b,d]furan-2-sulfonamido)acetate (125 mg).

### Step 9: Preparation of 2-(7-(5-tert-butyl-1,2,4-oxadiazol-3-yl)dibenzo[b,d]furan-2-sulfonamido)acetic acid

A solution of methyl 2-(7-(5-tert-butyl-1,2,4-oxadiazol-3-yl)dibenzo[b,d]furan-2-sulfonamido)acetate (125 mg) in THF (2 mL) and water (2 mL) was treated with LiOH (100 mg) and the resulting mixture was stirred at RT overnight. The organic solvent was removed under reduced pressure and the residue was dissolved in water (2 mL) and acidified with 1 N hydrochloric acid to pH ˜4. The resulting precipitate was filtered to give the crude product, which was purified with preparative HPLC to afford 2-(7-(5-tert-butyl-1,2,4-oxadiazol-3-yl)dibenzo[b,d]furan-2-sulfonamido)acetic acid (20 mg) as an off-white solid. ¹H NMR (400 MHz, DMSO-d₆) δ ppm 8.75 (d, J=2.02 Hz, 1H), 8.52 (d, J=8.08 Hz, 1H), 8.30 (s, 1H), 8.11 (dd, J=1.26 Hz, 1H), 7.99 (dd, 2H), 3.21-3.36 (m, 2H), 1.46-1.51 (s, 9H).

### Example 28A

### (R)-2-(7-(5-tert-butyl-1,2,4-oxadiazol-3-yl)dibenzo[b,d]furan-2-sulfonamido)-3-phenylpropanoic acid (Compound 275)

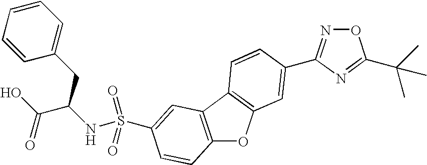

The title compound was prepared by the procedures described in Example 28, using D-phenylalanine methyl ester hydrochloride instead of glycine methyl ester hydrochloride in step 8. The compound was obtained as an off-white solid. ¹H NMR (400 MHz, MeOD) δ ppm 8.61 (d, J=1.52 Hz, 1H), 8.53 (s, 1H), 8.34-8.46 (m, 2H), 8.08 (dd, J=8.59, 2.02 Hz, 1H), 7.87 (d, J=8.59 Hz, 1H), 7.21-7.36 (m, 3H), 7.10-7.17 (m, 1H), 4.24-4.35 (m, 1H), 3.24-3.32 (m, 1H), 3.01-3.10 (m, 1H), 1.72-1.81 (s, 9H). MS calcd for C₂₇H₂₅N₃O₆S+H⁺: 520.15. found: 520.2.

### Example 28B

### (S)-2-(7-(5-tert-butyl-1,2,4-oxadiazol-3-yl)dibenzo[b,d]furan-2-sulfonamido)-3-methylbutanoic acid (Compound 276)

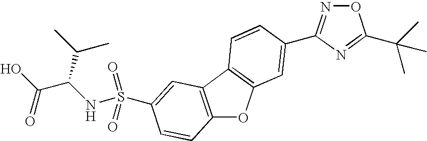

The title compound was prepared by the procedures described in Example 28, using L-valine methyl ester hydrochloride instead of glycine methyl ester hydrochloride in step 8. The compound was obtained as an off-white solid. ¹H NMR (400 MHz, MeOD) δ ppm 8.85 (d, J=2.02 Hz, 1H), 8.54 (s, 1H), 8.48 (t, J=8.59 Hz, 1H), 8.38 (dd, J=8.08, 1.26 Hz, 1H), 8.27 (dd, J=8.84, 2.02 Hz, 1H), 7.99 (d, J=8.84 Hz, 1H), 3.89 (d, 1H), 2.26-2.32 (m, 1H), 1.73-1.77 (m, 9H), 1.17 (dd, 6H). MS calcd for C₂₃H₂₅N₃O₆S+H⁺: 472.15. found: 472.3.

### Example 28C

### 2-(7-(5-tert-butyl-1,2,4-oxadiazol-3-yl)dibenzo[b,d]furan-2-sulfonamido)-2-methylpropanoic acid (Compound 277)

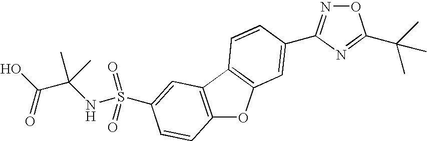

The title compound was prepared by the procedures described in Example 28, using 2-methyl-2-amino propanoic acid methyl ester hydrochloride instead of glycine methyl ester hydrochloride in step 8. The compound was obtained as an off-white solid. ¹H NMR (400 MHz, MeOD) δ ppm 8.88 (d, J=2.02 Hz, 1H), 8.54 (s, 1H), 8.50 (d, J=8.08 Hz, 1H), 8.38 (dd, J=8.21, 1.39 Hz, 1H), 8.31 (dd, J=8.72, 1.90 Hz, 1H), 8.00 (d, J=8.84 Hz, 1H), 1.72-1.79 (m, 9H), 1.61-1.66 (m, 6H). MS calcd for C₂₂H₂₃N₃O₆S+H⁺: 458.13. found: 458.2.

### Example 28D

### (R)-2-(7-(5-tert-butyl-1,2,4-oxadiazol-3-yl)dibenzo[b,d]furan-2-sulfonamido)-4-methylpentanoic acid (Compound 278)

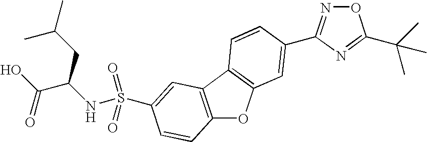

The title compound was prepared by the procedures described in Example 28, using D-leucine methyl ester hydrochloride instead of glycine methyl ester hydrochloride in step 8. The compound was obtained as an off-white solid. ¹H NMR (400 MHz, MeOD) δ ppm 8.84 (d, J=1.26 Hz, 1H), 8.54 (d, J=1.26 Hz, 1H), 8.49 (d, J=8.08 Hz, 1H), 8.38 (dd, J=8.08, 1.26 Hz, 1H), 8.27 (dd, J=8.72, 1.89 Hz, 1H), 8.00 (d, J=8.59 Hz, 1H), 4.13 (d, 1H), 3.67 (d, 1H), 2.57 (d, 1H), 2.19-2.31 (m, 1H), 1.74-1.77 (m, 9H), 1.12 (dd, 6H). MS calcd for C₂₄H₂₇N₃O₆S+H⁺: 486.16. found: 486.3.

### Example 28E

### (S)-2-(7-(5-tert-butyl-1,2,4-oxadiazol-3-yl)dibenzo[b,d]furan-2-sulfonamido)-4-methylpentanoic acid (Compound 279)

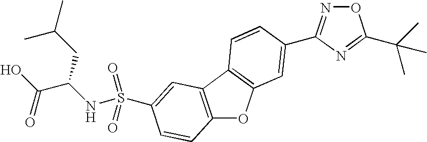

The title compound was prepared by the procedures described in Example 28, using L-leucine methyl ester hydrochloride instead of glycine methyl ester hydrochloride in step 8. The compound was obtained as an off-white solid. ¹H NMR (400 MHz, MeOD) δ ppm 8.84 (d, J=2.02 Hz, 1H), 8.54 (s, 1H), 8.47-8.51 (m, 1H), 8.38 (dd, J=8.21, 1.39 Hz, 1H), 8.27 (dd, J=8.84, 2.02 Hz, 1H), 8.00 (d, J=8.59 Hz, 1H), 4.12 (d, 1H), 3.66 (d, 1H), 2.57 (d, 1H), 2.20-2.32 (m, 1H), 1.94-2.07 (m, 1H), 1.74-1.77 (m, 9H), 1.10 (dd, 6H). MS calcd for C₂₄H₂₇N₃O₆S+H⁺: 486.16. found: 486.3.

### Example 28F

### (S)-2-(7-(5-tert-butyl-1,2,4-oxadiazol-3-yl)dibenzo[b,d]furan-2-sulfonamido)-2-(1H-indol-3-yl)acetic acid (Compound 280)

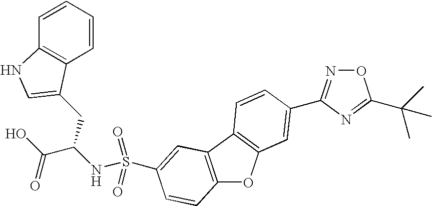

The title compound was prepared by the procedures described in Example 28, using L-tryptophan methyl ester hydrochloride instead of glycine methyl ester hydrochloride in step 8. The compound was obtained as an off-white solid. ¹H NMR (400 MHz, MeOD) δ ppm 8.51 (s, 1H), 8.26-8.42 (m, 2H), 7.86 (dd, J=8.72, 1.89 Hz, 1H), 7.60 (d, J=8.59 Hz, 1 H), 7.49 (d, J=7.33 Hz, 1H), 7.19 (s, 1H), 7.00-7.06 (m, 1H), 6.80-6.91 (m, 3H), 4.29-4.38 (m, 1H), 2.84-2.91 (m, 2H), 1.77 (s, 9H). MS calcd for C₂₉H₂₆N₄O₆S+H⁺: 559.16. found: 559.3.

### Example 28G

### (S)-2-(7-(5-tert-butyl-1,2,4-oxadiazol-3-yl)dibenzo[b,d]furan-2-sulfonamido)-2-phenylacetic acid (Compound 281)

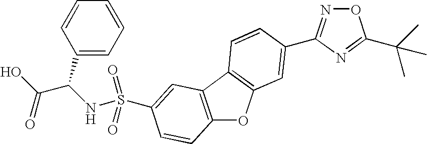

The title compound was prepared by the procedures described in Example 28, using L-phenylglycine methyl ester hydrochloride instead of glycine methyl ester hydrochloride in step 8. The compound was obtained as an off-white solid. ¹H NMR (400 MHz, MeOD) δ ppm 8.67 (d, J=1.77 Hz, 1H), 8.53 (s, 1H), 8.35-8.44 (m, 2H), 8.19 (dd, J=8.72, 1.89 Hz, 1H), 7.89 (d, J=8.84 Hz, 1H), 7.48 (d, J=7.58 Hz, 2H), 7.23-7.39 (m, 3H), 3.62-3.69 (m, 1H), 1.75 (s, 9H). MS calcd for C₂₆H₂₃N₃O₆S+H⁺: 506.13. found: 506.2.

### Example 28H

### (S)-2-(7-(5-tert-butyl-1,2,4-oxadiazol-3-yl)dibenzo[b,d]furan-2-sulfonamido)-3,3-dimethylbutanoic acid (Compound 282)

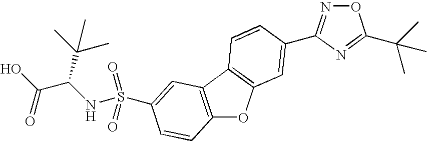

The title compound was prepared by the procedures described in Example 28, using L-tert-leucine methyl ester hydrochloride instead of glycine methyl ester hydrochloride in step 8. The compound was obtained as an off-white solid. ¹H NMR (400 MHz, MeOD) δ ppm 8.83 (d, J=1.26 Hz, 1H), 8.76 (s, 1H), 8.49-8.54 (m, 1H), 8.33-8.39 (m, 1H), 8.26 (dd, J=8.72, 1.89 Hz, 1H), 7.98 (d, J=9.35 Hz, 1H), 3.68 (d, 1H), 1.72-1.77 (m, 9H), 1.18-1.24 (m, 9H). MS calcd for C₂₄H₂₇N₃O₆S+H⁺: 486.16. found: 486.3.

### Example 29

### (S)-3-methyl-2-(8-(4-(4-(trifluoromethyl)phenyl)thiazol-2-yl)dibenzo[b,d ]furan-3-sulfonamido)butanoic acid (Compound 283)

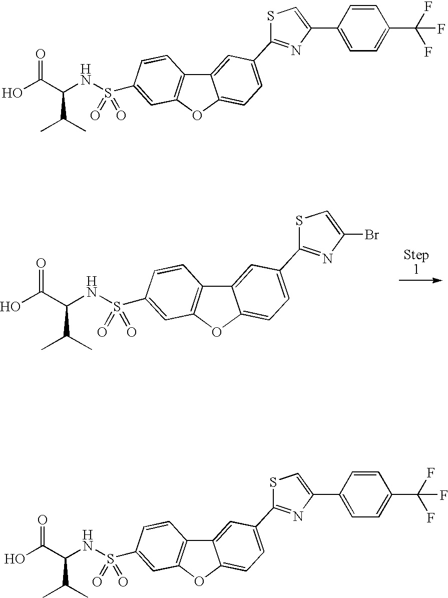

### Step 1: Preparation of (S)-3-methyl-2-(8-(4-(4-(trifluoromethyl)phenyl)thiazol-2-yl)dibenzo[b,d]furan-3-sulfonamido)butanoic acid

A mixture of (S)-2-(8-(4-bromothiazol-2-yl)dibenzo[b,d]furan-3-sulfonamido)-3-methylbutanoic acid (Compound 217, 50 mg, 0.12 mmol), 4-(trifluoromethyl)phenylboronic acid (25 mg, 0.13 mmol), PdCl₂(dppf).CH₂Cl₂ (3 mg, 0.003 mmol), K₃PO₄ (2 M solution in water) (0.4 mL) and DMF (2 ml) was heated at 80° C. for 3 hours. After cooling to RT, the reaction mixture was poured into ethyl acetate and water, the organic layer was separated, and the solvent was removed under reduced pressure. The crude residue was then purified by preparative HPLC to yield (S)-3-methyl-2-(8-(4-(4-(trifluoromethyl)phenyl)thiazol-2-yl)dibenzo[b,d]furan-3-sulfonamido)butanoic acid (15.3 mg). ¹H NMR (400 MHz, MeOD) δ ppm 0.90 (d, J=6.82 Hz, 3H), 0.97 (d, J=6.82 Hz, 3H), 1.97-2.15 (m, 1H), 3.72 (d, J=5.56 Hz, 1H), 7.70-7.82 (m, 3H), 7.86-7.95 (m, 1H), 8.03 (s, 1H), 8.11 (d, J=1.52 Hz, 1H), 8.20-8.33 (m, 4H), 8.77 (d, J=1.77 Hz, 1H). HRMS (ESI-FTMS): calcd for C₂₇H₂₁F₃N₂O₅S₂+H⁺: 575.09167. found: 575.0919.

### Example 29A

### (S)-2-(8-(4-(4-fluorophenyl)thiazol-2-yl)dibenzo[b,d]furan-3-sulfonamido)-3-methylbutanoic acid (Compound 284)

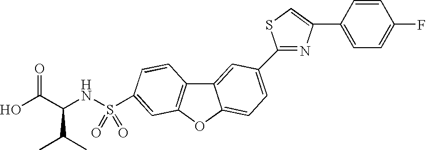

The title compound was prepared by the procedures described in Example 29, using 4-fluorophenylboronic acid instead of 4-(trifluoromethyl)phenylboronic acid. The compound was obtained as an off-white solid. ¹H NMR (400 MHz, MeOD) δ ppm 0.89 (d, J=6.82 Hz, 3H), 0.96 (d, J=6.57 Hz, 3H), 1.98-2.16 (m, 1H), 3.66 (d, J=5.56 Hz, 1H), 7.18-7.29 (m, 2H), 7.83 (d, J=8.84 Hz, 1H), 7.90-7.94 (m, 2H), 8.09-8.16 (m, 3H), 8.30 (dd, J=8.59, 1.77 Hz, 1H), 8.37 (d, J=8.08 Hz, 1H), 8.84 (d, J=1.52 Hz, 1H).

### Example 30

### (R)-3-methyl-2-(7-(thiazol-2-yl)dibenzo[b,d]thiophene-2-sulfonamido) butanoic acid (Compound 285)

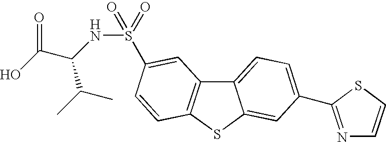

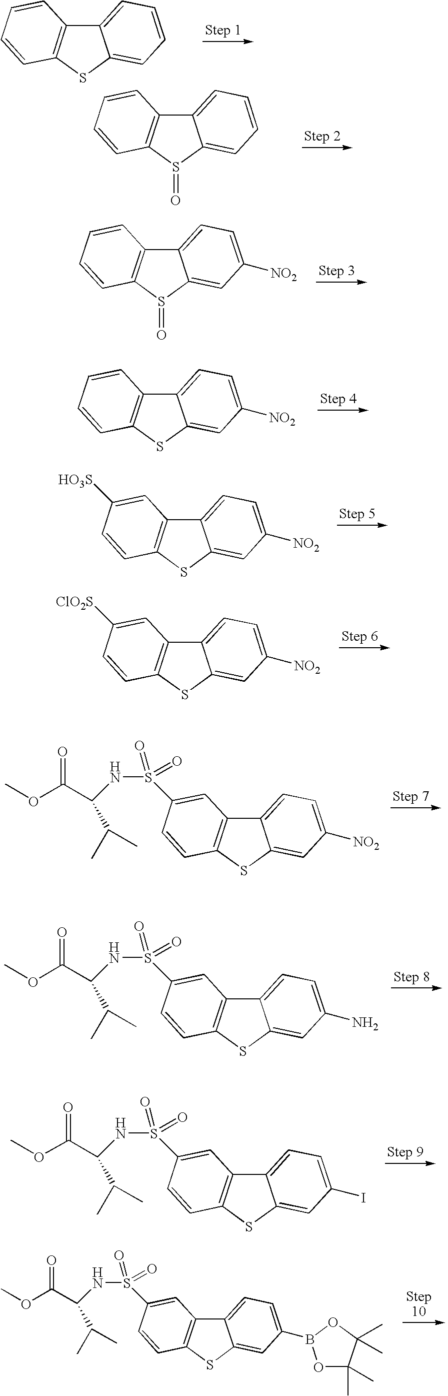

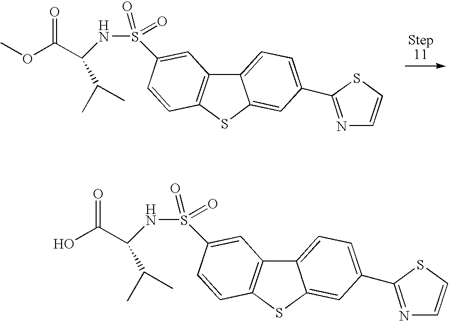

### Step 1: Preparation of dibenzo[b,d]thiophenesulfoxide

A fine powder of dibenzo[b,d]thiophene (110.4 g) was mixed with 1400 mL of dichloromethane. The resulting suspension was cooled in an ice bath, and MCPBA (147.6 g, 110 mmol) was added in small portions over 10 min. The reaction mixture (white suspension) was stirred at 0° C. for two hours and then filtered. The solid from the filtration was recrystallized from toluene. The product obtained was a mixture of dibenzo[b,d]thiophenesulfoxide and dibenzo[b,d]thiophenesulfone (42.3 g), which was used in the next step without further purification.

### Step 2: Preparation of 3-nitrodibenzo[b,d]thiophenesulfoxide

The product mixture of dibenzo[b,d]thiophenesulfoxide and dibenzo[b,d] thiophenesulfone (22 g) obtained in Step 1 was mixed with 50 mL of AcOH and 50 mL of conc. H₂SO₄. The resulting suspension was cooled in an ethanol/ice bath, and 55 mL of fuming HNO₃ (>90%) was added dropwise over 30 min. The reaction mixture was allowed to stir in an ice-water bath for five hours followed by filtration. The product was obtained as a mixture of 3-nitrodibenzo[b,d]thiophenesulfoxide and 3-nitrodibenzo[b,d]thiophenesulfone (29 g), which was used as such in the next step.

### Step 3: Preparation of 3-nitrodibenzo[b,d]thiophene

The product mixture of 3-nitrodibenzo[b,d]thiophenesulfoxide and 3-nitrodibenzo[b,d]thiophenesulfone (29 g) obtained in Step 2 was mixed with 290 mL of AcOH followed by dropwise addition of HBr (58 mL) over 30 min. The reaction mixture was allowed to stir at 40° C. for thirty minutes followed by filtration. The precipitate was dissolved in dichloromethane followed by a slow addition of hexanes to precipitate out the impurities. The desired product remains in solution, which was concentrated under reduced pressure to give 95% pure 3-nitrodibenzo[b,d]thiophene.

### Step 4: Preparation of 7-nitrodibenzo[b,d]thiophene-2-sulfonic acid

To a round-bottom flask containing 3-nitrodibenzo[b,d]thiophene (28 g) in 280 mL of TFA was slowly added chlorosulfonic acid (14 mL) at 0° C. The resulting suspension was allowed to warm to room temperature and stirred for 2 hours. It was then filtered, washed with TFA and dried to give 7-nitrodibenzo[b,d]thiophene-2-sulfonic acid as an off-white solid (31 g).

### Step 5: Preparation of 7-nitrodibenzo[b,d]thiophene-2-sulfonyl chloride

7-nitrodibenzo[b,d]thiophene-2-sulfonic acid (31 g) was mixed with 500 mL of thionyl chloride followed by slow addition of a few drops (90) of DMF. The mixture was heated and stirred in an 80° C. oil bath for 24 hours. The reaction mixture was filtered, and excess thionyl chloride in the filtrate was removed under reduced pressure. The crude product from the filtrate was isolated as a solid, which was triturated with ice water. The desired pure product 7-nitrodibenzo[b,d]thiophene-2-sulfonyl chloride (32 g) was obtained as an off-white solid.

### Step 6: Preparation of (R)-methyl3-methyl-2-(7-nitrodibenzo[b,d]thiophene-2-sulfonamido)butanoate

7-Nitrodibenzo[b,d]thiophene-2-sulfonyl chloride (25000 mg, 76.3 mmol) and (R)-methyl 2-amino-3-methylbutanoate hydrochloride (10900 mg, 83.4 mmol) were mixed with 300 mL of CH₂Cl₂ followed by slow addition of N,N-diisopropylethylamine (39500 mg, 305.2 mmol.) at 0° C. The mixture was stirred and allowed to warm to room temperature over 4 hours, whereupon it was diluted with ethyl acetate and water. The organic layer was separated and the solvent was removed under reduced pressure. The crude residue was purified by flash column chromatography, providing (R)-methyl3-methyl-2-(7-nitrodibenzo[b,d]thiophene-2-sulfonamido)butanoate as a white solid in 88% yield.

### Step 7: Preparation of (R)-methyl 2-(7-aminodibenzo[b,d]thiophene-2-sulfonamido)-3-methylbutanoate

(R)-Methyl3-methyl-2-(7-nitrodibenzo[b,d]thiophene-2-sulfonamido)butanoate (15 g) was mixed with 150 mL of EtOAc and 39 g of SnCl₂.H₂O (5 equivalents). The reaction mixture was heated to 50° C. for 5 hours, then was poured into ethyl acetate and water. The organic layer was separated and the solvent was removed under reduced pressure to give crude solid (R)-methyl 2-(7-aminodibenzo[b,d]thiophene-2-sulfonamido)-3-methylbutanoate in quantitative yield, which was used in the next step without further purification.

### Step 8: Preparation of (R)-methyl 2-(7-iododibenzo[b,d]thiophene-2-sulfonamido)-3-methylbutanoate

(R)-Methyl 2-(7-aminodibenzo[b,d]thiophene-2-sulfonamido)-3-methyl butanoate (12000 mg, 30.6 mmol) was mixed with hydrochloric acid (18% aqueous, 65 ml) and cooled to 0° C. An aqueous solution of sodium nitrite (1.0 M, 48 mL) was slowly added, and the reaction was stirred for 20 minutes followed by a very slow addition of a solution of sodium iodide (5045 mg, 33.7 mmol) in water (14 mL). The reaction was stirred for 20 minutes, whereupon water was added, and the resulting precipitate was collected via filtration to provide (R)-methyl 2-(7-iododibenzo[b,d]thiophene-2-sulfonamido)-3-methylbutanoate as a dark brown solid (13 g).

### Step 9: Preparation of (R)-methyl 3-methyl-2-(7-(4,4,5,5-tetramethyl-1,3,2-dioxaborolan-2-yl)dibenzo[b,d]thiophene-2-sulfonamido)butanoate

A mixture of (R)-methyl 2-(7-iododibenzo[b,d]thiophene-2-sulfonamido)-3-methylbutanoate (4000 mg, 7.93 mmol), 4,4,4′,4′,5,5,5′,5′-octamethyl-2,2′-bi(1,3,2-dioxaborolane) (2216 mg, 8.72 mmol), PdCl₂(dppf).CH₂Cl₂ (194 mg, 0.24 mmol), KOAc (2336 mg, 23.8 mmol) and DMSO (30 ml) was heated to 80° C. for 5 hours. After cooling to RT, the mixture was poured into ethyl acetate and water, the organic layer was separated, and the solvent removed under reduced pressure. The crude residue was purified by flash column chromatography to provide (R)-methyl 3-methyl-2-(7-(4,4,5,5-tetramethyl-1,3,2-dioxaborolan-2-yl)dibenzo[b,d]thiophene-2-sulfonamido)butanoate as a white solid (2 g).

### Step 10: Preparation of (R)-methyl 3-methyl-2-(7-(thiazol-2-yl)dibenzo[b,d]thiophene-2-sulfonamido)butanoate

A mixture of (R)-methyl 3-methyl-2-(7-(4,4,5,5-tetramethyl-1,3,2-dioxaborolan-2-yl)dibenzo[b,d]thiophene-2-sulfonamido)butanoate (100 mg, 0.2 mmol), 2-bromothiazole (35 uL, 0.4 mmol), PdCl₂(dppf).CH₂Cl₂ (17 mg, 0.02 mmol), K₃PO₄ (2 M solution in water) (0.6 mL, 1.2 mmol) and DMF (4 ml) was heated at 80° C. for 3 hours, then was cooled to RT and poured into ethyl acetate and water. The organic layer was separated, concentrated under reduced pressure, and the crude residue was purified by preparative HPLC to yield (R)-methyl 3-methyl-2-(7-(thiazol-2-yl)dibenzo[b,d]thiophene-2-sulfonamido)butanoate (40.7 mg).

### Step 11: Preparation of (R)-3-methyl-2-(7-(thiazol-2-yl)dibenzo[b,d]thiophene-2-sulfonamido)butanoic acid

A solution of (R)-methyl 3-methyl-2-(7-(thiazol-2-yl)dibenzo[b,d]thiophene-2-sulfonamido)butanoate (40.7 mg, 0.09 mmol) in THF/MeOH/water (2 mL) was treated with LiOH (5 equivalents), and the reaction was stirred overnight. Following the addition of water, the pH of the solution was adjusted to between 4-5, and the resulting precipitate was then filtered to yield (R)-3-methyl-2-(7-(thiazol-2-yl)dibenzo[b,d]thiophene-2-sulfonamido)butanoic acid as a white solid (14 mg). ¹H NMR (400 MHz, MeOD) δ ppm 0.94 (d, J=6.82 Hz, 3H), 1.00 (d, J=6.82 Hz, 3H), 2.02-2.16 (m, 1H), 3.78 (d, J=5.56 Hz, 1H), 7.60-7.66 (m, 1H), 7.77 (s, 1H), 7.90-8.01 (m, 2H), 8.03-8.16 (m, 2H), 8.41 (d, J=8.34 Hz, 1H), 8.54 (d, J=1.01 Hz, 1H), 8.75 (d, J=1.77 Hz, 1H). HRMS (ESI-FTMS): calcd for C₂₀H₁₈N₂O₄S₃+H⁺: 447.05014. found: 447.04966.

### Example 30A

### (R)-2-(7-(benzo[d]thiazol-2-yl)dibenzo[b,d]thiophene-2-sulfonamido)-3-methylbutanoic acid (Compound 286)

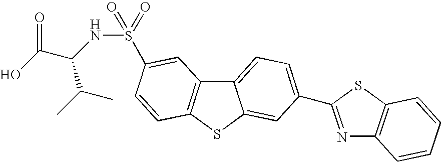

The title compound was prepared by the procedures described in Example 30, using 2-bromobenzo[d]thiazole instead of 2-bromothiazole. The compound was obtained as an off-white solid. ¹H NMR (400 MHz, MeOD) δ ppm 0.93 (d, J=6.82 Hz, 3H), 1.02 (d, J=6.82 Hz, 3H), 2.03-2.17 (m, 1H), 3.75 (d, J=4.55 Hz, 1H), 7.38-7.60 (m, 2H), 7.89-8.13 (m, 4H), 8.17-8.29 (m, 1H), 8.44 (d, J=8.59 Hz, 1H), 8.66 (d, J=1.01 Hz, 1H), 8.76 (d, J=1.77 Hz, 1H). HRMS (ESI-FTMS): calcd for C₂₄H₂₀N₂O₄S₃+H⁺: 497.06579. found: 497.06601.

### Example 31

### (R)-2-(7-(furan-2-yl)dibenzo[b,d]thiophene-2-sulfonamido)-3-methyl butanoic acid (Compound 287)

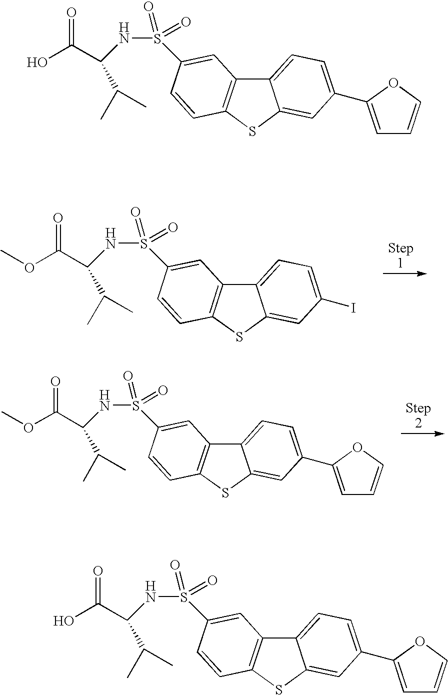

### Step 1: Preparation of (R)-methyl 2-(7-(furan-2-yl)dibenzo[b,d]thiophene-2-sulfonamido)-3-methylbutanoate

A mixture of (R)-methyl 2-(7-iododibenzo[b,d]thiophene-2-sulfonamido)-3-methylbutanoate (400 mg, 0.8 mmol) (an intermediate in the preparation of Example 30), 2-(furan-2-yl)-4,4,5,5-tetramethyl-1,3,2-dioxaborolane (310 mg, 1.6 mmol), PdCl₂(dppf).CH₂Cl₂ (68 mg, 0.08 mmol), K₃PO₄ (2 M solution in water) (2.4 mL) and DMF (16 mL), were heated at 80° C. for 3 hours. After cooling to RT, the mixture was poured into ethyl acetate and water, the organic layer was separated, concentrated under reduced pressure, and the crude residue was purified by preparative HPLC to yield (R)-methyl 2-(7-(furan-2-yl)dibenzo[b,d]thiophene-2-sulfonamido)-3-methylbutanoate (146.5 mg).

### Step 2: Preparation of (R)-2-(7-(furan-2-yl)dibenzo[b,d]thiophene-2-sulfonamido)-3-methylbutanoic acid

A solution of (R)-methyl 2-(7-(furan-2-yl)dibenzo[b,d]thiophene-2-sulfonamido)-3-methylbutanoate (146.5 mg, 0.33 mmol) in THF/MeOH/water (4 mL) was treated with LiOH (5 equivalents), and the reaction was stirred overnight. Following the addition of water, the pH of the solution was adjusted to between 4-5, and the resulting precipitate was then filtered to yield (R)-2-(7-(furan-2-yl)dibenzo[b,d]thiophene-2-sulfonamido)-3-methylbutanoic acid as a white solid (108.5 mg). ¹H NMR (400 MHz, MeOD) δ ppm 0.93 (d, J=6.57 Hz, 3H), 0.99 (d, J=6.82 Hz, 3H), 1.98-2.20 (m, 1H), 3.75 (d, J=5.31 Hz, 1H), 6.53-6.60 (m, 1H), 6.91 (d, J=3.28 Hz, 1H), 7.61 (d, J=1.77 Hz, 1H), 7.84-7.94 (m, 1H), 8.02 (d, J=8.34 Hz, 1H), 8.24 (d, J=1.26 Hz, 1H), 8.31 (d, J=8.34 Hz, 1H), 8.67 (d, J=1.77 Hz, 1 H). HRMS (ESI-FTMS): calcd for C₂₁H₁₉NO₅S₂+H⁺: 430.07774. found: 430.07738.

### Example 32

### (R)-2-(7-(5-chlorofuran-2-yl)dibenzo[b,d]thiophene-2-sulfonamido)-3-methylbutanoic acid (Compound 288)

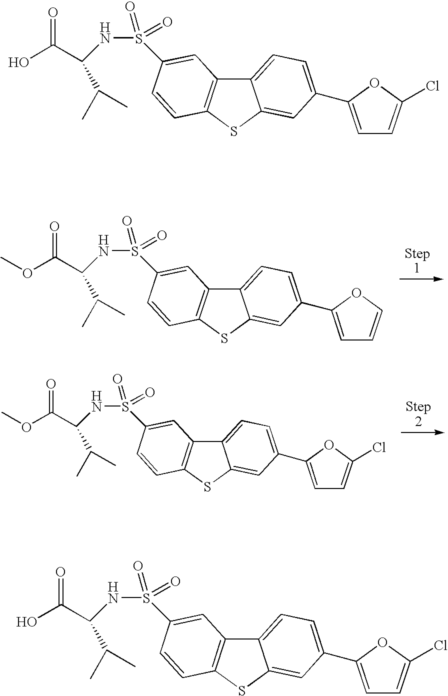

### Step 1: Preparation of (R)-methyl 2-(7-(5-chlorofuran-2-yl)dibenzo[b,d]thiophene-2-sulfonamido)-3-methylbutanoate

A solution of (R)-methyl 2-(7-(furan-2-yl)dibenzo[b,d]thiophene-2-sulfonamido)-3-methylbutanoate (50 mg, 0.11 mmol) (the penultimate in the preparation of Example 31) in CH₂Cl₂ (1 mL) was treated with N-chlorosuccinimide (NCS, 18 mg, 0.14 mmol) followed by a catalytic amount of TFA. The mixture was stirred at room temperature until no starting material was left according to LC-MS, whereupon DMSO (0.5 mL) was added and the reaction was stirred at room temperature for an additional 1 hour. Brine was added, the organic layer was separated, washed with water/brine, and was concentrated to yield the crude product as a brown solid which was purified by column chromatography to give (R)-methyl 2-(7-(5-chlorofuran-2-yl)dibenzo[b,d]thiophene-2-sulfonamido)-3-methylbutanoate as a white solid (24.5 mg).

### Step 2: Preparation of (R)-2-(7-(5-chlorofuran-2-yl)dibenzo[b,d]thiophene-2-sulfonamido)-3-methylbutanoic acid

A solution of (R)-methyl 2-(7-(5-chlorofuran-2-yl)dibenzo[b,d]thiophene-2-sulfonamido)-3-methylbutanoate (24.5 mg, 0.05 mmol) in THF/MeOH/water (2 mL) was treated with LiOH (5 equivalents) and the reaction was stirred overnight. Following the addition of water, the pH of the solution was adjusted to between 4-5, and the resulting precipitate was then filtered to yield (R)-2-(7-(5-chlorofuran-2-yl)dibenzo[b,d]thiophene-2-sulfonamido)-3-methylbutanoic acid as a white solid (10.3 mg). ¹H NMR (400 MHz, MeOD) δ ppm 0.93 (d, J=6.82 Hz, 3H), 1.00 (d, J=7.07 Hz, 1H), 1.98-2.20 (m, 1H), 3.75 (d, J=5.31 Hz, 1H), 6.38 (d, J=3.28 Hz, 1H), 6.90 (d, J=3.54 Hz, 1H), 7.96-8.03 (m, 3H), 8.16-8.20 (m, 1H), 8.29-8.30 (m, 1H), 8.65-8.68 (m, 1H). HRMS (ESI-FTMS): calcd for C₂₁H₁₈ClNO₅S₂+H⁺: 464.03877. found: 464.03995.

### Example 33

### (R)-3-methyl-2-(7-(5-phenylthiophen-2-yl)dibenzo[b,d]furan-2-sulfonamido)butanoic acid (Compound 289)

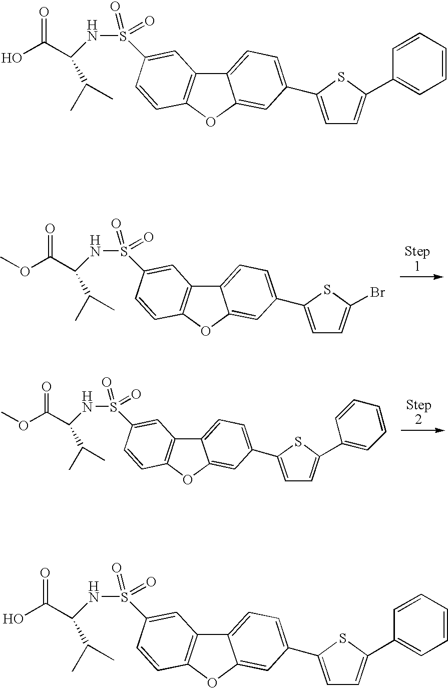

### Step 1: Preparation of (R)-3-methyl-2-(7-(5-phenylthiophen-2-yl)dibenzo[b,d]furan-2-sulfonamido)butanoate

A mixture of (R)-methyl 2-(7-(5-bromothiophen-2-yl)dibenzo[b,d]furan-2-sulfonamido)-3-methylbutanoate (an intermediate in the preparation of compound 144 described in Example 4) (43 mg, 0.082 mmol), phenylboronic acid (12 mg, 0.098 mmol), Pd(PPh₃)₄ (5 mg, 0.004 mmol), K₂CO₃ (23 mg, 0.164 mmol), DME (2 mL) and water (0.5 mL) was heated at 90° C. for 3 hours. After cooling to RT, the mixture was poured into ethyl acetate and water, the organic layer was separated, concentrated under reduced pressure, and the crude residue was purified by preparative HPLC to yield (R)-3-methyl-2-(7-(5-phenylthiophen-2-yl)dibenzo[b,d]furan-2-sulfonamido)butanoate (10 mg).

### Step 2: Preparation of (R)-3-methyl-2-(7-(5-phenylthiophen-2-yl)dibenzo[b,d]furan-2-sulfonamido)butanoic acid

A solution of (R)-3-methyl-2-(7-(5-phenylthiophen-2-yl)dibenzo[b,d]furan-2-sulfonamido)butanoate (10 mg, 0.019 mmol) in THF/MeOH/water (2 mL) was treated with LiOH (5 equivalents) and the reaction was stirred overnight. Following the addition of water, the pH of the solution was adjusted to between 4-5, and the precipitate obtained was then filtered to yield (R)-3-methyl-2-(7-(5-phenylthiophen-2-yl)dibenzo[b,d]furan-2-sulfonamido)butanoic acid as a white solid (1.4 mg). ¹H NMR (400 MHz, MeOD) δ ppm 0.92 (d, J=6.82 Hz, 3H), 0.98 (d, J=6.82 Hz, 3H), 1.97-2.13 (m, 1H), 3.74 (d, J=5.56 Hz, 1H), 7.30-7.41 (m, 1H), 7.43-7.50 (m, 2H), 7.53 (d, J=3.79 Hz, 1H), 7.65 (d, J=4.04 Hz, 1H), 7.73-7.89 (m, 4H), 7.98-8.07 (m, 2H), 8.23 (d, J=8.08 Hz, 1H), 8.60 (d, J=2.02 Hz, 1H). HRMS (ESI-FTMS): calcd for C₂₇H₂₃NO₅S₂+H⁺: 506.10904. found: 506.11097.

### Example 34

### (R)-2-(7-(5-chlorofuran-2-yl)dibenzo[b,d]furan-2-sulfonamido)-3-methyl butanoic acid (Compound 290)

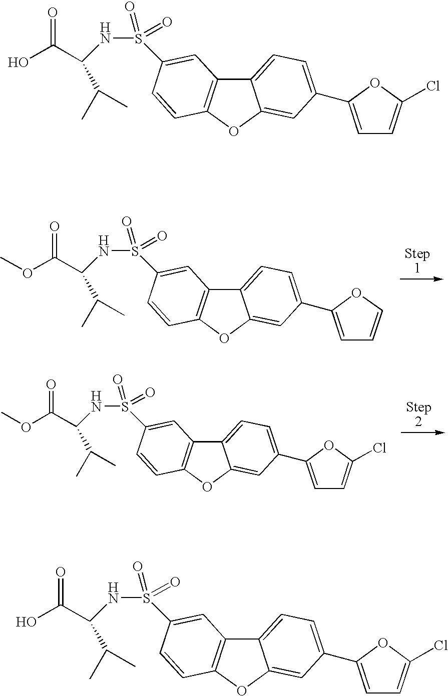

### Step 1: Preparation of (R)-methyl 2-(7-(5-chlorofuran-2-yl)dibenzo[b,d]furan-2-sulfonamido)-3-methylbutanoate

A solution of (R)-methyl 2-(7-(furan-2-yl)dibenzo[b,d]furan-2-sulfonamido)-3-methylbutanoate (123 mg, 0.29 mmol) (an intermediate in the preparation of Example 4) in CH₂Cl₂ (1 mL) was treated with N-chlorosuccinimide (NCS, 46 mg, 0.34 mmol) followed by a catalytic amount of TFA. The mixture was stirred at room temperature until no starting material was left according to LC-MS, whereupon DMSO (0.5 mL) was added and the reaction was stirred at room temperature for an additional 1 hour. Brine was added, the organic layer was separated, washed with water/brine, and was concentrated to yield the crude product as a brown solid which was purified by column chromatography to give (R)-methyl 2-(7-(5-chlorofuran-2-yl)dibenzo[b,d]furan-2-sulfonamido)-3-methylbutanoate as a white solid (78.5 mg).

### Step 2: Preparation of (R)-2-(7-(5-chlorofuran-2-yl)dibenzo[b,d]furan-2-sulfonamido)-3-methylbutanoic acid

A solution of (R)-methyl 2-(7-(5-chlorofuran-2-yl)dibenzo[b,d]furan-2-sulfonamido)-3-methylbutanoate (78.5 mg, 0.18 mmol) in THF/MeOH/water (4 mL) was treated with LiOH (5 equivalents) and the reaction was stirred overnight. Following the addition of water, the pH of the solution was adjusted to between 4-5, and the resulting precipitate was then filtered to yield (R)-2-(7-(5-chlorofuran-2-yl)dibenzo[b,d]furan-2-sulfonamido)-3-methylbutanoic acid as a white solid (45.3 mg). ¹H NMR (400 MHz, MeOD) δ ppm 0.91 (d, J=6.82 Hz, 3H), 0.97 (d, J=6.82 Hz, 3H), 1.96-2.11 (m, 1H), 3.72 (d, J=5.81 Hz, 1H), 6.42 (d, J=3.54 Hz, 1H), 6.98 (d, J=3.28 Hz, 1H), 7.68-7.80 (m, 2H), 7.90 (d, J=1.52 Hz, 1H), 7.98 (dd, J=8.84, 2.02 Hz, 1H), 8.13 (d, J=8.08 Hz, 1H), 8.53 (dd, J=2.02, 0.51 Hz, 1H). HRMS (ESI-FTMS): calcd for C₂₁H₁₈ClNO₆S+H⁺: 448.06161. found: 448.06073.

### Example 35

### (R)-2-(7-(benzo[d]oxazol-2-yl)dibenzo[b,d]furan-2-sulfonamido)-3-methyl butanoic acid (Compound 294)

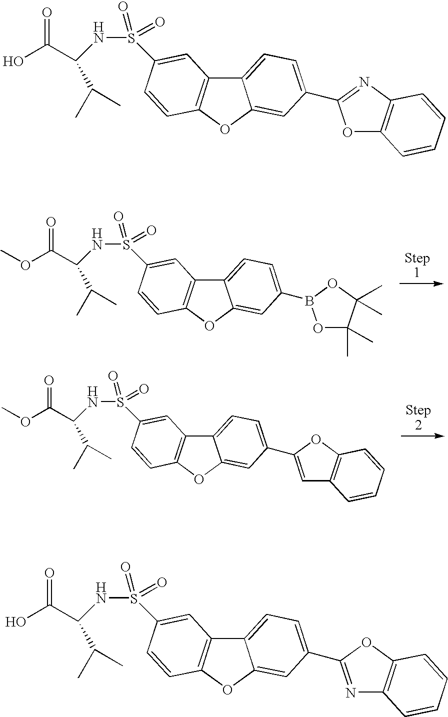

### Step 1: Preparation of (R)-2-(7-(benzo[d]oxazol-2-yl)dibenzo[b,d]furan-2-sulfonamido)-3-methylbutanoate

A mixture of (R)-methyl 3-methyl-2-(7-(4,4,5,5-tetramethyl-1,3,2-dioxaborolan-2-yl)dibenzo[b,d]furan-2-sulfonamido)butanoate (an intermediate in preparation of Example 22) (100 mg, 0.2 mmol), 2-chlorobenzo[d]oxazole (46 uL, 0.4 mmol), PdCl₂(dppf).CH₂Cl₂ (17 mg, 0.02 mmol), K₃PO₄ (2 M solution in water) (0.6 mL, 1.2 mmol) and DMF (4 ml) was heated at 120° C. for 20 minutes under microwave radiation. After cooling to RT, the mixture was poured into ethyl acetate and water, the organic layer was separated, concentrated under reduced pressure, and the crude residue was purified by preparative HPLC to yield (R)-2-(7-(benzo[d]oxazol-2-yl)dibenzo[b,d]furan-2-sulfonamido)-3-methylbutanoate (15 mg).

### Step 2: Preparation of (R)-2-(7-(benzo[d]oxazol-2-yl)dibenzo[b,d]furan-2-sulfonamido)-3-methylbutanoic acid

A solution of (R)-2-(7-(benzo[d]oxazol-2-yl)dibenzo[b,d]furan-2-sulfonamido)-3-methylbutanoate (15 mg, 0.03 mmol) in THF/MeOH/water (2 mL) was treated with LiOH (5 equivalents), and the reaction was stirred overnight at RT. Following the addition of water, the pH of the solution was adjusted to between 4-5, and the precipitate obtained was then filtered to yield (R)-2-(7-(benzo[d]oxazol-2-yl)dibenzo[b,d]furan-2-sulfonamido)-3-methyl butanoic acid as a white solid (7.6 mg). ¹H NMR (400 MHz, MeOD) δ ppm 0.92 (d, J=6.57 Hz, 3H), 1.01 (d, J=6.82 Hz, 3H), 2.00-2.15 (m, 1H), 3.68 (d, J=5.05 Hz, 1H), 7.37-7.50 (m, 2H), 7.70 (dd, 1H), 7.74-7.80 (m, 2H), 8.06 (dd, J=8.59, 2.02 Hz, 1H), 8.22-8.37 (m, 2H), 8.47 (s, 1H), 8.63 (d, J=1.77 Hz, 1H). HRMS (ESI-FTMS): calcd for C₂₄H₂₀N₂O₆S+H⁺: 465.11148. found: 465.11154.

### Example 35A

### (R)-2-(7-(5-chloro-4-(trifluoromethyl)thiazol-2-yl)dibenzo[b,d]furan-2-sulfonamido)-3-methylbutanoic acid (Compound 295)

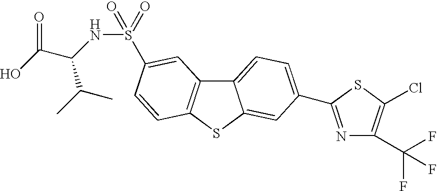

The title compound was prepared by the procedures described in Example 35, using 2-bromo-5-chloro-4-(trifluoromethyl)thiazole instead of 2-chlorobenzo[d]oxazole. The compound was obtained as an off-white solid. ¹H NMR (400 MHz, MeOD) δ ppm 0.93 (d, J=6.82 Hz, 3H), 0.99 (d, J=6.82 Hz, 3H), 1.98-2.20 (m, 1H), 3.74 (d, J=5.31 Hz, 1H), 7.70-7.78 (m, 2H), 7.93-8.08 (m, 2H), 8.17-8.26 (m, 2H), 8.60 (d, J=2.02 Hz, 1H). HRMS (ESI-FTMS): calcd for C₂₁H₁₆ClF₃N₂O₅S₂+H⁺: 533.02140. found: 533.02276.

### Example 35B

### (R)-2-(7-(6-methoxybenzo[d]thiazol-2-yl)dibenzo[b,d]furan-2-sulfonamido)-3-methylbutanoic acid (Compound 296)

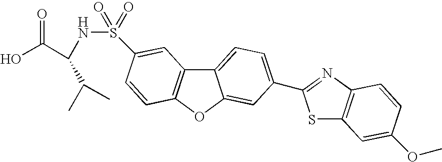

The title compound was prepared by the procedures described in Example 35, using 2-chloro-6-methoxybenzo[d]thiazole instead of 2-chlorobenzo[d]oxazole. The compound was obtained as an off-white solid. ¹H NMR (400 MHz, MeOD) δ ppm 0.92 (d, J=6.82 Hz, 3H), 1.01 (d, J=7.07 Hz, 3H), 1.89-2.10 (m, 1H), 3.70-3.90 (m, 1H), 3.94 (s, 3H), 7.16 (dd, J=8.84, 2.53 Hz, 1H), 7.46 (d, J=2.27 Hz, 1H), 7.73 (d, J=8.59 Hz, 1H), 7.92-8.20 (m, 4H), 8.32 (s, 1H), 8.57 (d, J=1.52 Hz, 1H). MS (LC-ESIMS) m/z 511.2 (MH⁺).

### Example 35C

### (R)-2-(7-(6-fluorobenzo[d]thiazol-2-yl)dibenzo[b,d]furan-2-sulfonamido)-3-methylbutanoic acid (Compound 297)

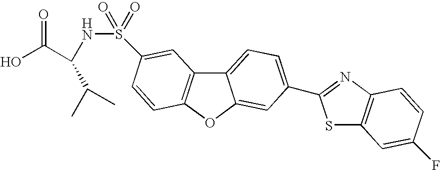

The title compound was prepared by the procedures described in Example 35, using 2-chloro-6-fluorobenzo[d]thiazole instead of 2-chlorobenzo[d]oxazole. The compound was obtained as an off-white solid. ¹H NMR (400 MHz, MeOD) δ ppm 0.92 (d, J=7.07 Hz, 3H), 2.04-2.14 (m, 1H), 3.44-3.60 (m, 1H), 7.24-7.39 (m, 1H), 7.74-7.79 (m, 1H), 7.99-8.09 (m, 2H), 8.14 (dd, J=8.21, 1.39 Hz, 1H), 8.25 (d, J=7.83 Hz, 1H), 8.36 (d, J=1.01 Hz, 1H), 8.61 (d, J=1.26 Hz, 1H). HRMS (ESI-FTMS): calcd for C₂₄H₁₉FN₂O₅S₂+H⁺: 499.07922. found: 499.07896.

### Example 35D

### (R)-3-methyl-2-(7-(6-methylbenzo[d]thiazol-2-yl)dibenzo[b,d]furan-2-sulfonamido)butanoic acid (Compound 298)

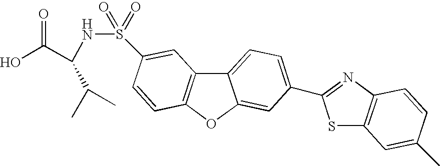

The title compound was prepared by the procedures described in Example 35, using 2-chloro-6-methylbenzo[d]thiazole instead of 2-chlorobenzo[d]oxazole. The compound was obtained as an off-white solid. ¹H NMR (400 MHz, MeOD) δ ppm 0.93 (d, J=6.82 Hz, 3H), 1.00 (d, J=6.82 Hz, 3H), 1.96-2.14 (m, 1H), 2.54 (s, 3H), 3.78 (d, J=5.31 Hz, 1H), 7.32-7.44 (m, 1H), 7.70-7.81 (m, 2H), 7.96 (d, J=8.34 Hz, 1H), 8.04 (dd, J=8.72, 1.89 Hz, 1 H), 8.07-8.25 (m, 2H), 8.30-8.38 (m, 1H), 8.58 (d, J=2.02 Hz, 1H). HRMS (ESI-FTMS): calcd for C₂₅H₂₂N₂O₅S₂+H⁺: 495.10429. found: 495.10418.

### Example 35E

### (R)-2-(7-(4-fluorobenzo[d]thiazol-2-yl)dibenzo[b,d]furan-2-sulfonamido)-3-methylbutanoic acid (Compound 299)

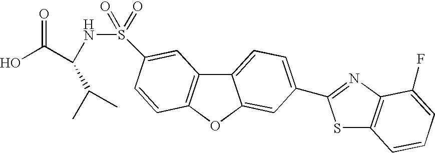

The title compound was prepared by the procedures described in Example 35, using 2-bromo-4-fluorobenzo[d]thiazole instead of 2-chlorobenzo[d]oxazole. The compound was obtained as an off-white solid. ¹H NMR (400 MHz, MeOD) δ ppm 0.94 (d, J=6.82 Hz, 3H), 1.01 (d, J=6.82 Hz, 3H), 2.00-2.19 (m, 1H), 3.78 (d, J=5.31 Hz, 1H), 7.18-7.33 (m, 1 H), 7.37-7.51 (m, 1H), 7.77 (dd, J=14.65, 8.34 Hz, 2H), 8.06 (dd, J=8.72, 1.89 Hz, 1H), 8.13-8.28 (m, 2H), 8.42 (s, 1H), 8.60 (d, J=2.02 Hz, 1H). HRMS (ESI-FTMS): calcd for C₂₄H₁₉FN₂O₅S₂+H⁺: 499.07922. found: 499.0790.

### Example 35F

### (R)-3-methyl-2-(7-(4,5,6-trifluorobenzo[d]thiazol-2-yl)dibenzo[b,d]furan-2-sulfonamido)butanoic acid (Compound 300)

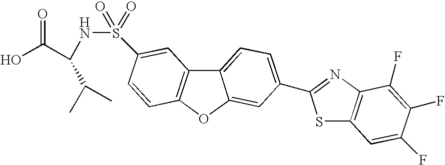

The title compound was prepared by the procedures described in Example 35, using 2-bromo-4,5,6-trifluorobenzo[d]thiazole instead of 2-chlorobenzo[d]oxazole. The compound was obtained as an off-white solid. ¹H NMR (400 MHz, MeOD) δ ppm 0.93 (d, J=6.82 Hz, 3H), 1.00 (d, J=6.82 Hz, 3H), 2.01-2.20 (m, 1H), 3.77 (d, J=5.31 Hz, 1H), 7.69-7.79 (m, 2H), 8.07 (dd, J=8.72, 1.89 Hz, 1H), 8.11-8.17 (m, 1H), 8.19-8.26 (m, 1H), 8.41 (dd, J=1.52, 0.51 Hz, 1H), 8.55-8.64 (m, 1H). HRMS (ESI-FTMS): calcd for C₂₄H₁₇F₃N₂O₅S₂+H⁺: 535.06037. found: 535.0598.

### Example 35G

### (R)-3-methyl-2-(7-(6-(trifluoromethoxy)benzo[d]thiazol-2-yl)dibenzo[b,d] furan-2-sulfonamido)butanoic acid (Compound 301)

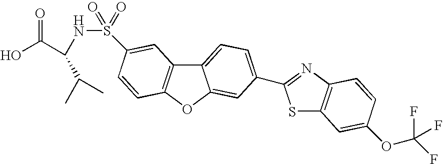

The title compound was prepared by the procedures described in Example 35, using 2-bromo-6-trifluoromethoxybenzo[d]thiazole instead of 2-chlorobenzo[d]oxazole. The compound was obtained as an off-white solid. ¹H NMR (400 MHz, MeOD) δ ppm 0.93 (d, J=6.82 Hz, 3H), 1.01 (d, J=6.82 Hz, 3H), 1.92-2.28 (m, 1H), 3.76 (d, J=5.31 Hz, 1H), 7.45 (d, J=7.83 Hz, 1H), 7.75 (d, J=8.84 Hz, 1H), 7.94 (s, 1H), 8.01-8.20 (m, 3H), 8.19-8.27 (m, 1H), 8.37 (s, 1H), 8.61 (d, J=2.02 Hz, 1H). HRMS (ESI-FTMS): calcd for C₂₅H₁₉F₃N₂O₆S₂+H⁺: 565.07094. found: 565.0707.

### Example 35H

### (R)-3-methyl-2-(7-(6-(trifluoromethyl)benzo[d]thiazol-2-yl)dibenzo[b,d] furan-2-sulfonamido)butanoic acid (Compound 302)

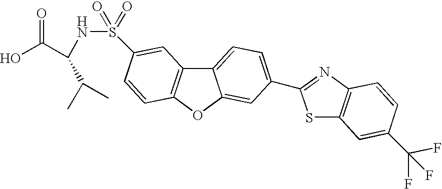

The title compound was prepared by the procedures described in Example 35, using 2-bromo-6-trifluoromethylbenzo[d]thiazole instead of 2-chlorobenzo[d]oxazole. The compound was obtained as an off-white solid. ¹H NMR (400 MHz, MeOD) δ ppm 0.93 (d, J=6.82 Hz, 3H), 1.01 (d, J=6.82 Hz, 3H), 1.94-2.23 (m, 1H), 3.77 (d, J=5.31 Hz, 1H), 7.72-7.84 (m, 2H), 8.07 (dd, J=8.72, 1.89 Hz, 1H), 8.15-8.29 (m, 3H), 8.35 (s, 1H), 8.44 (d, J=0.76 Hz, 1H), 8.61 (d, J=2.02 Hz, 1H).

### Example 36

### (S)-2-(8-ethynyldibenzo[b,d]furan-3-sulfonamido)-3-methylbutanoic acid (Compound 303)

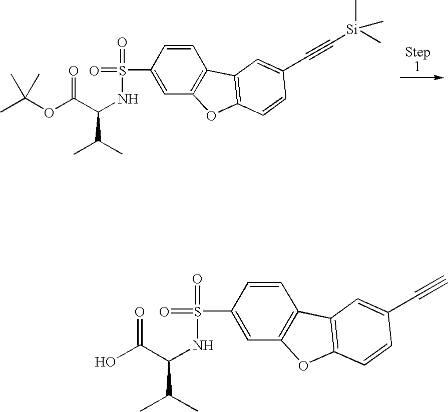

Step 1: The title compound was synthesized by treatment of (S)-tert-butyl 3-methyl-2-(8-((trimethylsilyl)ethynyl)dibenzo[b,d]furan-3-sulfonamido)butanoate (prepared following the procedures described in Example 6, using ethynyltrimethylsilane in replace of 3-methoxyprop-1-yne) in methylene chloride at room temperature for 6 hours. The desired product (S)-2-(8-ethynyldibenzo[b,d]furan-3-sulfonamido)-3-methylbutanoic acid was obtained as white powder after evaporation of the solvent and TFA (94%). ESIMS (m/z) 372.10 (MH⁺).

### Example 37

### (S)-2-(7-(5-chlorothiophen-2-yl)dibenzo[b,d]thiophene-3-sulfonamido)-3-methylbutanoic acid (Compound 304)

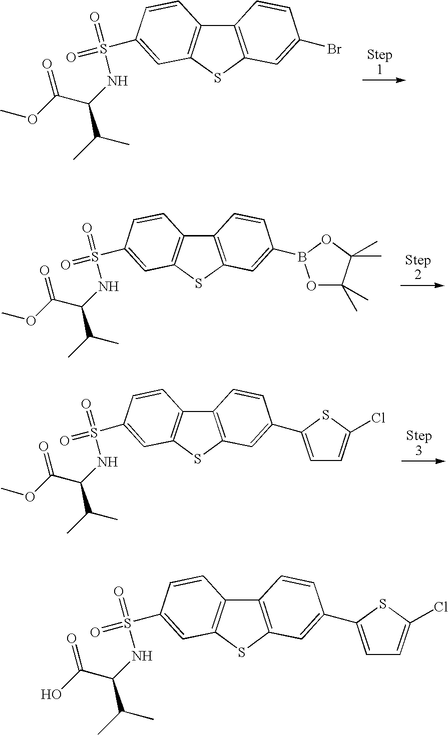

### Step 1: Preparation of (S)-methyl 3-methyl-2-(7-(4,4,5,5-tetramethyl-1,3,2-dioxaborolan-2-yl)dibenzo[b,d]thiophene-3-sulfonamido)butanoate

A mixture of (S)-methyl-2-(7-bromodibenzo[b,d]thiophene-3-sulfonamido)-3-methyl butanoate (456 mg, 1 mmol, an intermediate in the preparation of example 17), bis-(pinacolato)-diboron (762 mg, 3 mmol) and KOAc (295 mg, 3 mmol) were suspended in DMSO (10 mL), and the mixture was degassed by bubbling nitrogen through for 10 minutes. Following the addition of Pd(dppf)₂Cl₂ (23 mg, 0.05 mmol) and CH₂Cl₂ (5 mL), the mixture was heated at 80° C. for 4 hours, allowed to cool to RT, and then diluted with water (35 ml). The mixture was extracted with CH₂Cl₂ (2×20 mL), the organic phase was dried over Na₂SO₄, and concentrated under reduced pressure. The residue was purified by flash column chromatography (hexane/AcOEt 9:1 to 3:1), providing the desired product (191 mg, 38% yield) as a white solid.

### Step 2: Preparation of (S)-methyl 2-(7-(5-chlorothiophen-2-yl)dibenzo[b,d]thiophene-3-sulfonamido)-3-methylbutanoate

A solution of (S)-methyl-3-methyl-2-(7-(4,4,5,5-tetramethyl-1,3,2-dioxaborolan-2-yl)dibenzo[b,d]thiophene-3-sulfonamido)butanoate (191 mg, 0.38 mmol), 2-bromo-5-chlorothiophene (165 mg, 92 μl, 0.836 mmol) and K₂CO₃ (132 mg, 0.95 mmol) in a mixture of DME/water (20:1), and the solution was degassed by bubbling nitrogen through for 10 minutes. Following the addition of Pd(PPh₃)₄, the reaction mixture was heated at reflux for 4 hours, then was cooled to RT, diluted with ethyl acetate, and washed with brine. The organic phase was dried over Na₂SO₄, concentrated under reduced pressure, and the crude residue was purified by flash column chromatography (hexane/AcOEt 85:15 to 7:3) to provide the desired product (88 mg, 47% yield) as a white solid.

### Step 3: (S)-2-(7-(5-chlorothiophen-2-yl)dibenzo[b,d]thiophene-3-sulfonamido)-3-methylbutanoic acid

A solution of the ester prepared in step 2 (88 mg, 0.178 mmol) in 1:1 THF/H₂O (3 ml) was treated with LiOH (46 mg, 1.07 mmol), and the mixture was stirred at RT for 72 hours. The THF was removed under reduced pressure and the aqueous solution acidified with diluted HCl. The resulting precipitate was collected by filtration and then purified by preparative HPLC to provide the desired product (30 mg, 37% yield) as a white solid. ¹H NMR (300 MHz, MeOD) δppm 8.41 (dd, J=1.8, 0.6 Hz, 1H), 8.26-8.40 (m, 2H), 8.19 (d, J=1.2 Hz, 1H), 7.94 (dd, J=8.4, 1.6 Hz, 1H), 7.76 (dd, J=8.2, 1.8 Hz, 1H), 7.41 (d, J=3.8 Hz, 1H), 7.05 (d, J=4.1 Hz, 1H), 3.51 (d, J=4.4 Hz, 1H), 1.96-2.21 (m, 1H), 1.02 (d, J=6.7 Hz, 3H), 0.89 (d, J=6.7 Hz, 3H). ESIMS (m/z) 479.94 (MH⁺).

### Example 38

### (S)-2-(8-(4,5-dimethylthiazol-2-yl)dibenzo[b,d]furan-3-sulfonamido)-3-methylbutanoic acid (Compound 305)

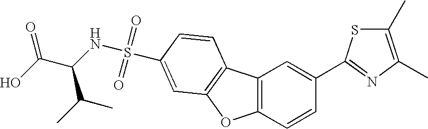

The title compound was prepared by the procedures described in Example 20, using 2-bromo-4,5-dimethylthiazole (its preparation is described below) instead of 2-bromothiazole. The title compound was obtained as an off-white solid. ¹H NMR (300 MHz, MeOD.) δ ppm 8.61 (d, 1H), 8.27 (d, J=8.2 Hz, 1H), 8.12 (dd, J=1.5, 0.6 Hz, 1H), 8.09 (dd, J=8.7, 1.9 Hz, 1H), 7.92 (dd, J=8.2, 1.5 Hz, 1H), 7.74 (dd, J=8.8, 0.6 Hz, 1H), 3.76 (d, J=5.6 Hz, 1H), 2.46 (s, 3H), 2.42 (s, 3H), 1.97-2.17 (m, 1H), 0.99 (d, J=6.7 Hz, 3H), 0.94 (d, J=6.7 Hz, 3H). ESIMS (m/z) 459.10 (MH⁺).

### Synthesis of 2-bromo-4,5-dimethylthiazole

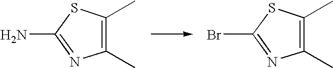

A solution of 4,5-dimethylthiazol-2-amine hydroboromide (4.94 g, 30 mmol) and isoamyl nitrite (4.42 ml, 33 mmol) in CH₃CN (125 ml) was treated with CuBr (6.5 g, 45 mmol), added portion-wise, and the reaction was stirred at RT for 4 hours. Silica gel (18 g) was added, the volatiles were removed under reduced pressure, and the crude residue was purified by flash column chromatography hexane/AcOEt 98:2 to 7:3. The brown oil obtained was triturated with pentane to give 1 g of pure crystalline product. ESIMS (m/z) 192.0, 194.2 (MH⁺).

### Example 39

### (S)-2-[7-(5,6-Dihydro-4H-cyclopentathiazol-2-yl-dibenzofuran-3-sulfonylamino]-3-methyl-butyric acid (Compound 306)

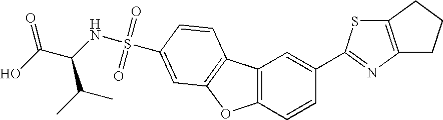

The title compound was prepared by the procedures described in Example 20, using 2-bromo-5,6-dihydro-4H-cyclopenta[d]thiazole (its preparation is described below) instead of 2-bromothiazole. The title compound was obtained as an off-white solid. ¹H NMR (300 MHz, DMSO-d6) δ ppm 12.51 (s, 1H), 8.80 (d, J=1.5 Hz, 1H), 8.50 (d, J=8.2 Hz, 1H), 8.13 (dd, J=8.7, 1.9 Hz, 1H), 8.15 (br. s., 1H), 8.09 (d, J=1.2 Hz, 1H), 7.78-7.94 (m, 2H), 3.56-3.68 (m, 1H), 2.97 (t, J=7.0 Hz, 2H), 2.85 (t, J=7.3 Hz, 2H), 2.43-2.49 (m, 2H), 1.86-2.03 (m, 1H), 0.84 (d, J=6.7 Hz, 3H), 0.82 (d, J=6.7 Hz, 3H). ESIMS (m/z) 471.08 (MH⁺).

### Preparation of the Suzuki synthon 2-bromo-5,6-dihydro-4H-cyclopenta[d]thiazole

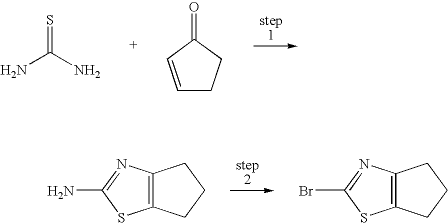

### Step 1: Preparation of 5,6-dihydro-4H-cyclopenta[d]thiazol-2-amine

A mixture of cyclopentanone (8.4 g, 0.1 mol), thiourea (15.22 g, 0.2 mol) and iodine (25.38 g, 0.1 mol) was heated overnight at 100° C., then isopropyl ether was added and the mixture heated at reflux for an additional 30 minutes. The solid was collected via filtration, washed with ether, and then dissolved in hot water. The solution was left to cool to RT, was then basified with concentrated ammonia, and extracted with ethyl acetate. The organic phase was dried over Na₂SO₄ and concentrated under reduced pressure to give the desired product (5.56 g 40% yield). ESIMS (m/z) 141.0 (MH⁺).

### Step 2: Preparation of 2-bromo-5,6-dihydro-4H-cyclopenta[d]thiazole

A solution of 5,6-dihydro-4H-cyclopenta[d]thiazol-2-amine (4 g, 28.5 mmol) and isoamyl nitrite (4.2 ml, 31.4 mmol) in CH₃CN (100 ml) was treated with CuBr (6.14 g, 42.8 mmol), added portion-wise, and the reaction was stirred at RT for 4 hours. Silica gel (15 g) was added, the volatiles were removed under reduced pressure, and the crude residue was purified by flash column chromatography (hexane/AcOEt, 98:2 to 9:1) to afford the desired product (779 mg, 14% yield). ESIMS (m/z): 206.0 (MH⁺).

### Example 40

### (S)-3-methyl-2-(8-(4,5,6,7-tetrahydrobenzo[d]thiazol-2-yl)dibenzo[b,d] furan-3-sulfonamido)butanoic acid (Compound 307)

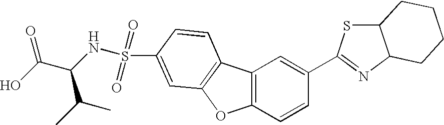

The title compound was prepared by the procedures described in Example 39, using 2-bromo-4,5,6,7-tetrahydrobenzo[d]thiazole instead of 2-bromo-5,6-dihydro-4H-cyclopenta[d] thiazole. The intermediate 2-bromo-4,5,6,7-tetrahydrobenzo[d]thiazole was prepared by the same method of Example 39 using cyclohexanone instead of cyclopentanone. The title compound was obtained as a white solid. ¹H NMR (300 MHz, DMSO-d₆) δppm 12.48 (br. s., 1H), 8.78 (d, J=1.8 Hz, 1H), 8.50 (d, J=8.2 Hz, 1H), 8.16 (d, J=9.5 Hz, 1H), 8.12 (dd, J=8.8, 1.9 Hz, 1H), 8.09 (d, J=1.5 Hz, 1H), 7.87 (d, J=8.8 Hz, 1H), 7.84 (dd, J=8.2, 1.5 Hz, 1H), 3.62 (dd, J=9.5, 6.0 Hz, 1H), 2.75-2.89 (m, 4H), 1.89-2.04 (m, 1H), 1.86 (br. s., 4H), 0.84 (d, J=6.7 Hz, 3H), 0.81 (d, J=6.7 Hz, 3H). ESIMS (m/z) 485.02 (MH⁺).

Crystalline forms of the compounds disclosed herein can be obtained using one or more of the following recrystallization procedures: (a) dissolving the compound in methanol (e.g., 31 mg compound in 0.6 mL methanol) at room temperature, adding water (e.g., 0.5 mL, HPLC grade) to the solution with stirring at room temperature, and isolating the resulting solids by filtration; (b) dissolving the compound in acetone (e.g., 32 mg compound in 0.5 mL acetone) at room temperature, adding heptane (e.g., 1.1 mL) to the solution with stirring at room temperature, and isolating the resulting solids by filtration; (c) dissolving the compound in ethyl acetate (e.g., 54 mg compound in 3 mL ethyl acetate) at room temperature, and evaporating the solvent in a vacuum oven maintained at 50° C.; and (d) dissolving in acetone (e.g., 46 mg compound in 0.5 mL acetone) at 50° C., adding heptane (e.g., 1.0 mL) to the solution with stirring at 50° C., cooling the solution mixture back to room temperature, and isolating the resulting solids by filtration.

### Example 41

### Assay for Pharmacological Activity

### MMP-12 FRET Assay

Compounds according to the present teachings were tested in an MMP-12 FRET assay as follows. To each well of black polystyrene 96-well plate was added assay buffer (50 mM HEPES (pH 7.4), 100 mM NaCl, 5 mM CaCl₂ and 0.005% Brij-35 (Polyoxyethyleneglycol dodecyl ether, Pierce cat#20150), purified human MMP-12 enzyme, and varied concentrations of test compounds (prepared by serial dilution of a stock solution in 100% DMSO). The plates were incubated at room temperature for 30 minutes. The enzymatic reactions were initiated by addition of a substrate, MCA-Pro-Leu-Gly-Leu-Dpa(DNP)-Ala-Arg, containing a fluorescent group (7-methoxycoumarin, MCA) and a 2,4-dinitrophenyl group (DNP), to a final concentration of 20 μM. The final DMSO concentration in the assay was 10%. The reaction was monitored for 30 minutes at room temperature and the initial rate of the cleavage reaction was determined using a fluorescence plate reader (λₑₓ: 325 nm, λₑₘ: 395 nm). Plots of the inhibitor concentration vs. the initial cleavage rate were fit to the following equation: y=Vₘₐₓ*(1−(xⁿ/(Kⁿ+xⁿ))), whereby x=inhibitor concentration, y=initial rate, Vₘₐₓ=initial rate in the absence of inhibitor, n=slope factor, and K=IC₅₀ for the inhibition curve.

The results obtained are summarized in Table 15 below.

TABLE 15

| Cmpd | IC50 | |
|---|---|---|
| No | (nM) | Name |
| 1 | 11 | (R)-2-(8-(4,4-dimethyl-2-oxo-2,4-dihydro-1H-benzo[d][1,3]oxazin-6- |
| | | yl)dibenzo[b,d]furan-3-sulfonamido)-3-methylbutanoic acid |
| 2 | 2.4 | (S)-2-(8-(3-(dimethylamino)prop-1-ynyl)dibenzo[b,d]furan-3- |
| | | sulfonamido)-3-methylbutanoic acid |
| 3 | <1.5 | (S)-3-methyl-2-(8-(pyridin-3-yl)dibenzo[b,d]furan-3- |
| | | sulfonamido)butanoic acid |
| 4 | 12.2 | (S)-2-(8-(4,4-dimethyl-2-oxo-2,4-dihydro-1H-benzo[d][1,3]oxazin-6- |
| | | yl)dibenzo[b,d]furan-3-sulfonamido)-3-methylbutanoic acid |
| 5 | 2.2 | (S)-3-methyl-2-(8-(4-methylthiophen-3-yl)dibenzo[b,d]furan-3- |
| | | sulfonamido)butanoic acid |
| 6 | 30.4 | (S)-2-(8-(3-methoxy-3-oxoprop-1-ynyl)dibenzo[b,d]furan-3- |
| | | sulfonamido)-3-methylbutanoic acid |
| 7 | <1.5 | (S)-2-(8-(furan-3-yl)dibenzo[b,d]furan-3-sulfonamido)-3- |
| | | methylbutanoic acid |
| 8 | <1.5 | (S)-2-(8-(1H-pyrrol-2-yl)dibenzo[b,d]furan-3-sulfonamido)-3- |
| | | methylbutanoic acid |
| 9 | 10.7 | (S)-2-(8-(3,5-dimethylisoxazol-4-yl)dibenzo[b,d]furan-3-sulfonamido)- |
| | | 3-methylbutanoic acid |
| 10 | <1.5 | (S)-2-(8-(6-methoxypyridin-3-yl)dibenzo[b,d]furan-3-sulfonamido)-3- |
| | | methylbutanoic acid |
| 11 | 15.6 | (S)-3-methyl-2-(8-(pyridin-3-yl)dibenzo[b,d]thiophene-3- |
| | | sulfonamido)butanoic acid |
| 12 | <1.5 | (S)-2-(8-(benzo[b]thiophen-3-yl)dibenzo[b,d]furan-3-sulfonamido)-3- |
| | | methylbutanoic acid |
| 13 | 1.7 | (S)-2-(8-(benzo[b]thiophen-2-yl)dibenzo[b,d]furan-3-sulfonamido)-3- |
| | | methylbutanoic acid |
| 14 | <1.5 | (S)-3-methyl-2-(8-(quinolin-6-yl)dibenzo[b,d]furan-3- |
| | | sulfonamido)butanoic acid |
| 15 | <1.5 | (S)-3-methyl-2-(8-((1-methyl-1H-imidazol-5- |
| | | yl)ethynyl)dibenzo[b,d]furan-3-sulfonamido)butanoic acid |
| 16 | <1.5 | (S)-3-methyl-2-(8-(pyridin-4-yl)dibenzo[b,d]furan-3- |
| | | sulfonamido)butanoic acid |
| 17 | <1.5 | (S)-3-methyl-2-(8-(5-methylthiophen-2-yl)dibenzo[b,d]furan-3- |
| | | sulfonamido)butanoic acid |
| 18 | <1.5 | (S)-3-methyl-2-(8-(1-methyl-1H-pyrazol-4-yl)dibenzo[b,d]furan-3- |
| | | sulfonamido)butanoic acid |
| 19 | 44 | (S)-2-(8-(3,5-dimethyl-1H-pyrazol-4-yl)dibenzo[b,d]furan-3- |
| | | sulfonamido)-3-methylbutanoic acid |
| 20 | 4 | (S)-2-(8-(1-isopentyl-1H-pyrazol-4-yl)dibenzo[b,d]furan-3- |
| | | sulfonamido)-3-methylbutanoic acid |
| 21 | <1.5 | (S)-3-methyl-2-(8-(1-propyl-1H-pyrazol-4-yl)dibenzo[b,d]furan-3- |
| | | sulfonamido)butanoic acid |
| 22 | 3 | (S)-2-(8-(1-benzyl-1H-pyrazol-4-yl)dibenzo[b,d]furan-3-sulfonamido)- |
| | | 3-methylbutanoic acid |
| 23 | <1.5 | (S)-2-(8-(1H-pyrazol-4-yl)dibenzo[b,d]furan-3-sulfonamido)-3- |
| | | methylbutanoic acid |
| 24 | 190 | (S)-3-methyl-2-(8-(4-methylthiophen-3-yl)dibenzo[b,d]thiophene-3- |
| | | sulfonamido)butanoic acid |
| 25 | 4.8 | (S)-2-(8-(furan-3-yl)dibenzo[b,d]thiophene-3-sulfonamido)-3- |
| | | methylbutanoic acid |
| 26 | 38 | (S)-3-methyl-2-(8-(thiophen-3-yl)dibenzo[b,d]thiophene-3- |
| | | sulfonamido)butanoic acid |
| 27 | 700 | (S)-2-(8-(3,5-dimethylisoxazol-4-yl)dibenzo[b,d]thiophene-3- |
| | | sulfonamido)-3-methylbutanoic acid |
| 28 | <1.5 | (S)-3-methyl-2-(8-(thiophen-3-yl)dibenzo[b,d]furan-3- |
| | | sulfonamido)butanoic acid |
| 29 | <1.5 | (S)-3-methyl-2-(8-(thiophen-2-yl)dibenzo[b,d]furan-3- |
| | | sulfonamido)butanoic acid |
| 30 | Not | (S)-2-(8-(3-formylfuran-2-yl)dibenzo[b,d]furan-3-sulfonamido)-3- |
| | tested | methylbutanoic acid |
| 31 | Not | (S)-2-(8-(3-formylthiophen-2-yl)dibenzo[b,d]furan-3-sulfonamido)-3- |
| | tested | methylbutanoic acid |
| 32 | <1.5 | (S)-2-(8-(5-acetylthiophen-2-yl)dibenzo[b,d]furan-3-sulfonamido)-3- |
| | | methylbutanoic acid |
| 33 | <1.5 | (S)-3-methyl-2-(8-(4-methylthiophen-2-yl)dibenzo[b,d]furan-3- |
| | | sulfonamido)butanoic acid |
| 34 | <1.5 | (S)-2-(8-(2-chlorothiophen-3-yl)dibenzo[b,d]furan-3-sulfonamido)-3- |
| | | methylbutanoic acid |
| 35 | <1.5 | (S,E)-2-(8-(2-cyclohexylvinyl)dibenzo[b,d]furan-3-sulfonamido)-3- |
| | | methylbutanoic acid |
| 36 | 1.8 | (S)-3-methyl-2-(8-(thiazol-2-yl)dibenzo[b,d]furan-3- |
| | | sulfonamido)butanoic acid |
| 37 | 1.9 | (S)-2-(8-(furan-2-yl)dibenzo[b,d]furan-3-sulfonamido)-3- |
| | | methylbutanoic acid |
| 38 | <1.5 | (S)-2-(8-(methoxyethynyl)dibenzo[b,d]furan-3-sulfonamido)-3- |
| | | methylbutanoic acid |
| 39 | 39 | (S)-2-(8-((diethylamino)ethynyl)dibenzo[b,d]furan-3-sulfonamido)-3- |
| | | methylbutanoic acid |
| 40 | 2.6 | (S)-3-methyl-2-(8-(1-methyl-1H-pyrazol-4-yl)dibenzo[b,d]thiophene- |
| | | 3-sulfonamido)butanoic acid |
| 41 | 77 | (S)-2-(8-(3,5-dimethyl-1H-pyrazol-4-yl)dibenzo[b,d]thiophene-3- |
| | | sulfonamido)-3-methylbutanoic acid |
| 42 | 7.8 | (S)-3-methyl-2-(8-(1-propyl-1H-pyrazol-4-yl)dibenzo[b,d]thiophene-3- |
| | | sulfonamido)butanoic acid |
| 43 | 88 | (S)-2-(8-(1-isopentyl-1H-pyrazol-4-yl)dibenzo[b,d]thiophene-3- |
| | | sulfonamido)-3-methylbutanoic acid |
| 44 | 65 | (S)-2-(8-(1-benzyl-1H-pyrazol-4-yl)dibenzo[b,d]thiophene-3- |
| | | sulfonamido)-3-methylbutanoic acid |
| 45 | 5.5 | (S)-2-(8-(1H-pyrazol-4-yl)dibenzo[b,d]thiophene-3-sulfonamido)-3- |
| | | methylbutanoic acid |
| 46 | 289 | (S)-2-(8-(benzo[b]thiophen-2-yl)dibenzo[b,d]thiophene-3- |
| | | sulfonamido)-3-methylbutanoic acid |
| 47 | 2.9 | (S)-2-(8-(5-acetylthiophen-2-yl)dibenzo[b,d]thiophene-3- |
| | | sulfonamido)-3-methylbutanoic acid |
| 48 | 118 | (S)-2-(8-(3-((dimethylamino)methyl)furan-2-yl)dibenzo[b,d]furan-3- |
| | | sulfonamido)-3-methylbutanoic acid |
| 49 | 228 | (S)-2-(8-(3-((dimethylamino)methyl)thiophen-2-yl)dibenzo[b,d]furan- |
| | | 3-sulfonamido)-3-methylbutanoic acid |
| 50 | 5.7 | (S)-2-(8-(5-(1-(dimethylamino)ethyl)thiophen-2-yl)dibenzo[b,d]furan- |
| | | 3-sulfonamido)-3-methylbutanoic acid |
| 51 | >1000 | (S)-2-(6-(2-chlorothiophen-3-yl)dibenzo[b,d]thiophene-3- |
| | | sulfonamido)-3-methylbutanoic acid |
| 52 | 19 | (S)-2-(8-(2-chlorothiophen-3-yl)dibenzo[b,d]thiophene-3- |
| | | sulfonamido)-3-methylbutanoic acid |
| 53 | 43 | (S)-2-(8-(furan-2-yl)dibenzo[b,d]thiophene-3-sulfonamido)-3- |
| | | methylbutanoic acid |
| 54 | 483 | (S)-2-[8-(6″-Chloro-[2,3′;6′,3″]terpyridin-5-yl)-dibenzothiophene-3- |
| | | sulfonylamino]-3-methyl-butanoic acid |
| 55 | 65 | (S)-2-(8-(6-methoxypyridin-3-yl)dibenzo[b,d]thiophene-3- |
| | | sulfonamido)-3-methylbutanoic acid |
| 56 | 8.4 | (S)-3-methyl-2-(8-(pyridin-4-yl)dibenzo[b,d]thiophene-3- |
| | | sulfonamido)butanoic acid |
| 57 | 21 | (S)-2-(8-(1H-pyrrol-2-yl)dibenzo[b,d]thiophene-3-sulfonamido)-3- |
| | | methylbutanoic acid |
| 58 | 18 | (S,E)-2-(8-(2-cyclohexylvinyl)dibenzo[b,d]thiophene-3-sulfonamido)- |
| | | 3-methylbutanoic acid |
| 59 | <1.5 | (S)-2-(8-(6′-chloro-2,3′-bipyridin-5-yl)dibenzo[b,d]furan-3- |
| | | sulfonamido)-3-methylbutanoic acid |
| 60 | <1.5 | (S)-2-(7-(furan-3-yl)dibenzo[b,d]thiophene-3-sulfonamido)-3- |
| | | methylbutanoic acid |
| 61 | 276 | (S)-2-(8-(6′-chloro-2,3′-bipyridin-5-yl)dibenzo[b,d]thiophene-3- |
| | | sulfonamido)-3-methylbutanoic acid |
| 62 | 44 | (S)-3-methyl-2-(8-(4-methylthiophen-2-yl)dibenzo[b,d]thiophene-3- |
| | | sulfonamido)butanoic acid |
| 63 | 33 | (S)-2-(8-(6-chloropyridin-3-yl)dibenzo[b,d]thiophene-3-sulfonamido)- |
| | | 3-methylbutanoic acid |
| 64 | <1.5 | (S)-2-(8-(6-chloropyridin-3-yl)dibenzo[b,d]furan-3-sulfonamido)-3- |
| | | methylbutanoic acid |
| 65 | 4.6 | (S)-2-(7-(3-methoxyprop-1-ynyl)dibenzo[b,d]thiophene-3- |
| | | sulfonamido)-3-methylbutanoic acid |
| 66 | <1.5 | (S,E)-3-methyl-2-(8-(prop-1-enyl)dibenzo[b,d]furan-3- |
| | | sulfonamido)butanoic acid |
| 67 | <1.5 | (S,Z)-3-methyl-2-(8-(prop-1-enyl)dibenzo[b,d]furan-3- |
| | | sulfonamido)butanoic acid |
| 68 | 13 | (S)-3-methyl-2-(8-(5-((methylamino)methyl)furan-2- |
| | | yl)dibenzo[b,d]furan-3-sulfonamido)butanoic acid |
| 69 | <1.5 | (S)-2-(8-cyclopentenyldibenzo[b,d]furan-3-sulfonamido)-3- |
| | | methylbutanoic acid |
| 70 | 5.4 | (S)-3-methyl-2-(8-(1,2,3,6-tetrahydropyridin-4-yl)dibenzo[b,d]furan-3- |
| | | sulfonamido)butanoic acid |
| 71 | <1.5 | (S)-2-(8-cyclopentyldibenzo[b,d]furan-3-sulfonamido)-3- |
| | | methylbutanoic acid |
| 72 | <1.5 | (S)-3-methyl-2-(8-(5-methylfuran-2-yl)dibenzo[b,d]furan-3- |
| | | sulfonamido)butanoic acid |
| 73 | <1.5 | (S)-2-(8-(5-chlorofuran-2-yl)dibenzo[b,d]furan-3-sulfonamido)-3- |
| | | methylbutanoic acid |
| 74 | <1.5 | (S)-2-(8-(5-chlorothiophen-2-yl)dibenzo[b,d]furan-3-sulfonamido)-3- |
| | | methylbutanoic acid |
| 75 | <1.5 | (S)-2-(8-(3,5-dichlorothiophen-2-yl)dibenzo[b,d]furan-3-sulfonamido)- |
| | | 3-methylbutanoic acid |
| 76 | 18 | (S)-2-(8-(N-isopropylcarbamimidoyl)dibenzo[b,d]furan-3- |
| | | sulfonamido)-3-methylbutanoic acid |
| 77 | 23 | (S)-2-(8-(4,5-dihydro-1H-imidazol-2-yl)dibenzo[b,d]furan-3- |
| | | sulfonamido)-3-methylbutanoic acid |
| 78 | <1.5 | (S)-2-(7-(furan-2-yl)dibenzo[b,d]furan-3-sulfonamido)-3- |
| | | methylbutanoic acid |
| 79 | <1.5 | (S)-2-(7-(furan-3-yl)dibenzo[b,d]furan-3-sulfonamido)-3- |
| | | methylbutanoic acid |
| 80 | <1.5 | (S)-2-(7-(5-chlorofuran-2-yl)dibenzo[b,d]furan-3-sulfonamido)-3- |
| | | methylbutanoic acid |
| 81 | <1.5 | (S)-3-methyl-2-(7-(thiophen-2-yl)dibenzo[b,d]furan-3- |
| | | sulfonamido)butanoic acid |
| 82 | 1.8 | (S)-2-(8-(N-hydroxycarbamimidoyl)dibenzo[b,d]furan-3-sulfonamido)- |
| | | 3-methylbutanoic acid |
| 83 | 2.8 | (S)-2-(8-(4,5-dihydrooxazol-2-yl)dibenzo[b,d]furan-3-sulfonamido)-3- |
| | | methylbutanoic acid |
| 84 | <1.5 | (S)-2-(7-(5-chlorothiophen-2-yl)dibenzo[b,d]furan-3-sulfonamido)-3- |
| | | methylbutanoic acid |
| 85 | <1.5 | (S)-2-(7-(3,5-dichlorothiophen-2-yl)dibenzo[b,d]furan-3-sulfonamido)- |
| | | 3-methylbutanoic acid |
| 86 | <1.5 | (S)-3-methyl-2-(7-(3,4,5-trichlorothiophen-2-yl)dibenzo[b,d]furan-3- |
| | | sulfonamido)butanoic acid |
| 87 | 19 | (S)-3-methyl-2-(8-(N-phenylcarbamimidoyl)dibenzo[b,d]furan-3- |
| | | sulfonamido)butanoic acid |
| 88 | 47 | (S)-2-(8-(N-benzylcarbamimidoyl)dibenzo[b,d]furan-3-sulfonamido)- |
| | | 3-methylbutanoic acid |
| 89 | 2.5 | (S)-2-(8-(2,5-dimethylthiophen-3-yl)dibenzo[b,d]furan-3- |
| | | sulfonamido)-3-methylbutanoic acid |
| 90 | 15 | (R)-2-(7-(3-methoxyprop-1-ynyl)dibenzo[b,d]thiophene-3- |
| | | sulfonamido)-3-methylbutanoic acid |
| 91 | 4 | (S)-3-methyl-2-(8-(5-methyl-1,2,4-oxadiazol-3-yl)dibenzo[b,d]furan-3- |
| | | sulfonamido)butanoic acid |
| 92 | <1.5 | (S)-3-methyl-2-(8-(5-(trifluoromethyl)-1,2,4-oxadiazol-3- |
| | | yl)dibenzo[b,d]furan-3-sulfonamido)butanoic acid |
| 93 | 68 | (S)-2-(8-(1,2,4-oxadiazol-3-yl)dibenzo[b,d]furan-3-sulfonamido)-3- |
| | | methylbutanoic acid |
| 94 | <1.5 | (S)-2-(8-(2-chlorofuran-3-yl)dibenzo[b,d]furan-3-sulfonamido)-3- |
| | | methylbutanoic acid |
| 95 | <1.5 | (S)-2-(8-(2,5-dichlorofuran-3-yl)dibenzo[b,d]furan-3-sulfonamido)-3- |
| | | methylbutanoic acid |
| 96 | 13 | (R)-2-(7-(furan-3-yl)dibenzo[b,d]furan-2-sulfonamido)-3- |
| | | methylbutanoic acid |
| 97 | <1.5 | (R)-3-methyl-2-(7-(thiophen-3-yl)dibenzo[b,d]furan-2- |
| | | sulfonamido)butanoic acid |
| 98 | <1.5 | (R)-2-(7-(furan-2-yl)dibenzo[b,d]furan-2-sulfonamido)-3- |
| | | methylbutanoic acid |
| 99 | 12 | (R)-3-methyl-2-(7-(4-methylthiophen-3-yl)dibenzo[b,d]furan-2- |
| | | sulfonamido)butanoic acid |
| 100 | 4.9 | (R)-2-(7-(benzo[b]thiophen-2-yl)dibenzo[b,d]furan-2-sulfonamido)-3- |
| | | methylbutanoic acid |
| 101 | 5.1 | (R)-2-(7-(6-chloropyridin-3-yl)dibenzo[b,d]furan-2-sulfonamido)-3- |
| | | methylbutanoic acid |
| 102 | <1.5 | (R)-2-(7-(6-methoxypyridin-3-yl)dibenzo[b,d]furan-2-sulfonamido)-3- |
| | | methylbutanoic acid |
| 103 | 16 | (R)-2-(7-(1H-pyrazol-4-yl)dibenzo[b,d]furan-2-sulfonamido)-3- |
| | | methylbutanoic acid |
| 104 | 59 | (R,E)-2-(7-(2-cyclohexylvinyl)dibenzo[b,d]furan-2-sulfonamido)-3- |
| | | methylbutanoic acid |
| 105 | 21.8 | (R)-2-(7-(5-acetylthiophen-2-yl)dibenzo[b,d]furan-2-sulfonamido)-3- |
| | | methylbutanoic acid |
| 106 | 400 | (S)-2-(8-(N,N-diethylcarbamimidoyl)dibenzo[b,d]furan-3- |
| | | sulfonamido)-3-methylbutanoic acid |
| 107 | 21 | (S)-2-(8-(4,5-dihydrothiazol-2-yl)dibenzo[b,d]furan-3-sulfonamido)-3- |
| | | methylbutanoic acid |
| 108 | 2 | (S)-2-(8-(N-methoxycarbamimidoyl)dibenzo[b,d]furan-3- |
| | | sulfonamido)-3-methylbutanoic acid |
| 109 | 640 | (S)-2-(8-(N,N′-diethylcarbamimidoyl)dibenzo[b,d]furan-3- |
| | | sulfonamido)-3-methylbutanoic acid |
| 110 | 425 | (S)-2-(8-(N-isopropyl-N-methylcarbamimidoyl)dibenzo[b,d]furan-3- |
| | | sulfonamido)-3-methylbutanoic acid |
| 111 | <1.5 | (S)-2-(8-(5-carbamoylthiophen-2-yl)dibenzo[b,d]furan-3- |
| | | sulfonamido)-3-methylbutanoic acid |
| 112 | 52 | (S)-5-(7-(N-(1-carboxy-2-methylpropyl)sulfamoyl)dibenzo[b,d]furan- |
| | | 2-yl)thiophene-2-carboxylic acid |
| 113 | 7.2 | (2S)-2-[8-(5-tert-Butyl-[1,2,4]oxadiazol-3-yl)-dibenzofuran-3- |
| | | sulfonylamino]-3-methyl-butanoic acid |
| 114 | 2.7 | (2S)-2-[8-(5-Isopropyl-[1,2,4]oxadiazol-3-yl)-dibenzofuran-3- |
| | | sulfonylamino]-3-methyl-butanoic acid |
| 115 | 290 | (R)-2-(7-(2,4-dimethoxypyrimidin-5-yl)dibenzo[b,d]furan-2- |
| | | sulfonamido)-3-methylbutanoic acid |
| 116 | 3.1 | (R)-2-(7-(1H-pyrrol-2-yl)dibenzo[b,d]furan-2-sulfonamido)-3- |
| | | methylbutanoic acid |
| 117 | 2.8 | (R)-3-methyl-2-(7-(1-methyl-1H-pyrazol-4-yl)dibenzo[b,d]furan-2- |
| | | sulfonamido)butanoic acid |
| 118 | 0.6 | (R)-3-methyl-2-(7-(thiophen-2-yl)dibenzo[b,d]furan-2- |
| | | sulfonamido)butanoic acid |
| 119 | 8.3 | (R)-2-(7-(benzofuran-2-yl)dibenzo[b,d]furan-2-sulfonamido)-3- |
| | | methylbutanoic acid |
| 120 | 2.2 | (R)-3-methyl-2-(7-(4-(trifluoromethyl)phenyl)dibenzo[b,d]furan-2- |
| | | sulfonamido)butanoic acid |
| 121 | 140 | (R)-3-methyl-2-(7-(1-methyl-1H-indol-2-yl)dibenzo[b,d]furan-2- |
| | | sulfonamido)butanoic acid |
| 122 | 6.9 | (R)-2-(7-(5-fluoro-1H-indol-2-yl)dibenzo[b,d]furan-2-sulfonamido)-3- |
| | | methylbutanoic acid |
| 123 | 2.9 | (2S)-2-[8-(5-Ethyl-[1,2,4]oxadiazol-3-yl)-dibenzofuran-3- |
| | | sulfonylamino]-3-methyl-butanoic acid |
| 124 | <1.5 | (S)-2-(8-(5-fluorothiophen-2-yl)dibenzo[b,d]furan-3-sulfonamido)-3- |
| | | methylbutanoic acid |
| 125 | 17 | (2S,2′S)-2,2′-[2,2′-bidibenzo[b,d]furan-7,7′- |
| | | diylbis(sulfonylimino)]bis(3-methylbutanoic acid |
| 126 | <1.5 | (S)-3-methyl-2-(8-(4-(trifluoromethyl)thiazol-2-yl)dibenzo[b,d]furan-3- |
| | | sulfonamido)butanoic acid |
| 127 | 175 | (S)-2-(8-(imino(pyrrolidin-1-yl)methyl)dibenzo[b,d]furan-3- |
| | | sulfonamido)-3-methylbutanoic acid |
| 128 | 73 | (S)-2-(8-(N-ethylcarbamimidoyl)dibenzo[b,d]furan-3-sulfonamido)-3- |
| | | methylbutanoic acid |
| 129 | <1.5 | (S)-2-(7-(furan-2-yl)dibenzo[b,d]thiophene-3-sulfonamido)-3- |
| | | methylbutanoic acid |
| 130 | 1900 | (S)-2-(8-(2H-tetrazol-5-yl)dibenzo[b,d]furan-3-sulfonamido)-3- |
| | | methylbutanoic acid |
| 131 | <1.5 | (S)-3-methyl-2-(8-(5-(trifluoromethyl)thiophen-2-yl)dibenzo[b,d]furan- |
| | | 3-sulfonamido)butanoic acid |
| 132 | 2 | (S)-3-methyl-2-(8-(2-methyl-2H-tetrazol-5-yl)dibenzo[b,d]furan-3- |
| | | sulfonamido)butanoic acid |
| 133 | 8 | (R)-2-(7-(5-tert-butyl-1,2,4-oxadiazol-3-yl)dibenzo[b,d]furan-2- |
| | | sulfonamido)-3-methylbutanoic acid |
| 134 | 2.5 | (S)-2-(8-(3,5-dichlorofuran-2-yl)dibenzo[b,d]furan-3-sulfonamido)-3- |
| | | methylbutanoic acid |
| 135 | <1.5 | (S)-3-methyl-2-(7-(5-methylfuran-2-yl)dibenzo[b,d]furan-3- |
| | | sulfonamido)butanoic acid |
| 136 | <1.5 | (S)-2-(7-(benzo[b]thiophen-2-yl)dibenzo[b,d]furan-3-sulfonamido)-3- |
| | | methylbutanoic acid |
| 137 | 11 | (S)-3-methyl-2-(7-(thiazol-2-yl)dibenzo[b,d]furan-3- |
| | | sulfonamido)butanoic acid |
| 138 | 2.8 | (R)-2-(7-(5-isopropyl-1,2,4-oxadiazol-3-yl)dibenzo[b,d]furan-2- |
| | | sulfonamido)-3-methylbutanoic acid |
| 139 | 9.2 | (R)-3-methyl-2-(7-(5-methyl-1,2,4-oxadiazol-3-yl)dibenzo[b,d]furan- |
| | | 2-sulfonamido)butanoic acid |
| 140 | 3.8 | (R)-2-(7-(5-ethyl-1,2,4-oxadiazol-3-yl)dibenzo[b,d]furan-2- |
| | | sulfonamido)-3-methylbutanoic acid |
| 141 | 14 | (R)-3-methyl-2-(7-(5-(trifluoromethyl)-1,2,4-oxadiazol-3- |
| | | yl)dibenzo[b,d]furan-2-sulfonamido)butanoic acid |
| 142 | 2.5 | (S)-3-methyl-2-(7-(5-methylthiophen-2-yl)dibenzo[b,d]furan-3- |
| | | sulfonamido)butanoic acid |
| 143 | 1.1 | (S)-2-(7-(benzofuran-2-yl)dibenzo[b,d]furan-3-sulfonamido)-3- |
| | | methylbutanoic acid |
| 144 | 2.2 | (R)-2-(7-(5-bromothiophen-2-yl)dibenzo[b,d]furan-2-sulfonamido)-3- |
| | | methylbutanoic acid |
| 145 | 29 | (R)-2-(7-(3,5-dimethylisoxazol-4-yl)dibenzo[b,d]furan-2- |
| | | sulfonamido)-3-methylbutanoic acid |
| 146 | 1.7 | (S)-3-methyl-2-(8-(5-methyl-1,3,4-thiadiazol-2-yl)dibenzo[b,d]furan-3- |
| | | sulfonamido)butanoic acid |
| 147 | <1.5 | (R)-2-(7-(5-cyclopropyl-1,2,4-oxadiazol-3-yl)dibenzo[b,d]furan-2- |
| | | sulfonamido)-3-methylbutanoic acid |
| 148 | <1.5 | (R)-2-(7-(5-cyclobutyl-1,2,4-oxadiazol-3-yl)dibenzo[b,d]furan-2- |
| | | sulfonamido)-3-methylbutanoic acid |
| 149 | 5.3 | (R)-2-(7-(5-isobutyl-1,2,4-oxadiazol-3-yl)dibenzo[b,d]furan-2- |
| | | sulfonamido)-3-methylbutanoic acid |
| 150 | 5 | (R)-3-methyl-2-(7-(5-phenyl-1,2,4-oxadiazol-3-yl)dibenzo[b,d]furan- |
| | | 2-sulfonamido)butanoic acid |
| 151 | <1.5 | (S)-2-(8-(benzo[d]thiazol-2-yl)dibenzo[b,d]furan-3-sulfonamido)-3- |
| | | methylbutanoic acid |
| 152 | <1.5 | (S)-3-methyl-2-(8-(pyrimidin-5-yl)dibenzo[b,d]furan-3- |
| | | sulfonamido)butanoic acid |
| 153 | <1.5 | (S)-2-(8-(2-methoxypyrimidin-5-yl)dibenzo[b,d]furan-3-sulfonamido)- |
| | | 3-methylbutanoic acid |
| 154 | 32 | (S)-2-(7-(5-isopropyl-1,2,4-oxadiazol-3-yl)dibenzo[b,d]furan-3- |
| | | sulfonamido)-3-methylbutanoic acid |
| 155 | 11 | (S)-2-(7-(5-tert-buty-1,2,4-oxadiazol-3-yl)dibenzo[b,d]furan-3- |
| | | sulfonamido)-3-methylbutanoic acid |
| 156 | 6.9 | (S)-3-methyl-2-(7-(5-methyl-1,2,4-oxadiazol-3-yl)dibenzo[b,d]furan-3- |
| | | sulfonamido)butanoic acid |
| 157 | 15 | (2S)-3-methyl-2-(8-(1-(2-methylbutyl)-1H-pyrazol-4- |
| | | yl)dibenzo[b,d]furan-3-sulfonamido)butanoic acid |
| 158 | 223 | (S)-3-methyl-2-(8-(1-(2-morpholinoethyl)-1H-pyrazol-4- |
| | | yl)dibenzo[b,d]furan-3-sulfonamido)butanoic acid |
| 159 | 6.2 | (S)-2-(8-(1-isobutyl-1H-pyrazol-4-yl)dibenzo[b,d]furan-3- |
| | | sulfonamido)-3-methylbutanoic acid |
| 160 | 22 | (S)-3-methyl-2-(8-(1,3,5-trimethyl-1H-pyrazol-4-yl)dibenzo[b,d]furan- |
| | | 3-sulfonamido)butanoic acid |
| 161 | 82 | (S)-3-methyl-2-(8-(5-methyl-3-phenylisoxazol-4-yl)dibenzo[b,d]furan- |
| | | 3-sulfonamido)butanoic acid |
| 162 | <1.5 | (S)-3-methyl-2-(8-(5-methyl-1-phenyl-1H-pyrazol-4- |
| | | yl)dibenzo[b,d]furan-3-sulfonamido)butanoic acid |
| 163 | 170 | (S)-3-methyl-2-(8-(4-methyl-2-phenylthiazol-5-yl)dibenzo[b,d]furan-3- |
| | | sulfonamido)butanoic acid |
| 164 | 12.3 | (S)-3-methyl-2-(8-(4-methyl-2-(4-(trifluoromethyl)phenyl)thiazol-5- |
| | | yl)dibenzo[b,d]furan-3-sulfonamido)butanoic acid |
| 165 | 1.5 | (S)-2-(7-(4-bromo-5-ethylthiophen-2-yl)dibenzo[b,d]furan-3- |
| | | sulfonamido)-3-methylbutanoic acid |
| 166 | 93 | (S)-2-(7-(2′,5-diethyl-2,3′-bithiophen-5′-yl)dibenzo[b,d]furan-3- |
| | | sulfonamido)-3-methylbutanoic acid |
| 167 | <1.5 | (R)-3-methyl-2-(7-(pyrimidin-5-yl)dibenzo[b,d]furan-2- |
| | | sulfonamido)butanoic acid |
| 168 | 1.5 | (R)-2-(7-(2-methoxypyrimidin-5-yl)dibenzo[b,d]furan-2-sulfonamido)- |
| | | 3-methylbutanoic acid |
| 169 | 11 | (R)-2-(7-(2,4-dimethylthiazol-5-yl)dibenzo[b,d]furan-2-sulfonamido)- |
| | | 3-methylbutanoic acid |
| 170 | 5.2 | (2R)-3-methyl-2-(7-(1-(2-methylbutyl)-1H-pyrazol-4- |
| | | yl)dibenzo[b,d]furan-2-sulfonamido)butanoic acid |
| 171 | 3.9 | (R)-3-methyl-2-(7-(1-propyl-1H-pyrazol-4-yl)dibenzo[b,d]furan-2- |
| | | sulfonamido)butanoic acid |
| 172 | 19 | (R)-3-methyl-2-(7-(1-(2-morpholinoethyl)-1H-pyrazol-4- |
| | | yl)dibenzo[b,d]furan-2-sulfonamido)butanoic acid |
| 173 | 80 | (R)-2-(7-(1-isobutyl-1H-pyrazol-4-yl)dibenzo[b,d]furan-2- |
| | | sulfonamido)-3-methylbutanoic acid |
| 174 | 227 | (R)-3-methyl-2-(7-(1,3,5-trimethyl-1H-pyrazol-4-yl)dibenzo[b,d]furan- |
| | | 2-sulfonamido)butanoic acid |
| 175 | 12 | (R)-2-(7-(1-benzyl-1H-pyrazol-4-yl)dibenzo[b,d]furan-2-sulfonamido)- |
| | | 3-methylbutanoic acid |
| 176 | 97 | (R)-3-methyl-2-(7-(4-methyl-2-phenylthiazol-5-yl)dibenzo[b,d]furan-2- |
| | | sulfonamido)butanoic acid |
| 177 | 740 | (R)-3-methyl-2-(7-(4-methyl-2-(4-(trifluoromethyl)phenyl)thiazol-5- |
| | | yl)dibenzo[b,d]furan-2-sulfonamido)butanoic acid |
| 178 | <1.5 | (R)-2-(8-(5-chlorofuran-2-yl)dibenzo[b,d]furan-3-sulfonamido)-3- |
| | | methylbutanoic acid |
| 179 | <1.5 | (S)-2-(8-(2-chlorothiazol-5-yl)dibenzo[b,d]furan-3-sulfonamido)-3- |
| | | methylbutanoic acid |
| 180 | <1.5 | (S)-2-(8-(2-chlorothiazol-4-yl)dibenzo[b,d]furan-3-sulfonamido)-3- |
| | | methylbutanoic acid |
| 181 | <1.5 | (S)-2-(7-(2-chlorothiazol-5-yl)dibenzo[b,d]furan-3-sulfonamido)-3- |
| | | methylbutanoic acid |
| 182 | <1.5 | (S)-2-(7-(5-chlorofuran-2-yl)dibenzo[b,d]thiophene-3-sulfonamido)-3- |
| | | methylbutanoic acid |
| 183 | | Absent |
| 184 | | Absent |
| 185 | 2 | (R)-3-methyl-2-(8-(5-(trifluoromethyl)-1,2,4-oxadiazol-3- |
| | | yl)dibenzo[b,d]furan-3-sulfonamido)butanoic acid |
| 186 | 1.9 | (S)-2-(7-(N-hydroxycarbamimidoyl)dibenzo[b,d]furan-3-sulfonamido)- |
| | | 3-methylbutanoic acid |
| 187 | 5.5 | (S)-2-(7-(5-cyclopropyl-1,2,4-oxadiazol-3-yl)dibenzo[b,d]furan-3- |
| | | sulfonamido)-3-methylbutanoic acid |
| 188 | 8.6 | (S)-2-(7-(5-(4-fluorophenyl)-1,2,4-oxadiazol-3-yl)dibenzo[b,d]furan-3- |
| | | sulfonamido)-3-methylbutanoic acid |
| 189 | 4.5 | (R)-3-methyl-2-(7-(5-neopentyl-1,2,4-oxadiazol-3- |
| | | yl)dibenzo[b,d]furan-2-sulfonamido)butanoic acid |
| 190 | 2.2 | (R)-2-(7-(5-cyclopentyl-1,2,4-oxadiazol-3-yl)dibenzo[b,d]furan-2- |
| | | sulfonamido)-3-methylbutanoic acid |
| 191 | 3.3 | (R)-2-(7-(5-(cyclopentylmethyl)-1,2,4-oxadiazol-3- |
| | | yl)dibenzo[b,d]furan-2-sulfonamido)-3-methylbutanoic acid |
| 192 | 7.2 | (R)-2-(7-(5-cyclohexyl-1,2,4-oxadiazol-3-yl)dibenzo[b,d]furan-2- |
| | | sulfonamido)-3-methylbutanoic acid |
| 193 | <1.5 | (S)-2-(7-(furan-2-yl)dibenzo[b,d]thiophene-3-sulfonamido)-3- |
| | | methylbutanoic acid |
| 194 | 4.6 | (S)-2-(8-(benzo[d]oxazol-2-yl)dibenzo[b,d]furan-3-sulfonamido)-3- |
| | | methylbutanoic acid |
| 195 | 7.3 | (S)-2-(2,2′-bidibenzo[b,d]furan-7-sulfonamido)-3-methylbutanoic acid |
| 196 | <1.5 | (S)-2-(8-(5-ethylthiophen-2-yl)dibenzo[b,d]furan-3-sulfonamido)-3- |
| | | methylbutanoic acid |
| 197 | <1.5 | (S)-3-methyl-2-(8-(5-propylthiophen-2-yl)dibenzo[b,d]furan-3- |
| | | sulfonamido)butanoic acid |
| 198 | 5.6 | (S)-2-(8-(5-tert-butylfuran-2-yl)dibenzo[b,d]furan-3-sulfonamido)-3- |
| | | methylbutanoic acid |
| 199 | <1.5 | (S)-3-methyl-2-(8-(5-(5-methyl-1,2,4-oxadiazol-3-yl)thiophen-2- |
| | | yl)dibenzo[b,d]furan-3-sulfonamido)butanoic acid |
| 200 | <1.5 | (S)-2-(8-(5-chloro-4-(trifluoromethyl)thiazol-2-yl)dibenzo[b,d]furan-3- |
| | | sulfonamido)-3-methylbutanoic acid |
| 201 | <1.5 | (S)-2-(8-(2,4-dimethylthiazol-5-yl)dibenzo[b,d]furan-3-sulfonamido)- |
| | | 3-methylbutanoic acid |
| 202 | <1.5 | (S)-3-methyl-2-(8-(2-methylthiazol-5-yl)dibenzo[b,d]furan-3- |
| | | sulfonamido)butanoic acid |
| 203 | 4.1 | (S)-2-(8-(6-chlorobenzo[d]thiazol-2-yl)dibenzo[b,d]furan-3- |
| | | sulfonamido)-3-methylbutanoic acid |
| 204 | 13 | (S)-2-(8-(2-isobutyl-4-methylthiazol-5-yl)dibenzo[b,d]furan-3- |
| | | sulfonamido)-3-methylbutanoic acid |
| 205 | | Absent |
| 206 | 110 | (S)-3-methyl-2-(8-(5-phenyl-3-(trifluoromethyl)-1H-pyrazol-4- |
| | | yl)dibenzo[b,d]furan-3-sulfonamido)butanoic acid |
| 207 | 8.4 | (S)-2-(8-(5-(1H-tetrazol-5-yl)thiophen-2-yl)dibenzo[b,d]furan-3- |
| | | sulfonamido)-3-methylbutanoic acid |
| 208 | 2.7 | (S)-2-(8-(6-methoxybenzo[d]thiazol-2-yl)dibenzo[b,d]furan-3- |
| | | sulfonamido)-3-methylbutanoic acid |
| 209 | <1.5 | (S)-2-(8-(6-fluorobenzo[d]thiazol-2-yl)dibenzo[b,d]furan-3- |
| | | sulfonamido)-3-methylbutanoic acid |
| 210 | 12 | (S)-3-methyl-2-(8-(6-methylbenzo[d]thiazol-2-yl)dibenzo[b,d]furan-3- |
| | | sulfonamido)butanoic acid |
| 211 | 295 | (S)-2-(8-(5-(isoxazol-5-yl)thiophen-2-yl)dibenzo[b,d]furan-3- |
| | | sulfonamido)-3-methylbutanoic acid |
| 212 | 86 | (S)-3-methyl-2-(8-(5-((4-methylpiperazin-1-yl)methyl)thiazol-2- |
| | | yl)dibenzo[b,d]furan-3-sulfonamido)butanoic acid |
| 213 | 40 | (S)-2-(8-(5-(((cyclopropylmethyl)(propyl)amino)methyl)thiazol-2- |
| | | yl)dibenzo[b,d]furan-3-sulfonamido)-3-methylbutanoic acid |
| 214 | 3.7 | (S)-2-(8-(5-((1H-pyrazol-1-yl)methyl)thiazol-2-yl)dibenzo[b,d]furan-3- |
| | | sulfonamido)-3-methylbutanoic acid |
| 215 | 2.4 | (S)-2-(8-(5-(hydroxymethyl)thiazol-2-yl)dibenzo[b,d]furan-3- |
| | | sulfonamido)-3-methylbutanoic acid |
| 216 | 4.5 | (S)-2-(8-(5-(isoxazol-3-yl)thiophen-2-yl)dibenzo[b,d]furan-3- |
| | | sulfonamido)-3-methylbutanoic acid |
| 217 | <1.5 | (S)-2-(8-(4-bromothiazol-2-yl)dibenzo[b,d]furan-3-sulfonamido)-3- |
| | | methylbutanoic acid |
| 218 | <1.5 | (S)-2-(8-(4-fluorobenzo[d]thiazol-2-yl)dibenzo[b,d]furan-3- |
| | | sulfonamido)-3-methylbutanoic acid |
| 219 | <1.5 | (S)-2-(8-(5-fluorobenzo[d]thiazol-2-yl)dibenzo[b,d]furan-3- |
| | | sulfonamido)-3-methylbutanoic acid |
| 220 | <1.5 | (S)-2-(8-(5,6-difluorobenzo[d]thiazol-2-yl)dibenzo[b,d]furan-3- |
| | | sulfonamido)-3-methylbutanoic acid |
| 221 | 1.7 | (S)-3-methyl-2-(8-(6-(trifluoromethoxy)benzo[d]thiazol-2- |
| | | yl)dibenzo[b,d]furan-3-sulfonamido)butanoic acid |
| 222 | 4.9 | (S)-3-methyl-2-(8-(4,5,6-trifluorobenzo[d]thiazol-2- |
| | | yl)dibenzo[b,d]furan-3-sulfonamido)butanoic acid |
| 223 | 13 | (S)-2-(8-(4-methoxybenzo[d]thiazol-2-yl)dibenzo[b,d]furan-3- |
| | | sulfonamido)-3-methylbutanoic acid |
| 224 | <1.5 | (S)-2-(8-(5-chlorothiazol-2-yl)dibenzo[b,d]furan-3-sulfonamido)-3- |
| | | methylbutanoic acid |
| 225 | <1.5 | (S)-2-(8-(5-methoxybenzo[d]thiazol-2-yl)dibenzo[b,d]furan-3- |
| | | sulfonamido)-3-methylbutanoic acid |
| 226 | 1.7 | (S)-2-(7-(benzo[d]thiazol-2-yl)dibenzo[b,d]furan-3-sulfonamido)-3- |
| | | methylbutanoic acid |
| 227 | 6.1 | (S)-2-(7-(benzo[d]oxazol-2-yl)dibenzo[b,d]furan-3-sulfonamido)-3- |
| | | methylbutanoic acid |
| 228 | <1.5 | (S)-3-methyl-2-(7-(5-(5-methyl-1,2,4-oxadiazol-3-yl)thiazol-2- |
| | | yl)dibenzo[b,d]furan-3-sulfonamido)butanoic acid |
| 229 | <1.5 | (S)-2-(7-(5-ethylthiophen-2-yl)dibenzo[b,d]furan-3-sulfonamido)-3- |
| | | methylbutanoic acid |
| 230 | <1.5 | (S)-2-(7-(2,4-dimethylthiazol-5-yl)dibenzo[b,d]furan-3-sulfonamido)- |
| | | 3-methylbutanoic acid |
| 231 | 3.4 | (S)-2-(7-(5-tert-butylfuran-2-yl)dibenzo[b,d]furan-3-sulfonamido)-3- |
| | | methylbutanoic acid |
| 232 | <1.5 | (S)-3-methyl-2-(7-(5-propylthiophen-2-yl)dibenzo[b,d]furan-3- |
| | | sulfonamido)butanoic acid |
| 233 | <1.5 | (S)-2-(7-(5-chloro-4-(trifluoromethyl)thiazol-2-yl)dibenzo[b,d]furan-3- |
| | | sulfonamido)-3-methylbutanoic acid |
| 234 | <1.5 | (S)-3-methyl-2-(7-(5-methylthiazol-2-yl)dibenzo[b,d]furan-3- |
| | | sulfonamido)butanoic acid |
| 235 | 11 | (S)-2-(7-(2-isobutyl-4-methylthiazol-5-yl)dibenzo[b,d]furan-3- |
| | | sulfonamido)-3-methylbutanoic acid |
| 236 | 12 | (S)-3-methyl-2-(7-(6-(trifluoromethyl)benzo[d]thiazol-2- |
| | | yl)dibenzo[b,d]furan-3-sulfonamido)butanoic acid |
| 237 | <1.5 | (S)-2-(7-(6-fluorobenzo[d]thiazol-2-yl)dibenzo[b,d]furan-3- |
| | | sulfonamido)-3-methylbutanoic acid |
| 238 | | Absent |
| 239 | <1.5 | (R)-2-(7-(5-ethylthiophen-2-yl)dibenzo[b,d]furan-2-sulfonamido)-3- |
| | | methylbutanoic acid |
| 240 | 19 | (R)-2-(7-(5-tert-butylfuran-2-yl)dibenzo[b,d]furan-2-sulfonamido)-3- |
| | | methylbutanoic acid |
| 241 | 170 | (S)-2-(7-(5-tert-butylfuran-2-yl)dibenzo[b,d]furan-2-sulfonamido)-3- |
| | | methylbutanoic acid |
| 242 | 8.4 | (S)-2-(7-(5-ethylthiophen-2-yl)dibenzo[b,d]furan-2-sulfonamido)-3- |
| | | methylbutanoic acid |
| 243 | 1.5 | (R)-3-methyl-2-(7-(5-propylthiophen-2-yl)dibenzo[b,d]furan-2- |
| | | sulfonamido)butanoic acid |
| 244 | 3.4 | (R)-2-(7-(2-isobutylthiazol-5-yl)dibenzo[b,d]furan-2-sulfonamido)-3- |
| | | methylbutanoic acid |
| 245 | 13 | (R)-2-(7-(2-isobutyl-4-methylthiazol-5-yl)dibenzo[b,d]furan-2- |
| | | sulfonamido)-3-methylbutanoic acid |
| 246 | 6.2 | (S)-3-methyl-2-(7-(5-propylthiophen-2-yl)dibenzo[b,d]furan-2- |
| | | sulfonamido)butanoic acid |
| 247 | 131 | (S)-2-(7-(2-isobutyl-4-methylthiazol-5-yl)dibenzo[b,d]furan-2- |
| | | sulfonamido)-3-methylbutanoic acid |
| 248 | <1.5 | (S)-2-(8-(2-isobutylthiazol-5-yl)dibenzo[b,d]furan-3-sulfonamido)-3- |
| | | methylbutanoic acid |
| 249 | 7.8 | (S)-2-(7-(2-isobutylthiazol-5-yl)dibenzo[b,d]furan-3-sulfonamido)-3- |
| | | methylbutanoic acid |
| 250 | 239 | (S)-2-(7-(1H-tetrazol-5-yl)dibenzo[b,d]furan-3-sulfonamido)-3- |
| | | methylbutanoic acid |
| 251 | 17 | 2-(8-(thiazol-2-yl)dibenzo[b,d]furan-3-sulfonamido)acetic acid |
| 252 | 12 | (S)-2-(8-(5-tert-butyl-1,2,4-oxadiazol-3-yl)dibenzo[b,d]furan-3- |
| | | sulfonamido)-4-methylpentanoic acid |
| 253 | 17 | (R)-2-(8-(5-tert-butyl-1,2,4-oxadiazol-3-yl)dibenzo[b,d]furan-3- |
| | | sulfonamido)-4-methylpentanoic acid |
| 254 | 21 | (S)-2-(8-(5-tert-butyl-1,2,4-oxadiazol-3-yl)dibenzo[b,d]furan-3- |
| | | sulfonamido)-2-phenylacetic acid |
| 255 | 21 | (R)-2-(8-(5-tert-butyl-1,2,4-oxadiazol-3-yl)dibenzo[b,d]furan-3- |
| | | sulfonamido)-2-phenylacetic acid |
| 256 | 129 | (R)-2-(8-(5-tert-butyl-1,2,4-oxadiazol-3-yl)dibenzo[b,d]furan-3- |
| | | sulfonamido)-3-(1H-indol-3-yl)propanoic acid |
| 257 | 9.1 | (S)-2-(8-(5-tert-butyl-1,2,4-oxadiazol-3-yl)dibenzo[b,d]furan-3- |
| | | sulfonamido)-3,3-dimethylbutanoic acid |
| 258 | 15 | (R)-2-(8-(5-tert-butyl-1,2,4-oxadiazol-3-yl)dibenzo[b,d]furan-3- |
| | | sulfonamido)-3-methylbutanoic acid |
| 259 | 1.6 | (S)-2-(8-(5-cyclopropyl-1,2,4-oxadiazol-3-yl)dibenzo[b,d]furan-3- |
| | | sulfonamido)-3-methylbutanoic acid |
| 260 | 1.7 | (S)-3-methyl-2-(8-(5-(tetrahydro-2H-pyran-4-yl)-1,2,4-oxadiazol-3- |
| | | yl)dibenzo[b,d]furan-3-sulfonamido)butanoic acid |
| 261 | 5.4 | (S)-3-methyl-2-(8-(5-neopentyl-1,2,4-oxadiazol-3- |
| | | yl)dibenzo[b,d]furan-3-sulfonamido)butanoic acid |
| 262 | <1.5 | (S)-2-(8-(5-cyclobutyl-1,2,4-oxadiazol-3-yl)dibenzo[b,d]furan-3- |
| | | sulfonamido)-3-methylbutanoic acid |
| 263 | 2.5 | (S)-2-(8-(5-cyclopentyl-1,2,4-oxadiazol-3-yl)dibenzo[b,d]furan-3- |
| | | sulfonamido)-3-methylbutanoic acid |
| 264 | <1.5 | (S)-3-methyl-2-(8-(5-(thiophen-2-yl)-1,2,4-oxadiazol-3- |
| | | yl)dibenzo[b,d]furan-3-sulfonamido)butanoic acid |
| 265 | 2.8 | (S)-3-methyl-2-(8-(5-phenyl-1,2,4-oxadiazol-3-yl)dibenzo[b,d]furan-3- |
| | | sulfonamido)butanoic acid |
| 266 | 4 | (S)-2-(8-(5-benzyl-1,2,4-oxadiazol-3-yl)dibenzo[b,d]furan-3- |
| | | sulfonamido)-3-methylbutanoic acid |
| 267 | 1.9 | (S)-2-(8-(5-(methoxymethyl)-1,2,4-oxadiazol-3-yl)dibenzo[b,d]furan- |
| | | 3-sulfonamido)-3-methylbutanoic acid |
| 268 | <1.5 | (2S)-3-methyl-2-(8-(5-(tetrahydrofuran-3-yl)-1,2,4-oxadiazol-3- |
| | | yl)dibenzo[b,d]furan-3-sulfonamido)butanoic acid |
| 269 | <1.5 | (S)-2-(8-(5-(2,4-difluorophenyl)-1,2,4-oxadiazol-3- |
| | | yl)dibenzo[b,d]furan-3-sulfonamido)-3-methylbutanoic acid |
| 270 | <1.5 | (S)-2-(8-(5-(2,4-dichlorophenyl)-1,2,4-oxadiazol-3- |
| | | yl)dibenzo[b,d]furan-3-sulfonamido)-3-methylbutanoic acid |
| 271 | 3.7 | (S)-3-methyl-2-(8-(5-(4-(trifluoromethyl)phenyl)-1,2,4-oxadiazol-3- |
| | | yl)dibenzo[b,d]furan-3-sulfonamido)butanoic acid |
| 272 | <1.5 | (S)-2-(8-(5-(4-fluorophenyl)-1,2,4-oxadiazol-3-yl)dibenzo[b,d]furan- |
| | | 3-sulfonamido)-3-methylbutanoic acid |
| 273 | 210 | (S)-7-(N-(1-carboxy-2-methylpropyl)sulfamoyl)dibenzo[b,d]furan-2- |
| | | carboxylic acid |
| 274 | 60 | 2-(7-(5-tert-butyl-1,2,4-oxadiazol-3-yl)dibenzo[b,d]furan-2- |
| | | sulfonamido)acetic acid |
| 275 | 4.1 | (R)-2-(7-(5-tert-butyl-1,2,4-oxadiazol-3-yl)dibenzo[b,d]furan-2- |
| | | sulfonamido)-3-phenylpropanoic acid |
| 276 | 34 | (S)-2-(7-(5-tert-butyl-1,2,4-oxadiazol-3-yl)dibenzo[b,d]furan-2- |
| | | sulfonamido)-3-methylbutanoic acid |
| 277 | 130 | 2-(7-(5-tert-butyl-1,2,4-oxadiazol-3-yl)dibenzo[b,d]furan-2- |
| | | sulfonamido)-2-methylpropanoic acid |
| 278 | 7.2 | (R)-2-(7-(5-tert-butyl-1,2,4-oxadiazol-3-yl)dibenzo[b,d]furan-2- |
| | | sulfonamido)-4-methylpentanoic acid |
| 279 | 61 | (S)-2-(7-(5-tert-butyl-1,2,4-oxadiazol-3-yl)dibenzo[b,d]furan-2- |
| | | sulfonamido)-4-methylpentanoic acid |
| 280 | 15 | (S)-2-(7-(5-tert-butyl-1,2,4-oxadiazol-3-yl)dibenzo[b,d]furan-2- |
| | | sulfonamido)-2-(1H-indol-3-yl)acetic acid |
| 281 | 90 | (S)-2-(7-(5-tert-butyl-1,2,4-oxadiazol-3-yl)dibenzo[b,d]furan-2- |
| | | sulfonamido)-2-phenylacetic acid |
| 282 | 150 | (S)-2-(7-(5-tert-butyl-1,2,4-oxadiazol-3-yl)dibenzo[b,d]furan-2- |
| | | sulfonamido)-3,3-dimethylbutanoic acid |
| 283 | <1.5 | (S)-3-methyl-2-(8-(4-(4-(trifluoromethyl)phenyl)thiazol-2- |
| | | yl)dibenzo[b,d]furan-3-sulfonamido)butanoic acid |
| 284 | 18 | (S)-2-(8-(4-(4-fluorophenyl)thiazol-2-yl)dibenzo[b,d]furan-3- |
| | | sulfonamido)-3-methylbutanoic acid |
| 285 | 220 | (R)-3-methyl-2-(7-(thiazol-2-yl)dibenzo[b,d]thiophene-2- |
| | | sulfonamido)butanoic acid |
| 286 | 130 | (R)-2-(7-(benzo[d]thiazol-2-yl)dibenzo[b,d]thiophene-2-sulfonamido)- |
| | | 3-methylbutanoic acid |
| 287 | 70 | (R)-2-(7-(furan-2-yl)dibenzo[b,d]thiophene-2-sulfonamido)-3- |
| | | methylbutanoic acid |
| 288 | 110 | (R)-2-(7-(5-chlorofuran-2-yl)dibenzo[b,d]thiophene-2-sulfonamido)-3- |
| | | methylbutanoic acid |
| 289 | 9.1 | (R)-3-methyl-2-(7-(5-phenylthiophen-2-yl)dibenzo[b,d]furan-2- |
| | | sulfonamido)butanoic acid |
| 290 | 4.4 | (R)-2-(7-(5-chlorofuran-2-yl)dibenzo[b,d]furan-2-sulfonamido)-3- |
| | | methylbutanoic acid |
| 291 | 5.2 | (R)-3-methyl-2-(7-(thiazol-2-yl)dibenzo[b,d]furan-2- |
| | | sulfonamido)butanoic acid |
| 292 | 43 | (R)-3-methyl-2-(7-(5-methyl-1,3,4-thiadiazol-2-yl)dibenzo[b,d]furan- |
| | | 2-sulfonamido)butanoic acid |
| 293 | 21 | (R)-2-(7-(benzo[d]thiazol-2-yl)dibenzo[b,d]furan-2-sulfonamido)-3- |
| | | methylbutanoic acid |
| 294 | 9.5 | (R)-2-(7-(benzo[d]oxazol-2-yl)dibenzo[b,d]furan-2-sulfonamido)-3- |
| | | methylbutanoic acid |
| 295 | 15 | (R)-2-(7-(5-chloro-4-(trifluoromethyl)thiazol-2-yl)dibenzo[b,d]furan-2- |
| | | sulfonamido)-3-methylbutanoic acid |
| 296 | 3.1 | (R)-2-(7-(6-methoxybenzo[d]thiazol-2-yl)dibenzo[b,d]furan-2- |
| | | sulfonamido)-3-methylbutanoic acid |
| 297 | 2.2 | (R)-2-(7-(6-fluorobenzo[d]thiazol-2-yl)dibenzo[b,d]furan-2- |
| | | sulfonamido)-3-methylbutanoic acid |
| 298 | 7.4 | (R)-3-methyl-2-(7-(6-methylbenzo[d]thiazol-2-yl)dibenzo[b,d]furan-2- |
| | | sulfonamido)butanoic acid |
| 299 | 15 | (R)-2-(7-(4-fluorobenzo[d]thiazol-2-yl)dibenzo[b,d]furan-2- |
| | | sulfonamido)-3-methylbutanoic acid |
| 300 | 51 | (R)-3-methyl-2-(7-(4,5,6-trifluorobenzo[d]thiazol-2- |
| | | yl)dibenzo[b,d]furan-2-sulfonamido)butanoic acid |
| 301 | 78 | (R)-3-methyl-2-(7-(6-(trifluoromethoxy)benzo[d]thiazol-2- |
| | | yl)dibenzo[b,d]furan-2-sulfonamido)butanoic acid |
| 302 | 28 | (R)-3-methyl-2-(7-(6-(trifluoromethyl)benzo[d]thiazol-2- |
| | | yl)dibenzo[b,d]furan-2-sulfonamido)butanoic acid |
| 303 | <1.5 | (S)-2-(8-ethynyldibenzo[b,d]furan-3-sulfonamido)-3-methylbutanoic |
| | | acid |
| 304 | 1.5 | (S)-2-(7-(5-chlorothiophen-2-yl)dibenzo[b,d]thiophene-3- |
| | | sulfonamido)-3-methylbutanoic acid |
| 305 | 1.6 | (S)-2-(8-(4,5-dimethylthiazol-2-yl)dibenzo[b,d]furan-3-sulfonamido)- |
| | | 3-methylbutanoic acid |
| 306 | <1.5 | (S)-2-[7-(5,6-Dihydro-4H-cyclopentathiazol-2-yl)-dibenzofuran-3- |
| | | sulfonylamino]-3-methyl-butyric acid |
| 307 | <1.5 | (S)-3-methyl-2-(8-(4,5,6,7-tetrahydrobenzo[d]thiazol-2- |
| | | yl)dibenzo[b,d]furan-3-sulfonamido)butanoic acid |
| 308 | <1.5 | (S)-2-(8-(benzo[d][1,3]dioxol-5-yl)dibenzo[b,d]furan-3-sulfonamido)- |
| | | 3-methylbutanoic acid |
| 309 | <1.5 | (S)-3-methyl-2-(8-phenyldibenzo[b,d]furan-3-sulfonamido)butanoic |
| | | acid |
| 310 | <1.5 | (S)-2-(8-(4-methoxyphenyl)dibenzo[b,d]furan-3-sulfonamido)-3- |
| | | methylbutanoic acid |
| 311 | 2.0 | (S)-3-methyl-2-(8-(4-(trifluoromethyl)phenyl)dibenzo[b,d]furan-3- |
| | | sulfonamido)butanoic acid |
| 312 | | Absent |
| 313 | <1.5 | (S)-3-methyl-2-(7-phenyldibenzo[b,d]thiophene-3- |
| | | sulfonamido)butanoic acid |
| 314 | 2.6 | (R)-3-methyl-2-(7-phenyldibenzo[b,d]thiophene-3- |
| | | sulfonamido)butanoic acid |

“not tested” indicates compounds were not subjected to assay due to instability
“absent” indicates that the compound number is not allocated to any compound.

Variations, modifications, and other implementations of what is described herein will occur to those of ordinary skill in the art without departing from the spirit and the essential characteristics of the present teachings. Accordingly, the scope of the invention is to be defined not by the preceding illustrative description but instead by the following claims, and all changes that come within the meaning and range of equivalency of the claims are intended to be embraced herein.

We claim:

1. A compound of formula I: 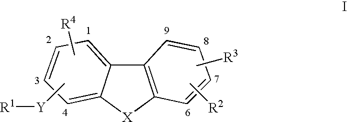
or a pharmaceutically acceptable salt or ester thereof, wherein:
X is O, S, S(O), or S(O)₂;
R¹—Y is a substituent at position C2 or C3 of formula I;
Y is S(O), or S(O)₂;
R¹ is an N-linked, free carboxyl or carboxyl-protected, natural or non-natural amino acid containing at least one alpha-amino hydrogen;
R² is a substituent at position C7 or C8 of formula I, selected from a) —C(O)OR⁶, b) —C(S)OR⁶, c) —C(S)R⁷, d) —C(S)NR⁷R⁸, e) —C(NR⁷)R⁷, f) —C(NR⁷)OR⁶, g) —C(NR⁷)NR⁷R⁸, h) a C₂₋₁₀ alkenyl group, i) a C₂₋₁₀ alkynyl group, j) a C₁₋₁₀ haloalkyl group, k) a C₃₋₁₄ cycloalkyl group, l) a 3-14 membered cycloheteroalkyl group and m) a 5-14 membered heteroaryl group, wherein the 3-14 membered cycloheteroalkyl group, or the 5-14 membered heteroaryl group is linked to the tricyclic core via a carbon ring atom, and each of h)-m) optionally is substituted with 1-4 —Z—R⁹ groups;
R³ and R⁴ independently are a) H, b) —CN, c) —NO₂, d) halogen, e) —OR⁶, f) —NR⁷R⁸, g) —S(O)ₘR⁷, h) —S(O)ₘOR⁶, i) —C(O)R⁷, j) —C(O)OR⁶, k) —C(O)NR⁷R⁸, l) —C(S)R⁷, m) —C(S)OR⁶, n) —C(S)NR⁷R⁸, o) —C(NR⁷)R⁷, p) —C(NR⁷)OR⁶, q) —C(NR⁷)NR⁷R⁸, r) a C₁₋₁₀ alkyl group, s) a C₂₋₁₀ alkenyl group, t) a C₂₋₁₀ alkynyl group, u) a C₁₋₁₀ haloalkyl group, v) a C₃₋₁₄ cycloalkyl group, w) a C₆₋₁₄ aryl group, x) a 3-14 membered cycloheteroalkyl group, or y) a 5-14 membered heteroaryl group, wherein each of r)-y) optionally is substituted with 1-4 —Z—R⁹ groups;
R⁶, at each occurrence, independently is a) H, b) —C(O)R⁷, c) —C(O)NR⁷R⁸, d) —C(S)R⁷, e) —C(S)NR⁷R⁸, f) —C(NR⁷)R⁷, g) —C(NR⁷)NR⁷R⁸, h) a C₁₋₁₀ alkyl group, i) a C₂₋₁₀ alkenyl group, j) a C₂₋₁₀ alkynyl group, k) a C₁₋₁₀ haloalkyl group, l) a C₃₋₁₄ cycloalkyl group, m) a C₆₋₁₄ aryl group, o) a 3-14 membered cycloheteroalkyl group, or p) a 5-14 membered heteroaryl group, wherein each of h)-p) optionally is substituted with 1-4 —Z—R⁹ groups;
R⁷ and R⁸, at each occurrence, independently are a) H, b) —OH, c) —NH₂, d) —S(O)ₘH, e) —S(O)ₘOH, f) —C(O)OH, g) —C(O)NH₂, h) —C(S)NH₂, i) —C(NH)NH₂, j) —OC₁₋₁₀ alkyl, k) —NH—C₁₋₁₀ alkyl, l) —N(C₁₋₁₀ alkyl)₂, m) —S(O)ₘ—C₁₋₁₀ q) —C(O)NH—C₁₋₁₀ alkyl, r) —C(O)N(C₁₋₁₀ alkyl)₂, s) —C(S)NH—C₁₋₁₀ alkyl, t) —C(S)N(C₁₋₁₀ alkyl)₂, u) —C(NH)—C₁₋₁₀ alkyl, v) —C(NH)—OC₁₋₁₀ alkyl, w) —C(NH)NH—C₁₋₁₀ alkyl, x) —O(NH)N(C₁₋₁₀ alkyl)₂, y) —C(NC₁₋₁₀ alkyl)-C₁₋₁₀ alkyl, z) —C(NC₁₋₁₀ alkyl)-OC₁₋₁₀ alkyl, aa) —C(NC₁₋₁₀ alkyl)NH—C₁₋₁₀ alkyl, ab) —C(NC₁₋₁₀ alkyl)N(C₁₋₁₀ alkyl)₂, ac) a C₁₋₁₀ alkyl group, ad) a C₂₋₁₀ alkenyl group, ae) a C₂₋₁₀ alkynyl group, af) a C₁₋₁₀ haloalkyl group, ag) a C₃₋₁₄ cycloalkyl group, ah) a C₆₋₁₄ aryl group, ai) a 3-14 membered cycloheteroalkyl group, or aj) a 5-14 membered heteroaryl group, wherein each of the C₁₋₁₀ alkyl group, the C₂₋₁₀ alkenyl group, the C₂₋₁₀ alkynyl group, the C₁₋₁₀ haloalkyl group, the C₃₋₁₄ cycloalkyl group, the C₆₋₁₄ aryl group, the 3-14 membered cycloheteroalkyl group, and the 5-14 membered heteroaryl group optionally is substituted with 1-4 —Z—R⁹ groups;
R⁹, at each occurrence, independently is a) halogen, b) —CN, c) —NO₂, d) oxo, wherein two R⁹ on a single carbon atom can be replaced, e) —O—Z—R¹⁰, f) —NR¹⁰—Z—R¹¹, g) —N(O)R¹⁰—Z—R¹¹, h) —S(O)ₘR¹⁰, i) —S(O)ₘO—Z—R¹⁰, j) —S(O)ₘNR¹⁰—Z—R¹¹, k) —C(O)R¹⁰, l) —C(O)O—Z—R¹⁰, m) —C(O)NR¹⁰—Z—R¹¹, n) —C(S)NR¹⁰—Z—R¹¹, o) —C(NR¹⁰)R¹⁰, p) —C(NR¹⁰)O—Z—R¹⁰, q) —C(NR¹⁰)NR¹⁰—Z—R¹¹, r) —Si(C₁₋₁₀ alkyl)₃, s) a C₁₋₁₀ alkyl group, t) a C₂₋₁₀ alkenyl group, u) a C₂₋₁₀ alkynyl group, v) a C₁₋₁₀ haloalkyl group, w) a C₃₋₁₄ cycloalkyl group, x) a C₆₋₁₄ aryl group, y) a 3-14 membered cycloheteroalkyl group, or z) a 5-14 membered heteroaryl group, wherein each of the C₁₋₁₀ alkyl group, the C₂₋₁₀ alkenyl group, the C₂₋₁₀ alkynyl group, the C₁₋₁₀ haloalkyl group, the C₃₋₁₄ cycloalkyl group, the C₆₋₁₄ aryl group, the 3-14 membered cycloheteroalkyl group, and the 5-14 membered heteroaryl group optionally is substituted with 1-4 —Z—R¹² groups;
R¹⁰ and R¹¹, at each occurrence, independently are a) H, b) —OH, c) —NH₂, d) —S(O)ₘH, e) —S(O)ₘOH, f) —C(O)OH, g) —C(O)NH₂, h) —C(S)NH₂, i) —C(NH)NH₂, j) —OC₁₋₁₀ alkyl, k) —NH—C₁₋₁₀ alkyl, l) —N(C₁₋₁₀ alkyl)₂, m) —S(O)ₘ—C₁₋₁₀ alkyl, n) —S(O)ₘ—OC₁₋₁₀ alkyl, o) —C(O)—C₁₋₁₀ alkyl, p) —C(O)—OC₁₋₁₀ alkyl, q) —C(O)NH—C₁₋₁₀ alkyl, r) —C(O)N(C₁₋₁₀ alkyl)₂, s) —C(S)NH—C₁₋₁₀ alkyl, t) —C(S)N(C₁₋₁₀ alkyl)₂, u) —C(NH)—C₁₋₁₀ alkyl, v) —C(NH)—OC₁₋₁₀ alkyl, w) —C(NH)NH—C₁₋₁₀ alkyl, x) —C(NH)N(C₁₋₁₀ alkyl)₂, y) —C(NC₁₋₁₀ alkyl)-C₁₋₁₀ alkyl, z) —C(NC₁₋₁₀ alkyl)-OC₁₋₁₀ alkyl, aa) —C(NC₁₋₁₀ alkyl)NH—C₁₋₁₀ alkyl, ab) —C(NC₁₋₁₀ alkyl)N(C₁₋₁₀ alkyl)₂, ac) a C₁₋₁₀ alkyl group, ad) a C₂₋₁₀ alkenyl group, ae) a C₂₋₁₀ alkynyl group, af) a C₁₋₁₀ haloalkyl group, ag) a C₃₋₁₄ cycloalkyl group, ah) a C₆₋₁₄ aryl group, ai) a 3-14 membered cycloheteroalkyl group, or aj) a 5-14 membered heteroaryl group, wherein each of the C₁₋₁₀ alkyl group, the C₂₋₁₀ alkenyl group, the C₂₋₁₀ alkynyl group, the C₁₋₁₀ haloalkyl group, the C₃₋₁₄ cycloalkyl group, the C₆₋₁₄ aryl group, the 3-14 membered cycloheteroalkyl group, and the 5-14 membered heteroaryl group optionally is substituted with 1-4 —Z—R¹² groups;
R¹², at each occurrence, independently is a) halogen, b) —CN, c) —NO₂, d) oxo, wherein two R¹² on a single carbon can be replaced e) —OH, f) —NH₂, g) —NH(C₁₋₁₀ alkyl), h) —N(C₁₋₁₀ alkyl)₂, i) —S(O)ₘH, j) —S(O)ₘ—C₁₋₁₀ alkyl, k) —S(O)ₘOH, l) —S(O)ₘ—OC₁₋₁₀ alkyl, m) —CHO, n) —C(O)—C₁₋₁₀ alkyl, o) —C(O)OH, p) —C(O)—OC₁₋₁₀ alkyl, q) —C(O)NH₂, r) —C(O)NH—C₁₋₁₀ alkyl, s) —C(O)N(C₁₋₁₀ alkyl)₂, t) —C(NH)H, u) —C(NH)—C₁₋₁₀ alkyl, v) —C(NH)OH, w) —C(NH)—OC₁₋₁₀ alkyl, x) —C(NH)NH₂, y) —C(NH)NH—C₁₋₁₀ alkyl, z) —C(NH)N(C₁₋₁₀ alkyl)₂, aa) —C(NC₁₋₁₀ alkyl)H, ab) —C(NC₁₋₁₀ alkyl)-C₁₋₁₀ alkyl, ac) —C(NC₁₋₁₀ alkyl)OH, ad) —C(NC₁₋₁₀ alkyl)-OC₁₋₁₀ alkyl, ae) —C(NC₁₋₁₀ alkyl)NH₂, af) —C(NC₁₋₁₀ alkyl)NH—C₁₋₁₀ alkyl, ag) —C(NC₁₋₁₀ alkyl)N(C₁₋₁₀ alkyl)₂, ah) —C(S)NH₂, ai) —C(S)NH—C₁₋₁₀ alkyl, aj) —C(S)N(C₁₋₁₀ alkyl)₂, ak) —S(O)ₘNH₂, al) —S(O)ₘNH(C₁₋₁₀ alkyl), am) —S(O)ₘN(C₁₋₁₀ alkyl)₂, an) —Si(C₁₋₁₀ alkyl)₃, ap) a C₁₋₁₀ alkyl group, aq) a C₂₋₁₀ alkenyl group, ar) a C₂₋₁₀ alkynyl group, as) a C₁₋₁₀ haloalkyl group, at) a C₃₋₁₄ cycloalkyl group, au) a C₆₋₁₄ aryl group, av) a 3-14 membered cycloheteroalkyl group, or aw) a 5-14 membered heteroaryl group; wherein each of ap) to av) is optionally substituted with 1-4 groups selected from halogen, —CN, —NO₂, —OH, —O(C₁₋₁₀ alkyl), —NH₂, —NH(C₁₋₁₀ alkyl), and —N(C₁₋₁₀ alkyl)₂;
Z, at each occurrence, independently is a) a divalent C₁₋₁₀ alkyl group, b) a divalent C₂₋₁₀ alkenyl group, c) a divalent C₂₋₁₀ alkynyl group, d) a divalent C₁₋₁₀ haloalkyl group, or e) —Z— is a bond; and
m, at each occurrence, independently is 0, 1, or 2.

2. The compound according to claim 1, or a pharmaceutically acceptable salt or ester thereof, wherein
R² is a) —C(S)OR⁶, b) —C(S)R⁷, c) —C(S)NR⁷R⁹, d) —C(NR⁷)R⁷, e) —C(NR⁷)OR⁶, f) —C(NR⁷)NR⁷R⁹, g) a C₂₋₁₀ alkenyl group, h) a C₂₋₁₀ alkynyl group, i) a C₃₋₁₄ cycloalkyl group, j) a 3-14 membered cycloheteroalkyl group, or k) a 5-14 membered heteroaryl group, wherein the 3-14 membered cycloheteroalkyl group, or the 5-14 membered heteroaryl group is linked to the tricyclic core via a carbon ring atom, and each of g)-k) optionally is substituted with 1-4 —Z—R⁹ groups.

3. The compound according to claim 1 or claim 2, or a pharmaceutically acceptable salt or ester thereof, wherein R² is —C(NR⁷)R⁷ or —C(NR⁷)NR⁷R⁹.

4. The compound according claim 1, or a pharmaceutically acceptable salt or ester thereof, wherein R² is —C(NH)R⁷, —C(NCH₃)R⁷, —C(NCH₂CH₃)R⁷, —C(NCH(CH₃)₂)R⁷, —C(NH)NR⁷R⁸, —C(NCH₃)NR⁷R⁸, —C(NCH₂CH₃)NR⁷R⁸, or —C(NCH(CH₃)₂)NR⁷R⁸; and R⁷ and R⁸ are independently selected from H, —OH, —OC₁₋₁₀ alkyl, a C₁₋₁₀ alkyl, and a 3-14 membered cycloheteroalkyl, wherein the C₁₋₁₀ alkyl, and the 3-14 membered cycloheteroalkyl optionally is substituted with 1-4 —Z—R⁹ groups.

5. The compound according to claim 1, or a pharmaceutically acceptable salt or ester thereof, wherein R² is a group selected from N-isopropylcarbamimidoyl, N-hydroxycarbamimidoyl, N-methoxycarbamimidoyl, N-methylcarbamimidoyl, N-ethyl carbamimidoyl, N-phenylcarbamimidoyl, N-benzylcarbamimidoyl, N,N-diethyl carbamimidoyl, N-methyl-N-isopropylcarbamimidoyl, N-ethyl-N′-ethylcarbamimidoyl, N-methylamido, N-ethylamido and imino(pyrrolidin-1-yl)methyl.

6. The compound according to claim 1, or a pharmaceutically acceptable salt or ester thereof, wherein R² is a group selected from C₂₋₁₀ alkenyl and C₂₋₁₀ alkynyl, wherein each group is optionally substituted with —O—Z—R¹⁰, —NR¹⁰—Z—R¹¹, —C(O)R¹⁰, —C(O)O—Z—R¹⁰, —C(O)NR¹⁰—Z—R¹¹, C₃₋₁₄ cycloalkyl, C₆₋₁₄ aryl, 3-14 membered cycloheteroalkyl, or 5-14 membered heteroaryl, wherein each of the C₃₋₁₄ cycloalkyl, the C₆₋₁₄ aryl, the 3-14 membered cycloheteroalkyl, and the 5-14 membered heteroaryl is optionally substituted with 1-4 —Z—R¹² groups.

7. The compound according to claim 1, or a pharmaceutically acceptable salt or ester thereof, wherein R² is a group is selected from 2-cyclopropylethenyl, 2-cyclobutylethenyl, 2-cyclopentylethenyl, 2-cyclohexyl ethenyl, 2-cycloheptylethenyl, methoxycarbonylethynyl, diethylaminoethynyl, 3-methoxypropynyl, 3-dimethyl aminopropynyl, 3-N,N-diethylaminopropynyl and (1-methylimidazol-2-yl)ethynyl, each of which optionally is substituted with 1-4 —Z—R¹² groups.

8. The compound according to any one of claims 1-2, or a pharmaceutically acceptable salt or ester thereof, wherein R² is a group selected from C₃₋₁₄ cycloalkyl and 3-14 membered cycloheteroalkyl, each of which optionally is substituted with 1-4 —Z—R⁹ groups.

9. The compound according to any one of claims 1-2, or a pharmaceutically acceptable salt or ester thereof, wherein R² is a group selected from cis-1-propenyl, trans-1-propenyl, cis-2-propenyl, trans-2-propenyl, cyclopropyl, cyclobutyl, cyclopentyl, cyclohexyl, cycloheptyl, cyclopentenyl, cyclohexenyl, 4,5-dihydro-1H-imidazol-2-yl, 4,5-dihydrooxazol-2-yl, 4,5-dihydrothiazol-2-yl, and 1,2,3,6-tetrahydropyridin-4-yl, each of which optionally is substituted with 1-4 —Z—R⁹ groups.

10. The compound according to any of claims 1-2, or a pharmaceutically acceptable salt or ester thereof, wherein R² is a 5-14 membered heteroaryl group optionally substituted with 1-4 —Z—R⁹ groups.

11. The compound according to claim 10, or a pharmaceutically acceptable salt or ester thereof, wherein R² is a 5-6 membered heteroaryl group having 1-4 ring members independently selected from O, S, and N, and wherein the 5-6 membered heteroaryl group optionally is substituted with 1-4 —Z—R⁹ groups.

12. The compound according to any one of claims 1-2 and 10-11, or a pharmaceutically acceptable salt or ester thereof, wherein R² is selected from furanyl, thienyl, pyrrolyl, oxazolyl, thiazolyl, imidazolyl, oxadiazolyl, thiadiazolyl, triazolyl, pyridinyl, pyrimidinyl, pyrazinyl, isoxazolyl, isoxadiazolyl, pyrazolyl, and tetrazolyl, each of which optionally is substituted with 1-4 —Z—R⁹ groups.

13. The compound according to claim 12, or a pharmaceutically acceptable salt or ester thereof, wherein R² is a furanyl or isoxazolyl or oxadiazolyl, group, each of which optionally is substituted with 1-4 —Z—R⁹ groups.

14. The compound according to claim 12, or a pharmaceutically acceptable salt or ester thereof, wherein R² is a thienyl or thiazolyl, group, each of which optionally is substituted with 1-4 —Z—R⁹ groups.

15. The compound according to claim 12, or a pharmaceutically acceptable salt or ester thereof, wherein R² is a pyrrolyl, imidazolyl, triazolyl or tetrazolyl group, each of which optionally is substituted with 1-4 —Z—R⁹ groups.

16. The compound according to any one of claims 13-15, or a pharmaceutically acceptable salt or ester thereof, wherein R² is substituted with 1-4 substituents independently selected from halogen, C₁₋₁₀ alkyl, C₁₋₁₀ haloalkyl, C₃₋₁₄ cycloalkyl, C₆₋₁₄ aryl, 3-14 membered cycloheteroalkyl, and 5-14 membered heteroaryl.

17. The compound according to any one of claims 1-2, or a pharmaceutically acceptable salt or ester thereof, wherein R² is an 8-14 membered heteroaryl group comprising a 5-6 membered heteroaryl ring fused with 1-2 rings independently selected from C₃₋₈ cycloalkyl, phenyl, 3-8 membered cycloheteroalkyl, and 5-8 membered heteroaryl, wherein the 5-6 membered heteroaryl group is selected from furanyl, thienyl, pyrrolyl, oxazolyl, thiazolyl, imidazolyl, oxadiazolyl, thiadiazolyl, triazolyl, pyridinyl, pyrimidinyl, pyrazinyl, isoxazolyl, pyrazolyl, and tetrazolyl; and wherein the 8-14 membered heteroaryl group is optionally substituted with 1-4 —Z—R⁹ groups.

18. The compound according to claim 17, or a pharmaceutically acceptable salt or ester thereof, wherein each of the C₃₋₈ cycloalkyl, phenyl, the 3-8 membered cycloheteroalkyl, and the 5-8 membered heteroaryl group is independently selected from cyclopentyl, cyclopentenyl, cyclohexyl, cyclohexenyl, phenyl, and pyridinyl.

19. The compound according to any one of claims 1-2 and 17-18, or a pharmaceutically acceptable salt or ester thereof, wherein R² is selected from benzoxazolyl, benzothiazolyl, benzimidazolyl, benzofuranyl, benzothienyl, indolyl, benzoindolyl, dibenzofuranyl, and dibenzothienyl.

20. The compound according to any one of claims 1-2 and 17-18, or a pharmaceutically acceptable salt or ester thereof, wherein R² is a 2-oxo-1H-benzo[d][1,3]oxazinyl group optionally substituted 1-3 —Z—R⁹ groups.

21. A compound of formula IE: 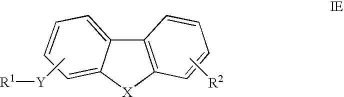
or a pharmaceutically acceptable salt or ester thereof, wherein:
X is O, S, S(O), or S(O)₂;
R¹—Y is a substituent at position C2 or C3 of formula IE;
Y is S(O), or S(O)₂;
R¹ is an N-linked valine with a free or protected carboxyl C-terminus, and
R² is phenyl or benzo[d][1,3]dioxole, optionally substituted with 1-4 groups selected from halogen, CF₃ and OCH₃.

22. A compound according to claim 21, or a pharmaceutically acceptable salt or ester thereof, wherein the compound is selected from the group consisting of:
(S)-2-(8-(benzo[d][1,3]dioxol-5-yl)dibenzo[b,d]furan-3-sulfonamido)-3-methylbutanoic acid;
(S)-3-methyl-2-(8-phenyldibenzo[b,d]furan-3-sulfonamido)butanoic acid;
(S)-2-(8-(4-methoxyphenyl)dibenzo[b,d]furan-3-sulfonamido)-3-methylbutanoic acid;
(S)-3-methyl-2-(8-(4-(trifluoromethyl)phenyl)dibenzo[b,d]furan-3-sulfonamido)butanoic acid;
(R)-3-methyl-2-(7-(4-(trifluoromethyl)phenyl)dibenzo[b,d]furan-2-sulfonamido)butanoic acid;
(S)-3-methyl-2-(7-phenyldibenzo[b,d]thiophene-3-sulfonamido)butanoic acid; and
(R)-3-methyl-2-(7-phenyldibenzo[b,d]thiophene-3-sulfonamido)butanoic acid.

23. The compound according to any one of claims 1-20, or a pharmaceutically acceptable salt or ester thereof, wherein formula I is selected from:

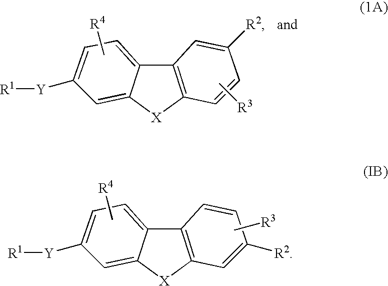

24. The compound according to any one of claims 1-20, or a pharmaceutically acceptable salt or ester thereof, wherein the compound of formula I is selected from:

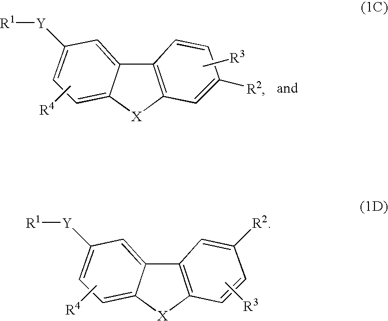

25. The compound according to any one of claims 1-20, 23 and 24, or a pharmaceutically acceptable salt or ester thereof, wherein R³ and R⁴ are hydrogen.

26. The compound according to any one of claims 1-21 and 23-25 or a pharmaceutically acceptable salt or ester thereof, wherein X is O.

27. The compound according to any one of claims 1-21 and 23-25, or a pharmaceutically acceptable salt or ester thereof, wherein X is S.

28. The compound according to any one of claims 1-21 and 23-27, or a pharmaceutically acceptable salt or ester thereof, wherein Y is S(O)₂.

29. The compound according to any one of claims 1-20 or 23-28, or a pharmaceutically acceptable salt or ester thereof, wherein R¹ is W—V—NH—, wherein:
W is a) —C(O)R¹³, b) —S(O)ₘR¹³, c) —S(O)ₘOR¹³, d) —S(O)ₘNR¹³R¹⁴, e) —C(O)OR¹³, f) —C(O)NR¹³R¹⁴, g) —C(S)R¹³, h) —C(S)OR¹⁴, i) —NR¹³R¹⁴, j) —C(NR¹³)NR¹³R¹⁴, k) —P(O)(OR¹³)₂, or l) —B(OR¹³)₂;
V is —CR¹³R¹⁵—, —CH₂OR¹³R¹⁵—, —(CH═CR¹⁵)—, or —BHR¹⁵—; R¹³ and R¹⁴, at each occurrence, independently are a) H, b) —OH, c) —SH, d) —S(O)₂OH, e) —C(O)OH, f) —C(O)NH₂, g) —C(S)NH₂, h) —O—C₁₋₁₀ alkyl, i) —S(O)ₘ—C₁₋₁₀ alkyl, j) —S(O)ₘ—OC₁₋₁₀ alkyl, k) —C(O)—C₁₋₁₀ alkyl, l) —C(O)—OC₁₋₁₀ alkyl, m) —C(O)NH—C₁₋₁₀ alkyl, n) —C(O)N(C₁₋₁₀ alkyl)₂, o) —C(S)NH—C₁₋₁₀ alkyl, p) —C(S)N(C₁₋₁₀ alkyl)₂, q) a C₁₋₁₀ alkyl group, r) a C₂₋₁₀ alkenyl group, s) a C₂₋₁₀ alkynyl group, t) a C₁₋₁₀ haloalkyl group, u) a C₃₋₁₄ cycloalkyl group, v) a C₆₋₁₄ aryl group, w) a 3-14 membered cycloheteroalkyl group, or x) a 5-14 membered heteroaryl group, wherein each of the C₁₋₁₀ alkyl group, the C₂₋₁₀ alkenyl group, the C₂₋₁₀ alkynyl group, the C₁₋₁₀ haloalkyl group, the C₃₋₁₄ cycloalkyl group, the C₆₋₁₄ aryl group, the 3-14 membered cycloheteroalkyl group, and the 5-14 membered heteroaryl group optionally is substituted with 1-4 —Z—R¹⁶ groups;
R¹³ and R¹⁴, at each occurrence, independently are a) H, b) —OH, c) —SH, d) —S(O)₂OH, e) —C(O)OH, f) —C(O)NH₂, g) —C(S)NH₂, h) —O—C₁₋₁₀ alkyl, i) —S(O)ₘ—C₁₋₁₀ alkyl, j)—S(O)ₘ—OC₁₋₁₀ alkyl, k) —C(O)—C₁₋₁₀ alkyl, l) —C(O)OC₁₋₁₀ alkyl, m) —C(O)NH—C₁₋₁₀ alkyl, n) —C(O)N(C₁₋₁₀ alkyl)₂, o) —C(S)NH—C₁₋₁₀ alkyl, p) —C(S)N(C₁₋₁₀ alkyl)₂, q) a C₁₋₁₀ alkyl group, r) a C₂₋₁₀ alkenyl group, s) a C₂₋₁₀ alkynyl group, t) a C₁₋₁₀ haloalkyl group, u) a C₃₋₁₄ cycloalkyl group, v) a C₆₋₁₄ aryl group, w) a 3-14 membered cycloheteroalkyl group, or x) a 5-14 membered heteroaryl group, wherein each of the C₁₋₁₀ alkyl group, the C₂₋₁₀ alkenyl group, the C₂₋₁₀ alkynyl group, the C₁₋₁₀ haloalkyl group, the C₃₋₁₄ cycloalkyl group, the C₆₋₁₄ aryl group, the 3-14 membered cycloheteroalkyl group, and the 5-14 membered heteroaryl group optionally is substituted with 1-5 —Z—R¹⁶ groups;
R¹⁵ is H or a side chain of a natural or non-natural amino acid; and
R¹⁶, at each occurrence, independently is a) halogen, b) —ON, c) —NO₂, d) oxo, where two R¹⁶ on a single carbon can be replaced, e) —OH, f) —O—C₁₋₁₀ alkyl, g) —NH₂, h) —NH(C₁₋₁₀ alkyl), i) —N(C₁₋₁₀ alkyl)₂, j) —S(O)ₘH, k) —S(O)ₘ—C₁₋₁₀ alkyl, l) —S(O)₂OH, m) —S(O)ₘOC₁₋₁₀ alkyl, n) —CHO, o) —C(O)—C₁₋₁₀ alkyl, p) —C(O)OH, q) —C(O)—OC₁₋₁₀ alkyl, r) —C(O)NH₂, s) —C(O)NH—C₁₋₁₀ alkyl, t) —C(O)N(C₁₋₁₀ alkyl)₂, u) —C(S)NH₂, v) —C(S)NH—C₁₋₁₀ alkyl, w) —C(S)N(C₁₋₁₀ alkyl)₂, x) —S(O)ₘNH₂, y) —S(O)ₘNH(C₁₋₁₀ alkyl), z) —S(O)ₘN(C₁₋₁₀ alkyl)₂, aa) —Si(C₁₋₁₀ alkyl)₃, ab) a C₁₋₁₀ alkyl group, ac) a C₂₋₁₀ alkenyl group, ad) a C₂₋₁₀ alkynyl group, ae) a C₁₋₁₀ haloalkyl group, af) a C₃₋₁₄ cycloalkyl group, ag) a C₆₋₁₄ aryl group, ah) a 3-14 membered cycloheteroalkyl group, or ai) a 5-14 membered heteroaryl group.

30. The compound of claim 29, or a pharmaceutically acceptable salt or ester thereof, wherein W is —C(O)OR¹³ and V is —CR¹³R¹⁵— or —CH₂CR¹³R¹⁵—; wherein R¹³ and R¹⁵ are different and the carbon atom to which R¹³ and R¹⁵ is each attached is a chiral center.

31. The compound according to any one of claim 1-20 or 23-30, or a pharmaceutically acceptable salt or ester thereof, wherein R¹ is an L-alpha-amino acid.

32. The compound according to any one of claim 1-20 or 23-30, or a pharmaceutically acceptable salt or ester thereof, wherein R¹ is a D-alpha-amino acid.

33. The compound according to any one of claim 1-20 or 23-29, or a pharmaceutically acceptable salt or ester thereof, wherein the amino acid is a beta-amino acid.

34. The compound according to claim 29 or claim 30, or a pharmaceutically acceptable salt or ester thereof, wherein R¹⁵ is an isopropyl group.

35. The compound according to claim 31 or a pharmaceutically acceptable salt or ester thereof, wherein R¹ is an N-linked L-valine.

36. The compound according to any one of claim 1-20, 23-32 or 34, or a pharmaceutically acceptable salt or ester thereof, wherein R¹ is an N-linked valine.

37. The compound of any of claims 1-36 wherein the pharmaceutically acceptable salt is an amine salt or a carboxylic acid salt.

38. A compound according to claim 1, or a pharmaceutically acceptable salt or ester thereof, wherein the compound is selected from the group consisting of:
(R)-2-(8-(4,4-dimethyl-2-oxo-2,4-dihydro-1H-benzo[d][1,3]oxazin-6-yl)dibenzo[b,d]furan-3-sulfonamido)-3-methylbutanoic acid;
(S)-2-(8-(3-(dimethylamino)prop-1-ynyl)dibenzo[b,d]furan-3-sulfonamido)-3-methylbutanoic acid;
(S)-3-methyl-2-(8-(pyridin-3-yl)dibenzo[b,d]furan-3-sulfonamido)butanoic acid;
(S)-2-(8-(4,4-dimethyl-2-oxo-2,4-dihydro-1H-benzo[d][1,3]oxazin-6-yl)dibenzo[b,d]furan-3-sulfonamido)-3-methylbutanoic acid;
(S)-3-methyl-2-(8-(4-methylthiophen-3-yl)dibenzo[b,d]furan-3-sulfonamido) butanoic acid;
(S)-2-(8-(3-methoxy-3-oxoprop-1-ynyl)dibenzo[b,d]furan-3-sulfonamido)-3-methylbutanoic acid;
(S)-2-(8-(furan-3-yl)dibenzo[b,d]furan-3-sulfonamido)-3-methylbutanoic acid;
(S)-2-(8-(1H-pyrrol-2-yl)dibenzo[b,d]furan-3-sulfonamido)-3-methylbutanoic acid;
(S)-2-(8-(3,5-dimethylisoxazol-4-yl)dibenzo[b,d]furan-3-sulfonamido)-3-methylbutanoic acid;
(S)-2-(8-(6-methoxypyridin-3-yl)dibenzo[b,d]furan-3-sulfonamido)-3-methylbutanoic acid;
(S)-3-methyl-2-(8-(pyridin-3-yl)dibenzo[b,d]thiophene-3-sulfonamido)butanoic acid;
(S)-2-(8-(benzo[b]thiophen-3-yl)dibenzo[b,d]furan-3-sulfonamido)-3-methylbutanoic acid;
(S)-2-(8-(benzo[b]thiophen-2-yl)dibenzo[b,d]furan-3-sulfonamido)-3-methylbutanoic acid;
(S)-3-methyl-2-(8-(quinolin-6-yl)dibenzo[b,d]furan-3-sulfonamido)butanoic acid;
(S)-3-methyl-2-(8-(1-methyl-1H-imidazol-5-yl)ethynyl)dibenzo[b,d]furan-3-sulfonamido)butanoic acid;
(S)-3-methyl-2-(8-(pyridin-4-yl)dibenzo[b,d]furan-3-sulfonamido)butanoic acid;
(S)-3-methyl-2-(8-(5-methylthiophen-2-yl)dibenzo[b,d]furan-3-sulfonamido)butanoic acid;
(S)-3-methyl-2-(8-(1-methyl-1H-pyrazol-4-yl)dibenzo[b,d]furan-3-sulfonamido)butanoic acid;
(S)-2-(8-(3,5-dimethyl-1H-pyrazol-4-yl)dibenzo[b,d]furan-3-sulfonamido)-3-methylbutanoic acid;
(S)-2-(8-(1-isopentyl-1H-pyrazol-4-yl)dibenzo[b,d]furan-3-sulfonamido)-3-methylbutanoic acid;
(S)-3-methyl-2-(8-(1-propyl-1H-pyrazol-4-yl)dibenzo[b,d]furan-3-sulfonamido)butanoic acid;
(S)-2-(8-(1-benzyl-1H-pyrazol-4-yl)dibenzo[b,d]furan-3-sulfonamido)-3-methylbutanoic acid;
(S)-2-(8-(1H-pyrazol-4-yl)dibenzo[b,d]furan-3-sulfonamido)-3-methylbutanoic acid;
(S)-3-methyl-2-(8-(4-methylthiophen-3-yl)dibenzo[b,d]thiophene-3-sulfonamido)butanoic acid;
(S)-2-(8-(furan-3-yl)dibenzo[b,d]thiophene-3-sulfonamido)-3-methylbutanoic acid;
(S)-3-methyl-2-(8-(thiophen-3-yl)dibenzo[b,d]thiophene-3-sulfonamido)butanoic acid;
(S)-2-(8-(3,5-dimethylisoxazol-4-yl)dibenzo[b,d]thiophene-3-sulfonamido)-3-methylbutanoic acid;
(S)-2-(8-(3,5-dimethylisoxazol-4-yl)dibenzo[b,d]thiophene-3-sulfonamido)-3-methylbutanoic acid;
(S)-3-methyl-2-(8-(4-methylthiophen-3-yl)dibenzo[b,d]furan-3-sulfonamido)butanoic acid;
(S)-3-methyl-2-(8-(4-methylthiophen-3-yl)dibenzo[b,d]furan-3-sulfonamido)butanoic acid;
(S)-2-(8-(3-formylthiophen-2-yl)dibenzo[b,d]furan-3-sulfonamido)-3-methylbutanoic acid;
(S)-2-(8-(5-acetylthiophen-2-yl)dibenzo[b,d]furan-3-sulfonamido)-3-methylbutanoic acid;
(S)-3-methyl-2-(8-(4-methylthiophen-2-yl)dibenzo[b,d]furan-3-sulfonamido)butanoic acid;
(S)-2-(8-(2-chlorothiophen-3-yl)dibenzo[b,d]furan-3-sulfonamido)-3-methylbutanoic acid;
(S,E)-2-(8-(2-cyclohexylvinyl)dibenzo[b,d]furan-3-sulfonamido)-3-methylbutanoic acid;
(S)-3-methyl-2-(8-(thiazol-2-yl)dibenzo[b,d]furan-3-sulfonamido)butanoic acid;
(S)-2-(8-(furan-2-yl)dibenzo[b,d]furan-3-sulfonamido)-3-methylbutanoic acid;
(S)-2-(8-(methoxyethynyl)dibenzo[b,d]furan-3-sulfonamido)-3-methylbutanoic acid;
(S)-2-(8-((diethylamino)ethynyl)dibenzo[b,d]furan-3-sulfonamido)-3-methylbutanoic acid;
(S)-3-methyl-2-(8-(1-methyl-1H-pyrazol-4-yl)dibenzo[b,d]thiophene-3-sulfonamido)butanoic acid;
(S)-2-(8-(3,5-dimethyl-1H-pyrazol-4-yl)dibenzo[b,d]thiophene-3-sulfonamido)-3-methylbutanoic acid;
(S)-3-methyl-2-(8-(1-propyl-1H-pyrazol-4-yl)dibenzo[b,d]thiophene-3-sulfonamido)butanoic acid;
(S)-2-(8-(1-isopentyl-1H-pyrazol-4-yl)dibenzo[b,d]thiophene-3-sulfonamido)-3-methylbutanoic acid;
(S)-2-(8-(1-benzyl-1H-pyrazol-4-yl)dibenzo[b,d]thiophene-3-sulfonamido)-3-methylbutanoic acid;
(S)-2-(8-(1H-pyrazol-4-yl)dibenzo[b,d]thiophene-3-sulfonamido)-3-methylbutanoic acid;
(S)-2-(8-(benzo[b]thiophen-2-yl)dibenzo[b,d]thiophene-3-sulfonamido)-3-methylbutanoic acid;
(S)-2-(8-(5-acetylthiophen-2-yl)dibenzo[b,d]thiophene-3-sulfonamido)-3-methylbutanoic acid;
(S)-2-(8-(3-((dimethylamino)methyl)furan-2-yl)dibenzo[b,d]furan-3-sulfonamido)-3-methylbutanoic acid;
(S)-2-(8-(3-((dimethylamino)methyl)thiophen-2-yl)dibenzo[b,d]furan-3-sulfonamido)-3-methylbutanoic acid;
(S)-2-(8-(5-(1-(dimethylamino)ethyl)thiophen-2-yl)dibenzo[b,d]furan-3-sulfonamido)-3-methylbutanoic acid;
(S)-2-(6-(2-chlorothiophen-3-yl)dibenzo[b,d]thiophene-3-sulfonamido)-3-methylbutanoic acid;
(S)-2-(8-(2-chlorothiophen-3-yl)dibenzo[b,d]thiophene-3-sulfonamido)-3-methylbutanoic acid;
(S)-2-(8-(furan-2-yl)dibenzo[b,d]thiophene-3-sulfonamido)-3-methylbutanoic acid;
(S)-2-[8-(6″-Chloro-[2,3′;6′,3″]terpyridin-5-yl)-dibenzothiophene-3-sulfonylamino]-3-methyl-butanoic acid;
(S)-2-(8-(6-methoxypyridin-3-yl)dibenzo[b,d]thiophene-3-sulfonamido)-3-methylbutanoic acid;
(S)-3-methyl-2-(8-(pyridin-4-yl)dibenzo[b,d]thiophene-3-sulfonamido)butanoic acid;
(S)-2-(8-(1H-pyrrol-2-yl)dibenzo[b,d]thiophene-3-sulfonamido)-3-methylbutanoic acid;
(S,E)-2-(8-(2-cyclohexylvinyl)dibenzo[b,d]thiophene-3-sulfonamido)-3-methylbutanoic acid;
(S)-2-(8-(6′-chloro-2,3′-bipyridin-5-yl)dibenzo[b,d]furan-3-sulfonamido)-3-methylbutanoic acid;
(S)-2-(7-(furan-3-yl)dibenzo[b,d]thiophene-3-sulfonamido)-3-methylbutanoic acid;
(S)-2-(8-(6′-chloro-2,3′-bipyridin-5-yl)dibenzo[b,d]thiophene-3-sulfonamido)-3-methylbutanoic acid;
(S)-3-methyl-2-(8-(4-methylthiophen-2-yl)dibenzo[b,d]thiophene-3-sulfonamido)butanoic acid;
(S)-2-(8-(6-chloropyridin-3-yl)dibenzo[b,d]thiophene-3-sulfonamido)-3-methylbutanoic acid;
(S)-2-(8-(6-chloropyridin-3-yl)dibenzo[b,d]furan-3-sulfonamido)-3-methylbutanoic acid;
(S)-2-(7-(3-methoxyprop-1-ynyl)dibenzo[b,d]thiophene-3-sulfonamido)-3-methylbutanoic acid;
(S,E)-3-methyl-2-(8-(prop-1-enyl)dibenzo[b,d]furan-3-sulfonamido)butanoic acid;
(S,Z)-3-methyl-2-(8-(prop-1-enyl)dibenzo[b,d]furan-3-sulfonamido)butanoic acid;
(S)-3-methyl-2-(8-(5-((methylamino)methyl)furan-2-yl)dibenzo[b,d]furan-3-sulfonamido)butanoic acid;
(S)-2-(8-cyclopentenyldibenzo[b,d]furan-3-sulfonamido)-3-methylbutanoic acid;
(S)-3-methyl-2-(8-(1,2,3,6-tetrahydropyridin-4-yl)dibenzo[b,d]furan-3-sulfonamido)butanoic acid;
(S)-2-(8-cyclopentyldibenzo[b,d]furan-3-sulfonamido)-3-methylbutanoic acid;
(S)-3-methyl-2-(8-(5-methylfuran-2-yl)dibenzo[b,d]furan-3-sulfonamido)butanoic acid;
(S)-2-(8-(5-chlorofuran-2-yl)dibenzo[b,d]furan-3-sulfonamido)-3-methylbutanoic acid;
(S)-2-(8-(5-chlorothiophen-2-yl)dibenzo[b,d]furan-3-sulfonamido)-3-methylbutanoic acid;
(S)-2-(8-(3,5-dichlorothiophen-2-yl)dibenzo[b,d]furan-3-sulfonamido)-3-methylbutanoic acid;
(S)-2-(8-(N-isopropylcarbamimidoyl)dibenzo[b,d]furan-3-sulfonamido)-3-methylbutanoic acid;
(S)-2-(8-(4,5-dihydro-1H-imidazol-2-yl)dibenzo[b,d]furan-3-sulfonamido)-3-methylbutanoic acid;
(S)-2-(7-(furan-2-yl)dibenzo[b,d]furan-3-sulfonamido)-3-methylbutanoic acid;
(S)-2-(7-(furan-3-yl)dibenzo[b,d]furan-3-sulfonamido)-3-methylbutanoic acid;
(S)-2-(7-(5-chlorofuran-2-yl)dibenzo[b,d]furan-3-sulfonamido)-3-methylbutanoic acid;
(S)-3-methyl-2-(7-(thiophen-2-yl)dibenzo[b,d]furan-3-sulfonamido)butanoic acid;
(S)-2-(8-(N-hydroxycarbamimidoyl)dibenzo[b,d]furan-3-sulfonamido)-3-methylbutanoic acid;
(S)-2-(8-(4,5-dihydrooxazol-2-yl)dibenzo[b,d]furan-3-sulfonamido)-3-methylbutanoic acid;
(S)-2-(7-(5-chlorothiophen-2-yl)dibenzo[b,d]furan-3-sulfonamido)-3-methylbutanoic acid;
(S)-2-(7-(3,5-dichlorothiophen-2-yl)dibenzo[b,d]furan-3-sulfonamido)-3-methylbutanoic acid;
(S)-3-methyl-2-(7-(3,4,5-trichlorothiophen-2-yl)dibenzo[b,d]furan-3-sulfonamido)butanoic acid;
(S)-3-methyl-2-(8-(N-phenylcarbamimidoyl)dibenzo[b,d]furan-3-sulfonamido)butanoic acid;
(S)-2-(8-(N-benzylcarbamimidoyl)dibenzo[b,d]furan-3-sulfonamido)-3-methylbutanoic acid;
(S)-2-(8-(2,5-dimethylthiophen-3-yl)dibenzo[b,d]furan-3-sulfonamido)-3-methylbutanoic acid;
(R)-2-(7-(3-methoxyprop-1-ynyl)dibenzo[b,d]thiophene-3-sulfonamido)-3-methylbutanoic acid;
(S)-3-methyl-2-(8-(5-methyl-1,2,4-oxadiazol-3-yl)dibenzo[b,d]furan-3-sulfonamido)butanoic acid;
(S)-3-methyl-2-(8-(5-(trifluoromethyl)-1,2,4-oxadiazol-3-yl)dibenzo[b,d]furan-3-sulfonamido)butanoic acid;
(S)-2-(8-(1,2,4-oxadiazol-3-yl)dibenzo[b,d]furan-3-sulfonamido)-3-methylbutanoic acid;
(S)-2-(8-(2-chlorofuran-3-yl)dibenzo[b,d]furan-3-sulfonamido)-3-methylbutanoic acid;
(S)-2-(8-(2,5-dichlorofuran-3-yl)dibenzo[b,d]furan-3-sulfonamido)-3-methylbutanoic acid;
(R)-2-(7-(furan-3-yl)dibenzo[b,d]furan-2-sulfonamido)-3-methylbutanoic acid;
(R)-3-methyl-2-(7-(thiophen-3-yl)dibenzo[b,d]furan-2-sulfonamido)butanoic acid;
(R)-2-(7-(furan-2-yl)dibenzo[b,d]furan-2-sulfonamido)-3-methylbutanoic acid;
(R)-3-methyl-2-(7-(4-methylthiophen-3-yl)dibenzo[b,d]furan-2-sulfonamido)butanoic acid;
(R)-2-(7-(benzo[b]thiophen-2-yl)dibenzo[b,d]furan-2-sulfonamido)-3-methylbutanoic acid;
(R)-2-(7-(6-chloropyridin-3-yl)dibenzo[b,d]furan-2-sulfonamido)-3-methylbutanoic acid;
(R)-2-(7-(6-methoxypyridin-3-yl)dibenzo[b,d]furan-2-sulfonamido)-3-methylbutanoic acid;
(R)-2-(7-(1H-pyrazol-4-yl)dibenzo[b,d]furan-2-sulfonamido)-3-methylbutanoic acid;
(R,E)-2-(7-(2-cyclohexylvinyl)dibenzo[b,d]furan-2-sulfonamido)-3-methylbutanoic acid;
(R)-2-(7-(5-acetylthiophen-2-yl)dibenzo[b,d]furan-2-sulfonamido)-3-methylbutanoic acid;
(S)-2-(8-(N,N-diethylcarbamimidoyl)dibenzo[b,d]furan-3-sulfonamido)-3-methylbutanoic acid;
(S)-2-(8-(4,5-dihydrothiazol-2-yl)dibenzo[b,d]furan-3-sulfonamido)-3-methylbutanoic acid;
(S)-2-(8-(N-methoxycarbamimidoyl)dibenzo[b,d]furan-3-sulfonamido)-3-methylbutanoic acid;
(S)-2-(8-(N,N′-diethylcarbamimidoyl)dibenzo[b,d]furan-3-sulfonamido)-3-methylbutanoic acid;
(S)-2-(8-(N-isopropyl-N-methylcarbamimidoyl)dibenzo[b,d]furan-3-sulfonamido)-3-methylbutanoic acid;
(S)-2-(8-(5-carbamoylthiophen-2-yl)dibenzo[b,d]furan-3-sulfonamido)-3-methylbutanoic acid;
(S)-5-(7-(N-(1-carboxy-2-methylpropyl)sulfamoyl)dibenzo[b,d]furan-2-yl)thiophene-2-carboxylic acid;
(2S)-2-[8-(5-tert-Butyl-[1,2,4]oxadiazol-3-yl)-dibenzofuran-3-sulfonylamino]-3-methyl-butanoic acid;
(2S)-2-[8-(5-Isopropyl-[1,2,4]oxadiazol-3-yl)-dibenzofuran-3-sulfonylamino]-3-methyl-butanoic acid;
(R)-2-(7-(2,4-dimethoxypyrimidin-5-yl)dibenzo[b,d]furan-2-sulfonamido)-3-methylbutanoic acid;
(R)-2-(7-(1H-pyrrol-2-yl)dibenzo[b,d]furan-2-sulfonamido)-3-methylbutanoic acid;
(R)-3-methyl-2-(7-(1-methyl-1H-pyrazol-4-yl)dibenzo[b,d]furan-2-sulfonamido)butanoic acid;
(R)-3-methyl-2-(7-(thiophen-2-yl)dibenzo[b,d]furan-2-sulfonamido)butanoic acid;
(R)-2-(7-(benzofuran-2-yl)dibenzo[b,d]furan-2-sulfonamido)-3-methylbutanoic acid;
(R)-3-methyl-2-(7-(4-(trifluoromethyl)phenyl)dibenzo[b,d]furan-2-sulfonamido)butanoic acid;
(R)-3-methyl-2-(7-(1-methyl-1H-indol-2-yl)dibenzo[b,d]furan-2-sulfonamido)butanoic acid;
(R)-2-(7-(5-fluoro-1H-indol-2-yl)dibenzo[b,d]furan-2-sulfonamido)-3-methylbutanoic acid;
(2S)-2-[8-(5-Ethyl-[1,2,4]oxadiazol-3-yl)-dibenzofuran-3-sulfonylamino]-3-methyl-butanoic acid;
(S)-2-(8-(5-fluorothiophen-2-yl)dibenzo[b,d]furan-3-sulfonamido)-3-methylbutanoic acid;
(2S,2′S)-2,2′-[2,2′-bidibenzo[b,d]furan-7,7′-diylbis(sulfonylimino)]bis(3-methylbutanoic acid;
(S)-3-methyl-2-(8-(4-(trifluoromethyl)thiazol-2-yl)dibenzo[b,d]furan-3-sulfonamido)butanoic acid;
(S)-2-(8-(imino(pyrrolidin-1-yl)methyl)dibenzo[b,d]furan-3-sulfonamido)-3-methylbutanoic acid;
(S)-2-(8-(N-ethylcarbamimidoyl)dibenzo[b,d]furan-3-sulfonamido)-3-methylbutanoic acid;
(S)-2-(7-(furan-2-yl)dibenzo[b,d]thiophene-3-sulfonamido)-3-methylbutanoic acid;
(S)-2-(8-(2H-tetrazol-5-yl)dibenzo[b,d]furan-3-sulfonamido)-3-methylbutanoic acid;
(S)-3-methyl-2-(8-(5-(trifluoromethyl)thiophen-2-yl)dibenzo[b,d]furan-3-sulfonamido)butanoic acid;
(S)-3-methyl-2-(8-(2-methyl-2H-tetrazol-5-yl)dibenzo[b,d]furan-3-sulfonamido)butanoic acid;
(R)-2-(7-(5-tert-butyl-1,2,4-oxadiazol-3-yl)dibenzo[b,d]furan-2-sulfonamido)-3-methylbutanoic acid;
(S)-2-(8-(3,5-dichlorofuran-2-yl)dibenzo[b,d]furan-3-sulfonamido)-3-methylbutanoic acid;
(S)-3-methyl-2-(7-(5-methylfuran-2-yl)dibenzo[b,d]furan-3-sulfonamido)butanoic acid;
(S)-2-(7-(benzo[b]thiophen-2-yl)dibenzo[b,d]furan-3-sulfonamido)-3-methylbutanoic acid;
(S)-3-methyl-2-(7-(thiazol-2-yl)dibenzo[b,d]furan-3-sulfonamido)butanoic acid;
(R)-2-(7-(5-isopropyl-1,2,4-oxadiazol-3-yl)dibenzo[b,d]furan-2-sulfonamido)-3-methylbutanoic acid;
(R)-3-methyl-2-(7-(5-methyl-1,2,4-oxadiazol-3-yl)dibenzo[b,d]furan-2-sulfonamido)butanoic acid;
(R)-2-(7-(5-ethyl-1,2,4-oxadiazol-3-yl)dibenzo[b,d]furan-2-sulfonamido)-3-methylbutanoic acid;
(R)-3-methyl-2-(8-(5-(trifluoromethyl)-1,2,4-oxadiazol-3-yl)dibenzo[b,d]furan-2-sulfonamido)butanoic acid;
(S)-3-methyl-2-(7-(5-methylthiophen-2-yl)dibenzo[b,d]furan-3-sulfonamido)butanoic acid;
(S)-2-(7-(benzofuran-2-yl)dibenzo[b,d]furan-3-sulfonamido)-3-methylbutanoic acid;
(R)-2-(7-(5-bromothiophen-2-yl)dibenzo[b,d]furan-2-sulfonamido)-3-methylbutanoic acid;
(R)-2-(7-(3,5-dimethylisoxazol-4-yl)dibenzo[b,d]furan-2-sulfonamido)-3-methylbutanoic acid;
(S)-3-methyl-2-(8-(5-methyl-1,3,4-thiadiazol-2-yl)dibenzo[b,d]furan-3-sulfonamido)butanoic acid;
(R)-2-(7-(5-cyclopropyl-1,2,4-oxadiazol-3-yl)dibenzo[b,d]furan-2-sulfonamido)-3-methylbutanoic acid;
(R)-2-(7-(5-cyclobutyl-1,2,4-oxadiazol-3-yl)dibenzo[b,d]furan-2-sulfonamido)-3-methylbutanoic acid;
(R)-2-(7-(5-isobutyl-1,2,4-oxadiazol-3-yl)dibenzo[b,d]furan-2-sulfonamido)-3-methylbutanoic acid;
(R)-3-methyl-2-(7-(5-phenyl-1,2,4-oxadiazol-3-yl)dibenzo[b,d]furan-2-sulfonamido)butanoic acid;
(S)-2-(8-(benzo[d]thiazol-2-yl)dibenzo[b,d]furan-3-sulfonamido)-3-methylbutanoic acid;
(S)-3-methyl-2-(8-(pyrimidin-5-yl)dibenzo[b,d]furan-3-sulfonamido)butanoic acid;
(S)-2-(8-(2-methoxypyrimidin-5-yl)dibenzo[b,d]furan-3-sulfonamido)-3-methylbutanoic acid;
(S)-2-(7-(5-isopropyl-1,2,4-oxadiazol-3-yl)dibenzo[b,d]furan-3-sulfonamido)-3-methylbutanoic acid;
(S)-2-(7-(5-tert-butyl-1,2,4-oxadiazol-3-yl)dibenzo[b,d]furan-3-sulfonamido)-3-methylbutanoic acid; and
(S)-3-methyl-2-(7-(5-methyl-1,2,4-oxadiazol-3-yl)dibenzo[b,d]furan-3-sulfonamido)butanoic acid;
(2S)-3-methyl-2-(8-(1-(2-methylbutyl)-1H-pyrazol-4-yl)dibenzo[b,d]furan-3-sulfonamido)butanoic acid;
(S)-3-methyl-2-(8-(1-(2-morpholinoethyl)-1H-pyrazol-4-yl)dibenzo[b,d]furan-3-sulfonamido)butanoic acid;
(S)-2-(8-(1-isobutyl-1H-pyrazol-4-yl)dibenzo[b,d]furan-3-sulfonamido)-3-methylbutanoic acid;
(S)-3-methyl-2-(8-(1,3,5-trimethyl-1H-pyrazol-4-yl)dibenzo[b,d]furan-3-sulfonamido)butanoic acid;
(S)-3-methyl-2-(8-(5-methyl-3-phenylisoxazol-4-yl)dibenzo[b,d]furan-3-sulfonamido)butanoic acid;
(S)-3-methyl-2-(8-(5-methyl-1-phenyl-1H-pyrazol-4-yl)dibenzo[b,d]furan-3-sulfonamido)butanoic acid;
(S)-3-methyl-2-(8-(4-methyl-2-phenylthiazol-5-yl)dibenzo[b,d]furan-3-sulfonamido)butanoic acid;
(S)-3-methyl-2-(8-(4-methyl-2-(4-(trifluoromethyl)phenyl)thiazol-5-yl)dibenzo[b,d]furan-3-sulfonamido)butanoic acid;
(S)-2-(7-(4-bromo-5-ethylthiophen-2-yl)dibenzo[b,d]furan-3-sulfonamido)-3-methylbutanoic acid;
(S)-2-(7-(2′,5-diethyl-2,3′-bithiophen-5′-yl)dibenzo[b,d]furan-3-sulfonamido)-3-methylbutanoic acid;
(R)-3-methyl-2-(7-(pyrimidin-5-yl)dibenzo[b,d]furan-2-sulfonamido)butanoic acid;
(R)-2-(7-(2-methoxypyrimidin-5-yl)dibenzo[b,d]furan-2-sulfonamido)-3-methylbutanoic acid;
(R)-2-(7-(2,4-dimethylthiazol-5-yl)dibenzo[b,d]furan-2-sulfonamido)-3-methylbutanoic acid;
(2R)-3-methyl-2-(7-(1-(2-methylbutyl)-1H-pyrazol-4-yl)dibenzo[b,d]furan-2-sulfonamido)butanoic acid;
(R)-3-methyl-2-(7-(1-propyl-1H-pyrazol-4-yl)dibenzo[b,d]furan-2-sulfonamido)butanoic acid;
(R)-3-methyl-2-(7-(1-(2-morpholinoethyl)-1H-pyrazol-4-yl)dibenzo[b,d]furan-2-sulfonamido)butanoic acid;
(R)-2-(7-(1-isobutyl-1H-pyrazol-4-yl)dibenzo[b,d]furan-2-sulfonamido)-3-methylbutanoic acid;
(R)-2-(7-(1-isobutyl-1H-pyrazol-4-yl)dibenzo[b,d]furan-2-sulfonamido)-3-methylbutanoic acid;
(R)-2-(7-(1-benzyl-1H-pyrazol-4-yl)dibenzo[b,d]furan-2-sulfonamido)-3-methylbutanoic acid;
(R)-3-methyl-2-(7-(4-methyl-2-phenylthiazol-5-yl)dibenzo[b,d]furan-2-sulfonamido)butanoic acid;
(R)-3-methyl-2-(7-(4-methyl-2-(4-(trifluoromethyl)phenyl)thiazol-5-yl)dibenzo[b,d]furan-2-sulfonamido)butanoic acid;
(R)-2-(8-(5-chlorofuran-2-yl)dibenzo[b,d]furan-3-sulfonamido)-3-methylbutanoic acid;
(S)-2-(8-(2-chlorothiazol-5-yl)dibenzo[b,d]furan-3-sulfonamido)-3-methylbutanoic acid;
(S)-2-(8-(2-chlorothiazol-4-yl)dibenzo[b,d]furan-3-sulfonamido)-3-methylbutanoic acid;
(S)-2-(7-(2-chlorothiazol-5-yl)dibenzo[b,d]furan-3-sulfonamido)-3-methylbutanoic acid;
(S)-2-(7-(5-chlorofuran-2-yl)dibenzo[b,d]thiophene-3-sulfonamido)-3-methylbutanoic acid;
(R)-3-methyl-2-(7-(5-methyl-1,2,4-oxadiazol-3-yl)dibenzo[b,d]furan-2-sulfonamido)butanoic acid;
(R)-2-(7-(5-isopropyl-1,2,4-oxadiazol-3-yl)dibenzo[b,d]furan-2-sulfonamido)-3-methylbutanoic acid;
(R)-3-methyl-2-(8-(5-(trifluoromethyl)-1,2,4-oxadiazol-3-yl)dibenzo[b,d]furan-3-sulfonamido)butanoic acid;
(S)-2-(7-(N-hydroxycarbamimidoyl)dibenzo[b,d]furan-3-sulfonamido)-3-methylbutanoic acid;
(S)-2-(7-(5-cyclopropyl-1,2,4-oxadiazol-3-yl)dibenzo[b,d]furan-3-sulfonamido)-3-methylbutanoic acid;
(S)-2-(7-(5-(4-fluorophenyl)-1,2,4-oxadiazol-3-yl)dibenzo[b,d]furan-3-sulfonamido)-3-methylbutanoic acid;
(R)-3-methyl-2-(7-(5-neopentyl-1,2,4-oxadiazol-3-yl)dibenzo[b,d]furan-2-sulfonamido)butanoic acid;
(R)-2-(7-(5-cyclopentyl-1,2,4-oxadiazol-3-yl)dibenzo[b,d]furan-2-sulfonamido)-3-methylbutanoic acid;
(R)-2-(7-(5-(cyclopentylmethyl)-1,2,4-oxadiazol-3-yl)dibenzo[b,d]furan-2-sulfonamido)-3-methylbutanoic acid;
(R)-2-(7-(5-cyclohexyl-1,2,4-oxadiazol-3-yl)dibenzo[b,d]furan-2-sulfonamido)-3-methylbutanoic acid;
(S)-2-(7-(furan-2-yl)dibenzo[b,d]thiophene-3-sulfonamido)-3-methylbutanoic acid;
(S)-2-(8-(benzo[d]oxazol-2-yl)dibenzo[b,d]furan-3-sulfonamido)-3-methylbutanoic acid;
(S)-2-(2,2′-bidibenzo[b,d]furan-7-sulfonamido)-3-methylbutanoic acid;
(S)-2-(8-(5-ethylthiophen-2-yl)dibenzo[b,d]furan-3-sulfonamido)-3-methylbutanoic acid;
(S)-3-methyl-2-(8-(5-propylthiophen-2-yl)dibenzo[b,d]furan-3-sulfonamido)butanoic acid;
(S)-2-(8-(5-tert-butylfuran-2-yl)dibenzo[b,d]furan-3-sulfonamido)-3-methylbutanoic acid;
(S)-3-methyl-2-(8-(5-(5-methyl-1,2,4-oxadiazol-3-yl)thiophen-2-yl)dibenzo[b,d]furan-3-sulfonamido)butanoic acid;
(S)-2-(8-(5-chloro-4-(trifluoromethyl)thiazol-2-yl)dibenzo[b,d]furan-3-sulfonamido)-3-methylbutanoic acid;
(S)-2-(8-(2,4-dimethylthiazol-5-yl)dibenzo[b,d]furan-3-sulfonamido)-3-methylbutanoic acid;
(S)-3-methyl-2-(8-(2-methylthiazol-5-yl)dibenzo[b,d]furan-3-sulfonamido)butanoic acid;
(S)-2-(8-(6-chlorobenzo[d]thiazol-2-yl)dibenzo[b,d]furan-3-sulfonamido)-3-methylbutanoic acid;
(S)-2-(8-(2-isobutyl-4-methylthiazol-5-yl)dibenzo[b,d]furan-3-sulfonamido)-3-methylbutanoic acid;
(S)-2-(8-(benzo[d]thiazol-2-yl)dibenzo[b,d]furan-3-sulfonamido)-3-methylbutanoic acid;
(S)-3-methyl-2-(8-(5-phenyl-3-(trifluoromethyl)-1H-pyrazol-4-yl)dibenzo[b,d]furan-3-sulfonamido)butanoic acid;
(S)-2-(8-(5-(1H-tetrazol-5-yl)thiophen-2-yl)dibenzo[b,d]furan-3-sulfonamido)-3-methylbutanoic acid;
(S)-2-(8-(6-methoxybenzo[d]thiazol-2-yl)dibenzo[b,d]furan-3-sulfonamido)-3-methylbutanoic acid;
(S)-2-(8-(6-fluorobenzo[d]thiazol-2-yl)dibenzo[b,d]furan-3-sulfonamido)-3-methylbutanoic acid;
(S)-3-methyl-2-(8-(6-methylbenzo[d]thiazol-2-yl)dibenzo[b,d]furan-3-sulfonamido)butanoic acid;
(S)-2-(8-(5-(isoxazol-5-yl)thiophen-2-yl)dibenzo[b,d]furan-3-sulfonamido)-3-methylbutanoic acid;
(S)-3-methyl-2-(8-(5-((4-methylpiperazin-1-yl)methyl)thiazol-2-yl)dibenzo[b,d]furan-3-sulfonamido)butanoic acid;
(S)-2-(8-(5-(((cyclopropylmethyl)(propyl)amino)methyl)thiazol-2-yl)dibenzo[b,d]furan-3-sulfonamido)-3-methylbutanoic acid;
(S)-2-(8-(5-((1H-pyrazol-1-yl)methyl)thiazol-2-yl)dibenzo[b,d]furan-3-sulfonamido)-3-methylbutanoic acid;
(S)-2-(8-(5-(hydroxymethyl)thiazol-2-yl)dibenzo[b,d]furan-3-sulfonamido)-3-methylbutanoic acid;
(S)-2-(8-(5-(isoxazol-3-yl)thiophen-2-yl)dibenzo[b,d]furan-3-sulfonamido)-3-methylbutanoic acid;
(S)-2-(8-(4-bromothiazol-2-yl)dibenzo[b,d]furan-3-sulfonamido)-3-methylbutanoic acid;
(S)-2-(8-(4-fluorobenzo[d]thiazol-2-yl)dibenzo[b,d]furan-3-sulfonamido)-3-methylbutanoic acid;
(S)-2-(8-(5-fluorobenzo[d]thiazol-2-yl)dibenzo[b,d]furan-3-sulfonamido)-3-methylbutanoic acid;
(S)-2-(8-(5,6-difluorobenzo[d]thiazol-2-yl)dibenzo[b,d]furan-3-sulfonamido)-3-methylbutanoic acid;
(S)-3-methyl-2-(8-(6-(trifluoromethoxy)benzo[d]thiazol-2-yl)dibenzo[b,d]furan-3-sulfonamido)butanoic acid;
(S)-3-methyl-2-(8-(4,5,6-trifluorobenzo[d]thiazol-2-yl)dibenzo[b,d]furan-3-sulfonamido)butanoic acid;
(S)-2-(8-(4-methoxybenzo[d]thiazol-2-yl)dibenzo[b,d]furan-3-sulfonamido)-3-methylbutanoic acid;
(S)-2-(8-(5-chlorothiazol-2-yl)dibenzo[b,d]furan-3-sulfonamido)-3-methylbutanoic acid;
(S)-2-(8-(5-methoxybenzo[d]thiazol-2-yl)dibenzo[b,d]furan-3-sulfonamido)-3-methylbutanoic acid;
(S)-2-(7-(benzo[d]thiazol-2-yl)dibenzo[b,d]furan-3-sulfonamido)-3-methylbutanoic acid;
(S)-2-(7-(benzo[d]oxazol-2-yl)dibenzo[b,d]furan-3-sulfonamido)-3-methylbutanoic acid;
(S)-3-methyl-2-(7-(5-(5-methyl-1,2,4-oxadiazol-3-yl)thiazol-2-yl)dibenzo[b,d]furan-3-sulfonamido)butanoic acid;
(S)-2-(7-(5-ethylthiophen-2-yl)dibenzo[b,d]furan-3-sulfonamido)-3-methylbutanoic acid;
(S)-2-(7-(2,4-dimethylthiazol-5-yl)dibenzo[b,d]furan-3-sulfonamido)-3-methylbutanoic acid;
(S)-2-(7-(5-tert-butylfuran-2-yl)dibenzo[b,d]furan-3-sulfonamido)-3-methylbutanoic acid;
(S)-3-methyl-2-(7-(5-propylthiophen-2-yl)dibenzo[b,d]furan-3-sulfonamido)butanoic acid;
(S)-2-(7-(5-chloro-4-(trifluoromethyl)thiazol-2-yl)dibenzo[b,d]furan-3-sulfonamido)-3-methylbutanoic acid;
(S)-3-methyl-2-(7-(5-methylthiazol-2-yl)dibenzo[b,d]furan-3-sulfonamido)butanoic acid;
(S)-2-(7-(2-isobutyl-4-methylthiazol-5-yl)dibenzo[b,d]furan-3-sulfonamido)-3-methylbutanoic acid;
(S)-3-methyl-2-(7-(6-(trifluoromethyl)benzo[d]thiazol-2-yl)dibenzo[b,d]furan-3-sulfonamido)butanoic acid;
(S)-2-(7-(6-fluorobenzo[d]thiazol-2-yl)dibenzo[b,d]furan-3-sulfonamido)-3-methylbutanoic acid;
(R)-3-methyl-2-(7-(thiazol-2-yl)dibenzo[b,d]furan-2-sulfonamido)butanoic acid;
(R)-2-(7-(5-ethylthiophen-2-yl)dibenzo[b,d]furan-2-sulfonamido)-3-methylbutanoic acid;
(R)-2-(7-(5-tert-butylfuran-2-yl)dibenzo[b,d]furan-2-sulfonamido)-3-methylbutanoic acid;
(S)-2-(7-(5-tert-butylfuran-2-yl)dibenzo[b,d]furan-2-sulfonamido)-3-methylbutanoic acid;
(S)-2-(7-(5-ethylthiophen-2-yl)dibenzo[b,d]furan-2-sulfonamido)-3-methylbutanoic acid;
(R)-3-methyl-2-(7-(5-propylthiophen-2-yl)dibenzo[b,d]furan-2-sulfonamido)butanoic acid;
(R)-2-(7-(2-isobutylthiazol-5-yl)dibenzo[b,d]furan-2-sulfonamido)-3-methylbutanoic acid;
(R)-2-(7-(2-isobutyl-4-methylthiazol-5-yl)dibenzo[b,d]furan-2-sulfonamido)-3-methylbutanoic acid;
(S)-3-methyl-2-(7-(5-propylthiophen-2-yl)dibenzo[b,d]furan-2-sulfonamido)butanoic acid;
(S)-2-(7-(2-isobutyl-4-methylthiazol-5-yl)dibenzo[b,d]furan-2-sulfonamido)-3-methylbutanoic acid;
(S)-2-(8-(2-isobutylthiazol-5-yl)dibenzo[b,d]furan-3-sulfonamido)-3-methylbutanoic acid;
(S)-2-(7-(2-isobutylthiazol-5-yl)dibenzo[b,d]furan-3-sulfonamido)-3-methylbutanoic acid;
(S)-2-(7-(1H-tetrazol-5-yl)dibenzo[b,d]furan-3-sulfonamido)-3-methylbutanoic acid;
2-(8-(thiazol-2-yl)dibenzo[b,d]furan-3-sulfonamido)acetic acid;
(S)-2-(8-(5-tert-butyl-1,2,4-oxadiazol-3-yl)dibenzo[b,d]furan-3-sulfonamido)-4-methylpentanoic acid;
(R)-2-(8-(5-tert-butyl-1,2,4-oxadiazol-3-yl)dibenzo[b,d]furan-3-sulfonamido)-4-methylpentanoic acid;
(S)-2-(8-(5-tert-butyl-1,2,4-oxadiazol-3-yl)dibenzo[b,d]furan-3-sulfonamido)-2-phenylacetic acid;
(R)-2-(8-(5-tert-butyl-1,2,4-oxadiazol-3-yl)dibenzo[b,d]furan-3-sulfonamido)-2-phenylacetic acid;
(R)-2-(8-(5-tert-butyl-1,2,4-oxadiazol-3-yl)dibenzo[b,d]furan-3-sulfonamido)-3-(1H-indol-3-yl)propanoic acid;
(S)-2-(8-(5-tert-butyl-1,2,4-oxadiazol-3-yl)dibenzo[b,d]furan-3-sulfonamido)-3,3-dimethylbutanoic acid;
(R)-2-(8-(5-tert-butyl-1,2,4-oxadiazol-3-yl)dibenzo[b,d]furan-3-sulfonamido)-3-methylbutanoic acid;
(S)-2-(8-(5-cyclopropyl-1,2,4-oxadiazol-3-yl)dibenzo[b,d]furan-3-sulfonamido)-3-methylbutanoic acid;
(S)-3-methyl-2-(8-(5-(tetrahydro-2H-pyran-4-yl)-1,2,4-oxadiazol-3-yl)dibenzo[b,d]furan-3-sulfonamido)butanoic acid;
(S)-3-methyl-2-(8-(5-neopentyl-1,2,4-oxadiazol-3-yl)dibenzo[b,d]furan-3-sulfonamido)butanoic acid;
(S)-2-(8-(5-cyclobutyl-1,2,4-oxadiazol-3-yl)dibenzo[b,d]furan-3-sulfonamido)-3-methylbutanoic acid;
(S)-2-(8-(5-cyclopentyl-1,2,4-oxadiazol-3-yl)dibenzo[b,d]furan-3-sulfonamido)-3-methylbutanoic acid;
(S)-3-methyl-2-(8-(5-(thiophen-2-yl)-1,2,4-oxadiazol-3-yl)dibenzo[b,d]furan-3-sulfonamido)butanoic acid;
(S)-3-methyl-2-(8-(5-phenyl-1,2,4-oxadiazol-3-yl)dibenzo[b,d]furan-3-sulfonamido)butanoic acid;
(S)-2-(8-(5-benzyl-1,2,4-oxadiazol-3-yl)dibenzo[b,d]furan-3-sulfonamido)-3-methylbutanoic acid;
(S)-2-(8-(5-(methoxymethyl)-1,2,4-oxadiazol-3-yl)dibenzo[b,d]furan-3-sulfonamido)-3-methylbutanoic acid;
(2S)-3-methyl-2-(8-(5-(tetrahydrofuran-3-yl)-1,2,4-oxadiazol-3-yl)dibenzo[b,d]furan-3-sulfonamido)butanoic acid;
(S)-2-(8-(5-(2,4-difluorophenyl)-1,2,4-oxadiazol-3-yl)dibenzo[b,d]furan-3-sulfonamido)-3-methylbutanoic acid;
(S)-2-(8-(5-(2,4-dichlorophenyl)-1,2,4-oxadiazol-3-yl)dibenzo[b,d]furan-3-sulfonamido)-3-methylbutanoic acid;
(S)-3-methyl-2-(8-(5-(4-(trifluoromethyl)phenyl)-1,2,4-oxadiazol-3-yl)dibenzo[b,d]furan-3-sulfonamido)butanoic acid;
(S)-2-(8-(5-(4-fluorophenyl)-1,2,4-oxadiazol-3-yl)dibenzo[b,d]furan-3-sulfonamido)-3-methylbutanoic acid;
(S)-7-(N-(1-carboxy-2-methylpropyl)sulfamoyl)dibenzo[b,d]furan-2-carboxylic acid;
2-(7-(5-tert-butyl-1,2,4-oxadiazol-3-yl)dibenzo[b,d]furan-2-sulfonamido)acetic acid;
(R)-2-(7-(5-tert-butyl-1,2,4-oxadiazol-3-yl)dibenzo[b,d]furan-2-sulfonamido)-3-phenylpropanoic acid;
(S)-2-(7-(5-tert-butyl-1,2,4-oxadiazol-3-yl)dibenzo[b,d]furan-2-sulfonamido)-3-methylbutanoic acid;
2-(7-(5-tert-butyl-1,2,4-oxadiazol-3-yl)dibenzo[b,d]furan-2-sulfonamido)-2-methylpropanoic acid;
(R)-2-(7-(5-tert-butyl-1,2,4-oxadiazol-3-yl)dibenzo[b,d]furan-2-sulfonamido)-4-methylpentanoic acid;
(S)-2-(7-(5-tert-butyl-1,2,4-oxadiazol-3-yl)dibenzo[b,d]furan-2-sulfonamido)-4-methylpentanoic acid;
(S)-2-(7-(5-tert-butyl-1,2,4-oxadiazol-3-yl)dibenzo[b,d]furan-2-sulfonamido)-2-(1H-indol-3-yl)acetic acid;
(S)-2-(7-(5-tert-butyl-1,2,4-oxadiazol-3-yl)dibenzo[b,d]furan-2-sulfonamido)-2-phenylacetic acid;
(S)-2-(7-(5-tert-butyl-1,2,4-oxadiazol-3-yl)dibenzo[b,d]furan-2-sulfonamido)-3,3-dimethylbutanoic acid;
(S)-3-methyl-2-(8-(4-(4-(trifluoromethyl)phenyl)thiazol-2-yl)dibenzo[b,d]furan-3-sulfonamido)butanoic acid;
(S)-2-(8-(4-(4-fluorophenyl)thiazol-2-yl)dibenzo[b,d]furan-3-sulfonamido)-3-methylbutanoic acid;
(R)-3-methyl-2-(7-(thiazol-2-yl)dibenzo[b,d]thiophene-2-sulfonamido)butanoic acid;
(R)-2-(7-(benzo[d]thiazol-2-yl)dibenzo[b,d]thiophene-2-sulfonamido)-3-methylbutanoic acid;
(R)-3-methyl-2-(7-(thiazol-2-yl)dibenzo[b,d]thiophene-2-sulfonamido)butanoic acid;
(R)-2-(7-(5-chlorofuran-2-yl)dibenzo[b,d]thiophene-2-sulfonamido)-3-methylbutanoic acid;
(R)-3-methyl-2-(7-(5-phenylthiophen-2-yl)dibenzo[b,d]furan-2-sulfonamido)butanoic acid;
(R)-2-(7-(5-chlorofuran-2-yl)dibenzo[b,d]furan-2-sulfonamido)-3-methylbutanoic acid;
(R)-3-methyl-2-(7-(thiazol-2-yl)dibenzo[b,d]furan-2-sulfonamido)butanoic acid;
(R)-3-methyl-2-(7-(5-methyl-1,3,4-thiadiazol-2-yl)dibenzo[b,d]furan-2-sulfonamido)butanoic acid;
(R)-2-(7-(benzo[d]thiazol-2-yl)dibenzo[b,d]furan-2-sulfonamido)-3-methylbutanoic acid;
(R)-2-(7-(benzo[d]oxazol-2-yl)dibenzo[b,d]furan-2-sulfonamido)-3-methylbutanoic acid;
(R)-2-(7-(5-chloro-4-(trifluoromethyl)thiazol-2-yl)dibenzo[b,d]furan-2-sulfonamido)-3-methylbutanoic acid;
(R)-2-(7-(6-methoxybenzo[d]thiazol-2-yl)dibenzo[b,d]furan-2-sulfonamido)-3-methylbutanoic acid;
(R)-2-(7-(6-fluorobenzo[d]thiazol-2-yl)dibenzo[b,d]furan-2-sulfonamido)-3-methylbutanoic acid;
(R)-3-methyl-2-(7-(6-methylbenzo[d]thiazol-2-yl)dibenzo[b,d]furan-2-sulfonamido)butanoic acid;
(R)-2-(7-(4-fluorobenzo[d]thiazol-2-yl)dibenzo[b,d]furan-2-sulfonamido)-3-methylbutanoic acid;
(R)-3-methyl-2-(7-(4,5,6-trifluorobenzo[d]thiazol-2-yl)dibenzo[b,d]furan-2-sulfonamido)butanoic acid;
(R)-3-methyl-2-(7-(6-(trifluoromethoxy)benzo[d]thiazol-2-yl)dibenzo[b,d]furan-2-sulfonamido)butanoic acid;
(R)-3-methyl-2-(7-(6-(trifluoromethyl)benzo[d]thiazol-2-yl)dibenzo[b,d]furan-2-sulfonamido)butanoic acid;
(S)-2-(8-ethynyldibenzo[b,d]furan-3-sulfonamido)-3-methylbutanoic acid;
(S)-2-(7-(5-chlorothiophen-2-yl)dibenzo[b,d]thiophene-3-sulfonamido)-3-methylbutanoic acid;
(S)-2-(8-(4,5-dimethylthiazol-2-yl)dibenzo[b,d]furan-3-sulfonamido)-3-methylbutanoic acid;
(S)-2-[7-(5,6-Dihydro-4H-cyclopentathiazol-2-yl)-dibenzofuran-3-sulfonylamino]-3-methyl-butyric acid; and
(S)-3-methyl-2-(8-(4,5,6,7-tetrahydrobenzo[d]thiazol-2-yl)dibenzo[b,d]furan-3-sulfonamido)butanoic acid;
and combinations thereof.

39. A compound of claim 30, or a pharmaceutically acceptable salt or ester thereof, wherein at least 75% of the compound has an S-configuration at the chiral center.

40. A compound of claim 30, or a pharmaceutically acceptable salt or ester thereof, wherein at least 75% of the compound has an R-configuration at the chiral center.

41. A pharmaceutical composition comprising the compound of any one of claims 1-38 or a pharmaceutically acceptable salt or ester thereof, and a pharmaceutically acceptable carrier or excipient.

42. A method for the treatment of a pathological condition or disorder mediated wholly or in part by one or more matrix metalloproteinases in a mammal in need of such treatment; wherein the method comprises administering to the mammal an effective amount of the compound of any one of claims 1-40, or a pharmaceutically acceptable salt or ester thereof, wherein the pathological condition or disorder is selected from rheumatoid arthritis, osteoarthritis, atherosclerosis, multiple sclerosis, spinal cord injury, fibrosis, lung cancer, skin cancer, asthma, chronic obstructive pulmonary disorder, obesity, and diabetes.

43. A method of inhibiting one or more matrix metalloproteinases in a mammal comprising administering to the mammal an effective amount of the compound of any one of claims 1-40 or a pharmaceutically acceptable salt or ester thereof.

44. The method of claim 42, 43 or 44, wherein the mammal is a human.

45. The method of claim 42, 43 or 44, wherein said one or more matrix metalloproteinases comprise MMP-12.

46. Use of a compound of any one of claims 1-41, or a pharmaceutically acceptable salt or ester thereof in the preparation of a medicament for the treatment of a pathological condition or disorder mediated wholly or in part by one or more matrix metalloproteinases in a mammal in need of such treatment; wherein the pathological condition or disorder is selected from rheumatoid arthritis, osteoarthritis, atherosclerosis, multiple sclerosis, spinal cord injury, fibrosis, lung cancer, skin cancer, asthma, chronic obstructive pulmonary disorder, obesity, and diabetes.
